(19) United States
(12) Patent Application Publication
(10) Pub. No.: US 2006/0234944 A1
(43) Pub. Date: Oct. 19, 2006

(54) Beta-secretase inhibitors and methods of use

(75) Inventors: Arun K. Ghosh, River Forest, IL (US); Jordan Tang, Edmond, OK (US); Geoffrey Bilcer, Chicago, IL (US); Wanpin Chang, Edmond, OK (US); Lin Hong, Oklahoma City, OK (US); Gerald Koelsch, Oklahoma City, OK (US); Jeff Loy, Norman, OK (US); Robert T. Turner III, Oklahoma City, OK (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oklahoma Medical Reseach Foundation, Oklahoma City, OK (US); The Board of Trustees of the University of Illinois, Urbana, IL (US)

(21) Appl. No.: 10/493,439

(22) PCT Filed: Oct. 23, 2002
(86) PCT No.: PCT/US02/34324

§ 371 Date: Aug. 16, 2004

Related U.S. Application Data
(60) Provisional application No. 60/335,952, filed on Oct. 23, 2001.

(51) Int. Cl. A61K 38/08; C07K 7/06

(52) U.S. Cl. 514/17; 530/330; 530/329

(57) ABSTRACT

Compounds inhibit memapsin 2 β-secretase activity and selectively inhibit memapsin 2 β-secretase activity relative to memapsin 1 β-secretase activity. The compounds are employed in methods to inhibit memapsin 2 β-secretase activity, in the treatment of Alzheimer's disease, in the inhibition of hydrolysis of a β-secretase site of a β-amyloid precursor protein and to decrease β-amyloid protein in in vitro samples and in mammals. Proteins of memapsin 2 associated with compounds of the invention are crystallized.

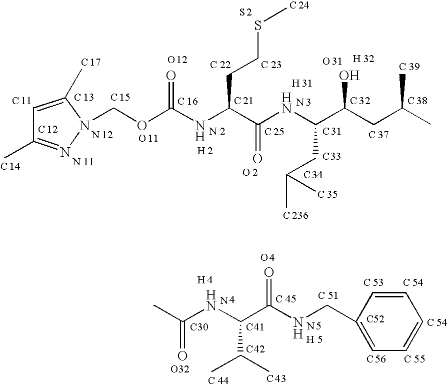

### RELATED APPLICATIONS

This application claims the benefit of U.S. Provisional Application Nos. 60/335,952, filed Oct. 23, 2001; 60/333,545, filed Nov. 27, 2001; 60/348,464, filed Jan. 14, 2002; 60/348,615, filed Jan. 14, 2002; 60/390,804, filed Jun. 20, 2002; 60/397,557, filed Jul. 19, 2002; and 60/397,619, filed Jul. 19, 2002, the teachings of all of which are incorporated herein by reference in their entirety.

### GOVERNMENT SUPPORT

The invention was supported, in whole or in part, by a National Institutes of Health grants AG-18933 and AI-38189. The Government has certain rights in the invention.

### BACKGROUND OF THE INVENTION

Alzheimer's disease is a progressive mental deterioration in a human resulting, inter alia, in loss of memory, confusion and disorientation Alzheimer's disease accounts for the majority of sentile dementias and is a leading cause of death in adults (Anderson, R. N., Natl. Vital Stat. Rep. 49:1-87 (2001), the teachings of which are incorporated herein in their entirety). Histologically, the brain of persons afflicted with Alzheimer's disease is characterized by a distortion of the intracellular neurofibrils and the presence of senile plaques composed of granular or filamentous argentophilic masses with an amyloid protein core, largely due to the accumulation of β-amyloid peptide (Aβ) in the brain. Aβ accumulation plays a role in the pathogenesis and progression of the disease (Selkoe, D. J., Nature 399: 23-31 (1999)) and is a proteolytic fragment of amyloid precursor protein (APP). APP is cleaved initially by β-secretase followed by γ-secretase to generate Aβ (Lin, X., et al., Proc. Natl. Acad. Sci. USA 97:1456-1460 (2000); De Stropper, B., et al., Nature 391:387-390 (1998)).

There is a need to develop effective compounds and methods for the treatment of Alzheimer's disease.

### SUMMARY OF THE INVENTION

The present invention is directed to compounds and pharmaceutical compositions containing compounds represented by Structural Formula I: 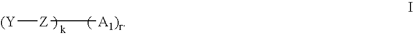
In Formula I, Y is a carrier molecule; Z is a bond, —OP(O)⁻₂O—, —C(O)OR₃₃—, C(O)NHR₃₃ or an amino acid sequence cleavable by a hydrolase; R₃₃ is a bond or an alkylene; k is 0 or an integer from 1 to about 100; r is an integer from 1 to about 100; and A₁, for each occurrence, is a compound represented by the following Formula II, or optical isomers, diastereomers, or pharmaceutically acceptable salts thereof: 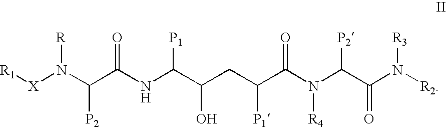
In Formula II, X is C═O or S(O)ₙ. n is 1 or 2. P₁ is an aliphatic group, a hydroxyalkyl, an aryl, an aralkyl, a heterocycloalkyl, or an alkylsulfanylalkyl. P₂, P₁′, and P₂′ are each, independently, a substituted or unsubstituted aliphatic group, a substituted or unsubstituted heteroalkyl, a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl. R is —H. R₁ is a substituted or unsubstituted aliphatic group, a substituted or unsubstituted alkoxy, a substituted or unsubstituted aryl, a substituted or unsubstituted aralkyl, a substituted or unsubstituted heterocycle, a substituted or unsubstituted heterocycloalkyl, a substituted or unsubstituted heterocyclooxy, a substituted or unsubstituted heterocycloalkoxy, a substituted or unsubstituted heteroaryl, a substituted or unsubstituted heteroaralkyl, a substituted or unsubstituted heteroaralkoxy, or —NR₅R₆. Alternatively, R₁, together with X, is a peptide or Y-Z-. R₄ is H; or R₄ and P₁′, together with the atoms connecting R₄ and P₁′, form a five or six membered heterocycle. R₂ and R₃ are each, independently, selected from the group consisting of H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl, and a substituted or unsubstituted heteroaralkyl; or one of R₂ and R₃, together with the nitrogen to which they are attached, is a peptide or Y-Z-. Alternatively, R₂ and R₃ together with the nitrogen to which they are attached form a substituted or unsubstituted heterocycle or a substituted or unsubstituted heteroaryl. R₅ and R₆ are each, independently, H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl or a substituted or unsubstituted heteroaralkyl. Alternatively, R and one of R₅ or R₆, together with X and the nitrogen atoms to which they are attached, form a 5-, 6-, or 7-membered substituted or unsubstituted heterocycle or substituted or unsubstituted heteroaryl ring. However, A₁ does not include the following compounds:

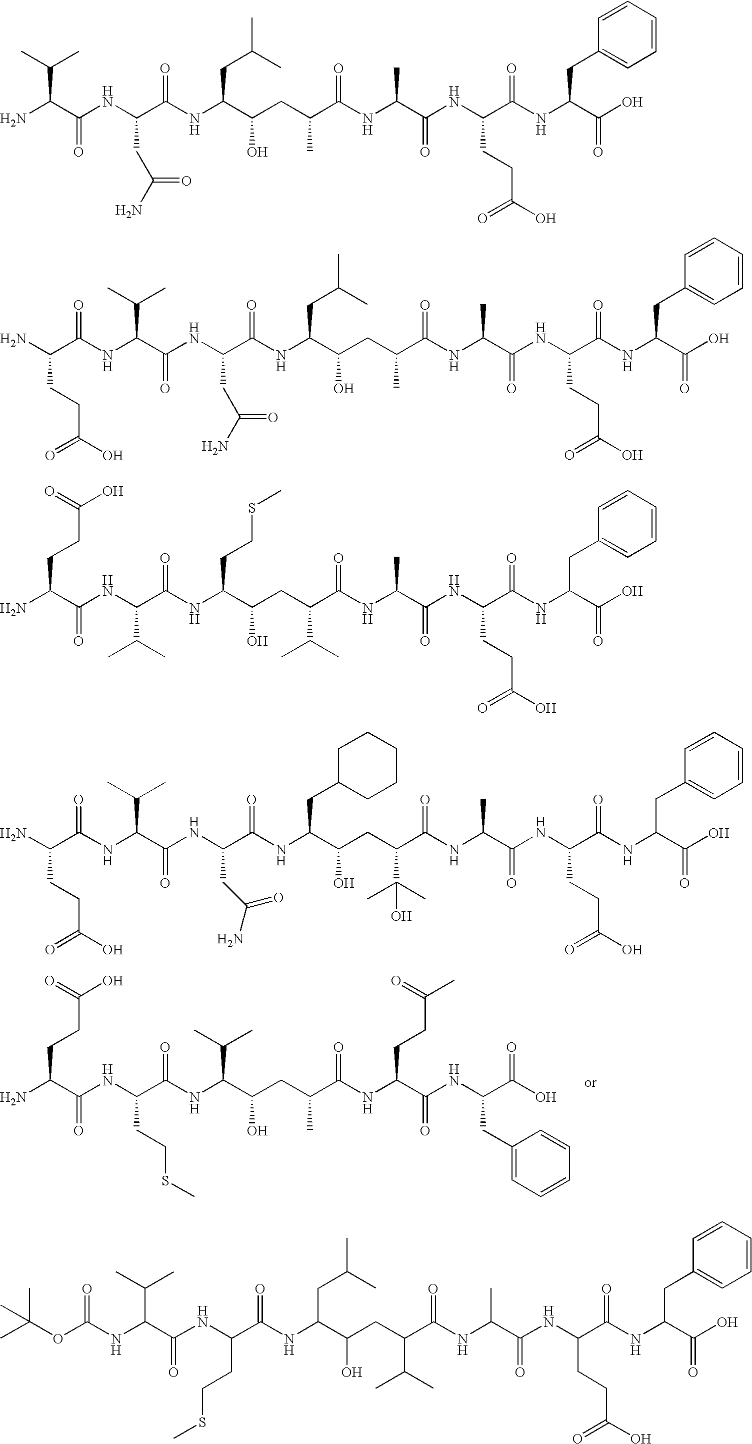

In one embodiment, the invention is directed to compounds and pharmaceutical compositions containing compounds represented by Formula III: 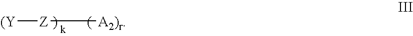
In Formula III, Y, Z, k and r are defined as in Formula I, and A₂, for each occurrence, is a compound represented by the following Formula IV, or optical isomers, diastereomers, or pharmaceutically acceptable salts thereof: 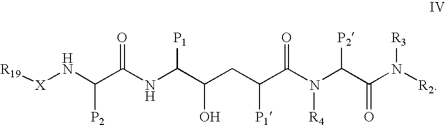
In Formula IV, X, P₁, P₂, P₁′, P₂′, R₂, R₃ and R₄ are defined as in Formula II, and R₁₉ an aliphatic group substituted with one or more substituents, wherein at least one substituent is a substituent selected from the group consisting of —NR₁₅C(O)R₁₆, —NR₁₅C(O)₂R₁₆ and —NR₁₅S(O)₂R₁₆. R₁₅ and R₁₆ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more substituents selected from the group consisting of an aliphatic group, hydroxy, —OR₉, a halogen, a cyano, a nitro, —NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀, an aryl, a heteroaryl, a heteroaralkyl, and a heterocycle. p is 0, 1, or 2. However, when R₁₉ is substituted with —NR₁₅C(O)R₁₆ or —NR₁₅C(O)₂R₁₆, —NR₂R₃ is not a group having the following structural formula:

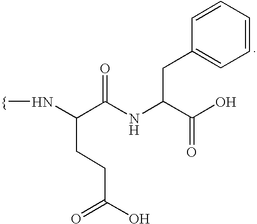

In another embodiment, the invention is directed to compounds and pharmaceutical compositions containing compounds that selectively inhibit hydrolysis of a memapsin 2 β-secretase site relative to a memapsin 1 β-secretase site. Compounds of the invention that selectively inhibit hydrolysis of a memapsin 2 β-secretase site relative to a memapsin 1 β-secretase site are represented by Formula V: 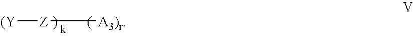
In Formula V, Y, Z, k and r are defined as in Formula I, and A₃, for each occurrence, is a compound represented by the following Formula II, or optical isomers, diastereomers, or pharmaceutically acceptable salts thereof:

In another embodiment, the invention is directed to compounds and pharmaceutical compositions containing compounds represented by Formula VI: 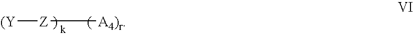
In Formula VI, Y, Z, k and r are defined as in Formula I and A₄, for each occurrence, is a compound represented by the following Formula VII, or optical isomers, diastereomers, or pharmaceutically acceptable salts thereof: 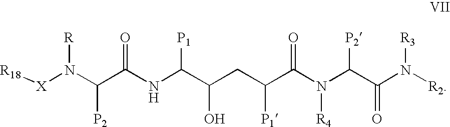
In Formula VII, X, P₁, P₂, P₁′, P₂′, R₂, R₃ and R₄ are defined as in Formula II, are defined as in Formula II, and R₁₈ is a substituted or unsubstituted heteroaralkoxy, a substituted or unsubstituted heteroaralkyl, or —NR₂₀R₂₁. R₂₀ and R₂₁ are each, independently, —H or a substituted or unsubstituted heteroaralkyl. Alternatively, R and one of R₂₀ or R₂₁, together with X and the nitrogen atoms to which they are attached, form a 5-, 6-, or 7-membered substituted or unsubstituted heterocycle or substituted or unsubstituted heteroaryl ring.

In another embodiment, the invention is directed to compounds and pharmaceutical compositions containing compounds represented by Formula VIII: 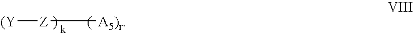
In Formula VIII, A₅, for each occurrence, in the compounds represented by Formula VIII is selected from the group of compounds in Table 1 or optical isomers, diastereomers, or pharmaceutically acceptable salts thereof.

In another embodiment, the present invention relates to a method of inhibiting hydrolysis of a β-secretase site of a β-amyloid precursor protein in an in vitro sample by administering to the in vitro sample a compound represented by Formula I, III, V, VI or VIII.

In another embodiment, the present invention relates to a method of decreasing β-amyloid protein (Walsh, D. M., et al., J. Biol. Chem. 274:25945-25952 (1999) and Liu, K., et al., Biochemistry 41:3128-3136 (2002)) in an in vitro sample by administering to the in vitro sample a compound represented by Formula I, III, V, VI or VIII.

In another embodiment, the present invention relates to a method of decreasing β-amyloid protein in a mammal by administering to the mammal a compound represented by Formula I, III, V, VI, or VIII.

In another embodiment, the present invention relates to a method of selectively inhibiting hydrolysis of a β-secretase site by memapsin 2 relative to memapsin 1 in an in vitro sample by administering to the in vitro sample a compound represented by Formula I, III, V, VI or VIII.

In another embodiment, the present invention relates to a method of selectively inhibiting hydrolysis of a β-secretase site by memapsin 2 relative to memapsin 1 in a mammal by administering to the mammal a compound represented by Formula I, III, V, VI or VIII.

In another embodiment, the present invention relates to a method of inhibiting hydrolysis of a β-secretase site of a β-amyloid precursor protein in a mammal by administering a compound represented by Formula I, III, V, VI or VIII.

In another embodiment, the present invention relates to a method of treating Alzheimer's disease in a mammal by administering to the mammal a compound represented by Formula I, III, V, VI, or VIII.

In another embodiment, the present invention relates to a crystallized protein selected from the group consisting of amino acid residues 1-456 of SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8.; and a compound represented by Formula I, III, V, VI or VIII. The crystallized protein has an x-ray diffraction resolution limit not greater than about 4.0 Å.

In another embodiment, the present invention relates to a crystallized protein comprising a protein of SEQ ID NO: 6 and a compound represented by Formula I, III, V, VI or VIII. The crystallized protein has an x-ray diffraction resolution limit not greater than about 4.0 Å.

In another embodiment, the present invention relates to a crystallized protein comprising a protein encoded by SEQ ID NO: 5 and a compound is represented by Formula I, III, V, V, or VIII. The crystallized protein has an x-ray diffraction resolution limit not greater than about 4.0 Å.

In another embodiment, the present invention relates to a crystallized complex comprising a protein selected from the group consisting of amino acid residues 1-456 SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and a compound in association with said protein, wherein said substrate is in association with said protein at an S₃′ binding pocket, an S₄′ binding pocket and an S₄ binding pocket. Preferably, the compound is a compound of Formula I, III, V, VI, or VIII.

In another embodiment, the present invention relates to a crystallized complex comprising a protein selected from the group consisting of amino acid residues 1-456 SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and a compound in association with said protein, wherein said compound is in association with said protein at an S₃ binding pocket. Preferably, the compound is a compound Formula V, VI, or VIII.

In another embodiment, the present invention relates to a crystallized complex comprising a protein selected from the group consisting of amino acid residues 1-456 SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and a compound represented by Formula V, VI, or VIII in association with said protein, wherein said compound is in association with said protein at an S₃ binding pocket.

The invention described herein provides compounds for inhibiting the activity of memapsin 2 (β-secretase) and methods of using the compounds, for example, to inhibit the hydrolysis of a β-secretase site of a β-amyloid precursor protein, treat Alzheimer's disease and decrease β-amyloid protein. Advantages of the claimed invention include, for example, the selectivity of compounds for inhibiting memapsin 2 activity relative to the activity memapsin 1 activity, thereby providing a specific inhibitor for β-secretase and treatment of diseases or conditions associated with β-secretase activity. The claimed methods, by employing memapsin 2 inhibitors, provide methods to inhibit a biological reaction which is involved in the accumulation or production of β-amyloid protein, a phenomenon associated with Alzheimer's disease in humans.

Thus, the compounds of the invention can be employed in the treatment of diseases or conditions associated with β-secretase activity, which can halt, reverse or diminish the progression of the disease or condition, in particular Alzheimer's disease.

### BRIEF DESCRIPTION OF THE FIGURES

FIGS. 1A, 1B, 1C, 1D, 1E, 1F, 1G, and 1H depict the preference index of memapsin 1 for amino acid residues (single-letter code) in the eight position (P₁, P₂, P₃, P₄, P₁′, P₂′, P₃′ and P₄′, respectively) of memapsin 2 substrate mixtures.

FIGS. 2A, 2B, 2C, 2D, 2E, 2F, 2G and 2H depict the preference of memapsin 2 for amino acid residues (single-letter code) in eight positions (P₁, P₂, P₃, P₄, P₁′, P₂′, P₃′, P₄′, respectively) of memapsin 2 substrate mixtures.

FIG. 3 depicts the selectivity of inhibitors (GT-1017, GT-1026, OM00-3 and GT-113) for inhibition of memapsin 2 activity relative to inhibition of memapsin 1 activity.

FIG. 4 depicts the nucleic acid sequence of memapsin 1 (GenBank Index (GI): 21040358; SEQ ID NO: 1).

FIG. 5 depicts the deduced amino acid sequence (GI: 19923395; SEQ ID NO: 2) of the nucleic acid sequence of memapsin 1 (GI: 21040358; SEQ ID NO: 1). The transmembrane domain at amino acids 467-494 is underlined.

FIG. 6 depicts the nucleic acid sequence of promemapsin 1-T1 (SEQ ID NO: 3).

FIG. 7 depicts the deduced amino acid sequence (SEQ ID NO: 4) of promemapsin 1-T1 nucleotide sequence (SEQ ID NO: 3). Vector (pET-11) sequence (residues 1-14) is underlined.

FIG. 8 depicts the nucleic acid sequence of memapsin 2 (GI #21040369; SEQ ID NO: 5).

FIG. 9 depicts the deduced amino acid sequence (GI: 6912266; SEQ ID NO: 6) of the nucleic acid sequence of memapsin 2 (GI: 21040369; SEQ ID NO: 5). Amino acid residues 1-21 indicate the signal peptide. Amino acid residues 22-45 indicate the propeptide. Amino acid residues 455-480 (underlined) indicate the transmembrane domain of memapsin 2.

FIG. 10 depicts the nucleic acid sequence of promemapsin 2-T1 (SEQ ID NO: 7).

FIG. 11 depicts the deduced amino acid of promemapsin 2-T1 (SEQ ID NO: 8) encoded by the nucleic acid sequence of promemapsin 2-T1 (SEQ ID NO: 7). Vector (pET-11) sequence (amino acid residues 1-15) is underlined.

FIG. 12 depicts the numbering scheme of the amino acid sequence of memapsin 2 (SEQ ID NO: 9) generally employed in crystal structure determinations. Residues are numbered from the amino terminal Leu 28P through Val 48P, continuing with the adjacent Glu 1 and numbering consecutively through Thr 393. Amino acid Glu 1 corresponds to the amino terminal of mature pepsin.

FIG. 13 depicts the average B Factors for inhibitor residues (P₄, P₃, P₂, P₁, P₁′, P₂′, P₃′, and P₄′) for the inhibitors OM99-2 and OM00-3.

FIG. 14 is a schematic representation of the inhibitor compound OM00-3 and its interactions with memapsin 2 as determined from crystallization complexes of memapsin 2 and OM00-3. The memapsin 2 residues contacting the OM00-3 (distance less than 4.5 Å) are shown in bold cased letters. The dotted lines depicted between the atom of OM00-3 and amino acid residues of memapsin 2 are hydrogen bond interactions. Interactions between the inhibitor OM99-2 and amino acid residues of memapsin 2 which differ from the OM00-3 complex are depicted in italicized letters.

FIGS. 15A and 15B depict the amino acid residue preference at positions P₃′ and P₄′ with the P₂′ amino acid residues of alanine (stippled bars) or valine (solid bars).

FIG. 16 depicts the interaction between the inhibitors compounds OM00-3 and OM99-2 and memapsin 2 in a crystal complex of memapsin 2 and OM00-3 or OM99-2. The side chains of the compounds are depicted as P₁, P₂, P₃, P₄, P₁′, P₂′, P₃′ and P₄′.

FIG. 17 illustrates the inhibition of memapsin 2 activity by the carrier peptide-inhibitor conjugate CPI-2.

FIGS. 18A, 18B and 18C depict entry of the carrier peptide-inhibitor conjugate CPI-1 (4, 40 or 400 nM), CPI-2, and fluorescein (Fs) alone into HeLa cells. Untreated cells are labeled “cells only.”

FIGS. 19A, 19B and 19C depict the flow cytometry analysis of whole blood cells, splenocytes and brain cells isolated from mice twenty minutes, two hours or eight hours, respectively, after intraperintoneal injection of 25 nM CPI-1 (shaded area in panel A and unshaded area in panels B or C) or fluorescein control (unshaded area in panel A and shaded area in B and C).

FIG. 20 depicts the Flow cytometry analysis of the entry of CPI-1 (25 nM) into the brain of mice following the administration of the carrier peptide-inhibitor conjugate CPI-1 (shaded area) and fluorescein control (unshaded area).

FIG. 21A depicts a dose-dependent decrease in plasma β-amyloid protein two hours after administration of carrier peptide-inhibitor conjugate CPI-3 (16, 80, 400 μg) to transgenic mice. Significant differences are depicted by the asterisks (*, P<0.01).

FIG. 21B depicts the sustained inhibition of plasma levels of β-amyloid protein in transgenic animals receiving carrier peptide-inhibitor conjugate CPI-3 or OM00-3 compared to DMSO alone treatment. A significant difference in values compared to DMSO controls is indicated by a single asterisk (*, P<0.05) or double asterisks (**, P<0.01) and was determined by the Student's t-test.

FIG. 21C depicts a decrease in the plasma levels of β-amyloid protein in transgenic mice following the administration of the carrier peptide-inhibitor conjugate CPI-3 (400 kg), OM00-3 (400 μg), peptide (400 μg), OM00-3 and peptide (400 μg) compared to PBS and DMSO controls. A significant difference in values compared to controls was determined by the Student's t-test and is indicated by the asterisks (*, P<0.01).

FIG. 21D depicts a decline in plasma levels of β-amyloid protein (Aβ) in transgenic mice receiving four injections (arrows) of carrier peptide inhibitor conjugate CPI-3, peptide or PBS. Significant differences are depicted by the asterisks (*, P<0.01).

FIGS. 22A and 22B depict a decrease in the plasma levels of β-amyloid protein (Aβ) following the administration of the inhibitor compounds MMI-138, MMI-165 and MMI-185 to transgenic tg2576 mice.

FIG. 23 depicts the amino acid sequence of amyloid precursor protein (GenBank Accession No: P05067, GI: 112927; SEQ ID NO: 10). The β-secretase site at amino acid residues 667-676 is underlined. The β-secretase cleavage site between amino acid residues 671 and 672 is depicted by the arrow.

FIG. 24 shows the active site region of the crystal structure of MMI-138 (shown as the darker bonds) complexed to memapsin 2 (shown as lighter bonds).

FIG. 25 is a structural schematic of MMI-138 showing the atoms of MMI-138 numbered to correspond to the atoms named in the atomic coordinates of the crystal structure of the complex between MMI-138 and memapsin 2.

FIGS. 26A, 26B, 26C and 26D depict the amino acid residue preference at positions P₅, P₆, P₇ and P₈, respectively, of memapsin 2 substrates.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention. The teachings of all of the references cited herein are incorporated by reference in their entirety.

The term “aliphatic” as used herein means straight-chain, branched C₁-C₁₂ or cyclic C₃-C₁₂ hydrocarbons which are completely saturated or which contain one or more units of unsaturation but which are not aromatic. For example, suitable aliphatic groups include substituted or unsubstituted linear, branched or cyclic alkyl, alkenyl, alkynyl groups and hybrids thereof such as (cycloalkyl)alkyl, (cycloalkenyl)alkyl or (cycloalkyl)alkenyl. The terms “alkyl”, used alone or as part of a larger moiety, includes both straight, branched, or cyclic saturated hydrocarbon chains containing one to twelve carbon atoms. Preferably, alkyl groups are straight chain hydrocarbons having from one to about four carbons.

An alkylene, as used herein, is an alkyl group that has two points of attachment to another moiety, such as methylene.

A heteroalkyl, as used herein, is an alkyl group in which one or more carbon atoms is replaced by a heteroatom. A preferred heteroalkyl is methoxymethoxy.

A hydroxyalkyl, as used herein, is an alkyl group that is substituted with one or more hydroxy groups.

The term “aryl” used alone or as part of a larger moiety as in “aralkyl” or “aralkoxy”, are carbocyclic aromatic ring systems (e.g. phenyl), fused polycyclic aromatic ring systems (e.g., naphthyl and anthracenyl) and aromatic ring systems fused to carbocyclic non-aromatic ring systems (e.g., 1,2,3,4-tetrahydronaphthyl and indanyl) having five to about fourteen carbon atoms.

The term “heteroatom” refers to any atom ohter than carbon or hydrogen. Preferred heteroatoms are nitrogen, oxygen, sulfur, and phosphorus and includes, for example, any oxidized form of nitrogen and sulfur, and the quaternized form of any basic nitrogen.

The term “heterocycle”, as used herein includes non-aromatic ring systems having five to fourteen members, preferably five to ten, in which one or more ring carbons, preferably one to four, are each replaced by a heteroatom. Examples of heterocyclic rings include, tetrahydrofuranyl, tetrahydropyrimidin-2-one, pyrrolidin-2-one, hexahydro-cyclopenta[b]furanyl, hexahydrofuro[2,3-b]furanyl, tetrahydropyranyl, tetrahydropyranone, [1,3]-dioxanyl, [1,3]-dithianyl, tetrahydrothiophenyl, morpholinyl, thiomorpholinyl, pyrrolidinyl, pyrrolidinone, piperazinyl, piperidinyl, and thiazolidinyl. Also included within the scope of the term “heterocycle”, as it is used herein, are groups in which a non-aromatic heteroatom-containing ring is fused to one or more aromatic or non-aromatic rings, such as in an indolinyl, chromanyl, phenantrhidinyl, or tetrahydroquinolinyl, where the radical or point of attachment is on the non-aromatic heteroatom-containing ring. Preferred heterocycles are tetrahydrofuranyl, tetrahydropyranyl, pyrrolidinyl, tetrahydropyrimidin-2-one, and pyrrolidin-2-one.

The term “heteroaryl”, used alone or as part of a larger moiety as in “heteroaralkyl” or “heteroarylalkoxy”, refers to aromatic ring system having five to fourteen members and having at least one heteroatom. Preferably a heteroaryl has from one to about four heteroatoms. Examples of heteroaryl rings include pyrazolyl, furanyl, imidazolyl, isoxazolyl, oxadiazolyl, oxazolyl, pyrrolyl, pyridyl, pyrimidinyl, purinyl, pyridazinyl, pyrazinyl, thiazolyl, thiadiazolyl, isothiazolyl, triazolyl, thienyl, 4,6-dihydro-thieno[3,4-c]pyrazolyl, 5,5-dioxide-4,6-dihydrothieno[3,4-c]pyrazolyl, thianaphthenyl, 1,4,5,6,-tetrahydrocyclopentapyrazolyl, carbazolyl, benzimidazolyl, benzothienyl, benzofuranyl indolyl, azaindolyl, indazolyl, quinolinyl benzotriazolyl, benzothiazolyl, benzothiadiazolyl, benzooxazolyl, benzimidazolyl, isoquinolinyl, isoindolyl, acridinyl, and benzoisazolyl. Preferred heteroaryl groups are pyrazolyl, furanyl, pyridyl, quinolinyl, indolyl and imidazolyl.

A heteroazaaryl is a heteroaryl in which at least one of the heteroatoms is nitrogen. Preferred heteroazaaryl groups are pyrazolyl, imidazolyl, isoxazolyl, oxadiazolyl, oxazolyl, pyrrolyl, pyridyl, pyrimidyl, pyridazinyl, thiazolyl, triazolyl, benzimidazolyl, quinolinyl, benzotriazolyl benzooxazolyl, benzimidazolyl, isoquinolinyl, indolyl, isoindolyl and benzoisazolyl. Pyrazolyl is a most preferred heteroazaaryl.

An aralkyl group, as used herein, is an aryl substituent that is linked to a compound by a straight chain or branched alkyl group having from one to twelve carbon atoms. Preferred aralkyl groups are benzyl and indanylmethyl.

An heterocycloalkyl group, as used herein, is a heterocycle substituent that is linked to a compound by a straight chain or branched alkyl group having from one to twelve carbon atoms. Preferred heterocycloalkyl groups are tetrahydrofuranylmethyl and pyrrolidinylmethyl.

An heteroaralkyl group, as used herein, is a heteroaryl substituent that is linked to a compound by a straight chain or branched alkyl group having from one to twelve carbon atoms. Preferred heteroaralkyl groups are pyrazolylmethyl, 2-pyrazolylethyl, 2-pyrazolyl-1-methylethyl, and 2-pyrazolyl-1-isopropylethyl.

An alkoxy group, as used herein, is a straight chain or branched or cyclic C₁-C₁₂ or a cyclic C₃-C₁₂ alkyl group that is connected to a compound via an oxygen atom. Examples of alkoxy groups include but are not limited to methoxy, ethoxy, propoxy, isopropoxy, and t-butoxy.

A heterocyclooxy, as used herein, is a heterocyclic group that is attached to a molecule via an oxygen substituent.

A aralkoxy group, as used herein, is a aralkyl group that is attached to a compound via an oxygen substituent on the C₁-C₁₂ alkyl portion of the aralkyl. A preferred arylalkoxy is phenylmethoxy.

A heteroaralkoxy group, as used herein, is a heteroaralkyl group that is attached to a compound via an oxygen substituent on the C₁-C₁₂ alkyl portion of the heteroaralkyl. A preferred arylalkoxy are pyrazolylmethoxy and 2-pyrazolylethoxy.

A heterocycloalkoxy group, as used herein, is a heterocycloalkyl group that is attached to a compound via an oxygen substituent on the C₁-C₁₂ alkyl portion of the heteroaralkyl.

An alklysulfanylalkyl group, as used herein, is a sulfur atom that is linked to two C₁-C₁₂ alkyl groups, wherein one of the alkyl groups is also linked to a compound.

A halogen is a —F, —Cl, —Br, or —I.

A haloalkyl is an alkyl group that is substituted by one or more halogens.

A haloalkoxy is an alkoxy group that is substituted with one or more halogens.

An aryl (including aralkyl, aralkoxy and the like) or heteroaryl (including heteroaralkyl and heteroaralkoxy and the like) may contain one or more substituents. Examples of suitable substituents include aliphatic groups, aryl groups, haloalkoxy groups, heteroaryl groups, halo, hydroxy, OR₂₄, COR₂₄, COOR₂₄, NHCOR₂₄, OCOR₂₄, benzyl, haloalkyl (e.g., trifluoromethyl and trichloromethyl), cyano, nitro, SO₃⁻, SH, SR₂₄, NH₂, NHR₂₄, NR₂₄R₂₅, NR₂₄S(O)₂—R₂₅, and COOH, wherein R₂₄ and R₂₅ are each, independently, an aliphatic group, an aryl group, or an aralky group. Other substituents for an aryl or heteroaryl group include —R₂₆, —OR₂₆, —SR₂₆, 1,2-methylene-dioxy, 1,2-ethylenedioxy, protected OH (such as acyloxy), phenyl (Ph), substituted Ph, —O(Ph), substituted —O(Ph), —CH₂(Ph), substituted —CH₂CH₂(Ph), substituted —CH₂CH₂(Ph), —NR₂₆R₂₇, —NR₂₆CO₂R₂₇, —NR₂₆NR₂₇C(O)R₂₈, —NR₂₆R₂₇C(O)NR₂₈R₂₉, —NR₂₆NR₂₇CO₂R₂₈, —C(O)C(O)R₂₆, —C(O)CH₂C(O)R₂₆, —CO₂R₂₆, —C(O)R₂₆, —C(O)NR₂₆R₂₇, —OC(O)NR₁₆R₂₇, —S(O)₂R₂₆, —SO₂NR₂₆R₂₇, —S(O)R₂₆, —NR₂₆SO₂NR₂₆R₂₇, —NR₂₆SO₂R₂₇, —C(═S)NR₂₆R₂₇, —C(═NH)—NR₂₆R₂₇, —(CH₂)_{y}NHC(O)R₂₆, wherein R₂₆, R₂₇ and R₂₈ are each, independently, hydrogen, a substituted or unsubstituted heteroaryl or heterocycle, phenyl (Ph), substituted Ph, —O(Ph), substituted —O(Ph), —CH₂(Ph), or substituted —CH₂(Ph); and y is 0-6. Examples of substituents on the aliphatic group or the phenyl group include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl. Preferred substitutents for a heteroaryl group such as a pyrazole group, are a substituted or unsubstituted aliphatic group, —OR₉, —R₂₃—O—R₉, a halogen, a cyano, a nitro, NR₉R₁₀, guanidino, OPO₃⁻², PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀ a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl, wherein R₉ and R₁₀ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more aliphatic groups.

An aliphatic group, an alkylene, the carbon atoms of a heteroalkyl, and a heterocycle (including heterocycloalkyl, hetorcyclooxy, and heterocycloalkoxy) may contain one or more substituents. Examples of suitable substituents on the saturated carbon of an aliphatic group of a heterocycle include those listed above for an aryl or heteroaryl group and the following: ═O, ═S, ═NNHR₂₉, ═NNR₂₉R₃₀, ═NNHC(O)R₂₉, ═NNHCO₂(alkyl), ═NNHSO₂(alkyl), or ═NR₂₉, where each R₂₉ and R₃₀ are each, independently, selected from hydrogen, an unsubstituted aliphatic group or a substituted aliphatic group. Examples of substituents on the aliphatic group include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, thioalkyl, nitro, cyano, carboxy, alkoxycarbonyl, alkylcarbonyl, hydroxy, haloalkoxy, or haloalkyl.

Suitable substitutents on the nitrogen of a non-aromatic heterocycle or on an unsaturated nitrogen of a heteroaryl include —R₃₁, —NR₃₁R₃₂, —C(O)R₃₁, —CO₂R₃₁, —C(O)C(O)R₃₁, —C(O)CH₂C(O)R₃₁, —SO₂R₃₁, —SO₂NR₃₁R₃₂, —C(═S)NR₃₁R₃₂, —C(—NH)—NR₃₁R₃₂, and —NR₃₁SO₂R₃₂; wherein R₃₁ and R₃₂ are each, independently, hydrogen, an aliphatic group, a substituted aliphatic group, phenyl (Ph), substituted Ph, —O(Ph), substituted —O(Ph), —CH₂(Ph), or a heteroaryl or hetero cycle. Examples of substituents on the aliphatic group or the phenyl ring include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

A hydrophobic group is a group that does not reduce the solubility of a compound in octane or increases the solubility of a compound in octane. Examples of hydrophobic groups include aliphatic groups, aryl groups, and aralkyl groups.

As used herein, the term “natural amino acid” refers to the twenty-three natural amino acids known in the art, which are as follows (denoted by their three letter acronym): Ala, Arg, Asn, Asp, Cys, Cys-Cys, Glu, Gln, Gly, His, Hyl, Hyp, Ile, Leu, Lys, Met, Phe, Pro, Ser, Thr, Trp, Tyr, and Val. The term “side-chain of an amino acid”, as used herein, is the substituent on the alpha-carbon of a natural amino acid.

The term “non-natural amino acid” refers to compounds of the formula NH₂—C(R₃₂)₂—COOH, where R₃₂ for each occurrence is, independently, any side chain moiety recognized by those skilled in the art; examples of non-natural amino acids include, but are not limited to: hydroxyproline, homoproline, 4-amino-phenylalanine, norleucine, cyclohexylalanine, α-aminoisobutyric acid, N-methyl-alanine, N-methyl-glycine, N-methyl-glutamic acid, tert-butylglycine, α-aminobutyric acid, tert-butylalanine, ornithine, α-aminoisobutyric acid, 2-aminoindane-2-carboxylic acid, etc. and the derivatives thereof, especially where the amine nitrogen has been mono- or di-alkylated.

A peptide substituent is a sequence of natural or non-natural amino acids that are linked together via an amide bond which is formed by reaction of the α-amine of one amino acid with the α-carboxylic acid of an adjacent amino acid. Preferably, a peptide sequence includes only natural amino acids. In one embodiment, a peptide substituent is a sequence of about 6 natural amino acids. In another embodiment, a peptide substituent is a sequence of 2 natural amino acids. In yet another embodiment, a peptide substituent is 1 natural amino acid.

A “transition state isostere,” or “isostere;” as used herein, is a compound having a sequence of two or more natural or non-natural amino acids, wherein at least one amide linkage between two consecutive amino acids has been modified such that the —NH— group of the amide has been replaced with a —CH₂— and the carbonyl of the amide group has been replaced with a —CH(OH)—. This isostere is also referred to herein as a “hydroxyethylene isostere” because the amide linkage between a pair of amino acids of a peptide is modified to form a hydroxyethylene linkage between the amino acids. A hydroxyethylene group is an isostere of the transition state of hydrolysis of an amide bond. Preferably, an isostere has only one modified amide linkage. The hydroxyethylene component of a peptide isostere is also referenced herein as “*” or “Ψ”. For example, the representation of the di-isostere Leucine*Alanine, Leu*Ala, L*A, or LΨA each refer to the following structure: 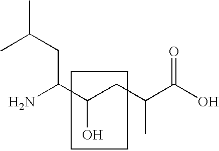
where the boxed portion of the molecule represents the hydroxyethylene component of the molecule.

“Binding pockets” or “binding subsites” or subsites” refer to locations in an enzyme or protease that interact with functional groups or side chains of a compound or substrate bond thereto. The subsites in memapsin 1 and memapsin 2 are labeled S_{q} and S_{q}′ and interact with or otherwise accommodate side chains P_{q} and P_{q}′ of a peptide substrate or peptide isostere, such as the compounds of the invention, such that the P_{q} side chain of the peptide substrate or peptide inhibitor interact with amino acid residues in the S_{q} subsite of the enzyme. q is an integer that increases distally relative to the scissile bond of a peptide substrate that is cleaved by the enzyme or relative to the hydroxyethyl group of a hydroxyethyl isosteric inhibitor, such as the compounds of the invention, according to the nomenclature of Schecter and Berger (Schechter, I., Berger, A. Biochem. Biophys. Res. Commun. (1967), 27:157-162). The composition of a subsite is a listing of the amino acids of the enzyme or protease which are within an interacting distance of the compound when the compound is bound to the subsite, or otherwise form a contiguous solvent accessible surface, indicated by their numbers in the amino acid sequence. Representative references to aspartic protease subsites include: Davies, D. R, Annu. Rev. Biophys. Biophys. Chem., 19:189-215 (1990) and Bailey, D. and Cooper, J. B., Protein Science, 3:2129-2143 (1994), the teachings of which are incorporated herein by reference in their entirety. More specifically, a subsite is defined by defining a group of atoms of the enzyme which represent a contiguous or noncontiguous surface that is accessible to a water molecule, with that surface having the potential for an interaction with a functional group or side chain of a peptide substrate or a peptide inhibitor, such as the compounds of the invention, when the peptide substrate or a peptide inhibitor is bound to the subsite.

An “interacting distance” is defined as a distance appropriate for van der Waals interactions, hydrogen bonding, or ionic interactions, as described in fundamental texts, such as Fersht, A., “Enzyme Structure and Mechanism,” (1985), W.H. Freeman and Company, New York. Generally, atoms within 4.5 Å of each other are considered to be within interacting distance of each other.

In many of the compounds of the invention, the amino acid residues whose side chains would be labeled P₃, P₄, etc. when using the above nomenclature have been replaced by a chemical group that is not an amino acid. Thus, in the compounds of formulas II, IV and VII, R₁ together with X, R₁₉ together with X, and R₁₈ together with X, respectively, may include amino acid residues but also include other chemical groups as defined above (see definition of R₁, R₁₉ and R₁₈). When R₁ together with X, R₁₉ together with X, or R₁₈ together with X, in the compounds of the invention is a peptide group, the side chains of the peptide group are labeled P₃, P₄, etc. and bind in the enzyme subsites S₃ and S₄ as in the nomenclature described above. When R₁ together with X, R₁₉ together with X, or R₁₈ together with X, in the compounds of the invention is a non-peptide moiety, these groups may also bind in the S₃ and/or S₄ subsite of the enzyme.

A “substrate” is a compound that may bind to the active site cleft of the enzyme according to the following scheme:
E+S [symbol] E•S
In the above reaction scheme, “E” is an enzyme, “S” is a substrate, and “E•S” is a complex of the enzyme bound to the substrate. Complexation of the enzyme and the substrate is a reversible reaction.

Compounds of Formulas II, IV, VII and the compounds in Table 1 may exist as salts with pharmaceutically acceptable acids. The present invention includes such salts. Examples of such salts include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures_, succinates, benzoates and salts with amino acids such as glutamic acid. These salts may be prepared by methods known to those skilled in the art.

Certain compounds of Formulas II, IV, VII and the compounds in Table 1 which have acidic substituents may exist as salts with pharmaceutically acceptable bases. The present invention includes such salts. Example of such salts include sodium salts, potassium salts, lysine salts and arginine salts. These salts may be prepared by methods known to those skilled in the art.

Certain compounds of Formulas II, IV, VII and the compounds in Table 1 may contain one or more chiral centres, and exist in different optically active forms. When compounds of Formulas II, IV, VII or the compounds in Table 1 contain one chiral centre, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of Formulas II, IV, VII or a compound in Table 1 contain more than one chiral center, it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of Formula I and mixtures thereof.

Certain compounds of Formulas II, IV, VII and the compounds in Table 1 may exist in zwitterionic form and the present invention includes each zwitterionic form of compounds of Formula (I) and mixtures thereof.

In a preferred embodiment, the compounds of Formula II or IV, separately or with their respective pharmaceutical compositions, have an R₁ or R₁₉, respectively, group that together with X is an natural or non-natural amino acid derivative. The compounds of this embodiment are preferably represented by Formula IX: 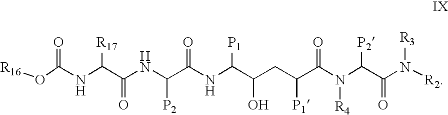
In Formula IX, P₁, P₂, P₁′, P₂′, R₂, R₃ and R₄ are defined as in Formula II. R₁₆ is defined as in Formula IV, and R₁₇ is a substituted or unsubstituted aliphatic group.

In another preferred embodiment, the compounds of Formula II or VII, separately or with their respective pharmaceutical compositions, have an R₁ or R₁₈ group, respectively, that is a substituted or unsubstituted heteroaralkoxy or a substituted or unsubstituted heteroaralkyl. The compounds of this embodiment are preferably represented by Formula X: 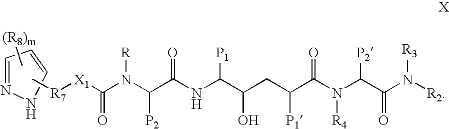
In Formula X, P₁, P₂, P₁′, P₂′, R₂, R₃ and R₄ are defined as in Formula II. X₁ is —O—, —NR₂₂— or a covalent bond. R₇ is a substituted or unsubstituted alkylene. m is 0, 1, 2, or 3. R₈ is a substituted or unsubstituted aliphatic group, —OR₉, —R₂₃—O—R₉, a halogen, a cyano, a nitro, NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀ a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl. p is 0, 1 or 2. R₉ and R₁₀ are defined as in Formula IV. R₂₃ is a substituted or unsubstituted alkylene. R₂₂ is —H. Alternatively, R and R₂₂, together with X and the nitrogen atoms to which they are attached, form a 5-, 6-, or 7-membered substituted or unsubstituted heterocycle or substituted or unsubstituted heteroaryl ring.

In one preferred embodiment, R₁ of Formula II is —OR₁₅ or —NR₁₅R₁₆. R₁₅ and R₁₆ are defined as in Formula IV.

In another preferred embodiment, R₁ of Formula II is a substituted aliphatic group. More preferably, R₁ is an aliphatic group that is substituted with one or more substituents selected from the group consisting of —NR₁₅C(O)₂R₁₆, —NR₁₅C(O)R₁₆, and —NR₁₅S(O)₂R₁₆. R₁₅ and R₁₆ are defined as in Formula IV.

In another preferred embodiment, R₁ of Formula II together with X is a peptide represented by Formula XI: 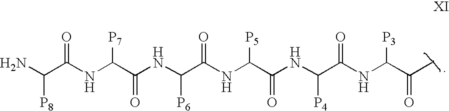
In Formula XI, P₃ and P₄ are each, independently, an amino acid side chain. P₅ is an amino acid side chain selected from the group consisting of tryptophan side chain, methionine side chain, and leucine side chain. P₆ is tryptophan side chain. P₇ is an amino acid side chain selected from the group consisting of tryptophan side chain, tyrosine side chain; and glutamate side chain. P₈ is an amino acid side chain selected from the group consisting of tryptophan side chain, tyrosine side chain; and glutamate side chain. More preferably, P₅, P₆, P₇, and P₈ are each a tryptophan side chain.

In another preferred embodiment, P₁ of Formula II, IV, or VII is an aliphatic group. More preferably, P₁ is selected from the group consisting of isobutyl, hydroxymethyl, cyclopropylmethyl, cyclobutylmethyl, phenylmethyl, cyclopentylmethyl, and heterocycloalkyl.

In another preferred embodiment, P₂′ of Formula II, IV, or VII is a hydrophobic group. More preferably, P₂′ is isopropyl or isobutyl.

In another preferred embodiment, P₂ of Formula II, IV, or VII is a hydrophobic group. More preferably, P₂ is —R₁₁SR₁₂, —R₁₁S(O)R₁₂, —R₁₁S(O)₂R₁₂, —R₁₁C(O)NR₁₂R₁₃, —R₁₁OR₁₂, —R₁₁OR₁₄OR₁₃, or a hetercycloalkyl, wherein the heterocycloalkyl is optionally substituted with one or more alkyl groups. R₁₁ and R₁₄ are each, independently, an alkylene. R₁₂ and R₁₃ are each, independently, H, an aliphatic group, an aryl, an arakyl, a heterocycle, a heterocyclalkyl, a heteroaryl, or a heteroaralkyl. Even more preferably, P₂ is —CH₂CH₂SCH₃, —CH₂CH₂S(O)CH₃, —CH₂CH₂S(O)₂CH₃, —CH₂C(O)NH₂, —CH₂C(O)NHCH₂CH═CH₂, tetrahydrofuran-2-yl, tetrahydrofuran-2-yl-methyl, tetrahydrofuran-3-yl, tetrahydrofuran-3-yl-methyl, pyrrolidin-2-yl-methyl, pyrrolidin-3-yl-methyl, or —CH₂CH₂OCH₂OCH₃.

In another preferred embodiment, R₂ is H and R₃ together with the nitrogen to which it is attached is a peptide in Formula II, IV or VII.

In another preferred embodiment, R₂ is H and R₃ is selected from the group consisting of 2-furanylmethyl, phenylmethyl, indan-2-yl, n-butyl, isopropyl, isobutyl, 1-fluoromethyl-2-fluoroethyl, indol-3-yl, and 3-pyridylmethyl in Formula II, IV or VII.

In another preferred embodiment, R₂ and R₃ in Formula II, IV or VII, together with the nitrogen to which they are attached, form morpholino, piperazinyl or piperidinyl, wherein the morpholino, piperazinyl and piperidinyl are optionally substituted with one or more aliphatic groups.

In another embodiment, R₁ of formula II is a substituted or unsubstituted heteroaralkoxy or a substituted or unsubstituted heteroaralkyl.

In another preferred embodiment, R₁ of Formula I or R₁₈ or Formula VII is a substituted or unsubstituted heteroaralkoxy or a substituted or unsubstituted heteroaralkyl in which the heteroaryl group of the heteroaralkoxy or heteroaralkyl is selected from the group consisting of substituted or unsubstituted pyrazolyl, substituted or unsubstituted furanyl, substituted or unsubstituted imidazolyl, substituted or unsubstituted isoxazolyl, substituted or unsubstituted oxadiazolyl, substituted or unsubstituted oxazolyl, substituted or unsubstituted pyrrolyl, substituted or unsubstituted pyridyl, substituted or unsubstituted pyrimidyl, substituted or unsubstituted pyridazinyl, substituted or unsubstituted thiazolyl, substituted or unsubstituted triazolyl, substituted or unsubstituted thienyl, substituted or unsubstituted 4,6-dihydro-thieno[3,4-c]pyrazolyl, substituted or unsubstituted 5,5-dioxide-4,6-dihydrothieno[3,4-c]pyrazolyl, substituted or unsubstituted thianaphthenyl, substituted or unsubstituted carbazolyl, substituted or unsubstituted benzimidazolyl, substituted or unsubstituted benzothienyl, substituted or unsubstituted benzofuranyl, substituted or unsubstituted indolyl, substituted or unsubstituted quinolinyl, substituted or unsubstituted benzotriazolyl, substituted or unsubstituted benzothiazolyl, substituted or unsubstituted benzooxazolyl, substituted or unsubstituted benzimidazolyl, substituted or unsubstituted isoquinolinyl, substituted or unsubstituted isoindolyl, substituted or unsubstituted acridinyl, and substituted or unsubstituted benzoisazolyl. In a more preferred embodiment, R₁ of Formula I or R₁₈ or Formula VII is a substituted or unsubstituted heteroaralkoxy or a substituted or unsubstituted heteroaralkyl in which the heteroaryl group of the heteroaralkoxy or heteroaralkyl is a heteroazaaryl. In an even more preferred embodiment, the heteroazaaryl is selected from the group consisting of substituted or unsubstituted pyrazolyl, substituted or unsubstituted imidazolyl, substituted or unsubstituted isoxazolyl, substituted or unsubstituted oxadiazolyl, substituted or unsubstituted oxazolyl, substituted or unsubstituted pyrrolyl, substituted or unsubstituted pyridyl, substituted or unsubstituted pyrimidyl, substituted or unsubstituted pyridazinyl, substituted or unsubstituted thiazolyl, substituted or unsubstituted triazolyl, substituted or unsubstituted benzimidazolyl, substituted or unsubstituted quinolinyl, substituted or unsubstituted benzotriazolyl, substituted or unsubstituted benzooxazolyl, substituted or unsubstituted benzimidazolyl, substituted or unsubstituted isoquinolinyl, substituted or unsubstituted indolyl, substituted or unsubstituted isoindolyl, and substituted or unsubstituted benzoisazolyl.

In another preferred embodiment, the compounds of the invention do not include a carrier molecule. In this embodiment, k is 0 and r is 1 in Formula I, III, V, or VIII.

In another preferred embodiment of the invention, k is 1 and r is 1 in Formula I, III, V, or VIII. In this embodiment, each isosteric inhibitor is attached to one carrier molecule.

The compounds of the invention (also referred to herein as “an inhibitor(s)” or “an inhibitor compound(s)”) are referenced by a number. The inhibitors are also referred to as “GT-1” followed by a numeric designation (e.g., GT-1138), “MM” followed by a numeric designation (e.g., MM 138), “MMI” followed by a numeric designation (e.g., MMI-138) or “OM” followed by a numeric designation (e.g., OM-138). The designations “GT-1,” “MM,” “MMI” and “OM,” as described herein, are equivalent. Likewise, use of the numerical value following the designation “GT-1,” “MM,” “MMI” and “OM” without “GT-1,” “MM,” “MMI” and “OM” refer to the same compound with the “GT-1,” “MM,” “MMI” and “OM” prefix; Thus, for example, “GT-1138,” “MM 138,” “MMI-138,” “OM-138” and “138” refer to the same inhibitor compound.

In another embodiment, the invention includes a method of selectively inhibiting memapsin 2 β-secretase activity relative to memapsin 1 β-secretase activity, comprising the step of administering the compounds of the invention. The selective inhibition of memapsin 2 β-secretase activity compared to memapsin 1 β-secretase activity can be in an in vitro sample or in a mammal.

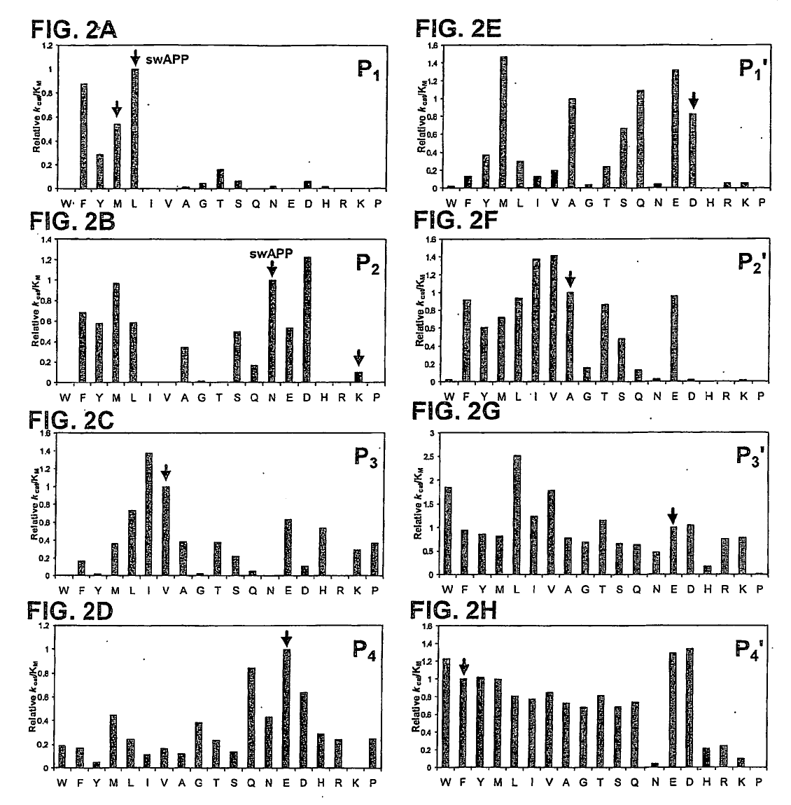

“Selectively inhibiting” or “selective inhibition,” as used herein, refers to a greater ability of a compound of the invention to inhibit, prevent or diminish the β-secretase activity of memapsin 2 than the ability of the same compound to inhibit, prevent or diminish β-secretase activity of memapsin 1 under the same conditions, as measured by the percent inhibition (“% inh”) of each. “Percent inhibition” is calculated as follows: % inh=(1−Vi/Vo)×100. For example, as shown in FIG. 2 and Table 9, the inhibitor compound 138 (also referred to herein as MMI-1-138 and GT-1138) inhibits the enzymatic activity of memapsin 2 in a manner that is about 60 fold greater than the inhibition of compound 138 on memapsin 1 β-secretase activity (compare Kᵢ 14.2 nM for memapsin 2 and Kᵢ 811.5 nM for memapsin 1). Thus, compound 138 is a selective inhibitor for memapsin 2 relative to memapsin 1 or selectively inhibits memapsin 2 β-secretase activity relative to memapsin 1 β-secretase activity.

“Relative to memapsin 1,” as used herein, refers to the β-secretase activity of memapsin 2 compared to the β-secretase activity of memapsin 1. The ability of an inhibitor compound of the invention to inhibit β-secretase activity can be assessed by determining the extent to which a compound inhibits memapsin 2 cleaving of a β-secretase site of a β-amyloid precursor protein compared to the extent to which the same compound inhibits memapsin 1 cleaving of a β-secretase site of a β-amyloid precursor protein. These data can be expressed, for example, as Kᵢ, K_{i apparent}, Vi/Vo, or percentage inhibition and depict the inhibition of a compound for memapsin 2 β-secretase activity relative to memapsin 1 β-secretase activity. For example, if the Kᵢ of a reaction between an inhibitor compound of the invention and memapsin 1 is 1000 and the Kᵢ of a reaction between an inhibitor compound of the invention and memapsin 2 is 100, the inhibitor compound inhibits the β-secretase activity of memapsin 2 ten (10) fold, relative to memapsin 1.

Kᵢ is the inhibition equilibrium constant which indicates the ability of compounds to inhibit the β-secretase activity of memapsin 2 and memapsin 1. Numerically lower Kᵢ values indicate a higher affinity of the compounds of the invention for memapsin 2 or memapsin 1. The K₁ value is independent of the substrate, and converted from Kᵢ apparent.

Kᵢ apparent is determined in the presence of substrate according to established techniques (see, for example, Bieth, J., Bayer-Symposium V: Proteinase Inhibitors, pp. 463-469, Springer-Verlag, Berlin (1994)).

Vi/Vo depicts the ratio of initial cleavage velocities of the substrate FS-2 (Ermolieff, et al., Biochemistry 40:12450-12456 (2000)) by memapsin 1 or memapsin 2 in the absence (Vo) or presence (Vi) of a compound of the invention. A Vi/Vo value of 1.0 indicates that a compound of the invention does not inhibit the β-secretase activity of the enzyme memapsin 1 or memapsin 2. A Vi/Vo value less than 1.0 indicates that a compound of the invention inhibits β-secretase activity of the enzyme memapsin 1 or memapsin 2. The Vi/Vo values depicted in Table 1 were determined at conditions under which the enzyme and inhibitor concentrations were equal (e.g., about 80 nM, 100 nM).

TABLE 1

| Compound | Structure | | | | |
|---|---|---|---|---|---|
| MMI-001 | 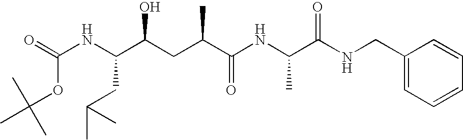 | | | | |
| MMI-002 | 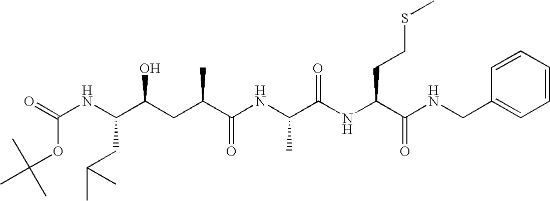 | | | | |
| MMI-003 | 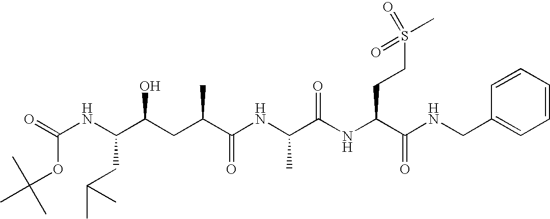 | | | | |
| MMI-004 | 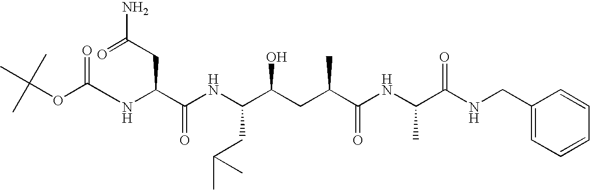 | | | | |
| MMI-005 | 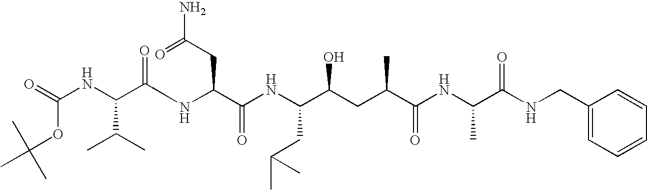 | | | | |
| MMI-006 | 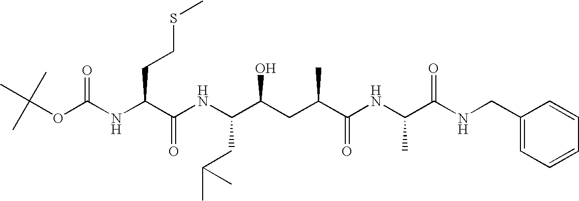 | | | | |
| MMI-007 | 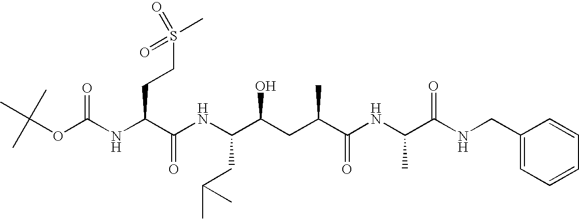 | | | | |
| MMI-008 | 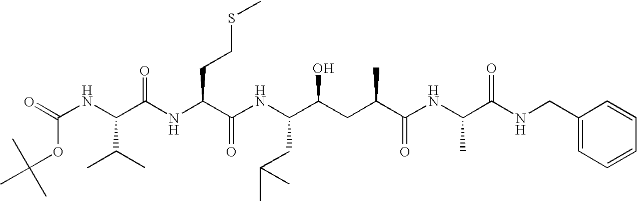 | | | | |
| MMI-009 | 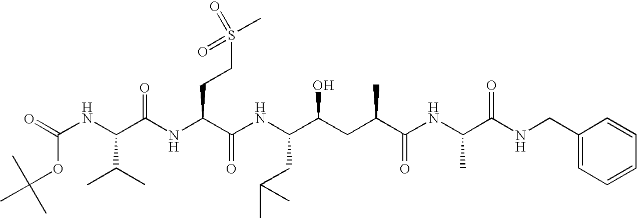 | | | | |
| MMI-010 | 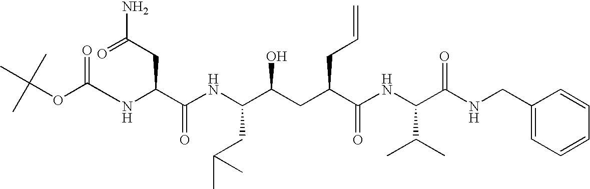 | | | | |
| MMI-011 | 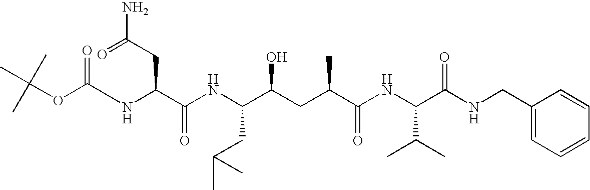 | | | | |
| MMI-012 | 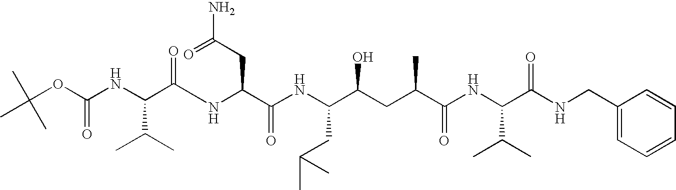 | | | | |
| MMI-013 | 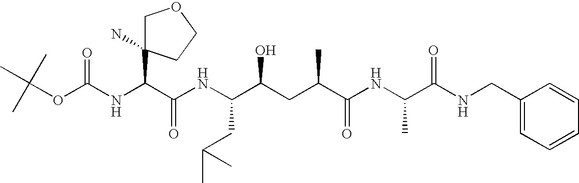 | | | | |
| MMI-014 | 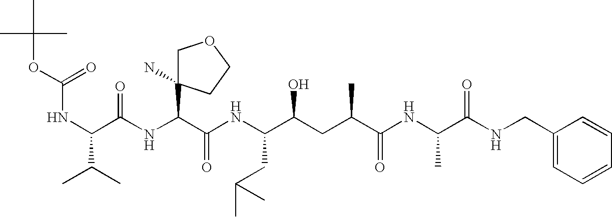 | | | | |
| MMI-015 | 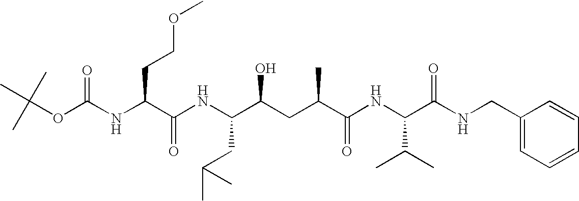 | | | | |
| MMI-016 | 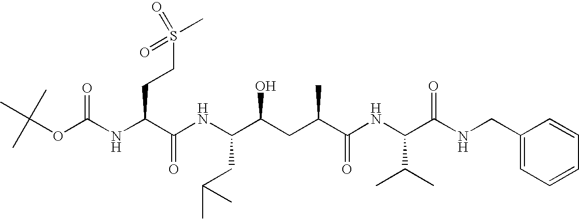 | | | | |
| MMI-017 | 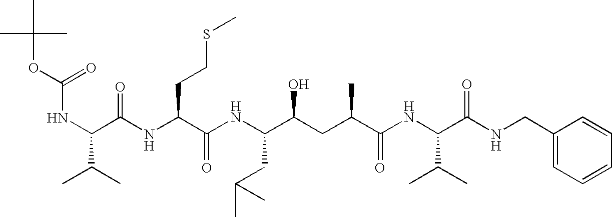 | | | | |
| MMI-018 | 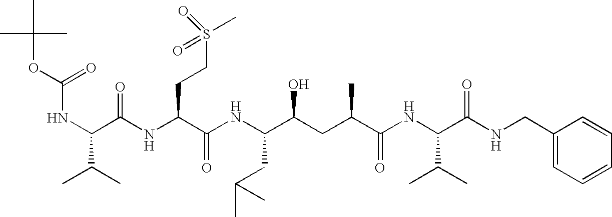 | | | | |
| MMI-019 | 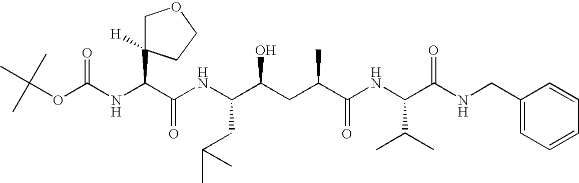 | | | | |
| MMI-020 | 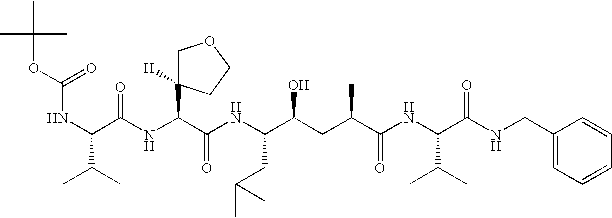 | | | | |
| MMI-021 | 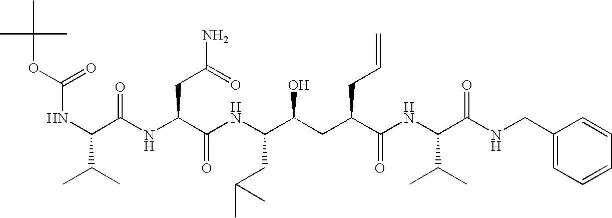 | | | | |
| MMI-022 | 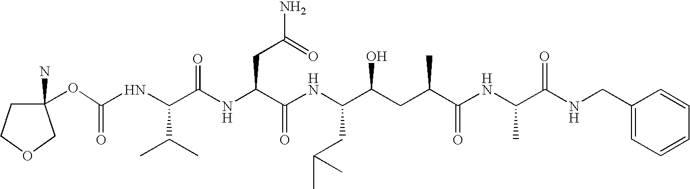 | | | | |
| MMI-023 | 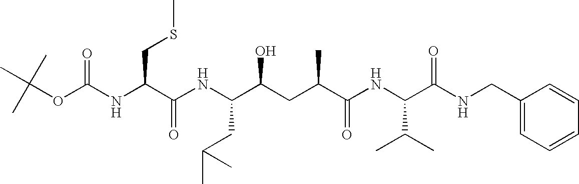 | | | | |
| MMI-024 | 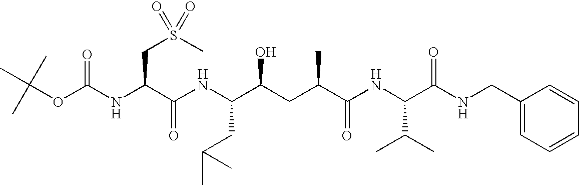 | | | | |
| MMI-025 | 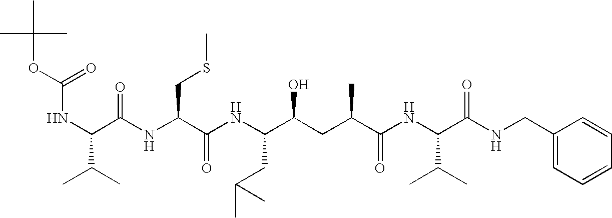 | | | | |
| MMI-026 | 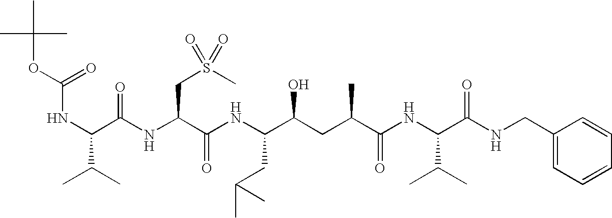 | | | | |
| MMI-027 | 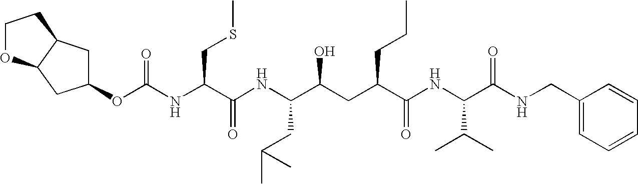 | | | | |
| MMI-028 | 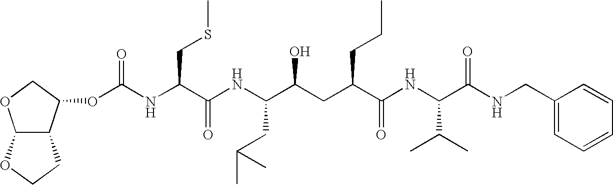 | | | | |
| MMI-029 | 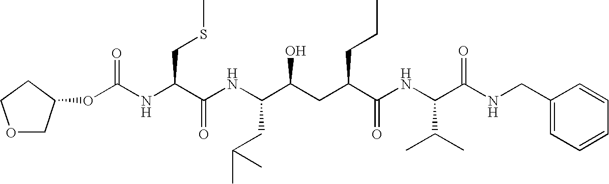 | | | | |
| MMI-030 | 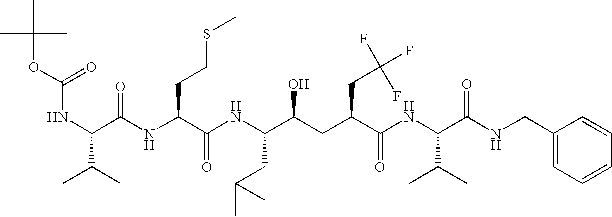 | | | | |
| MMI-031 | 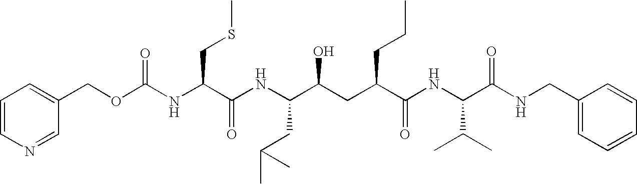 | | | | |
| MMI-032 | 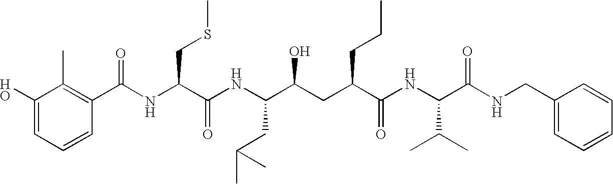 | | | | |
| MMI-033 | 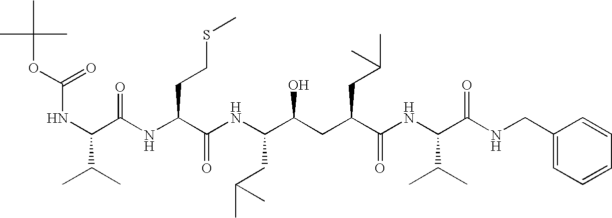 | | | | |
| MMI-034 | 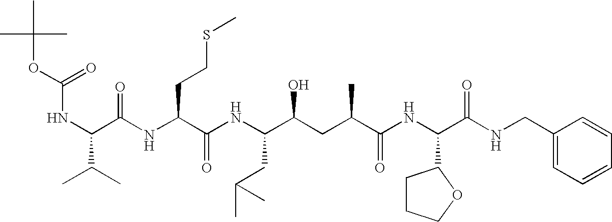 | | | | |
| MMI-035 | 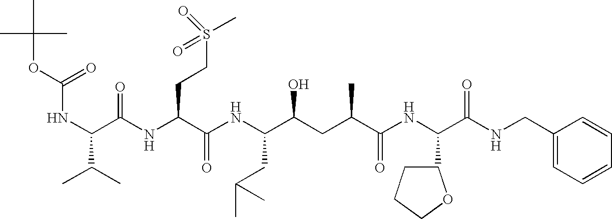 | | | | |
| MMI-036 | 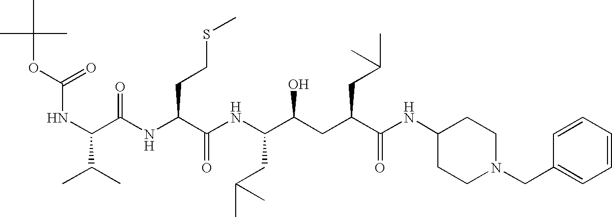 | | | | |
| MMI-037 | 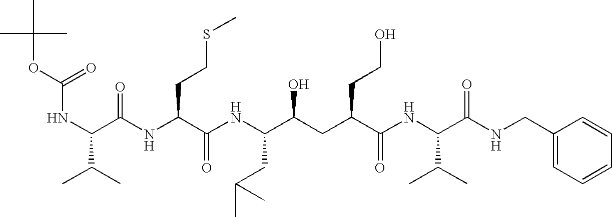 | | | | |
| MMI-038 | 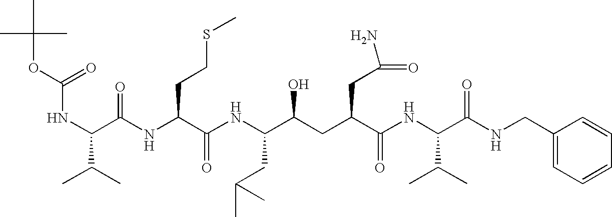 | | | | |
| MMI-039 | 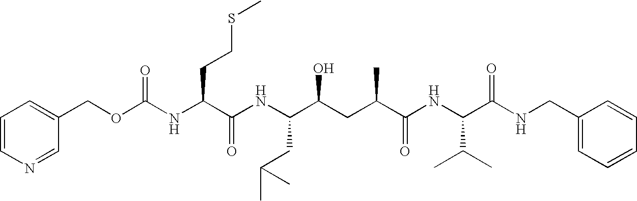 | | | | |
| MMI-040 | 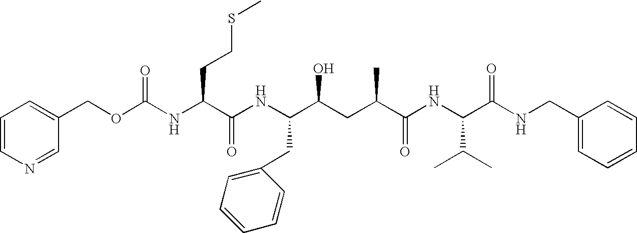 | | | | |
| MMI-041 | 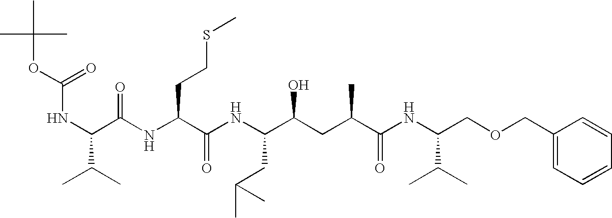 | | | | |
| MMI-042 | 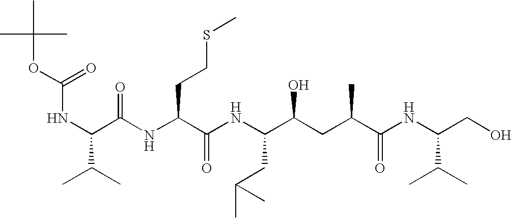 | | | | |
| MMI-043 | 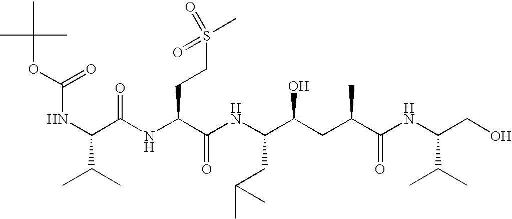 | | | | |
| MMI-044 | 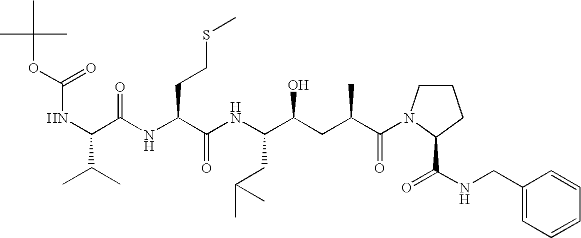 | | | | |
| MMI-045 | 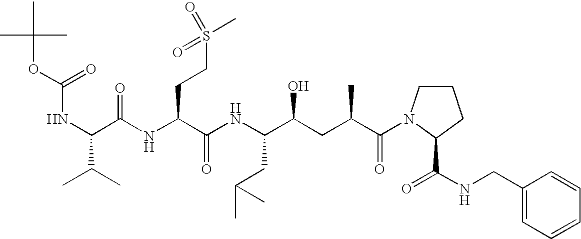 | | | | |
| MMI-046 | 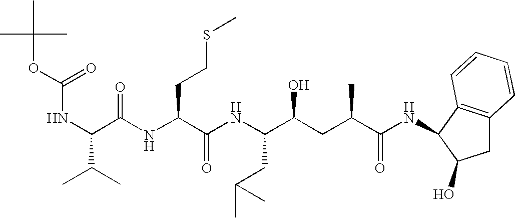 | | | | |
| MMI-047 | 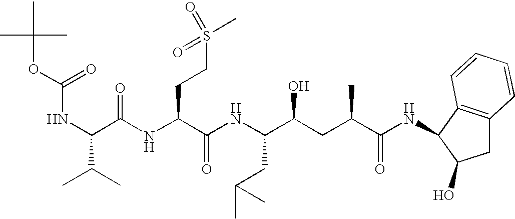 | | | | |
| MMI-048 | 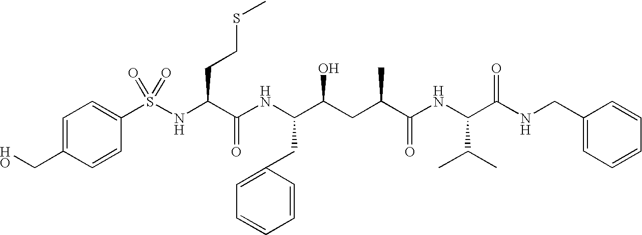 | | | | |
| MMI-049 | 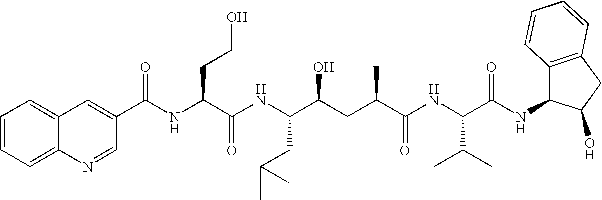 | | | | |
| MMI-050 | 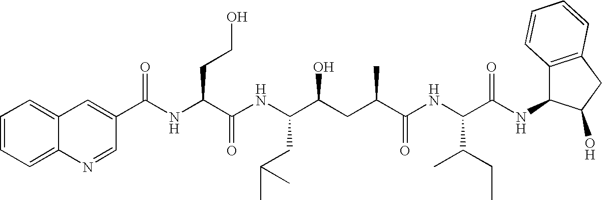 | | | | |
| MMI-051 | 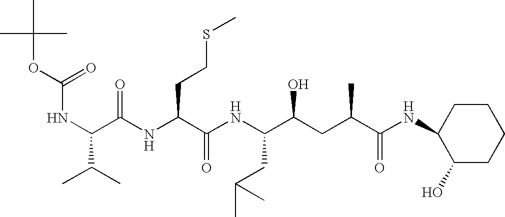 | | | | |
| MMI-052 | 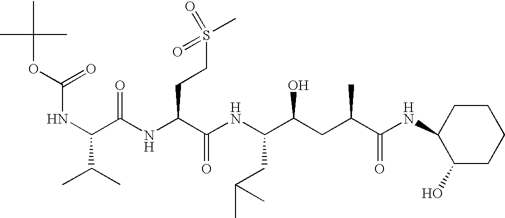 | | | | |
| MMI-053 | 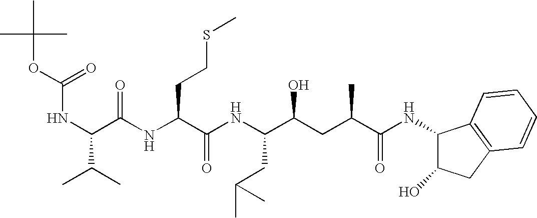 | | | | |
| MMI-054 | 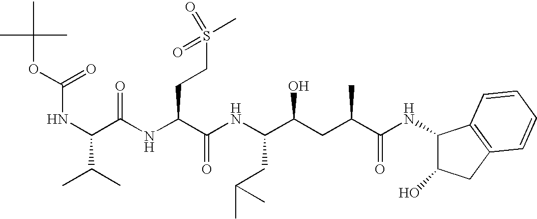 | | | | |
| MMI-055 | 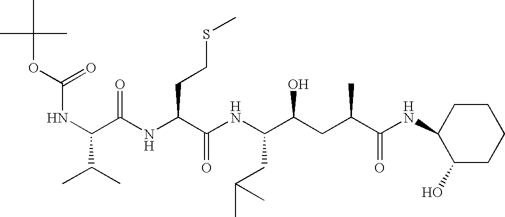 | | | | |
| MMI-056 | 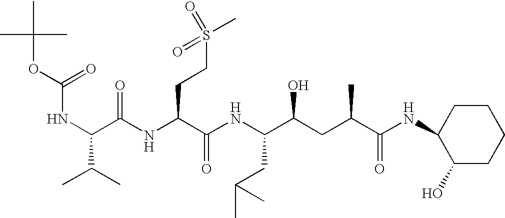 | | | | |
| MMI-057 | 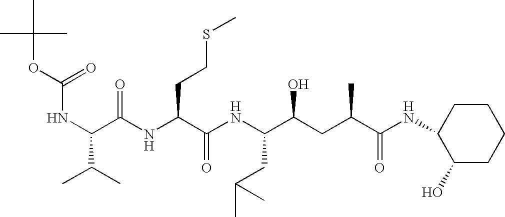 | | | | |
| MMI-058 | 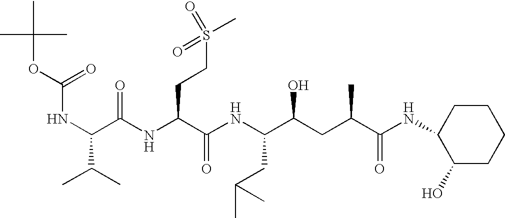 | | | | |
| MMI-059 | 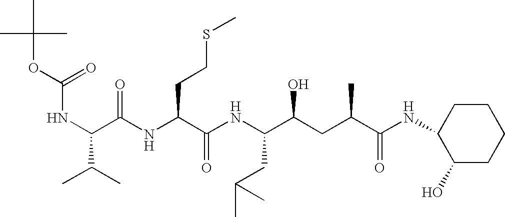 | | | | |
| MMI-060 | 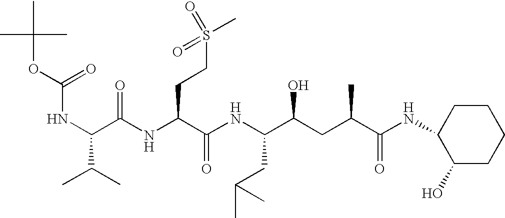 | | | | |
| MMI-061 | 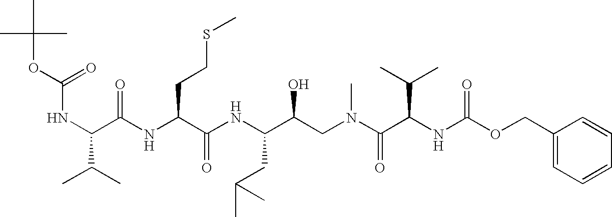 | | | | |
| MMI-062 | 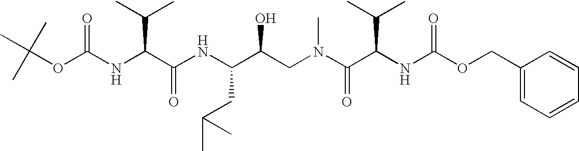 | | | | |
| MMI-063 | 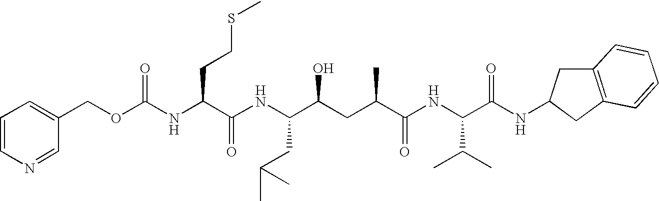 | | | | |
| MMI-064 | 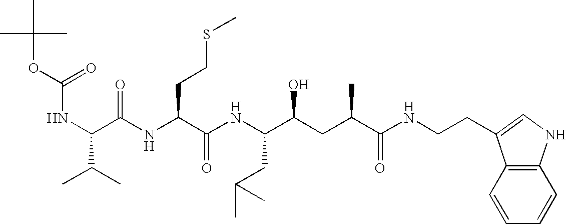 | | | | |
| MMI-065 | 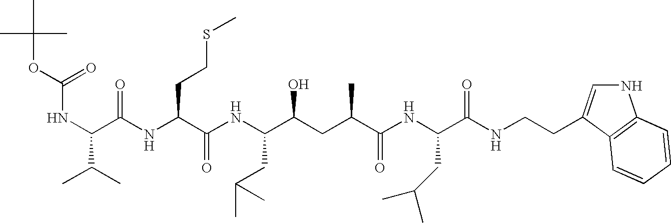 | | | | |
| MMI-066 | 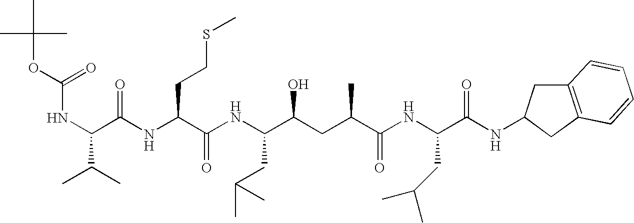 | | | | |
| MMI-067 | 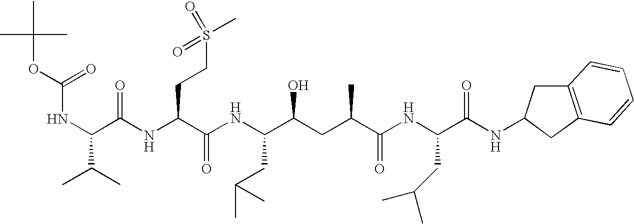 | | | | |
| MMI-068 | 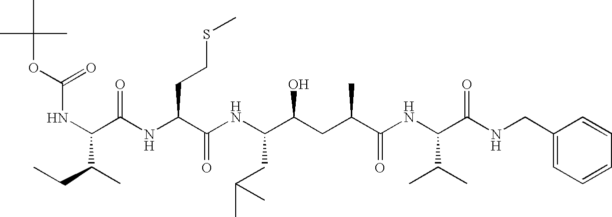 | | | | |
| MMI-069 | 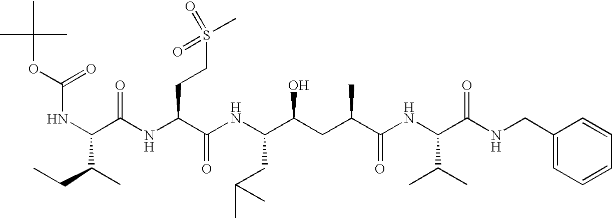 | | | | |
| MMI-070 | 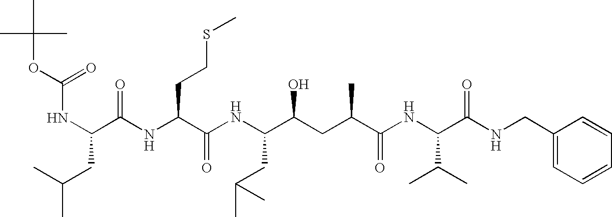 | | | | |
| MMI-071 | 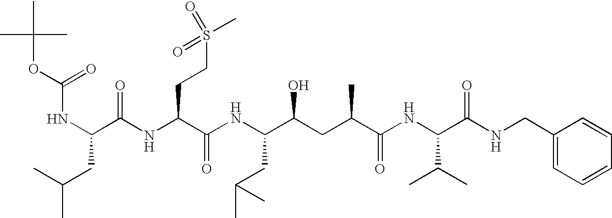 | | | | |
| MMI-072 | 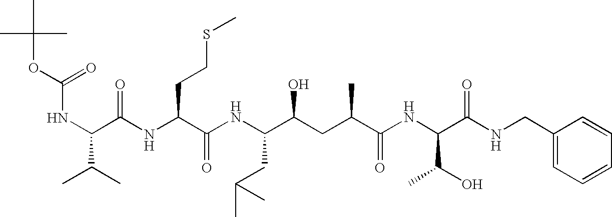 | | | | |
| MMI-073 | 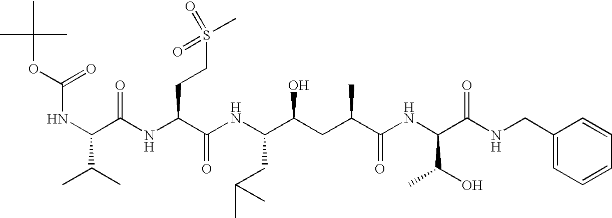 | | | | |
| MMI-074 | 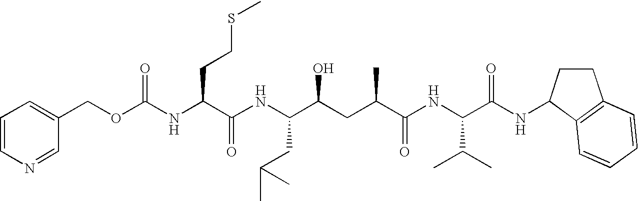 | | | | |
| MMI-075 | 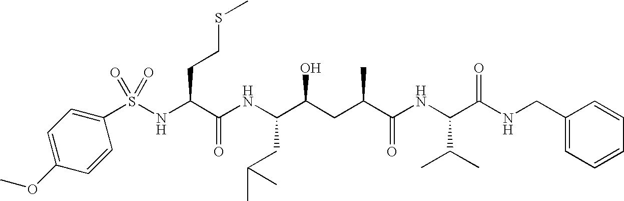 | | | | |
| MMI-076 | 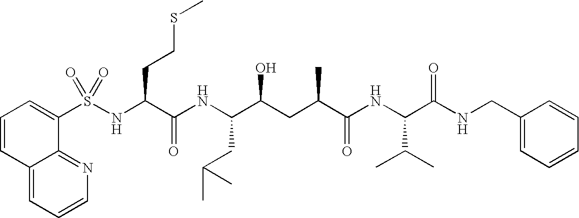 | | | | |
| MMI-077 | 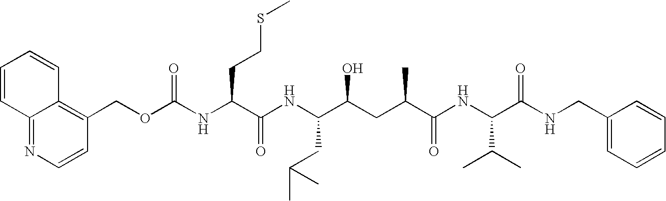 | | | | |
| MMI-078 | 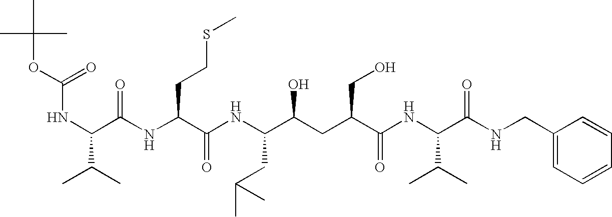 | | | | |
| MMI-079 | 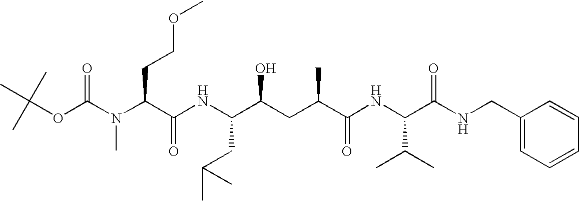 | | | | |
| MMI-080 | 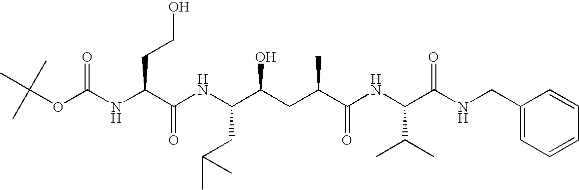 | | | | |
| MMI-081 | 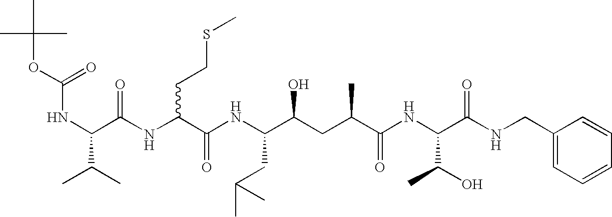 | | | | |
| MMI-082 | 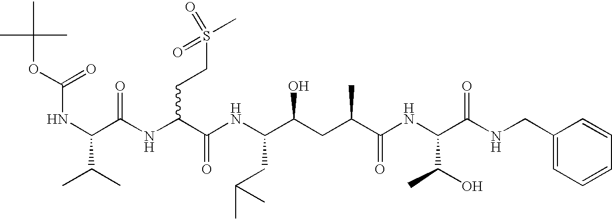 | | | | |
| MMI-083 | 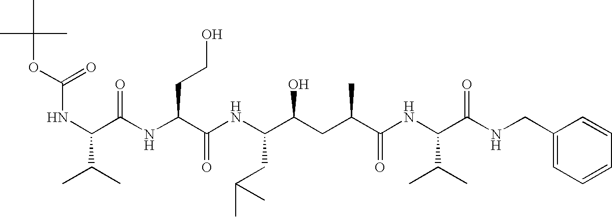 | | | | |
| MMI-084 | 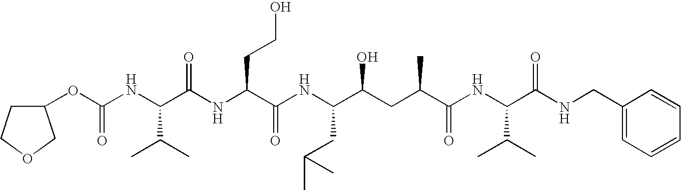 | | | | |
| MMI-085 | 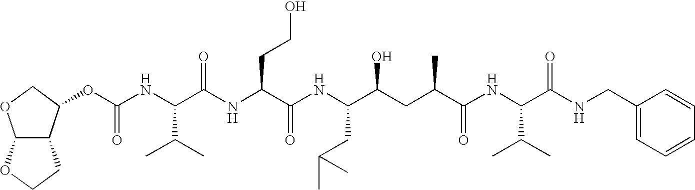 | | | | |
| MMI-086 | 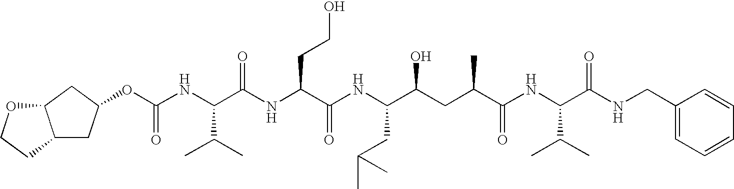 | | | | |
| MMI-087 | 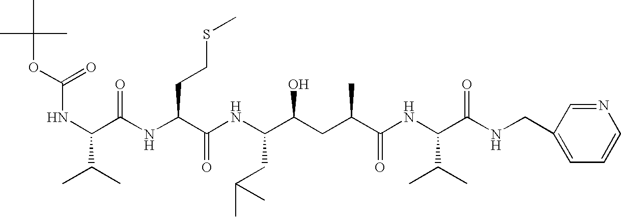 | | | | |
| MMI-088 | 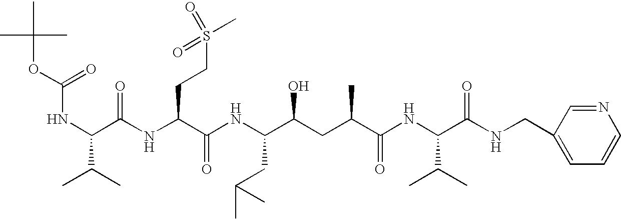 | | | | |
| MMI-089 | 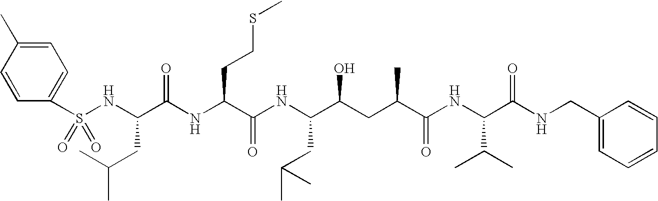 | | | | |
| MMI-090 | 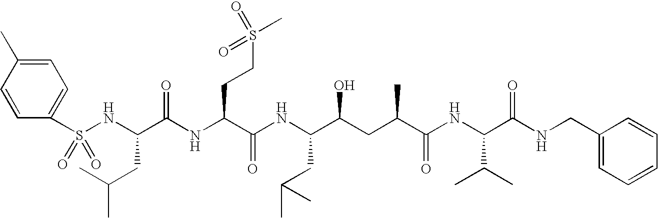 | | | | |
| MMI-091 | 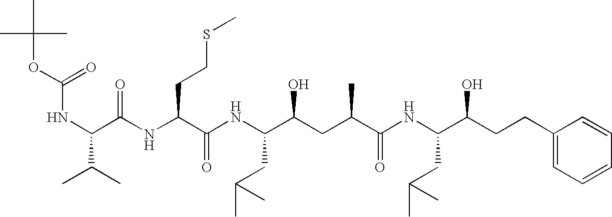 | | | | |
| MMI-092 | 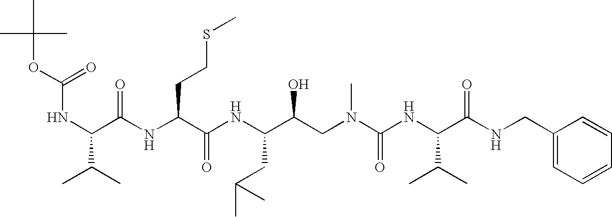 | | | | |
| MMI-093 | 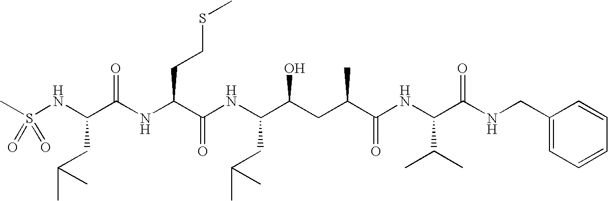 | | | | |
| MMI-094 | 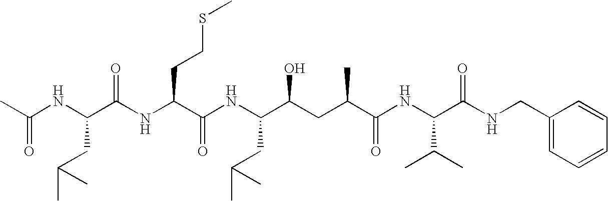 | | | | |
| MMI-095 | 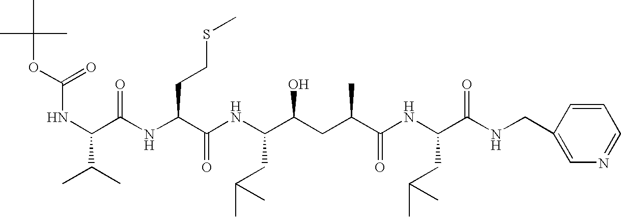 | | | | |
| MMI-096 | 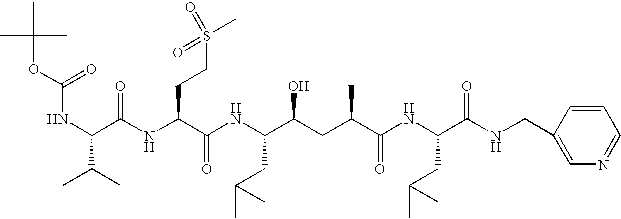 | | | | |
| MMI-097 | 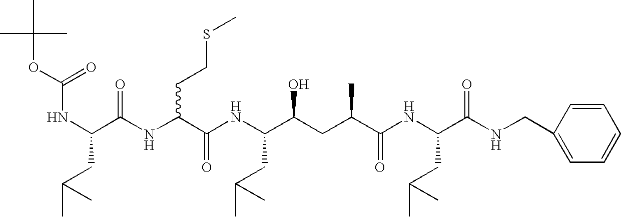 | | | | |
| MMI-098 | 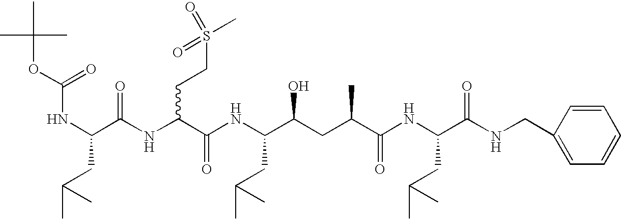 | | | | |
| MMI-099 | 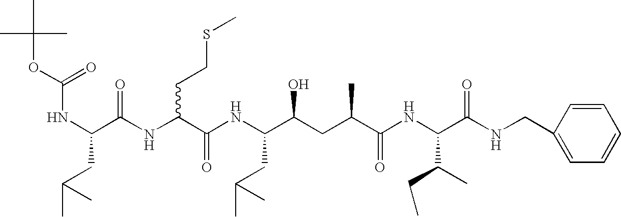 | | | | |
| MMI-100 | 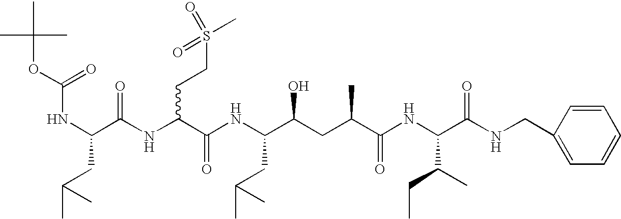 | | | | |
| MMI-101 | 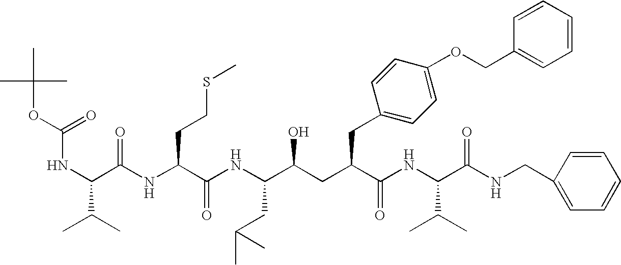 | | | | |
| MMI-102 | 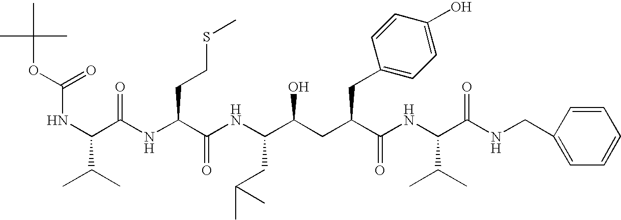 | | | | |
| MMI-103 | 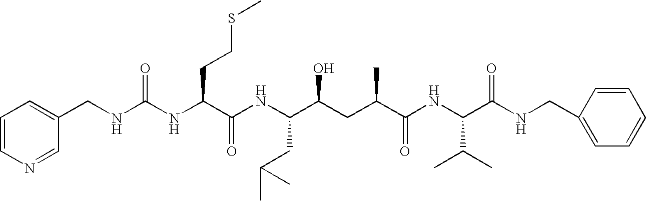 | | | | |
| MMI-104 | 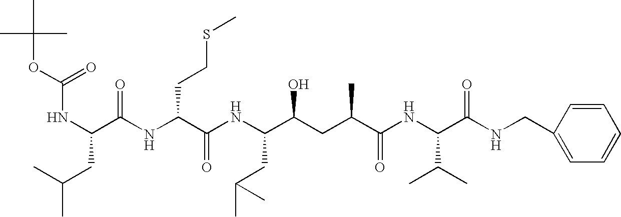 | | | | |
| MMI-105 | 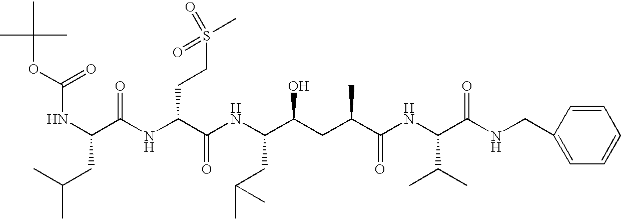 | | | | |
| MMI-106 | 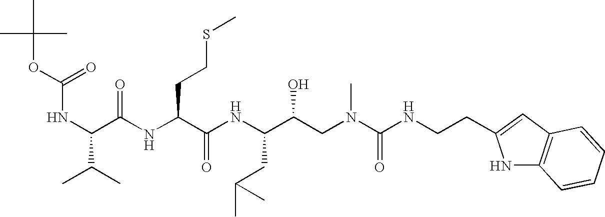 | | | | |
| MMI-107 | 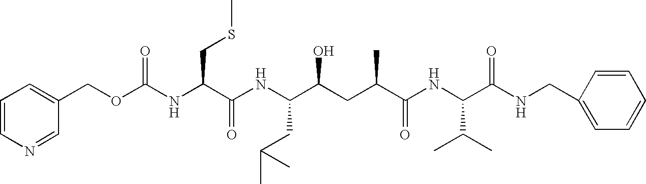 | | | | |
| MMI-108 | 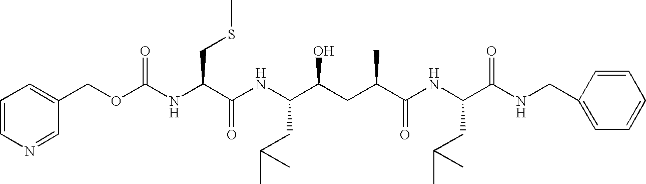 | | | | |
| MMI-109 | 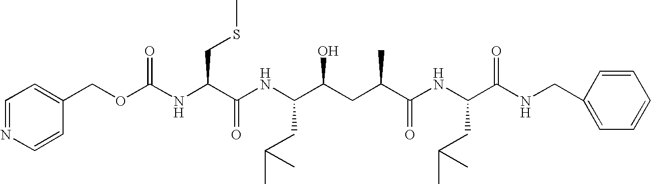 | | | | |
| MMI-110 | 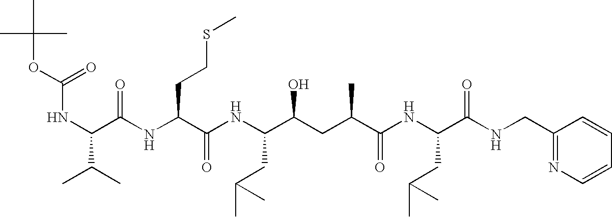 | | | | |
| MMI-111 | 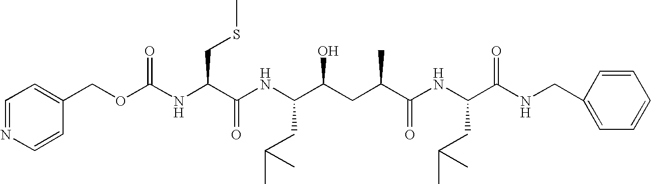 | | | | |
| MMI-112 | 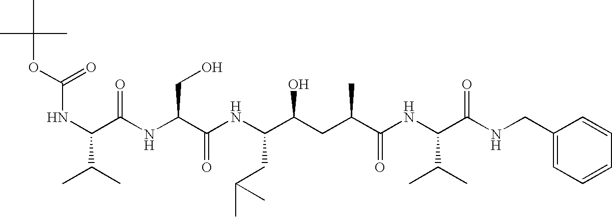 | | | | |
| MMI-113 | 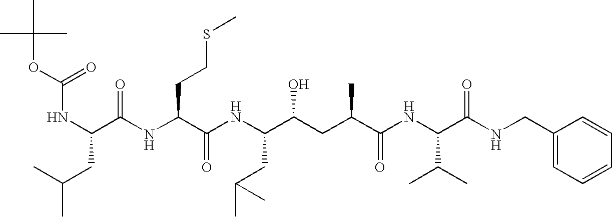 | | | | |
| MMI-114 | 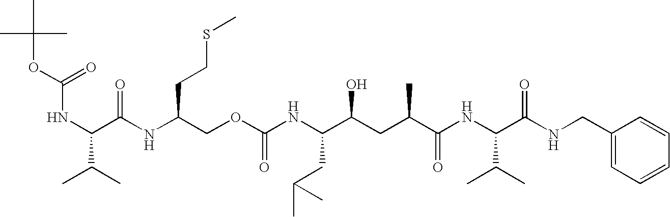 | | | | |
| MMI-115 | 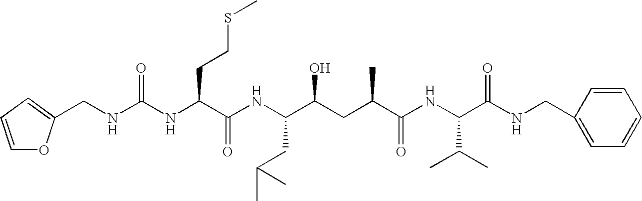 | | | | |
| MMI-116 | 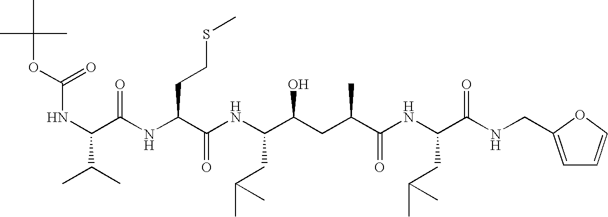 | | | | |
| MMI-117 | 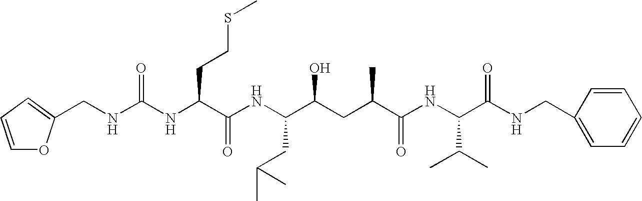 | | | | |
| MMI-118 | 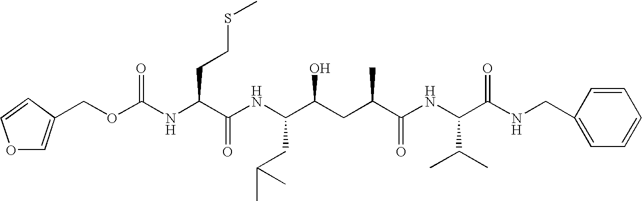 | | | | |
| MMI-119 | 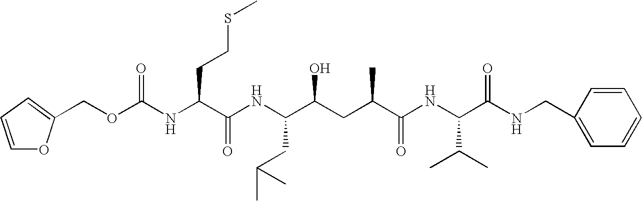 | | | | |
| MMI-120 | 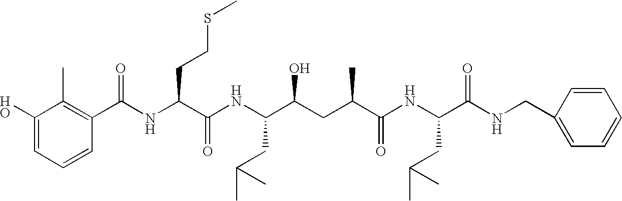 | | | | |
| MMI-121 | 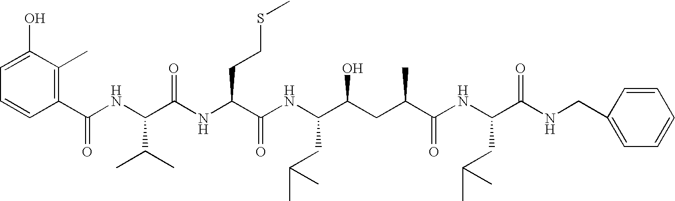 | | | | |
| MMI-122 | 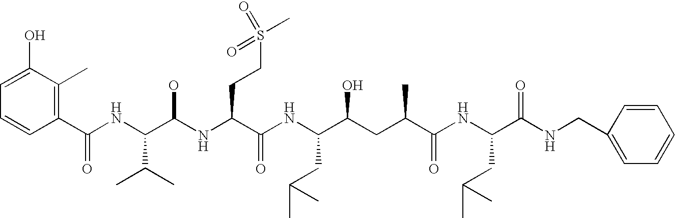 | | | | |
| MMI-123 | 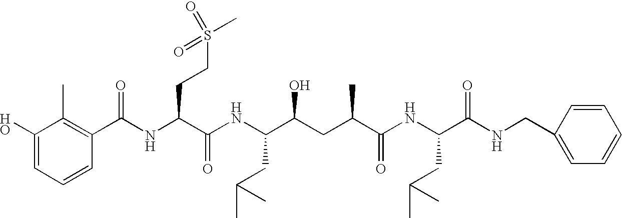 | | | | |
| MMI-124 | 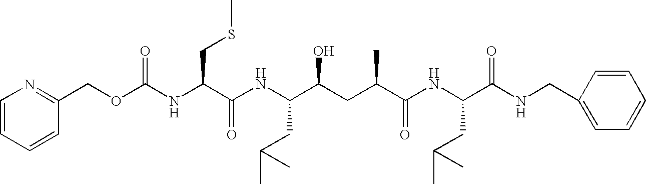 | | | | |
| MMI-125 | 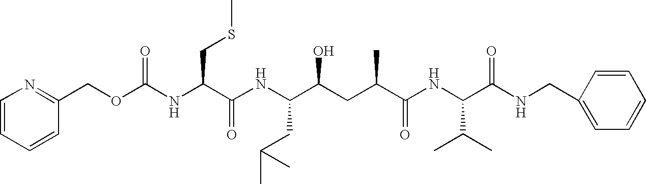 | | | | |
| MMI-126 | 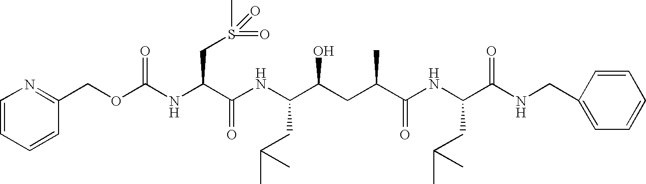 | | | | |
| MMI-127 | 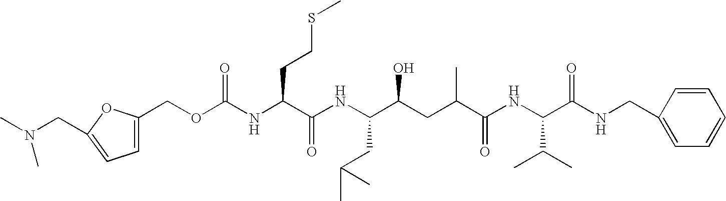 | | | | |
| MMI-128 | 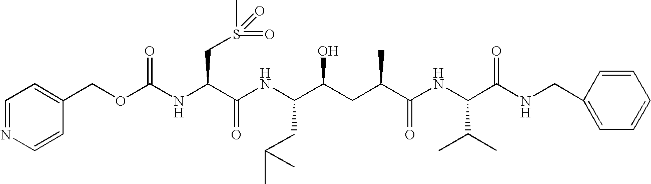 | | | | |
| MMI-129 | 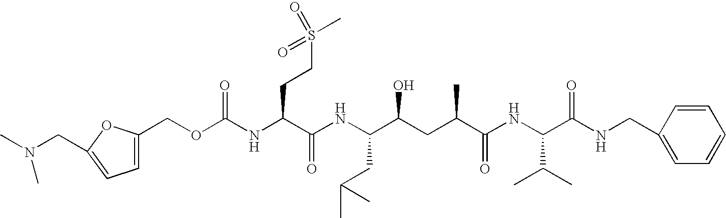 | | | | |
| MMI-130 | 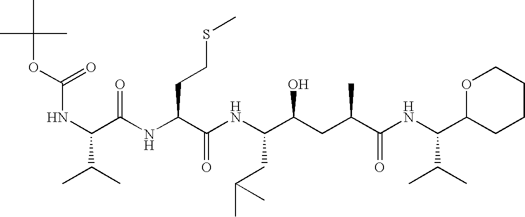 | | | | |
| MMI-131 | 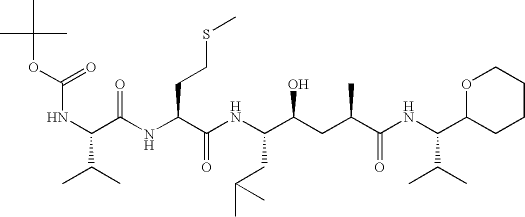 | | | | |
| MMI-132 | 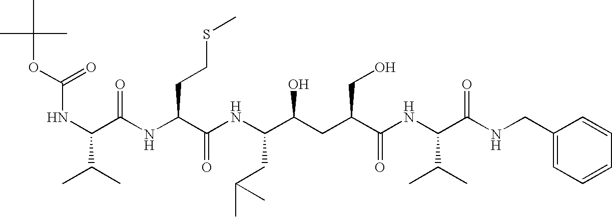 | | | | |
| MMI-133 | 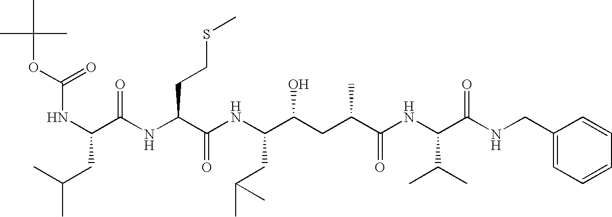 | | | | |
| MMI-134 | 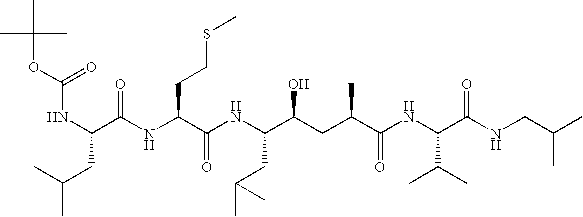 | | | | |
| MMI-135 | 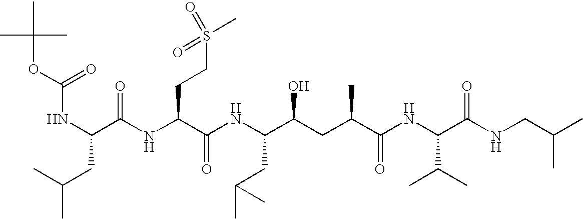 | | | | |
| MMI-136 | 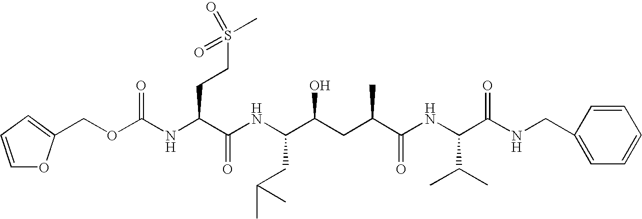 | | | | |
| MMI-137 | 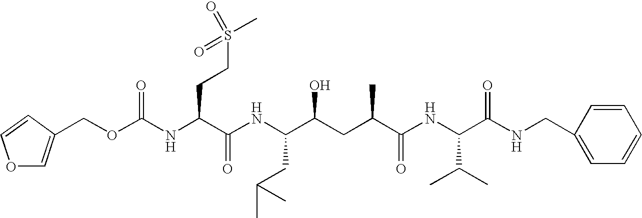 | | | | |
| MMI-138 | 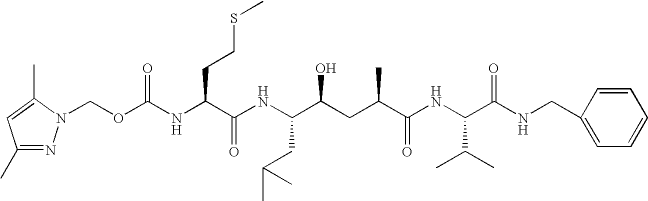 | | | | |
| MMI-139 | 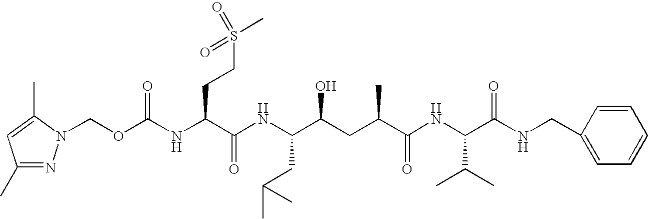 | | | | |
| MMI-140 | 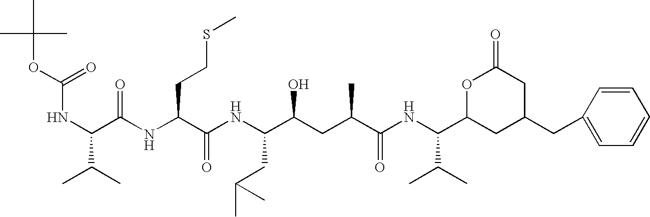 | | | | |
| MMI-141 | 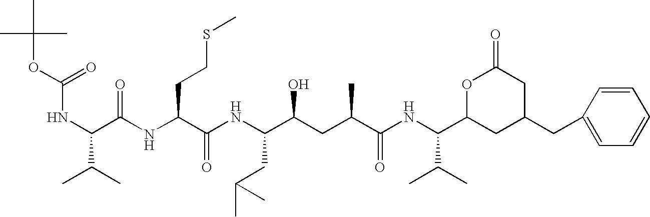 | | | | |
| MMI-142 | 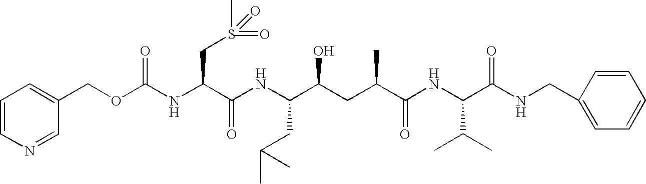 | | | | |
| MMI-143 | 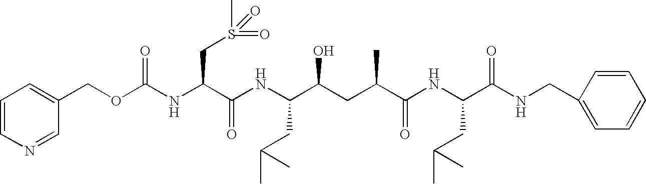 | | | | |
| MMI-144 | 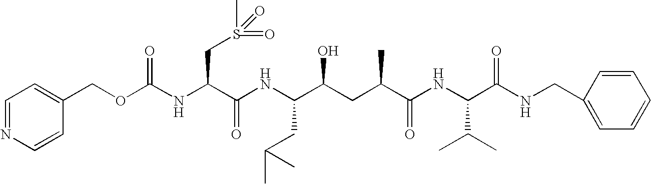 | | | | |
| MMI-145 | 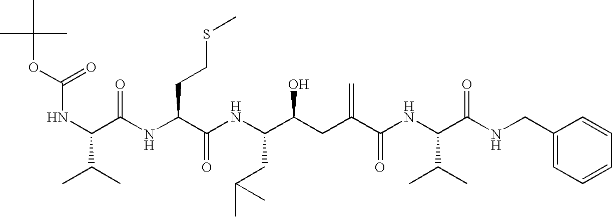 | | | | |
| MMI-146-A | 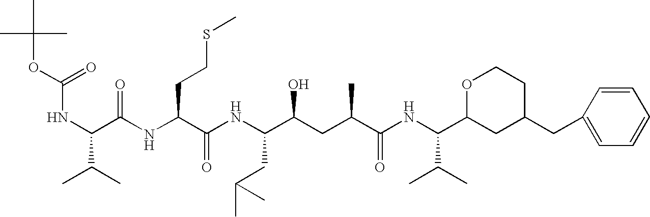 | | | | |
| MMI-146-S | 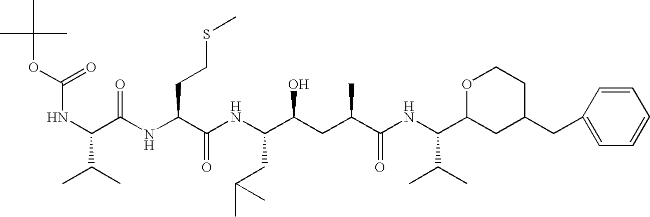 | | | | |
| MMI-147 | 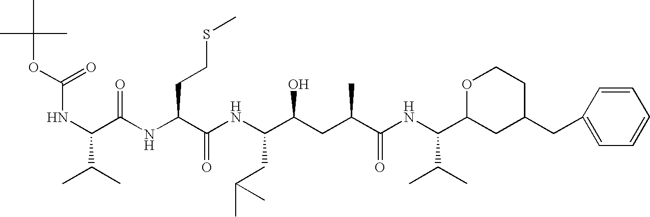 | | | | |
| MMI-148 | 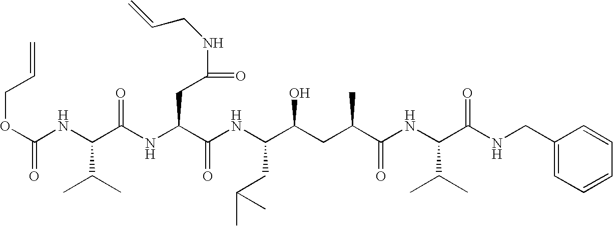 | | | | |
| MMI-149 | 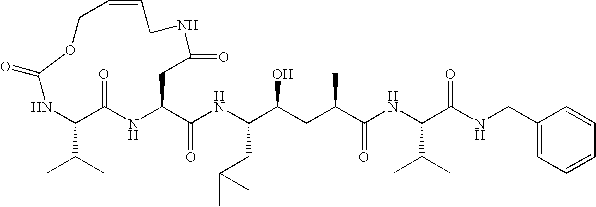 | | | | |
| MMI-150 | 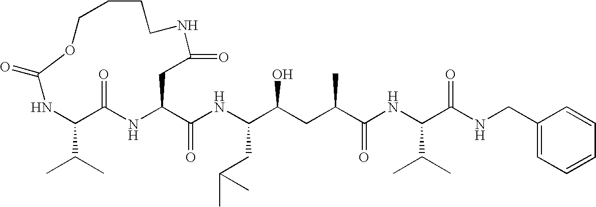 | | | | |
| MMI-151 | 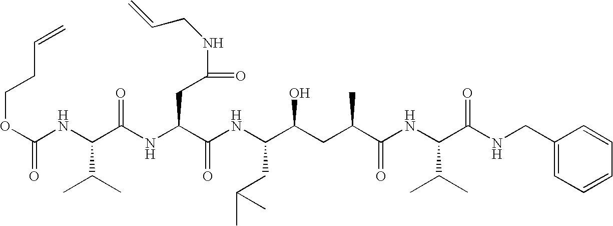 | | | | |
| MMI-152 | 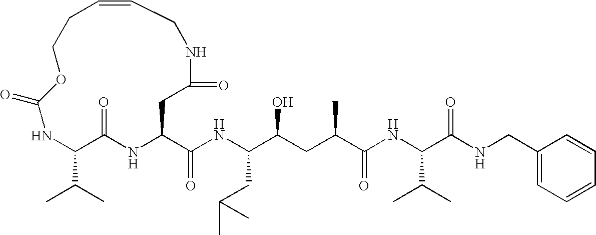 | | | | |
| MMI-153 | 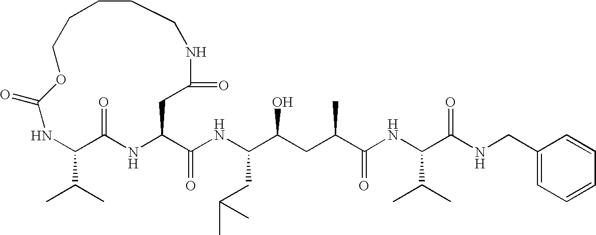 | | | | |
| MMI-154 | 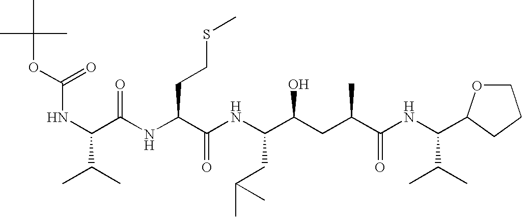 | | | | |
| MMI-155 | 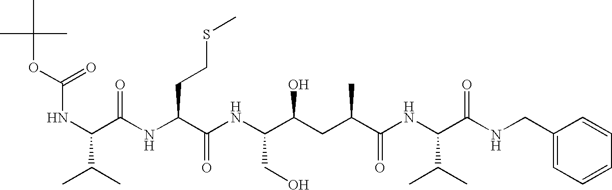 | | | | |
| MMI-156 | 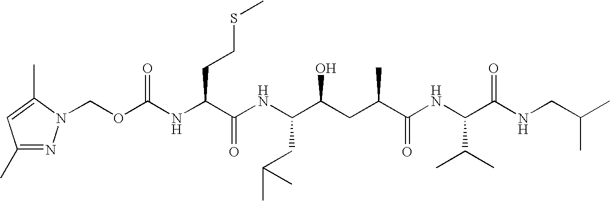 | | | | |
| MMI-157 | 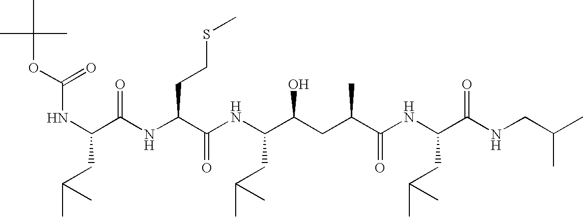 | | | | |
| MMI-158 | 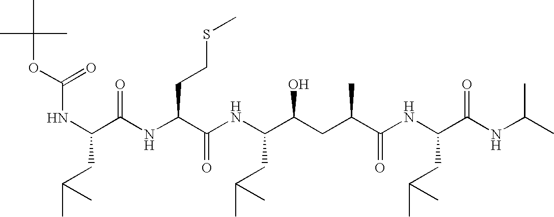 | | | | |
| MMI-159 | 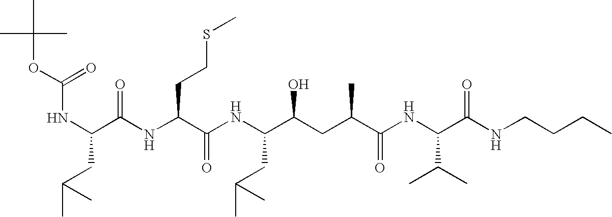 | | | | |
| MMI-160 | 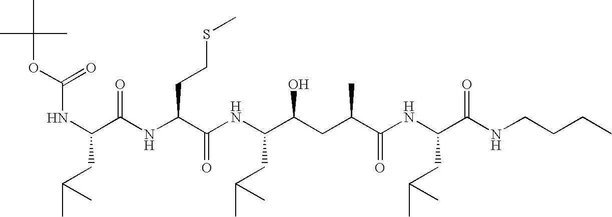 | | | | |
| MMI-161 | 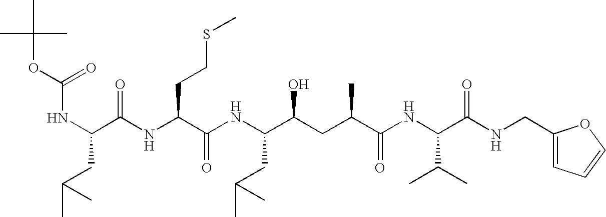 | | | | |
| MMI-162 | 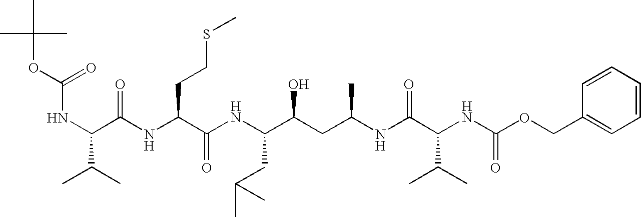 | | | | |
| MMI-163 | 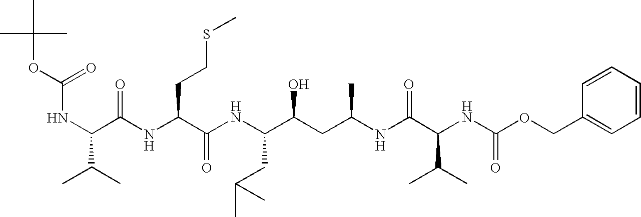 | | | | |
| MMI-164 | 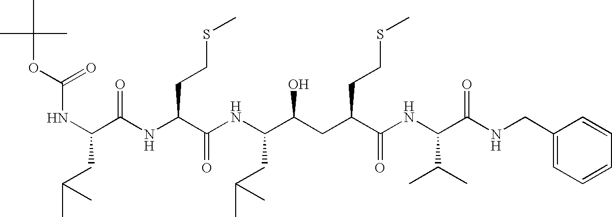 | | | | |
| MMI-165 | 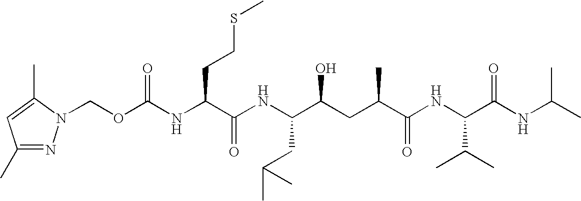 | | | | |
| MMI-166 | 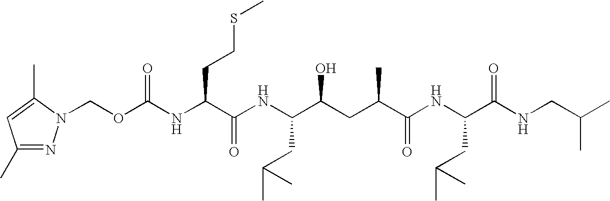 | | | | |
| MMI-167 | 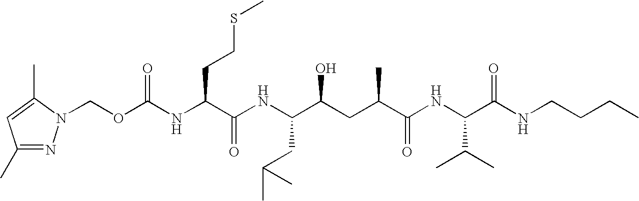 | | | | |
| MMI-168 | 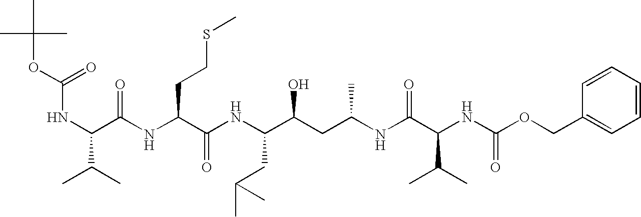 | | | | |
| MMI-169 | 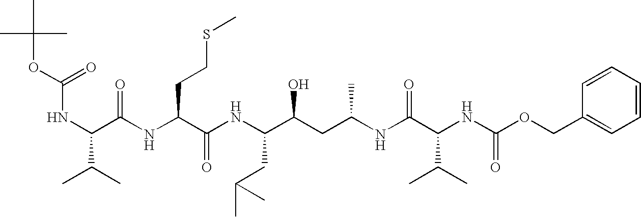 | | | | |
| MMI-170 | 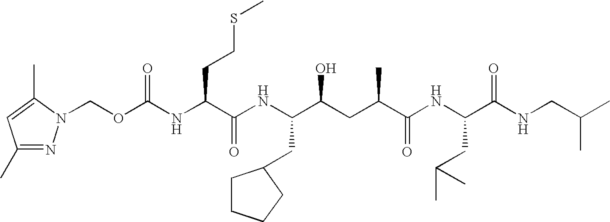 | | | | |
| MMI-171 | 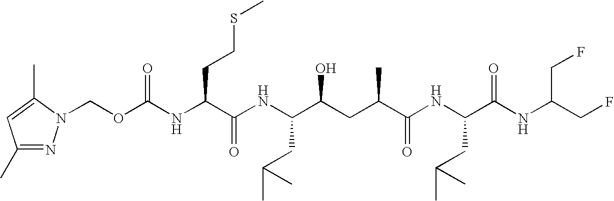 | | | | |
| MMI-172 | 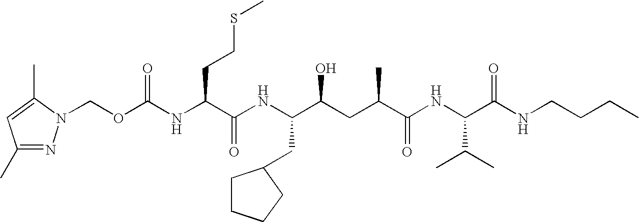 | | | | |
| MMI-173 | 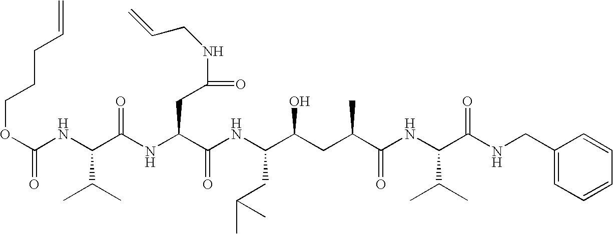 | | | | |
| MMI-174 | 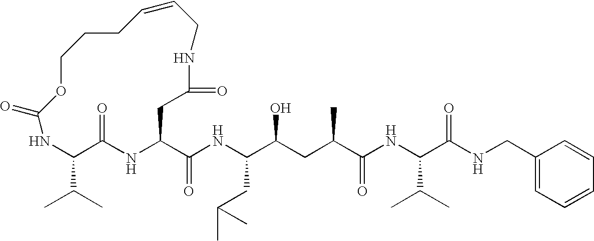 | | | | |
| MMI-175 | 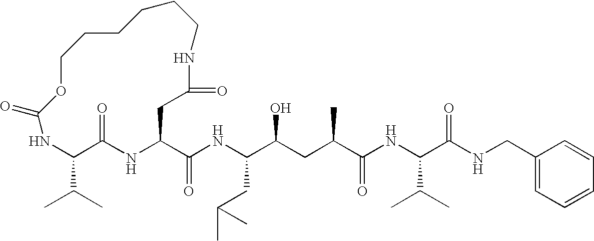 | | | | |
| MMI-176 | 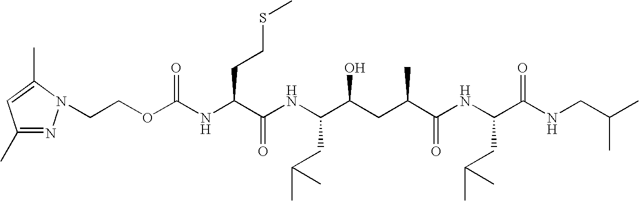 | | | | |
| MMI-177 | 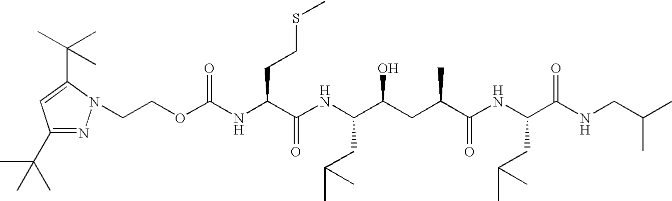 | | | | |
| MMI-178 | 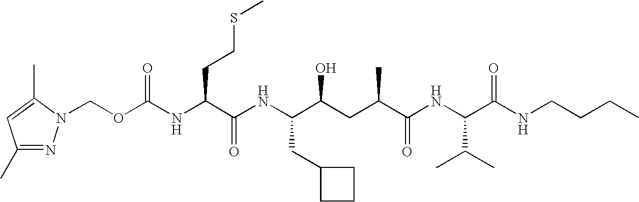 | | | | |
| MMI-179 | 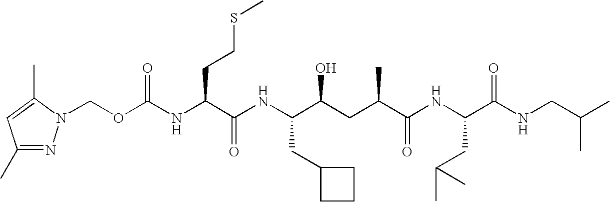 | | | | |
| MMI-180 | 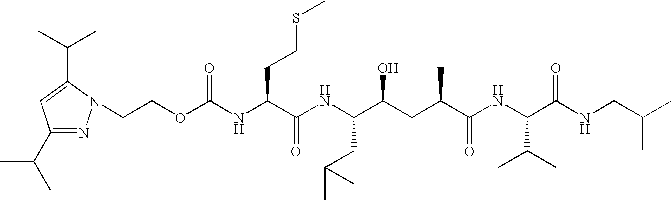 | | | | |
| MMI-181 | 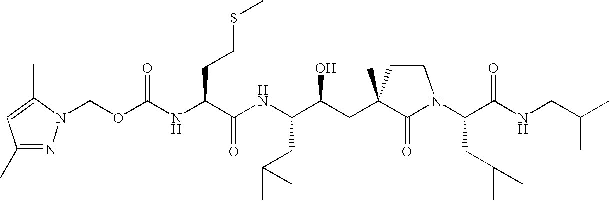 | | | | |
| MMI-182 | 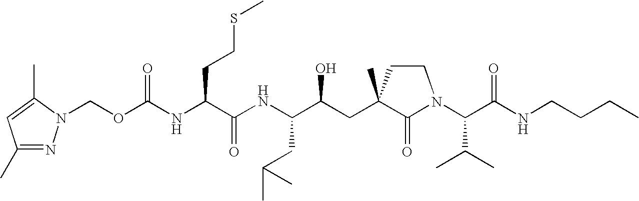 | | | | |
| MMI-183 | 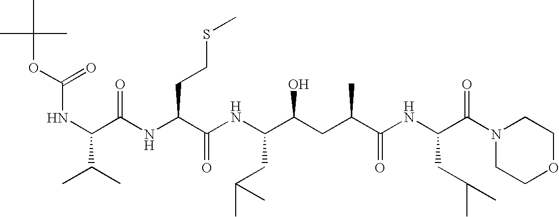 | | | | |
| MMI-184 | 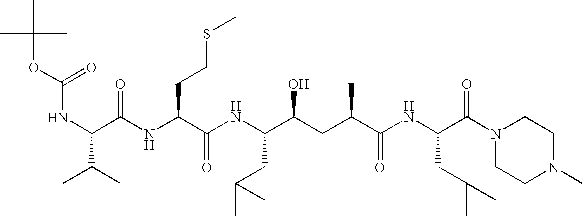 | | | | |
| MMI-185 | 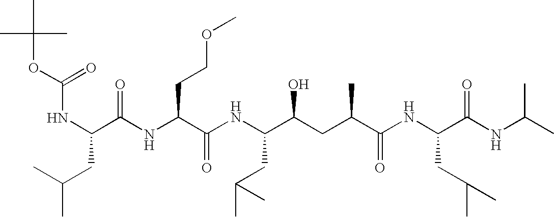 | | | | |
| MMI-186 | 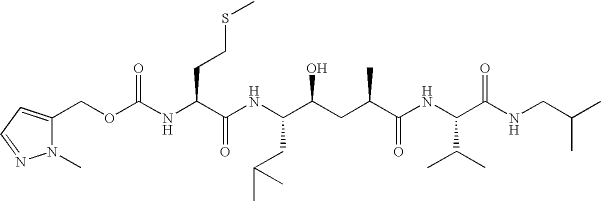 | | | | |
| MMI-187 | 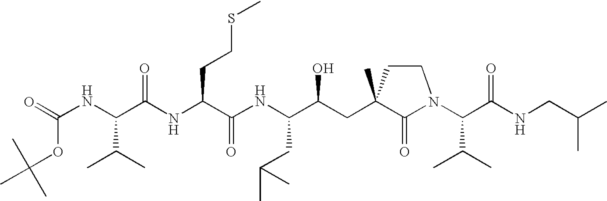 | | | | |
| MMI-188 | 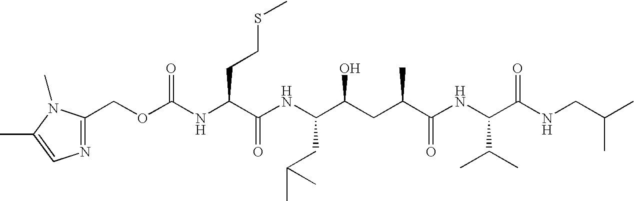 | | | | |
| MMI-189 | 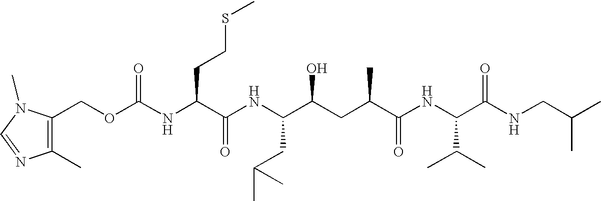 | | | | |
| MMI-190 | 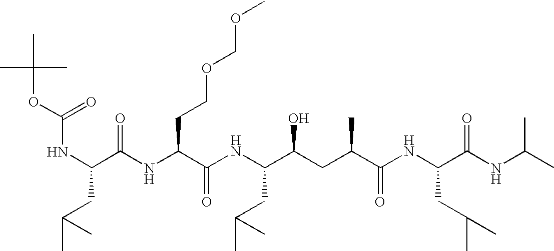 | | | | |
| MMI-191 | 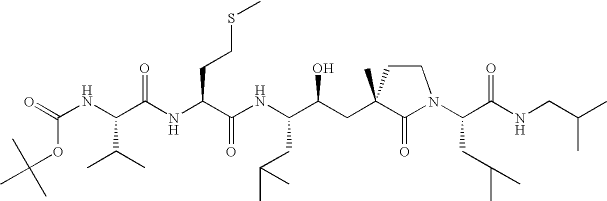 | | | | |
| MMI-192 | 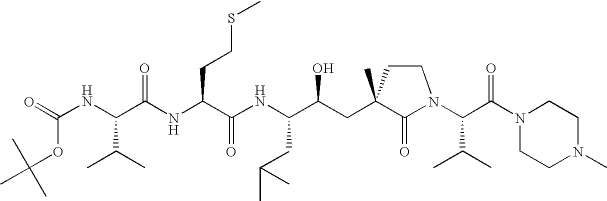 | | | | |
| MMI-193 | 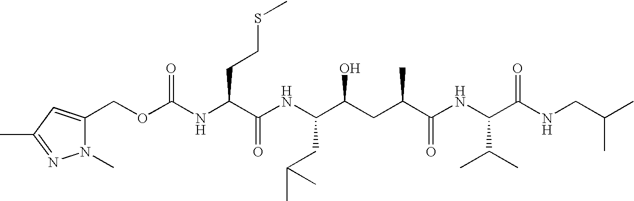 | | | | |
| MMI-194 | 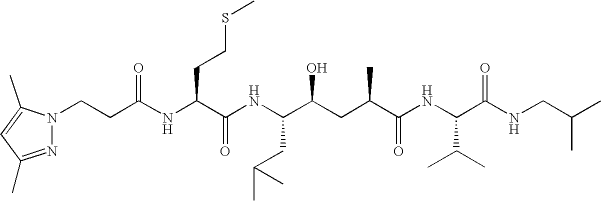 | | | | |
| MMI-195 | 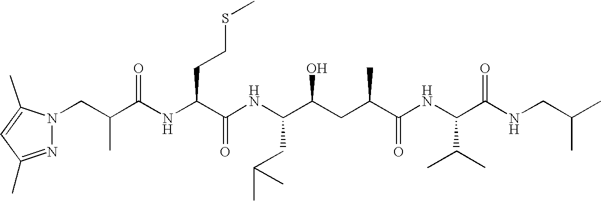 | | | | |
| MMI-196 | 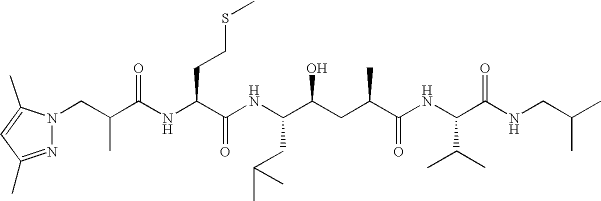 | | | | |
| MMI-197 | 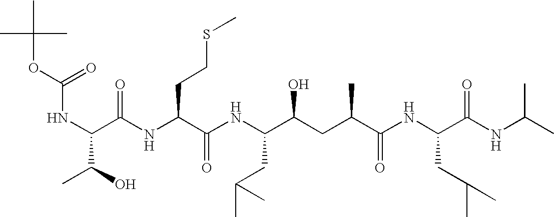 | | | | |
| MMI-198 | 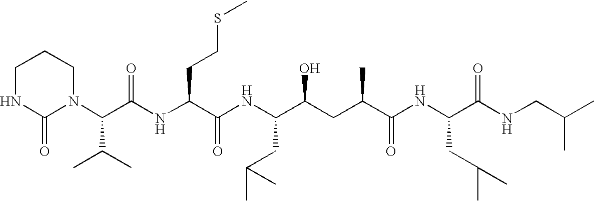 | | | | |
| MMI-199 | 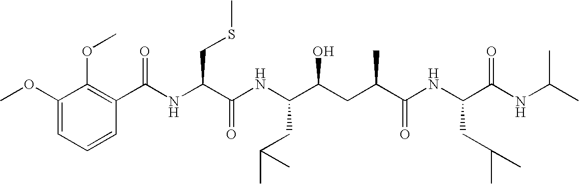 | | | | |
| MMI-200 | 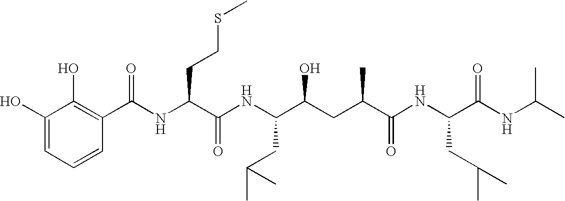 | | | | |
| MMI-201 | 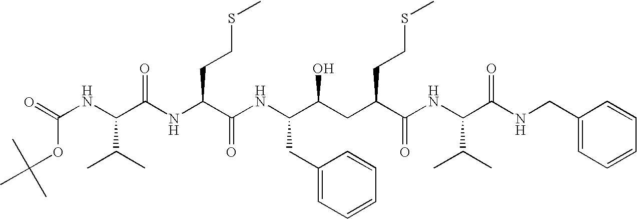 | | | | |
| MMI-202 | 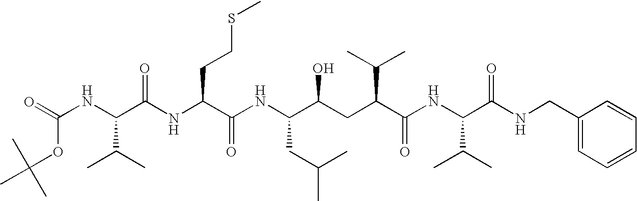 | | | | |
| MMI-203 | 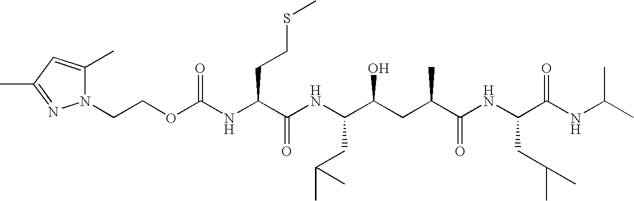 | | | | |
| MMI-204 | 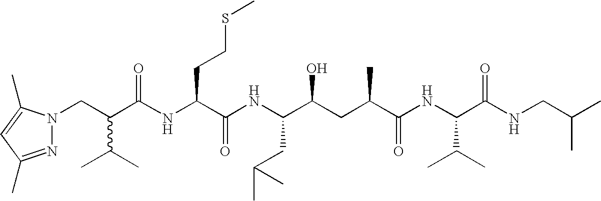 | | | | |
| MMI-205 | 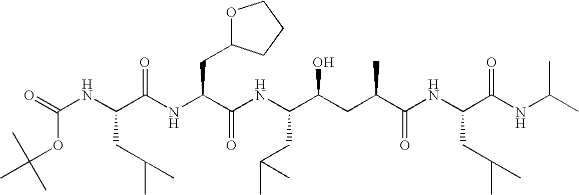 | | | | |
| MMI-206 | 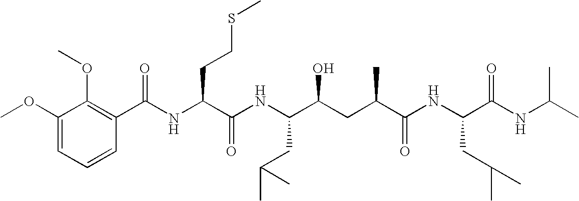 | | | | |
| MMI-207 | 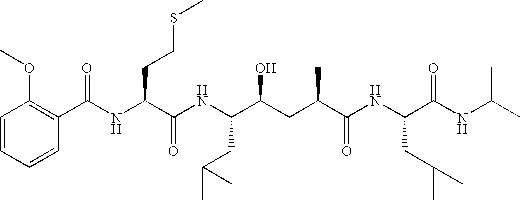 | | | | |
| MMI-208 | 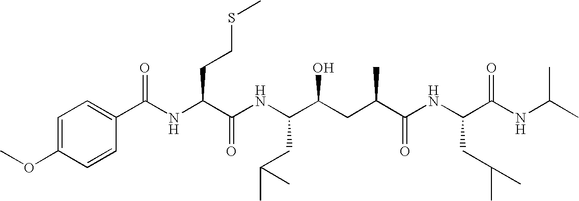 | | | | |
| MMI-209 | 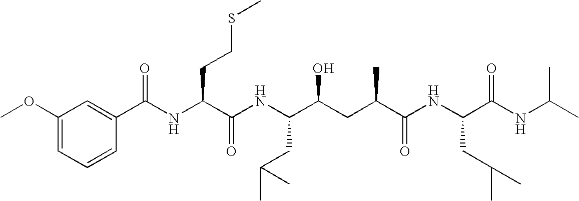 | | | | |
| MMI-210 | 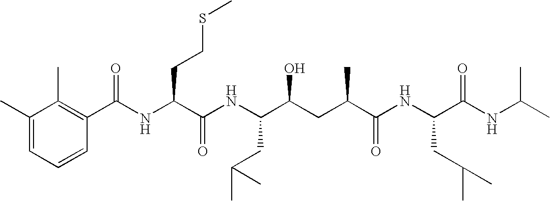 | | | | |
| MMI-211 | 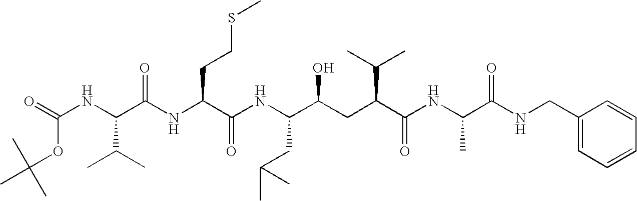 | | | | |
| MMI-212 | 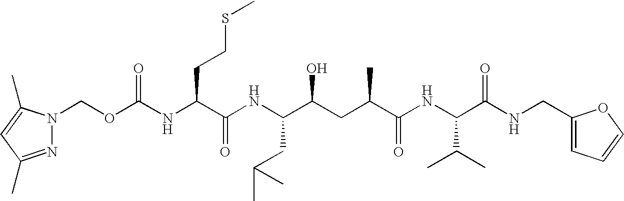 | | | | |
| MMI-213 | 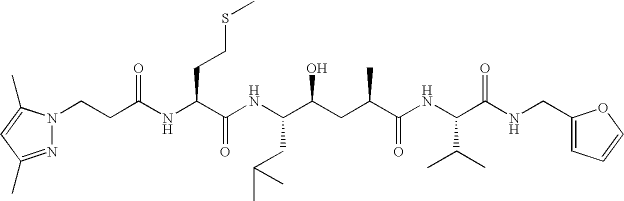 | | | | |
| MMI-214 | 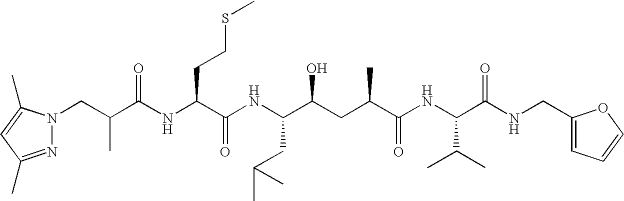 | | | | |
| MMI-215 | 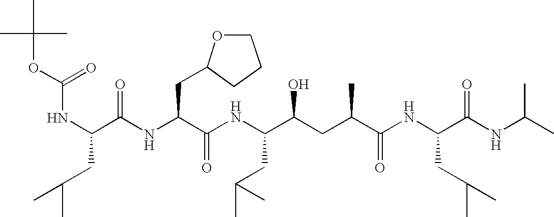 | | | | |
| MMI-216 | 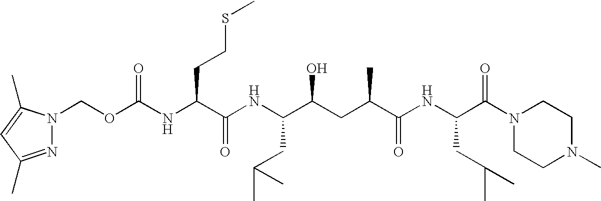 | | | | |
| MMI-217 | 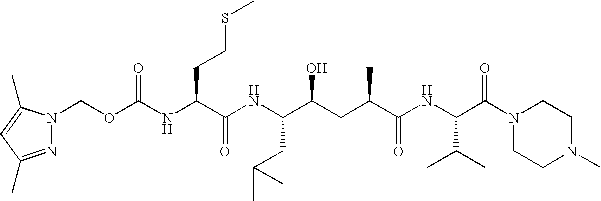 | | | | |
| MMI-218 | 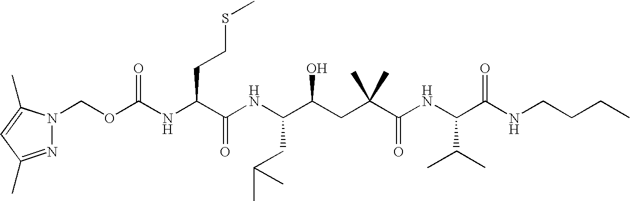 | | | | |
| MMI-219 | 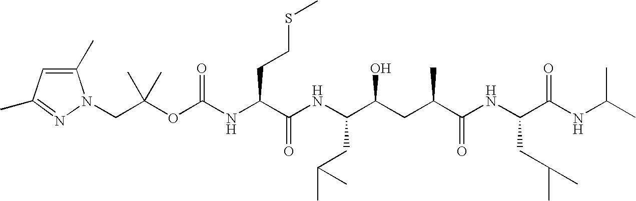 | | | | |
| MMI-220 | 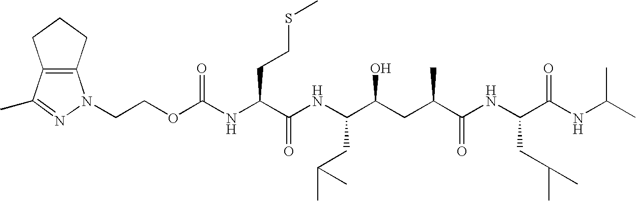 | | | | |
| MMI-221 | 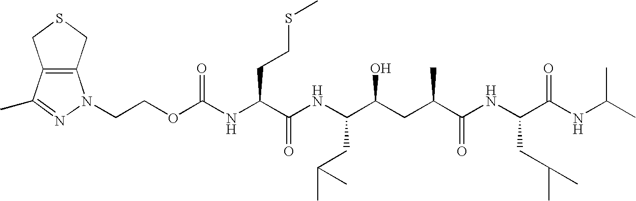 | | | | |
| MMI-222 | 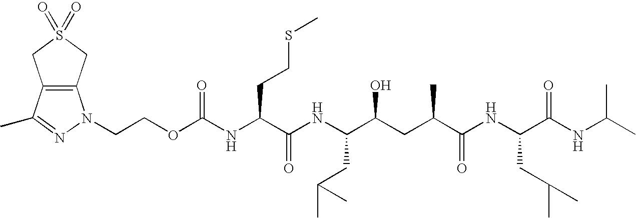 | | | | |
| MMI-223 | 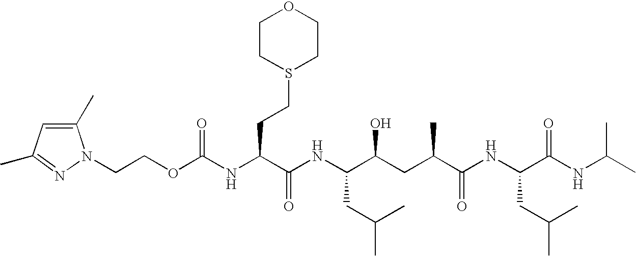 | | | | |
| MMI-224 | 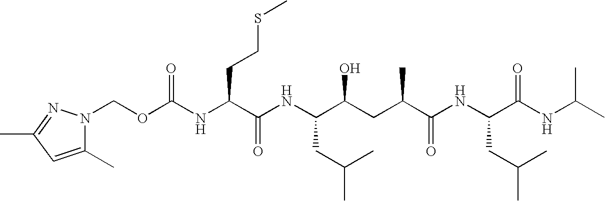 | | | | |
| MMI-225 | 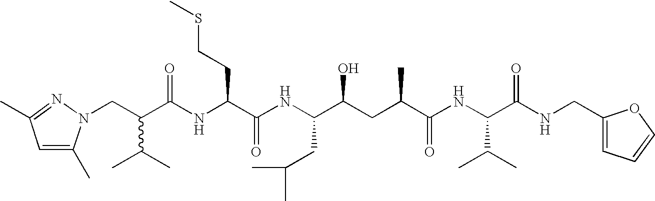 | | | | |
| MMI-226 | 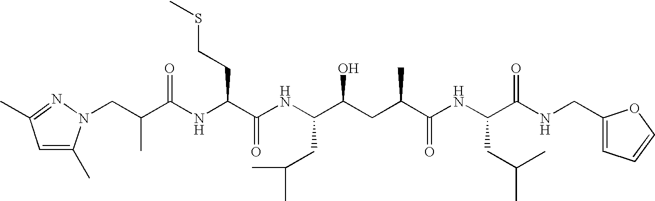 | | | | |
| MMI-227 | 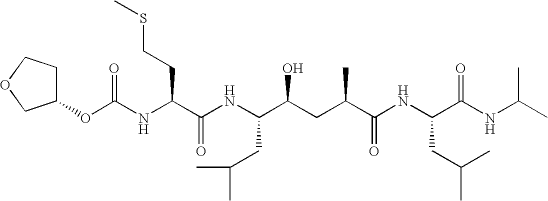 | | | | |
| MMI-228 | 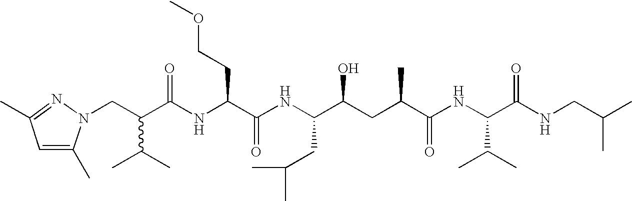 | | | | |
| MMI-229 | 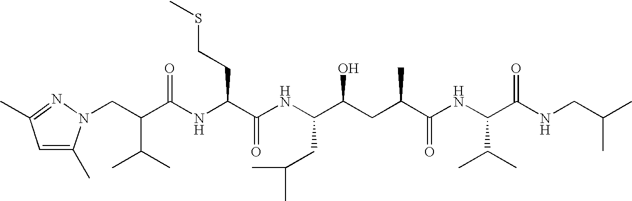 | | | | |
| | | | | Ki, [I] < 100 | |
| Compound | Ki (nM) | Ki app (nM) | Ki app error | (nM) | Vi/Vo |
| MMI-001 | 2373693 | 3738100 | | | |
| MMI-002 | 273647 | 430940 | | | |
| MMI-003 | 1661795 | 2617000 | | | |
| MMI-004 | 32653 | 51422 | | | |
| MMI-005 | 78.9 | 126.2 | | | |
| MMI-006 | 40188 | 63288 | | | |
| MMI-007 | 31672 | 49877 | | | |
| MMI-008 | 46 | 73.6 | | | |
| MMI-009 | 56.2 | 89.9 | | | |
| MMI-010 | 18671 | 29403 | | | |
| MMI-011 | 3099 | 4880 | | | |
| MMI-012 | 5.68 | 9.1 | | | |
| MMI-013 | 115100 | 184160 | | | |
| MMI-014 | 1736.12 | 2777.8 | | | |
| MMI-015 | 5650 | 9040 | | | |
| MMI-016 | 3525 | 5640 | | | |
| MMI-017 | 2.4 | 3.9 | 0.91 | | |
| MMI-018 | 7.81 | 12.5 | | | |
| MMI-019 | 9062.5 | 14500 | | | |
| MMI-020 | 1003.2 | 1605.2 | | | |
| MMI-021 | 70.12 | 112.2 | | | |
| MMI-022 | 7354 | 11766 | | | |
| MMI-023 | 9546 | 15273 | | | |
| MMI-024 | 902 | 1443.3 | | | |
| MMI-025 | 40.9 | 65.45 | | | |
| MMI-026 | 9.97 | 15.95 | | | |
| MMI-027 | 7216 | 11547.3 | | | |
| MMI-028 | 6167 | 9867 | | | |
| MMI-029 | 1060 | 1696.6 | | | |
| MMI-030 | 5323 | 8517 | | | |
| MMI-031 | 539.3 | 863.7 | | | |
| MMI-032 | 838 | 1341 | | | |
| MMI-033 | 10187 | 16300 | | | |
| MMI-034 | 139.3 | 222.8 | | | |
| MMI-035 | 266.4 | 426.3 | | | |
| MMI-036 | N.I. | N.I. | | | |
| MMI-037 | 64.8 | 103.7 | | | |
| MMI-038 | 66.25 | 106 | | | 0.4 |
| MMI-039 | 872.5 | 1396.1 | | | |
| MMI-040 | 1294 | 2071.4 | | | |
| MMI-041 | N.I. | N.I. | | | |
| MMI-042 | 22802 | 36484.1 | | | |
| MMI-043 | 30742 | 49188.9 | | | |
| MMI-044 | N.I. | N.I. | | | |
| MMI-045 | N.I. | N.I. | | | |
| MMI-046 | 93669 | 149870 | | | |
| MMI-047 | 69137 | 110620 | | | |
| MMI-048 | 34249 | 54799 | | | |
| MMI-049 | 123750 | 198000 | | | |
| MMI-050 | 91250 | 146000 | | | |
| MMI-051 | N.I. | N.I. | | | |
| MMI-052 | 3619 | 5790 | | | |
| MMI-053 | N.I. | N.I. | | | |
| MMI-054 | 293125 | 469000 | | | |
| MMI-055 | 394375 | 631000 | | | |
| MMI-056 | 128125 | 205000 | | | |
| MMI-057 | 45812 | 73300 | | | |
| MMI-058 | 255000 | 408000 | | | |
| MMI-059 | 41437 | 66300 | | | |
| MMI-060 | 63119 | 100990 | | | |
| MMI-061 | 9661875 | 15459000 | | | |
| MMI-062 | N.I. | N.I. | | | |
| MMI-063 | | | | | |
| MMI-064 | | | | | |
| MMI-065 | 42.9 | 68.7 | | | |
| MMI-066 | 13.25 | 21.2 | | | |
| MMI-067 | 45.9 | 73.5 | | | |
| MMI-068 | 24.1 | 38.6 | | | |
| MMI-069 | | | | | |
| MMI-070 | 3.06 | 4.9 | | | |
| MMI-071 | 1.18 | 1.9 | | | |
| MMI-072 | | | | | 0.9 |
| MMI-073 | | | | | 0.48 |
| MMI-074 | | | | | |
| MMI-075 | | | | | |
| MMI-076 | | | | | |
| MMI-077 | | | | | |
| MMI-078 | | | | | 0.63 |
| MMI-079 | | | | | |
| MMI-080 | | | | | |
| MMI-081 | | | | | 0.87 |
| MMI-082 | | | | | 0.72 |
| MMI-083 | | | | | |
| MMI-084 | | | | | |
| MMI-085 | | | | | |
| MMI-086 | | | | | |
| MMI-087 | 565.47 | 904.75 | 77.7 | | |
| MMI-088 | 646.3 | 1034.1 | 342.1 | | |
| MMI-089 | 196.66 | 314.65 | 27.4 | | |
| MMI-090 | 194.43 | 311.1 | | | |
| MMI-091 | | | | | |
| MMI-092 | | | | | |
| MMI-093 | 7.45 | 11.92 | 5.6 | | |
| MMI-094 | 40.06 | 64.1 | 4.4 | | |
| MMI-095 | | | | | |
| MMI-096 | | | | | |
| MMI-097 | 51.71 | 82.75 | 11 | | |
| MMI-098 | | | | | |
| MMI-099 | 181.8 | 289.9 | 32.9 | | |
| MMI-100 | | | | | |
| MMI-101 | | | | | |
| MMI-102 | | | | | |
| MMI-103 | | | | | |
| MMI-104 | 519.54 | 831.27 | 126.3 | | |
| MMI-105 | 125.44 | 200.71 | 17.7 | | |
| MMI-106 | | | | | |
| MMI-107 | | | | | |
| MMI-108 | | | | | |
| MMI-109 | | | | | |
| MMI-110 | | | | | |
| MMI-111 | | | | | |
| MMI-112 | | | | | |
| MMI-113 | 62.14 | 99.87 | 12.3 | | |
| MMI-114 | 275.6 | 440.96 | 52.7 | | |
| MMI-115 | 235.62 | 377.84 | 59.1 | | 1.04 |
| MMI-116 | 30.6 | 49 | 1.9 | | 0.29 |
| MMI-117 | | | | | 0.57 |
| MMI-118 | 194.4 | 311.09 | 24.7 | | 0.48 |
| MMI-119 | | | | | 0.38 |
| MMI-120 | 1125 | 1800 | 210 | 175.3 | 0.66 |
| MMI-121 | 5968 | 9549 | 304 | | |
| MMI-122 | | | | | 1.17 |
| MMI-123 | | | | | 0.61 |
| MMI-124 | | | | | 0.93 |
| MMI-125 | | | | | 0.76 |
| MMI-126 | | | | | 0.72 |
| MMI-127 | 4277.5 | 6844 | 388 | | |
| MMI-128 | | | | | 0.66 |
| MMI-129 | | | | | 1.01 |
| MMI-130 | 68625 | 109800 | 11580 | | |
| MMI-131 | 58050 | 92880 | 11410 | | |
| MMI-132 | 67.12 | 107.4 | 5.6 | | 0.46 |
| MMI-133 | 0.33 | 0.52 | 0.07 | | 0.09 |
| MMI-134 | 2.18 | 3.481 | 0.24 | | |
| MMI-135 | 6.46 | 10.34 | 0.08 | | 0.12 |
| MMI-136 | | | | | 0.87 |
| MMI-137 | | | | | 0.85 |
| MMI-138 | 8.8 | 14.2 | 8.8 | | |
| MMI-139 | | | | | 0.5 |
| MMI-140 | 24212.5 | 38740 | 2118 | | |
| MMI-141 | 18775 | 30040 | 1720 | | |
| MMI-142 | | | | | 0.92 |
| MMI-143 | | | | | 1 |
| MMI-144 | | | | | 1.03 |
| MMI-145 | | | | | 0.84 |
| MMI-146-A | | | | | 1.07 |
| MMI-146-S | | | | | 1.04 |
| MMI-147 | | | | | 1.1 |
| MMI-148 | 16.2 | 25.92 | 2.1 | | 0.46 |
| MMI-149 | | | | | 0.66 |
| MMI-150 | | | | | 0.8 |
| MMI-151 | | | | | 0.56 |
| MMI-152 | 12.85 | 20.57 | 2.1 | | 0.47 |
| MMI-153 | | | | | 0.68 |
| MMI-154 | | | | | 0.1 |
| MMI-155 | 1.28 | 2.06 | 3.2 | | 0.08 |
| MMI-156 | 1.89 | 3.03 | 3.3 | | 0.32 |
| MMI-157 | | | | | |
| MMI-158 | 0.33 | 0.524 | 0.1 | | 0.08 |
| MMI-159 | 0.5 | 0.8 | 0.12 | | 0.07 |
| MMI-160 | 2.59 | 4.154 | 0.43 | | 0.19 |
| MMI-161 | 0.43 | 0.68 | 0.097 | | 0.06 |
| MMI-162 | | | | | 0.98 |
| MMI-163 | | | | | 0.57 |
| MMI-164 | 7.98 | 12.77 | 8.1 | | 0.29 |
| MMI-165 | 15.31 | 24.5 | 3.6 | | 0.4 |
| MMI-166 | 67.43 | 107.89 | 13.9 | | 0.76 |
| MMI-167 | 22.84 | 36.55 | 2.7 | | 0.56 |
| MMI-168 | | | | | 0.75 |
| MMI-169 | | | | | 1.1 |
| MMI-170 | | | | | 1.08 |
| MMI-171 | 39.95 | 63.95 | 13 | | 0.55 |
| MMI-172 | 279.37 | 447 | | | 1.05 |
| MMI-173 | | | | | |
| MMI-174 | | | | | |
| MMI-175 | | | | | |
| MMI-176 | 69.1 | 110.6 | 7.9 | | 1 |
| MMI-177 | | | | | 1.16 |
| MMI-178 | 15.3 | 24.48 | 3.2 | | 0.69 |
| MMI-179 | | | | | |
| MMI-180 | | | | | 0.74 |
| MMI-181 | 245.5 | 392.82 | | | 0.53 |
| MMI-182 | 280 | 447.1 | 232.5 | 72.7 | 0.66 |
| MMI-183 | 4210 | 6736 | | 108 | 0.64 |
| MMI-184 | 121.25 | 194 | | 64 | 0.6 |
| MMI-185 | 4.52 | 7.23 | 3.7 | | |
| MMI-186 | 135.55 | 216.88 | 56.4 | | |
| MMI-187 | N.I. | N.I. | | | |
| MMI-188 | 143.81 | 230.1 | 38.5 | | |
| MMI-189 | 223.63 | 357.81 | 18.1 | | |
| MMI-190 | 18.22 | 29.15 | 6.9 | | |
| MMI-191 | N.I. | N.I. | | | |
| MMI-192 | 233.65 | 373.84 | 38.1 | | |
| MMI-193 | 180.15 | 288.25 | 83.4 | | |
| MMI-194 | 38.94 | 62.31 | 13.2 | | |
| MMI-195 | 282.1 | 451.3 | 30.3 | | |
| MMI-196 | 18 | 28.8 | 4.7 | | |
| MMI-197 | 59.1 | 94.55 | 5.1 | | |
| MMI-198 | 472.6 | 756.1 | 97 | | |
| MMI-199 | 602.2 | 963.5 | 402 | 190 | |
| MMI-200 | 955.8 | 1529.3 | 707 | | |
| MMI-201 | 36.24 | 57.99 | 8.9 | | |
| MMI-202 | 429.6 | 687.5 | 34.32 | | |
| MMI-203 | 225.4 | 360.7 | 18.7 | | |
| MMI-204 | 17.1 | 27.37 | 5.46 | | |
| MMI-205 | 30.5 | 48.8 | 13.7 | | |
| MMI-206 | 757.8 | 1212.4 | 348.5 | 292 | |
| MMI-207 | 988.5 | 1581.6 | 452.5 | 1826 | |
| MMI-208 | 1218.6 | 1949.9 | 895 | 406.6 | |
| MMI-209 | 812.5 | 1300.5 | 351 | | |
| MMI-210 | 562.1 | 899.37 | 255 | | |
| MMI-211 | 475.9 | 761.47 | 54.1 | | |
| MMI-212 | 44.46 | 71.13 | 9.46 | | |
| MMI-213 | 52.88 | 84.62 | 11.7 | | |
| MMI-214 | 11.88 | 19.01 | 5.62 | | |
| MMI-215 | 19.3 | 30.91 | 5.78 | | |
| MMI-216 | 753.1 | 1204.9 | 243.2 | | |
| MMI-217 | 670.1 | 1072.17 | 220.25 | | |
| MMI-218 | 1122.5 | 1796.9 | 1032.3 | 226 | |
| MMI-219 | 888.9 | 1422.24 | 208.5 | | |
| MMI-220 | 2805.6 | 4489.06 | 3384 | | |
| MMI-221 | 1192 | 1907.21 | 615.35 | | |
| MMI-222 | 1529.38 | 2447.06 | 631.48 | | |
| MMI-223 | 5276.8 | 8424 | 4763 | | |
| MMI-224 | 43.8 | 70 | 4.3 | | |
| MMI-225 | 25.7 | 41 | 4.0 | | |
| MMI-226 | 68.9 | 110 | 6.6 | | |
| MMI-227 | 451.6 | 721 | 79 | | |
| MMI-228 | 110.2 | 176 | 12 | | |
| MMI-229 | | | | | |

Where stereochemistry is not shown, the compound is a mixture of isomers. N.I., no inhibition.

The standard error for the Kᵢ apparent is the error from the nonlinear regression of the Vi/Vo data measured at different concentrations of the compounds of the invention (e.g., between about 10 nM to about 1000 nM) employing well-known techniques (see, for example, Bieth, J., Bayer-Symposium V: Proteinase Inhibitors, pp. 463-469, Springer-Verlag, Berlin (1994)).

The Kᵢₐₚₚ (apparent Kᵢ) values of inhibitors against memapsins 1 and 2 were determined employing previously described procedures (Ermolieff, J., et al., Biochemistry 39: 12450-12456 (2000), the teachings of which are incorporated herein by reference in their entirety). The relationship of Kᵢ (independent of substrate concentration) to Kᵢₐₚₚ is a function of substrate concentration in the assay and the Kₘ for cleavage of the substrate by either memapsin 1 or memapsin 2 by the relationship: Kᵢₐₚₚ=Kᵢ(1+[S]/Kₘ).

“Memapsin 1” or “memapsin 1 protein,” as defined herein, refers to a protein that includes amino acids 58-461 of SEQ ID NO: 4.

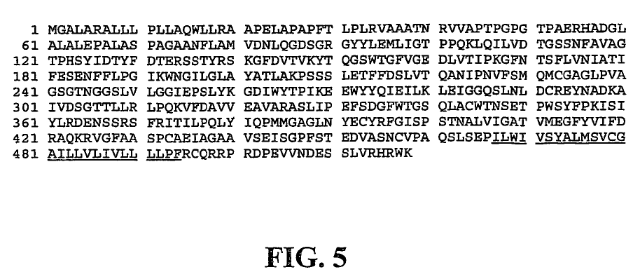

In one embodiment, memapsin 1 includes a transmembrane protein (SEQ ID NO: 2 (FIG. 5)). The transmembrane domain of SEQ ID NO: 2 (FIG. 5) is amino acid residues 467-494. The signal peptide of SEQ ID NO: 2 (FIG. 5) is amino acid residues 1-20. The propeptide of SEQ ID NO: 2 (FIG. 5) is amino acid residues 21-62.

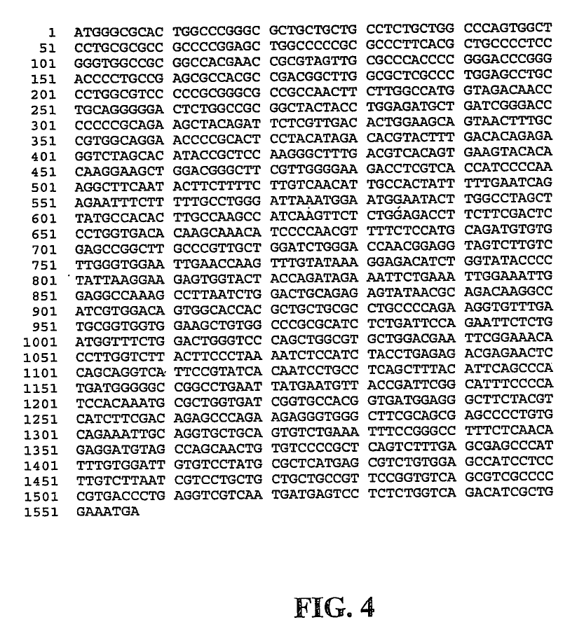

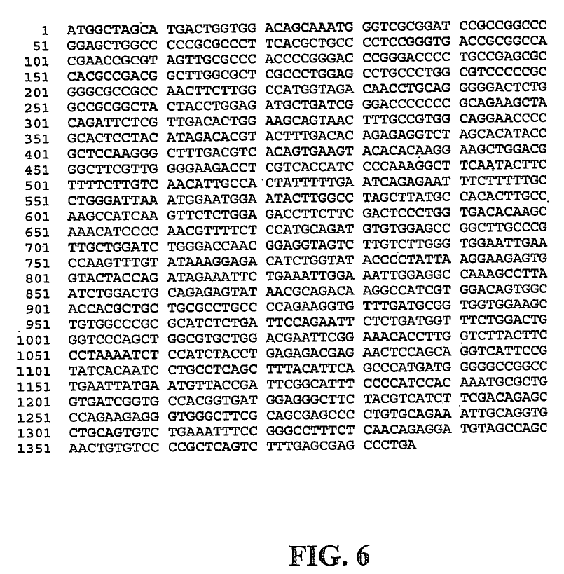

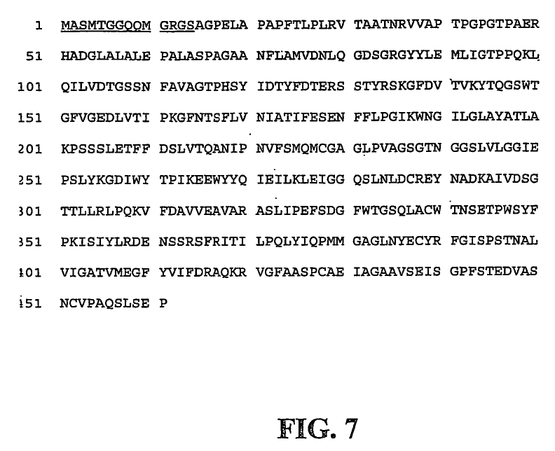

Constructs encoding memapsin 1 can be expressed in host cells (e.g., mammalian host cells such as HeLa cells or 293 cells or E. coli host cells). The nucleic acid sequence encoding the promemapsin 1-T1 (SEQ ID NO: 3 (FIG. 6)) employed herein has a G at position 47 instead of a C in SEQ ID NO: 1 (FIG. 4) and an A at position 91 instead of a G in SEQ ID NO: 1 (FIG. 4). The two nucleic acid differences result in a glycine residue at amino acid residue 16 (SEQ ID NO: 4 (FIG. 7)) instead of an alanine (at position 21 of SEQ ID NO: 2 (FIG. 5)); and a threonine at amino acid residue 31 (SEQ ID NO: 4 FIG. 7)) instead of an alanine (at position 36 of SEQ ID NO: 2 (FIG. 5)).

A nucleic acid construct encoding promemapsin 1-T1 (SEQ ID NO: 4 (FIG. 7)) was expressed in E. coli, the protein purified from inclusion bodies and autocatalytically activated by incubation at pH 3-5 for 30 minutes (37° C.) to obtain memapsin 1 with an amino terminus of alanine (amino acid residue 58 of SEQ ID NO: 4 (FIG. 7)), which was employed in assays to assess the inhibition of memapsin 2 relative to memapsin 1 by compounds of the invention.

“Memapsin 2” or “memapsin 2 protein,” is any protein that includes an amino acid sequence identified herein that includes the root word “memapsin 2,” or any sequence of amino acids, regardless of whether it is identified with a SEQ ID NO, that is identified herein as having been derived from a protein that is labeled with a term that includes the root word memapsin 2 (e.g., amino acid residues 1-456 of SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and the various equivalents derived from SEQ ID NO: 9) and can hydrolyze a peptide bond. Generally, memapsin 2 is capable of cleaving a β-secretase site (e.g., the Swedish mutation of APP SEVNLDAEFR, SEQ ID NO: 11; the native APP SEVKMDAEFR, SEQ ID NO: 12). In one embodiment, memapsin 2 consists essentially of an amino acid sequence that results from activation, such as spontaneous activation, autocatalytic activation, or activation with a protease, such as clostripain, of a longer sequence. Embodiments of memapsin 2 that consist essentially of an amino acid fragment that results from such activation are those that have the ability to hydrolyze a peptide bond. Crystallized forms of memapsin 2 are considered to continue to be memapsin 2 despite any loss of β-secretase activity during crystallization. Embodiments of memapsin 2 are also referred to as BACE, ASP2 and β-secretase.

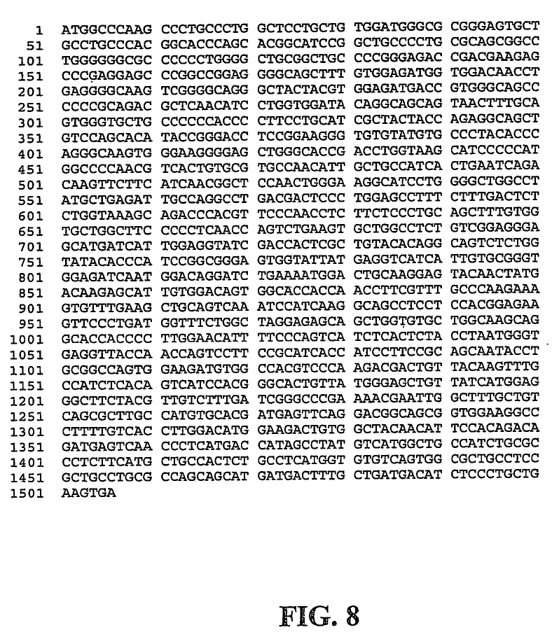

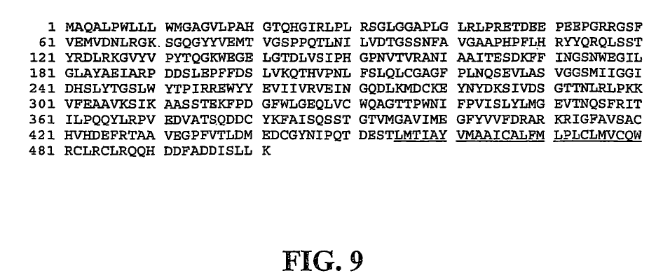

In one embodiment, memapsin 2 is a transmembrane protein (SEQ ID NO: 6 (FIG. 9)) and is encoded by the nucleic acid sequence SEQ ID NO: 5 (FIG. 8). The transmembrane domain of this embodiment (SEQ ID NO: 6 (FIG. 9)) of memapsin 2 is amino acid residues 455-480.

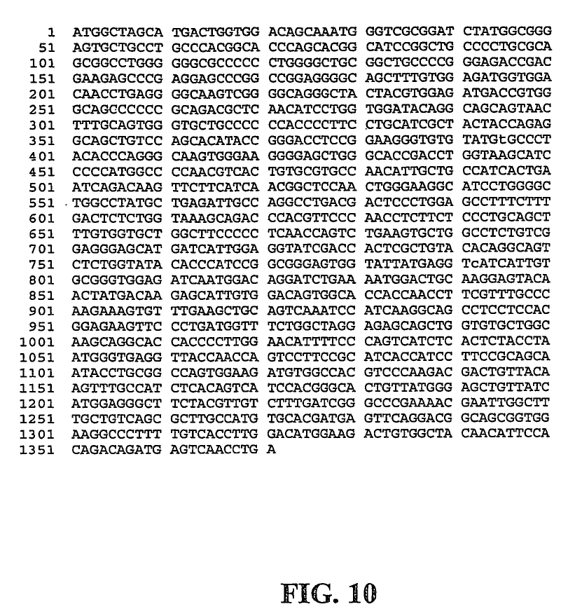

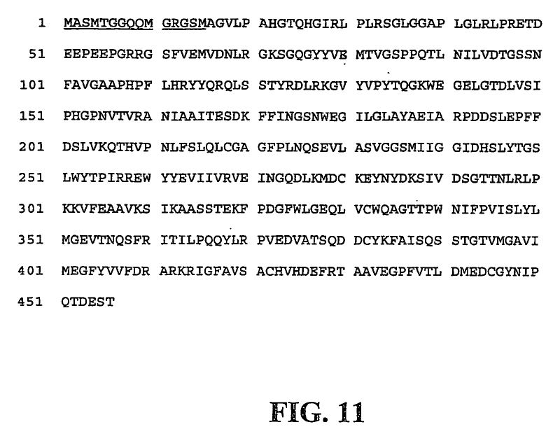

In another embodiment, memapsin 2 is promemapsin 2-T1 (nucleic acid sequence SEQ ID NO: 7 (FIG. 10); amino acid sequence SEQ ID NO: 8 (FIG. 11)) and can be derived from nucleotides 40-1362 of SEQ ID NO: 5 (FIG. 8).

The nucleic acid construct of the resulting promemapsin 2-T1 SEQ ID NO: 7 (FIG. 10) was expressed in E. coli, protein purified and spontaneously activated to memapsin 2. Spontaneously activated memapsin 2 includes amino acid residues 43-456 of SEQ ID NO: 8 (FIG. 11) and amino acid residues 45-456 of SEQ ID NO: 8 (FIG. 11). The spontaneously activated memapsin 2 was employed in assays to assess the inhibition of memapsin 2 relative to memapsin 1 by compounds of the invention and in some crystallization studies.

It is also envisioned that promemapsin 2-T1 can be expressed in E. coli and autocatalytically activated to generate memapsin 2 which includes amino acid residues 16-456 of SEQ ID NO: 8 (FIG. 11) and amino acid residues 27-456 of SEQ ID NO: 8 (FIG. 11).

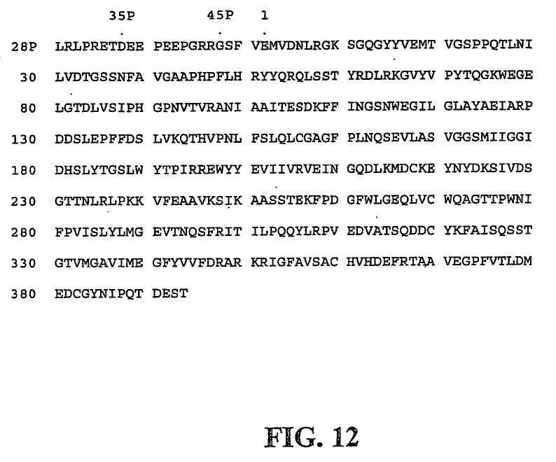

A memapsin 2 including amino acid residues 60-456 of SEQ ID NO: 8 (FIG. 11); and amino acid residues 28P-393 of SEQ ID NO: 9 (FIG. 12) can also be employed in crystallization studies. The memapsin 2 of amino acid residues 60-456 of SEQ ID NO: 8 (FIG. 11) was used in crystallization studies with the inhibitor compound MMI-138.

Compounds that selectively inhibit memapsin 2 β-secretase activity relative to memapsin 1 β-secretase activity are useful to treat diseases or conditions or biological processes association with memapsin 2 β-secretase activity rather than diseases or conditions or biological processes associated with both memapsin 1 and memapsin 2 β-secretase activity. Since both memapsin 1 and memapsin 2 cleave amyloid precursor protein (APP) at a β-secretase site to form β-amyloid protein (also referred to herein as Aβ, Abeta or β-amyloid peptide), memapsin 1 and memapsin 2 have β-secretase activity (Hussain, I., et al., J. Biol. Chem. 276:23322-23328 (2001), the teachings of which are incorporated herein in their entirety). However, the β-secretase activity of memapsin 1 is significantly less than the β-secretase activity of memapsin 2 (Hussain, I., et al., J. Biol. Chem. 276:23322-23328 (2001), the teachings of which are incorporated herein in their entirety). Memapsin 2 is localized in the brain, and pancreas, and other tissues (Lin, X., et al., Proc. Natl. Acad. Sci. USA 97:1456-1460 (2000), the teachings of which are incorporated herein in their entirety) and memapsin 1 is localized preferentially in placentae (Lin, X., et al., Proc. Natl. Acad. Sci. USA 97:1456-1460 (2000), the teachings of which are incorporated herein in their entirety). Alzheimer's disease is associated with the accumulation of Aβ in the brain as a result of cleaving of APP by β-secretase (also referred to herein as memapsin 2, ASP2 and BACE). Thus, methods employing the compounds which selectively inhibit memapsin 2 β-secretase activity relative to memapsin 1 β-secretase activity are important in the treatment of memapsin 2-related diseases, such as Alzheimer's disease. Selective inhibition of memapsin 2 β-secretase activity makes the compounds of the invention suitable drug candidates for use in the treatment of Alzheimer's disease.

In yet another embodiment, the invention is a method of treating Alzheimer's disease in a mammal (e.g., a human) comprising the step of administering to the mammal the compounds of the invention. The mammals treated with the compounds of the invention can be human primates, nonhuman primates and non-human mammals (e.g., rodents, canines). In one embodiment, the mammal is administered a compound that inhibits β-secretase (inhibits memapsin 1 and memapsin 2 β-secretase activity). In another embodiment, the mammal is administered a compound that selectively inhibits memapsin 2 β-secretase activity and has minimal or no effect on inhibiting memapsin 1 β-secretase activity.

In an additional embodiment, the invention is a method of inhibiting hydrolysis of a β-secretase site of a β-amyloid precursor protein in a mammal, comprising the step of administering to the mammal the compounds of the invention.

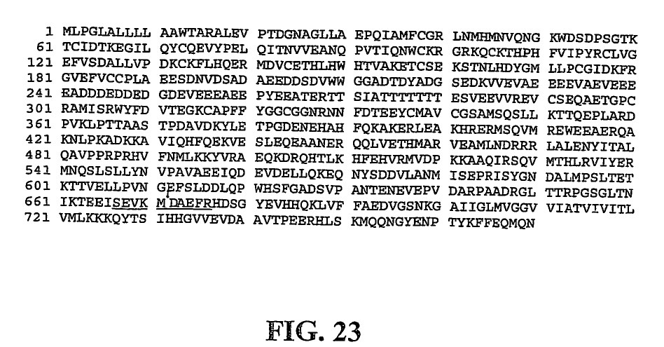

A “β-secretase site” is an amino acid sequence that is cleaved (i.e., hydrolyzed) by memapsin 1 or memapsin 2 (also referred to herein as β-secretase and ASP2). In a specific embodiment, a β-secretase site is an amino acid sequence cleaved by a protein having the sequence 43-456 of SEQ ID NO: 8 (FIG. 11). β-amyloid precursor protein (APP; FIG. 23, SEQ ID NO: 10) is cleaved at a β-secretase site (arrow, FIG. 23) to generate β-amyloid protein. In one embodiment of the invention, the β-secretase site includes the amino acid sequence SEVKM/DAEFR (SEQ ID NO: 12) also shown as amino acid residues 667-676 of SEQ ID NO: 10 (FIG. 23). β-secretase cleaves SEVKM/DAEFR (SEQ ID NO: 12) between methionine (M) and aspartic acid (D). In another embodiment of the invention, the β-secretase site includes the amino acid sequence of the Swedish mutation SEVNL/DAEFR (SEQ ID NO: 11). β-secretase cleaves SEVNL/DAEFR (SEQ ID NO: 11) between the leucine (L) and aspartic acid (D). Compounds of the invention inhibit the hydrolysis of the β-secretase site of the β-amyloid precursor protein. A β-secretase site can be any compound which includes SEVKMDAEFR (SEQ ID NO: 12) or SEVNL/DAEFR (SEQ ID NO: 11). A forward slash (“/”) indicates that the amide bond between the flanking amino acid residues is cleaved by memapsin 2.

In another embodiment, the compounds of the invention are administered to a mammal to inhibit the hydrolysis of a β-secretase site of a β-amyloid precursor protein. In another embodiment, the compounds are administered to an in vitro sample to inhibit the hydrolysis of a β-secretase site of a β-amyloid precursor protein.

“In vitro sample,” as used herein, refers to any sample that is not in the entire mammal. For example, an in vitro sample can be a test tube in vitro combination of memapsin 2 and an inhibitor compound of the invention; or can be an in vitro cell culture (e.g., Hela cells, 293 cells) to which the inhibitor compounds and/or memapsin proteins (memapsin 1 or 2) are added.

In a further embodiment, the invention is a method of decreasing the amount or production of β-amyloid protein in an in vitro sample or a mammal comprising the step of administering the compounds of the invention. The amount of β-amyloid protein or a decrease in the production of β-amyloid protein can be measured using standard techniques including western blotting and ELISA assays. A decrease in β-amyloid protein or a decrease in the production of β-amyloid protein can be measured, for example, in cell culture media in an in vitro sample or in a sample obtained from a mammal. The sample obtained from the mammal can be a fluid sample, such as a plasma or serum sample; or can be a tissue sample, such as a brain biopsy.

The compounds of the invention can be administered with or without a carrier molecule. “Carrier molecule,” as used herein, refers to a cluster of atoms held together by covalent bonds (the molecule) that are attached or conjugated to a compound or compounds of the invention. To penetrate the blood brain barrier (BBB), the carrier molecule must be relatively small (e.g., less than about 500 daltons) and relatively hydrophobic. The compounds of the invention may be attached or conjugated to the carrier molecule by covalent interactions (e.g., peptide bonds) or by non-covalent interactions (e.g., ionic bonds, hydrogen bonds, van der Waals attractions). In addition, carrier molecules may be attached to any functional group on a compound of the invention. For example, a carrier molecule may be attached to an amine group at the amine terminus of a peptide inhibitor of the invention. For example, R₁ of Formula II may be a carrier molecule. A carrier molecule may be attached to a carboxylic acid group at the carboxylic acid terminus of a peptide inhibitor of the invention. For example, NR₃R₃ of Formula II may be a carrier molecule. Alternatively, the carrier molecule may be attached to a side chain (e.g., P₁, P₂, P₃, P₄, P₅, P₆, P₇, P₈, P₁′, P₂′, P₃′, P₄′, etc.) of an amino acid residue that is a component of the compounds of the invention.

The confocal microscopic images of cells incubated with CPI-1 revealed that inhibitors of the invention were not evenly distributed inside the cells. Some high fluorescence intensity was associated with intracellular vesicular structures including endosomes and lysosomes. These images indicated that the inhibitor was trapped inside of these subcellular compartments. This indicated that when CPI-1 enters lysosomes and endosomes, the carrier peptide moiety, in this case tat, was modified by proteases within lysosome or endosome resulting in an inhibitor that was unable to exit the lysosomal or endosomal compartment.

Lysosomes and endosomes contain many proteases, including hydrolase such as cathepsins A, B, C, D, H and L. Some of these are endopeptidase, such as cathepsins D and H. Others are exopeptidases, such as cathepsins A and C, with cathepsin B capable of both endo- and exopeptidase activity. The specificities of these proteases are sufficiently broad to hydrolyze a tat peptide away from the inhibitor compound, thus, hydrolyzing the carrier peptide away from the isosteric inhibitor.

These facts make it possible to use tat and other carrier peptides for specific delivery of pharmaceutical agents, such as the ocmpound of Formula II, IV, VII, or a compound in Table 1 to lysosomes and endosomes. For example, a compound of Formula II, IV, VII or a compound in Table 1 to be delivered is chemically linked to a carrier peptide like tat to make a conjugated drug. When administered to a mammal by a mechanism such as injections, the conjugated compound will penetrate cells and permeate to the interior of lysosomes and endosomes. The proteases in lysosomes and endosomes will then hydrolyze tat. The conjugated compound will lose its ability to escape from lysosomes and endosomes.

The carrier peptide can be tat or other basic peptides, such as oligo-L-arginine, that are hydrolyzable by lysosomal and endosomal proteases. Specific peptide bonds susceptible for the cleavage of lysosomal or endosomal proteases may be installed in the linkage peptide region between a compound of Formula II, IV, VII or a compound in Table 1 and the carrier peptides. This will facilitate the removal of carrier peptide from the compound. For example, dipeptides Phe-Phe, Phe-Leu, Phe-Tyr and others are cleaved by cathepsin D.

Furthermore, the dissociable carrier molecule may be an oligosaccharide unit or other molecule linked to the compound by phosphoester or lipid-ester or other hydrolyzable bonds which are cleaved by glycosidases, phosphatases, esterases, lipases, or other hydrolases in the lysosomes and endosomes.

This type of drug delivery may be used to deliver the inhibitors of the invention to lysosomes and endosomes where memapsin 2 is found in high concentrations. This drug delivery system may also be used for the treatment of diseases such as lysosome storage diseases.

In one embodiment, the carrier molecule is a peptide, such as the tat-peptide Tyr-Gly-Arg-Lys-Lys-Arg-Arg-Gln-Arg-Arg-Arg (SEQ ID NO: 13) (Schwarze, S. R., et al., Science 285:1569-1572 (1999), the teachings of which are incorporated herein in their entirety) or nine arginine residues Arg-Arg-Arg-Arg-Arg-Arg-Arg-Arg-Arg (SEQ ID NO: 14) (Wender, P. A., et al., Proc. Natl. Acad. Sci. USA 97:13003-13008 (2000), the teachings of which are incorporated herein in their entirety). In another embodiment, the carrier molecule includes cationic molecules (i.e., molecules that are ionized at physiologic pH) and preferably polycationic molecules. Preferred functional groups that form cations include guanidine, amino, or imidizole. Carrier molecules include saccharides or lipids that contain about 1-10 of the following functional groups: guanidine, amino, or imidizole. Carrier molecules also include peptides of length about 10 amino acids, consisting of a combination of about 1-10 lysine, 1-10 arginine, or 1-10 histidine residues, or 1-10 residues of amino acids that contain the following functional groups: guanidine, amino, or imidizole. Carrier molecules also include other constructions that are not peptides but contain the side chains of amino acids, consisting of a combination of about 1-10 lysine, 1-10 lysine, 1-10 arginine, or 1-10 histidine side chains, or 1-10 side chains that contain the following functional groups: guanidine, amino, or imidizole. When a compound of the invention is conjugated or attached to a carrier molecule, the resulting conjugate is referred to herein as a “Carrier Peptide-Inhibitor” conjugate or “CPI.” The CPI conjugate can be administered to an in vitro sample or to a mammal thereby serving as a transport vehicle for a compound or compounds of the invention into a cell in an in vitro sample or in a mammal. The carrier molecules and CPI conjugates result in an increase in the ability of the compounds of the invention to effectively penetrate cells and the blood brain barrier to inhibit memapsin 2 from cleaving APP to subsequently generate Aβ.

In another embodiment, the invention is a pharmaceutical composition of the compounds of the invention. The pharmaceutical composition of the compounds of the invention, with or without a carrier molecule, or the compounds of the invention, with or without a carrier molecule, can be administered to a mammal by enteral or parenteral means. Specifically, the route of administration is by intraperitoneal (i.p.) injection; oral ingestion (e.g., tablet, capsule form) or intramuscular injection. Other routes of administration as also encompassed by the present invention, including intravenous, intraarterial, or subcutaneous routes, and nasal administration. Suppositories or transdermal patches can also be employed.

The compounds of the invention can be administered alone or can be coadministered to the patient. Coadministration is meant to include simultaneous or sequential administration of the compounds individually or in combination (more than one compound): Where the compounds are administered individually it is preferred that the mode of administration is conducted sufficiently close in time to each other (for example, administration of one compound close in time to administration of another compound) so that the effects on decreasing β-secretase activity or β-amyloid production are maximal. It is also envisioned that multiple routes of administration (e.g., intramuscular, oral, transdermal) can be used to administer the compounds of the invention.

The compounds can be administered alone or as admixtures with a pharmaceutically suitable carrier. “Pharmaceutically suitable carrier,” as used herein refers to conventional excipients, for example, pharmaceutically, physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds of the invention. The preparations can also be combined, when desired, with other active substances to reduce metabolic degradation. A preferred method of administration of the compounds is oral administration, such as a tablet or capsule. The compounds of the invention when administered alone, or when combined with an admixture, can be administered in a single or in more than one dose over a period of time to confer the desired effect (e.g., decreased β-amyloid protein).

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

The dosage and frequency (single or multiple doses) administered to a mammal can vary depending upon a variety of factors, including of a disease that results in increased activity of memapsin 2 or increased accumulation of β-amyloid protein, whether the mammal suffers from another disease, and its route of administration; size, age, sex, health, body weight, body mass index, and diet of the recipient; nature and extent of symptoms of the disease being treated (e.g., Alzheimer's disease), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

In an additional embodiment, the invention is a crystallized protein comprising SEQ ID NO: 6 (FIG. 9) and a compound, wherein the compound is a compound of the invention and wherein the crystallized protein has an x-ray diffraction resolution limit not greater than about 4 Å (e.g., about 3.5 Å, about 3.0 Å, about 2.5 Å, about 2.0 Å, about 1.5 Å, about 1.0 Å, about 0.5 Å).

In yet another embodiment, the invention is a crystallized protein comprising a protein selected from the group consisting of amino acid residues 1-456 of SEQ ID NO: 8 (FIG. 11); amino acid residues 16-456 of SEQ ID NO: 8 (FIG. 11); amino acid residues 27-456 of SEQ ID NO: 8 (FIG. 11); amino acid residues 43-456 of SEQ ID NO: 8 (FIG. 11); and amino acid residues 45-456 of SEQ ID NO: 8 (FIG. 11) and a compound, wherein the compound is a compound of the invention, and wherein the crystallized protein has an x-ray diffraction resolution limit not greater than about 4.0 Å (e.g., about 3.5 Å, about 3.0 Å, about 2.5 Å, about 2.0 Å, about 1.5 Å, about 1.0 Å, about 0.5 Å).

The crystallized protein is formed employing techniques described herein (infra). Briefly, a nucleic acid construct encoding amino acids of SEQ ID NO: 6 (FIG. 9), amino acids 1-456 of SEQ ID NO: 8 (FIG. 11), amino acid residues 16-456 of SEQ ID NO: 8 (FIG. 11); amino acid residues 27-456 of SEQ ID NO: 8 (FIG. 11); amino acid residues 43-456 of SEQ ID NO: 8 (FIG. 11); or amino acid residues 45-456 of SEQ ID NO: 8 (FIG. 11) can be generated, expressed in E. coli, purified from inclusion bodies and crystallized with a compound or compounds of the invention. The diffraction resolution limit of the crystallized protein was determined. In an embodiment of the invention, the crystallized protein has an x-ray diffraction resolution limit not greater than about 2 Å. The diffraction resolution limit of the crystallized protein can be determined employing standard x-ray diffraction techniques.

In still another embodiment, the invention is a crystallized protein comprising a protein of SEQ ID NO: 6 (FIG. 9) and a compound, wherein the compound is a compound of the invention and wherein the crystallized protein has an x-ray diffraction resolution limit not greater than about 4.0 Å (e.g., 3.5 Å, 3.0 Å, 2.5 Å, 2.0 Å, 1.5 Å, 1.0 Å, 0.5 Å). SEQ ID NO: 6 is the amino acid sequence of memapsin 2 protein (also referred to herein as BACE, ASP2, β-secretase). As discussed above, the crystallized protein is formed employing techniques described herein (infra). Briefly, a nucleic acid construct encoding SEQ ID NO: 6 is generated, is expressed in a host cell, such as a mammalian host cell (e.g., Hela cell, 293 cell) or a bacterial host cell (e.g., E. coli), is purified and is crystallized with a compound or compounds of the invention. The diffraction resolution limit of the crystallized protein can be determined, for example, by x-ray diffraction or neutron diffraction techniques. SEQ ID NO: 6 can optionally lack a transmembrane domain. The transmembrane of memapsin 2 is amino acid residues 455-480 of SEQ ID NO: 6 (FIG. 9), which is the amino acid sequence LMTIAYVMAAICALFMLPLCLMVCQW (SEQ ID NO: 6) which is encoded by nucleic acids 1363-1440 of SEQ ID NO: 5 (FIG. 8). In a particular embodiment, the crystallized protein has an x-ray diffraction resolution limit not greater than about 2 Å.

In yet another embodiment, the invention is a crystallized protein comprising a protein encoded by SEQ ID NO: 5 (FIG. 8) and a compound, wherein the compound is a compound of the invention and wherein the crystallized protein has an x-ray diffraction resolution limit not greater than about 4.0 Å (e.g., 3.5 Å, 3.0 Å, 2.5 Å, 2.0 Å, 1.5 Å, 1.0 Å, 0.5 Å). SEQ ID NO: 5 is the nucleic acid sequence which encodes memapsin 2 protein (FIG. 8). As discussed above, the crystallized protein is formed employing techniques described herein (infra). Briefly, the nucleic acid construct of SEQ ID NO: 5 is expressed in a host cell, such as a mammalian host cell (e.g., Hela cell, 293 cell) or a bacterial host cell (e.g., E. coli) and the encoded protein is purified. The purified protein is crystallized with a compound or compounds of the invention. The diffraction resolution limit of the crystallized protein can be determined. SEQ ID NO: 5 can optionally lack the nucleic acids which encode the transmembrane domain of memapsin 2. The transmembrane domain is encoded by encoded nucleic acids 1363-1440 of SEQ ID NO: 5. In a particular embodiment, the crystallized protein has an x-ray diffraction resolution limit not greater than about 2 Å.

An embodiment of the invention includes compounds that selectively inhibit memapsin 2 activity relative to memapsin 1. The compounds of the invention are employed in methods to decrease β-secretase activity, to decrease the accumulation of β-amyloid protein and in the treatment of diseases or conditions associated with β-secretase activity and β-amyloid protein accumulation. The compounds of the invention can be employed in methods to treat Alzheimer's disease in a mammal.

The present invention relates to the discovery of compounds that inhibit memapsin 2 (also referred to as BACE or ASP2). An embodiment of the invention includes compounds that selectively inhibit memapsin 2 activity relative to memapsin 1. The compounds of the invention can be employed in methods to decrease β-secretase activity, to decrease the accumulation of β-amyloid protein and in the treatment of diseases or conditions associated with β-secretase activity and β-amyloid protein accumulation. The compounds of the invention can be employed in methods to treat Alzheimer's disease in a mammal.

The present invention is further illustrated by the following examples, which are not intended to be limiting in any way.

### EXEMPLIFICATION

### Example 1

### Inhibitors Selective for Memapsin 2

Inhibitors were designed, constructed and evaluated for their ability to selectively inhibit memapsin 2 relative to memapsin 1.

Materials and Methods

Expression and Purification of the Catalytic Domain of Memapsin 1

The protease domain of memapsin 1 (amino acid residues 15-461 of SEQ ID NO: 4 (FIG. 7)) was expressed in E. coli as previously described for memapsin 2 (Lin, X., et al., Proc. Natl. Acad. Sci. USA 97:1456-1460 (2000), the teachings of which are incorporated herein by reference in their entirety).

FIG. 5 depicts the deduced amino acid sequence of memapsin 1.

The E. coli produced promemapsin 1-T1 (amino acid residues 1-461 of SEQ ID NO: 4 (FIG. 7)) as inclusion bodies which were recovered and washed as previously described (Lin, X., et al., Methods in Enzymol. 241:195-224 (1994), the teachings of which are incorporated herein by reference in their entirety), dissolved in 8 M urea, 10 mM β-mercaptoethanol, 0.1 mM oxidized glutathione, 1 mM reduced glutathione, and refolded by dilution into 20 fold volume of 20 mM Tris base, 10% glycerol with adjustment of pH from 10 to 9 and then to 8 over 48 hours. The recombinant promemapsin 1-T1 (amino acid residues 1-461 of SEQ ID NO: 4 (FIG. 7)) was further purified by Sephacryl S-300™ and ResourceQ™ columns, the latter in 0.4 M urea, 20 mM Tris HCl, pH 8.0 and eluted with a 0-1.0 M NaCl gradient in the same buffer. Promemapsin 1-T1 was converted to memapsin 1 (amino acid residues 58-461 of SEQ ID NO: 4 (FIG. 7)) auto-catalytically at pH 4 (Hussain, I., et al., J Biol. Chem. 276:23322-23328 (2001), the teachings of which are incorporated herein by reference in their entirety).

Expression of Memapsin 2 Employed in Inhibition Studies

Memapsin 2 (amino acid residues 1-456 of SEQ ID NO: 8 (FIG. 11)) was produced recombinantly in E. coli. (Lin, X., et al., Proc. Natl. Acad. Sci. USA 97:1456-1460 (2000)).

Memapsin 2 (FIG. 12; SEQ ID NO: 9) was obtained by spontaneous activation of refolded promemapsin 2-T1 by incubation at 4° C. in the refolding buffer (0.4 M urea, 20 mM Tris HCl, 0.5 mM DTT, 0.5 mM 2-mercaptoethanol, 50 μM glutathione (reduced), 5 μM glutathione (oxidized), pH 8.0) for 2 weeks prior to purification by gel filtration (Hong, et al., Science 290:150-153 (2000)). Promemapsin 2-T1 (amino acid residues 1-456 of SEQ ID NO: 8 (FIG. 11)) was spontaneously activated to memapsin 2 (amino acids 43-456 of SEQ ID NO: 8 (FIG. 11) and amino acids 45-456 of SEQ ID NO: 8 (FIG. 11)) and employed to determine selective inhibition and generally used in crystallization studies.

Memapsin 2 Specificity

Design of the Defined Substrate Mixtures

Peptide sequence EVNLAAEF (SEQ ID NO: 15), known to be a memapsin 2 substrate (Ghosh A. K., et al., J. Am. Chem. Soc. 122:3522-3523 (2000), the teachings of which are incorporated herein by reference in their entirety) was used as a template structure to study residue preferences in substrate mixtures. For characterization of each of the eight subsites, separate substrate mixtures were obtained by addition of an equimolar mixture of 6 or 7 amino acid derivatives in the appropriate cycle of solid-state peptide synthesis (Research Genetics, Invitrogen, Huntsville, Ala.). The resulting mixture of 6 or 7 peptides differed only by 1 amino acid at a single position. At each position, 19 varied amino acids (less cysteine) were accommodated in three substrate mixtures, requiring 24 substrate mixtures to characterize eight positions. A substrate of known k_{cat}/K_{M} was also added to each mixture to serve as an internal standard. To facilitate the analysis in MALDI-TOF MS (Matrix Assisted Laser Desorption/Ionization Time of Flight Mass Spectrometry), the template sequence was extended by 4 residues at the C-terminus (EVNLAAEFWHDR; SEQ ID NO: 16) for variations at P₁′, P₂′, P₃′, and P₄′ and at the N-terminus (RWHHEVNLAAEF; SEQ ID NO: 17) to study positions P₁, P₂, P₃, and P₄. “Substrate mixtures,” as referred to herein, are mixtures of variants of SEQ ID NO: 16 and 17, as described above. An example of a substrate mixture is set forth below in Table 2.

TABLE 2

MEMAPSIN 2 SUBSTRATE MIXTURES FOR THE
DETERMINATION OF RESIDUE PREFERENCES
IN POSITIONS P₁, P₂, P₃AND P₄.

| | | |
|---|---|---|
| Mixture | Sequence^{a} | Amino Acid Mixture^{b} |

| | | |
|---|---|---|
| APP-P₁A | RWHHEVN[mix]AAEF | A V L Q M F Y |
| APP-P₁B | RWHHEVN[mix]AAEF | F I D E G S T |
| APP-P₁C | RWHHEVN[mix]AAEF | F N P K R H W |
| APP-P₂A | RWHHEV[mix]LAAEF | V L N Q M F Y |
| APP-P₂B | RWHHEV[mix]LAAEF | G A S T N D E |
| APP-P₂C | RWHHEV[mix]LAAEF | P I N K H R W |
| APP-P₃A | RWHHE[mix]NLAAEF | V L N Q M F Y |
| APP-P₃B | RWHHE[mix]NLAAEF | G A S V T D E |
| APP-P₃C | RWHHE[mix]NLAAEF | P V I K H R W |
| APP-P₄A | RWHH[mix]VNLAAEF | A S T I D Q E |
| APP-P₄B | RWHH[mix]VNLAAEF | G V L K E R W |
| APP-P₄C | RWHH[mix]VNLAAEF | P N E M H F Y |

^{a}[mix]indicates an equimolar mixture of amino acid derivatives is incorporated at that position in the synthesis, resulting in a mixture of peptides which vary at that position by the amino acid in the mixture.
^{b}Mixture of amino acid derivatives added in the [mix]position.

Initial Rate Determination by MALDI-TOF Mass Spectrometry

Substrate mixtures were dissolved at 2 mg/ml in 10% glacial acetic acid and diluted into 0.009 M NaOH to obtain a mixture of substrates in the M range at pH 4.1. After equilibration at 25° C., the reactions were initiated by the addition of an aliquot of memapsin 2. Aliquots were removed at time intervals, and combined with an equal volume of MALDI-TOF matrix (α-hydroxycinnamic acid in acetone, 20 mg/ml) and immediately spotted in duplicate onto a stainless-steel MALDI sample plate. MALDI-TOF mass spectrometry was performed on a PE Biosystems Voyager DE instrument at the Molecular Biology Resource Center on campus. The instrument was operated at 25,000 accelerating volts in positive mode with a 150 ns delay. Ions with a mass-to-charge ratio (m/z) were detected in the range of 650-2000 atomic mass units. Data were analyzed by the Voyager Data Explorer module to obtain ion intensity data for mass species of substrates and corresponding products in a given mixture. Relative product formation was calculated as the ratio of signal intensity of the product to the sum of signal intensities of both product and the corresponding substrate. The quantitative aspect of this analysis was established as follows. From a mixture consisting of seven substrate peptides, EVNLXAEFWHDR (SEQ ID NO: 18) (X=amino acids A, S, T, L D, E, and F), their hydrolytic peptide products, XAEFWHDR (SEQ ID NO: 19), were prepared by complete hydrolysis. A series of mock partial digestions was prepared by combining known amounts of the substrate mixture with the hydrolysate, and each was subjected to MALDI-TOF/S analysis. The ratios of product to sum of product and substrate peptide from observed intensity data correlated with the expected ratios for each pair of peptides in the mixture (average slope 1.04±0.01; average intercept 0.019±0.021; average correlation coefficient 0.987±0.006). Relative product formed per unit time was obtained from non-linear regression analysis of the data representing the initial 15% formation of product using the model
1−e^{-kT}
where k is the relative hydrolytic rate constant and T is time in seconds. The initial relative hydrolytic rates of unknown substrates were converted to the relative k_{cat}/K_{M} by the equation
Relative k_{cat}/K_{M}=vₓ/vₛ
where vₓ and vₛ are the initial hydrolytic rates of a substrate x the reference substrates. For convenience of discussion, the relative k_{cat}/Kₘ value is also referred to as preference index.
Random Sequence Inhibitor Library

The combinatorial inhibitor library was based on the sequence of OM99-2, EVNL*AAEF (SEQ ID NO: 20; “*” represents hydroxyethylene transition-state isostere and is equivalent to E as used herein), with random amino acids (less cysteine) at 4 positions, P₂, P₃, P₂′ and P₃′. Di-isostere Leu*Ala was used in a single step of synthesis, thus fixed the structures at positions P₁ and P₁′. Peptides were synthesized by solid-state peptide synthesis method and left attached on the resin beads. By using the ‘split-synthesis’ procedure (Lam, K. S., et al. Nature 354:82-84 (1991)), each of the resin beads contained only one sequence while the sequence differed from bead to bead. The overall library sequence was

(SEQ ID NO:21)
Gly-Xx1-Xx2-Leu*Ala-Xx3-Xx4-Phe-Arg-Met-Gly-Gly-(Resin bead)
P₄ P₃ P₂ P₁ P₁′ P₂′ P₃′ P₄′

where Xxa residues (where a represents either 1, 2, 3, or 4) are randomized at each position with 19 amino acids. A shorter version of the peptides, starting at P₂′ (sequence: Xx3-Xx4-Phe-Arg-Met-Gly-Gly-(Resin bead) (SEQ ID NO: 22)), was also present in each bead with a ratio to the longer sequence at about 7:3. Without isostere, the short sequence would not bind memapsin 2 with significant strength but its presence was convenient for identifying the residues at P₂′ and P₃′ by automated Edman degradation. The residues were identified from the randomized positions as follows:

| | |
|---|---|
| Edman cycle #: | 1 2 3 4 |
| Sequence 1, from the long | Gly Xx1 Xx2 |
| sequence: | |
| Sequence 2, from the short | Xx3 Xx4 Phe Arg |
| sequence: | |

The assignment of Xx3 and Xx2 had no ambiguity since they are the only unknown residue at cycle 1 and 3, respectively. Amino acids Xx1 and Xx4 were assigned from their relative amounts. The presence of a methionine was designed to permit MS/MS identification of peptide fragments from released following CNBr cleavage.

Probing of the Random Sequence Library

About 130,000 individual beads, representing one copy of the library and estimated to be contained in 1.1 ml of settled beads, was hydrated in buffer A (50 mM Na acetate, 0.1% Triton X-100, 0.4 M urea, 0.02% Na azide, 1 mg/ml bovine serum albumin, pH 3.5; filtered with a 5 micron filter). The beads were soaked in 3% bovine serum albumin in buffer A for 1 h, to block the non-specific binding, and rinsed twice with the same buffer. Recombinant memapsin 2 was diluted into buffer A to 4 nM and incubated with the library for 1 hour. A single stringency wash was performed which included 6.7 μM transition-state isosteric inhibitor OM99-2 in buffer B (50 mM Na acetate, 0.1% Triton X-100, 0.02% sodium azide, 1 mg/ml BSA, pH 5.5; filtered with 5-micron filter), followed by two additional washes with buffer B without OM99-2. Affinity-purified IgG specific for recombinant memapsin 2 was diluted 100-fold in buffer B and incubated 30 minutes with the library. Following three washes with buffer B, affinity-purified anti-goat/alkaline phosphatase conjugate was diluted into buffer B (1:200) and incubated for 30 min, with three subsequent washes. A single tablet of alkaline phosphatase substrate (BCIP/NBT; Sigma) was dissolved in 10 ml water and 1 ml applied to the beads and incubated 1 hour. Beads were resuspended in 0.02% sodium azide in water and examined under a dissecting microscope. Darkly stained beads were graded by sight, individually isolated, stripped in 8 M urea for 24 h, and destained in dimethylformamide. The sequence determination of the beads were carried out in an Applied Biosystem Protein Sequencer at the Molecular Biology Resource Center on campus. The phenylthiohydantoin-amino acids were quantified using reversed-phase high-pressure liquid chromatography.

Synthesis of Inhibitor OM00-3

Inhibitor OM00-3 (ELDL*AVEF, SEQ ID NO: 23) was synthesized using the method as described by Ghosh, et al. (Ghosh, A. K., et al., J. Am. Chem. Soc. 122:3522-3523 (2000)).

Determination of Kinetic Parameters

The kinetic parameters, K_{M} and k_{cat}, using single peptide substrate, and Kᵢ against free inhibitors, were determined as previously described (Ermolieff, J. et al., Biochemistry 39:12450-12456 (2000)).

Kᵢ is the inhibition equilibrium constant which indicates the ability of compounds to inhibit the β-secretase activity of memapsin 2 and memapsin 1. Numerically lower Kᵢ values indicate a higher affinity of the compounds of the invention for memapsin 2 or memapsin 1. The Kᵢ value is independent of the substrate, and converted from Kᵢ apparent.

Kᵢ apparent is determined in the presence of substrate according to established techniques (see, for example, Bieth, J., Bayer-Symposium V: Proteinase Inhibitors, pp. 463-469, Springer-Verlag, Berlin (1994)).

Vi/Vo depicts the ratio of initial cleavage velocites of the substrate FS-2 (Ermolieff, et al., Biochemistry 40:12450-12456 (2000)) by memapsin 1 or memapsin 2 in the absence (Vo) or presence (Vi) of a compound of the invention. A Vi/Vo value of 1.0 indicates that a compound of the invention does not inhibit the β-secretase activity of the enzyme memapsin 1 or memapsin 2. A Vi/Vo value less than 1.0 indicates that a compound of the invention inhibits β-secretase activity of the enzyme memapsin 1 or memapsin 2. The Vi/Vo values depicted in Table 1 were determined at conditions under which the enzyme and inhibitor concentrations were equal (e.g., about 80 nM, 100 nM).

The standard error for the Kᵢ apparent is the error from the nonlinear regression of the Vi/Vo data measured at different concentrations of the compounds of the invention (e.g., between about 10 nM to about 1000 nM) employing well-known techniques (see, for example, Bieth, J., Bayer-Symposium V: Proteinase Inhibitors, pp. 463-469, Springer-Verlag, Berlin (1994)).

Results and Discussion

Determination of Substrate Side Chain Preference in Memapsin 2 Subsites

The residue preferences at each subsite for different substrate side chains are defined by the relative k_{cat}/K_{M} values, which are related to the relative initial hydrolysis rates of these mixtures of competing substrates under the condition that the substrate concentration is lower than K_{M} (Fersht, A., Enzyme Structure and Mechanism, 2^{nd} edition, W.H. Freeman, New York (1985)). This method is a less laborious method to determine the residue preference by measuring the initial velocity of substrate mixtures and has been used to analyze the specificity of other aspartic proteases (Koelsch, G., et al., Biochim. Biophys. Acta 1480:117-131 (2000); Kassel, D. B., et al., Anal. Biochem. 228:259-266 (1995)). The rate determination was improved by the use of MALDI-TOF/MS ion intensities for quantitation of relative amounts of products and substrates.

The substrate side chain preference, reported as preference index in eight subsites of memapsin 2 is depicted in FIGS. 2A, 2B, 2C, 2D, 2E, 2F, 2G, 2H. On both the P side and the P′ side, the side chains proximal to the scissile bond (P₁ and P₁′) are more stringent than the distal side chains (P₄ and P₄′). This is in evidence when the preference indexes of the non-preferred residues (background levels) are compared to the preferred residues. The lack of stringency is more pronounced for the four side chains on the P′ side, especially for P₃′ and P₄′ where the background is relatively high.

In the familial Alzheimer's disease caused by the Swedish mutation of APP (SEVNLDAEFR, SEQ ID NO: 11), the change of P₂-P₁ from Lys-Met to Asn-Leu results in an increase of about 60 fold of the k_{cat}/K_{M} of memapsin 2 cleavage indicating that the greatest increase in catalytic efficiency is derived from the change in P₂ (FIGS. 2A-2H). An Asp or Met at this position accompanied by a P₁ Leu may elevate the Aβ production and cause Alzheimer's disease.

Side Chain Preference Determined from a Combinatorial Inhibitor Library

The preference of memapsin 2 binding to side chains was also determined using a combinatorial library. The base-sequence of the library was derived from OM99-2: EVNL*AAEF (SEQ ID NO: 20) (“*” designates hydroxyethylene transition-state isostere), in which the P₃, P₂, P₂′ and P₃′ (boldface) were randomized with all amino acids except cysteine. After incubating the bead library with memapsin 2 and stringent selection of washing with OM99-2 solution, about 65 beads from nearly 130,000 beads were darkly stained, indicating strong memapsin 2 binding. The residues at the four randomized positions were determined for the ten most intensely stained beads. Table 3 shows that there is a clear consensus at these positions. This consensus is not present in the sequence of two negative controls (Table 3). To confirm this, a new inhibitor, OM00-3: ELDL*AVEF (SEQ ID NO: 23), was designed based on the consensus and synthesized. OM00-3 was found to inhibit memapsin 2 with Kᵢ of 0.31 nM, nearly five-fold lower than the Kᵢ of OM99-2. In addition, the residue preferences determined at P₃, P₂ and P₂′ of the inhibitors agreed well with the results from substrate studies (FIGS. 2A-2H).

TABLE 3

Observed residues at four side chain
positions from ten strong memapsin 2-
binding beads selected from a combina-
torial inhibitor library^{a}

| | | | | |
|---|---|---|---|---|
| ID | P₃ | P₂ | P₂′ | P₃′ |

| | | | | |
|---|---|---|---|---|
| 1 | Leu | Asp | Val | Glu |
| 2 | Leu | Glu | Val | Glu |
| 3 | Leu | Asp | Val | Glu |
| 4 | Leu | Asp | Val | Glu |
| 5 | Leu | Asp | Val | Gln |
| 6 | fle | Asp | Ala | Gln |
| 7 | lie | Asp | Val | Tyr |
| 8 | Leu | Glu | Val | Gln |
| 9 | Leu | Phe | Val | Glu |
| 10 | Phe/ | Ser | Val | Phe/ |
| | Ile | | | Ile |
| Neg1^{b} | Phe | Met | Asn | Arg |
| Neg2^{b} | Asp | Phe | | Ser |

^{a}Libraxy template: Gly-Xx₁-Xx₂-Leu*Ala-Xx₃-Xx₄-Phe-Arg-Met-Gly-Gly-Resin (SEQ ID NO: 21), wherein Xx₁ corresponds to an amino acid residue with side chain P₃; Xx₂ corresponds to an amino acid residue with side chain P₂; Xx₃ corresponds to an amino acid residue with side chain P₂′; and Xx₄ corresponds to an amino acid residue with side chain P₃′.
^{b}Neg 1 and Neg 2 are two randomly selected beads with no memapsin 2 binding capacity.

The Determination of Relative k_{cat}/Kₘ of Substrates in Substrate Mixtures

The relative initial hydrolysis rates of individual peptides in a mixture of substrates was determined. Since these relative rates are proportional to their kcat/Km values, they are taken as residue preferences when the substrates in the mixture differ only by one residues. The preference index was calculated from the relative initial hydrolitic rates of mixed substrates and is proportionate to the relative k_{cat}/Kₘ. The design of substrate mixtures and the condition of experiments are as described above.

Since memapsin 1 hydrolyzes some of the memapsin 2 cleavage sites (Farzan, M., et al., Proc. Natl. Acad. Sci., USA 97:9712-9717 (2000), the teachings of which are incorporated herein by reference in their entirety), the substrate mixture successfully used for studying subsite specificity of memapsin 2 (template sequence EVNLAAEF, SEQ ID NO: 15, was adopted for this study. Each substrate mixture contained six or seven peptides which differed only by one amino acid at a single position. At each position, each of the 19 natural amino acids (cysteine was not employed to prevent, for example, dimer formation by disulfide bonds) was accommodated in three substrate mixtures. A substrate of known kcat/Km value was also added to each set to serve as an internal standard for normalization of relative initial rates and the calculation of kcat/Km value of other substrates.

For four P′ side chain (P₁′, P₂′, P₃′ and P₄′), the template sequence was extended by four amino acid residues at the C-terminus (EVNLAAEFWHDR (SEQ ID NO: 16)) to facilitate detection in MALDI-TOF MS. Likewise, four additional amino acid residues were added to the N-terminus to characterize four P side chain (RWHHEVNLAAEF, SEQ ID NO: 17). The procedure and conditions for kinetic experiments were essentially as previously described for memapsin 2 (supra). The amount of substrate and hydrolytic products were quantitatively determined using MALDI-TOF mass spectrometry as described above. The relative kit values are reported as preference index.

Probing Random Sequence Inhibitor Library

The combinatorial inhibitor library was based on the sequence of OM99-2: EVNLΨAAEF (SEQ ID NO: 24), where letters represent amino acids in single letter code and Ψ represents a hydroxyethylene transition-state isostere, as previously described (U.S. Application Nos. 60/141,363, filed Jun. 28, 1999; 60/168,060, filed Nov. 30, 1999; 60/177,836, filed Jan. 25, 2000; 60/178,368, filed Jan. 27, 2000; 60/210,292, filed Jun. 8, 2000; 09/603,713, filed Jun. 27, 2000; 09/604,608, filed Jun. 27, 2000; 60/258,705, filed Dec. 28, 2000; 60/275,756, filed Mar. 14, 2001; PCT/US00/17742, WO 01/00665, filed Jun. 27, 2000; PCT/US00/17661, WO 01/00663, filed Jun. 27, 2000; U.S. patent application entitled “Compounds which Inhibit Beta-Secretase Activity and Methods of Use Thereof,” filed Oct. 22, 2002 and having Attorney Docket No. 2932.1001-003; and Ghosh, et al. (Ghosh, A. K., et al., J. Am. Chem. Soc. 122:3522-3523 (2000), the teachings of all of which are hereby incorporated by reference in their entirety). Four positions, P₂, P₃, P₂′ and P₃′, were filled with random amino acids residues (less cysteine). Positions P₁ and P₁′ were fixed due to the use of diisostere LeuΨAla in a single step of solid-state peptide synthesis of inhibitors (Ghosh, A. K., et al., J. Am. Chem. Soc. 122:3522-3523 (2000), the teachings of which are incorporated herein by reference in their entirety). By using the split-synthesis procedure (Lam, K. S., et al., Nature 354:82-84 (1991), the teachings of which are incorporated herein by reference in their entirety), each of the resin beads contained only one sequence while the sequence differed among beads. The overall library sequence was: Gly-Xx1-Xx2-LeuΨAla-Xx3-Xx4-Phe-Arg-Met-Gly-Gly- (Resin bead) (SEQ ID NO: 25).

Probing the binding of memapsin 1 to the combinatorial library and the sequence determination of the inhibitors was performed as described above. Affinity purified antibodies against memapsin 2 were used since the antibodies cross react with proteins memapsin 1 and memapsin 2.

Preparation of Inhibitors

Inhibitors of the invention are prepared by synthesis of the isostere portion of the inhibitor followed by coupling to a peptide having one or more an amino acids and/or modified amino acids.

I. Preparation of Leucine-Alanine Isostere 6

A leucine-alanine isostere unit is included in inhibitors MMI-001-MMI-009; MMI-011-MMI-020; MMI-022-MMI-026; MMI-034-MMI-035; MMI-039; MMI-041-MMI-047; MMI-049-MMI-060; MMI-063-MMI-077; MMI-079-MMI-091; MMI-093-MMI-100; MMI-103-MMI-105; MMI-107-MMI-131; MMI-133-MMI-144; MMI-146-MMI-154; MMI-156-MMI-163; MMI-165-MMI-167; MMI-171; MMI-173-MMI-177; MMI-180; MMI-183-MMI-86; MMI-188-MMI-90; MMI-193-MMI-200; MMI-203-MMI-210; MMI-212-MMI-217; MMI-219-MMI-130. The leucine-alanine isostere was prepared using the method shown in Scheme 1.

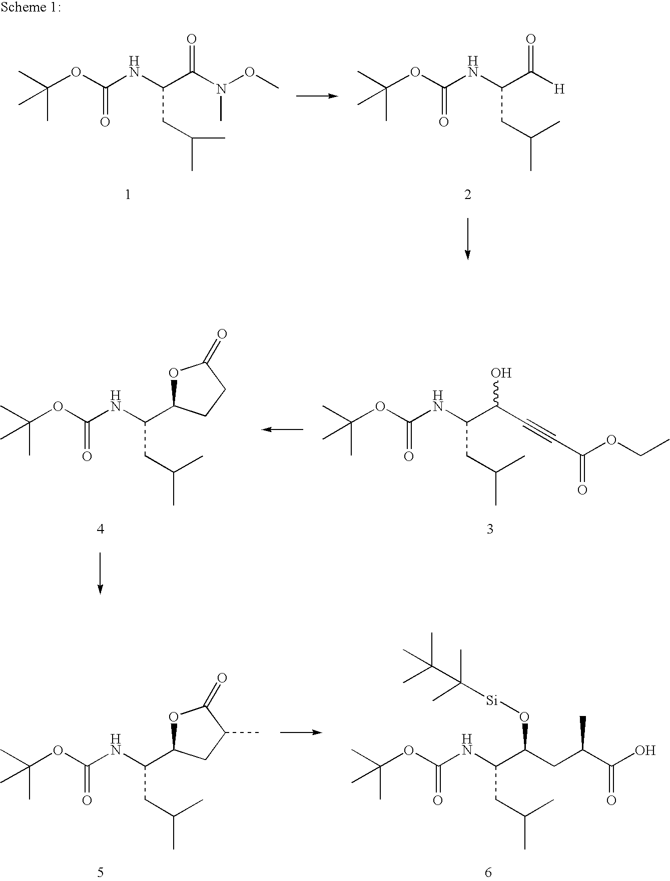

### A. N-(tert-Butoxycarbonyl)-L-leucine-N′-methoxy-N′-methylamide (1)

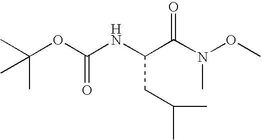

To a stirred solution of N,O-dimethylhydroxyamine hydrochloride (5.52 g, 56.6 mmol) in dry dichloromethane (25 mL) under a N₂ atmosphere at 0° C., was added N-methylpiperidine (6.9 mL, 56.6 mmol) dropwise. The resulting mixture was stirred at 0° C. for 30 minutes. In a separate flask, commercially available N-(t-butyloxycarbonyl)-L-leucine (11.9 g, 51.4 mmol) was dissolved in a mixture of tetrahydrofuran (THF) (45 mL) and dichloromethane (180 mL) under a N₂ atmosphere. The resulting solution was cooled to −20° C. To this solution was added 1-methylpiperidine (6.9 mL, 56.6 mmol) followed by isobutyl chloroformate (7.3 mL, 56.6 mmol) dropwise. The resulting mixture was stirred for 5 minutes at −20° C. and the above solution of N,O-dimethyl-hydroxylamine was added dropwise. The reaction mixture was stirred at −20° C. for 30 minutes followed by warming to room temperature. The reaction was quenched with water and the layers were separated. The aqueous layer was extracted with CH₂Cl₂ (3 times). The combined organic layers were washed with 10% citric acid, saturated sodium bicarbonate, brine, dried over Na₂SO₄ and concentrated under reduced pressure. Flash column chromatography (25% ethyl acetate (EtOAc) in hexanes) yielded 1 (13.8 g, 97%). [α]_{D}²³ −23 (c 1.5, MeOH); ¹H-NMR (400 MHZ, CDCl₃) δ 5.06 (d, 1H, J=9.1 Hz), 4.70 (m, 1H), 3.82 (s, 3H), 3.13 (s, 3H), 1.70 (m, 1H), 1.46-1.36 (m, 2H) 1.41 (s, 9H), 0.93 (dd, 6H, J=6.5, 14.2 Hz); ¹³C-NMR (100 MHZ, CDCl₃) δ 173.9, 155.6, 79.4, 61.6, 48.9, 42.1, 32.1, 28.3, 24.7, 23.3, 21.5; IR (neat) 3326, 2959, 2937, 2871, 1710, 1666, 1502, 1366, 1251, 1046 cm⁻¹; HRMS m/z (M+H)⁺ calc'd for C₁₃H₂₇N₂O₄ 275.1971, found 275.1964.

### B. N-(tert-Butoxycarbonyl)-L-Leucinal (2)

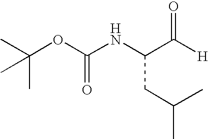

To a stirred suspension of lithium aluminum hydride (LAH) (770 mg, 20.3 mmol) in diethyl ether (60 mL) at −40° C. under N₂ atmosphere, was added dropwise a solution of 1 (5.05 g, 18.4 mmol) in diethyl ether (20 mL). The resulting reaction mixture was stirred for 30 minutes followed by quenching with 10% aqueous NaHSO₄ (30 mL) and warming to room temperature for 30 minutes. This solution was filtered and the filter cake was washed with diethyl ether (two times). The combined organic layers were washed with saturated sodium bicarbonate, brine, dried over MgSO₄ and concentrated under reduced pressure to afford 2 (3.41 g) which was used immediately without further purification. Crude ¹H-NMR (400 MHZ, CDCl₃) δ 9.5 (s, 1H), 4.9 (s, 1H), 4.2 (m, 1H), 1.8-1.6 (m, 2H), 1.44 (s, 9H), 1.49-1.39 (m, 1H), 0.96 (dd, 6H, J=2.7, 6.5 Hz).

### C. Ethyl (4S,5S)- and (4R,5S)-5-[(tert-Butoxycarbonyl)amino]-4-hydroxy-7-methyloct-2-ynoate (3)

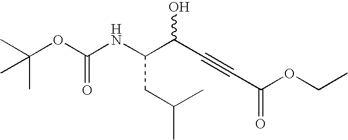

To a stirred solution of ethyl propiolate (801 mL) in THF (2 mL) at −78° C. was added a 1.0 M solution of lithium hexamethyldisilazide (7.9 mL) dropwise over a 5 minutes period. The mixture was stirred for 30 min, after which N-(tert-butoxycarbonyl)-L-leucinal 2 (or N-Boc-L-leucinal) (1.55 g, 7.2 mmol) in 8 mL of dry THF was added. The resulting mixture was stirred at −78° C. for 30 minutes. The reaction was quenched with saturated aqueous NH₄Cl at −78° C. followed by warming to room temperature. Brine was added and the layers were separated. The organic layer was dried over Na₂SO₄ and concentrated under reduced pressure. Flash column chromatography (15% EtOAc in hexanes) yielded a mixture of acetylenic alcohols 3 (68%). ¹H-NMR (300 MHZ, CDCl₃) δ 4.64 (d, 1H, J=9.0 Hz), 4.44 (br s, 1H), 4.18 (m, 2H), 3.76 (m, 1H), 1.63 (m, 1H), 1.43-1.31 (m, 2H), 1.39 (s, 9H), 1.29-1.18 (m, 3H), 0.89 (m, 6H); IR (neat) 3370, 2957, 2925, 2854, 1713, 1507, 1367, 1247, 1169, 1047 cm⁻¹.

### D. (5S,1′S)-5-[1′-[(tert-Butoxycarbonyl)amino]-3′-methylbutyl]dihydrofuran-2(3H)-one (4)

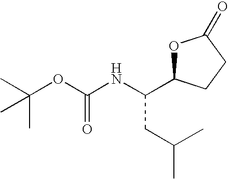

To a stirred solution of 3 (1.73 g, 5.5 mmol) in methanol (MeOH) (20 mL) was added 10% Pd/C (1.0 g). The resulting mixture was placed under a hydrogen balloon and stirred for 1 hour. After this period, the reaction was filtered through a pad of Celite and the filtrate was concentrated under reduced pressure. The residue was dissolved in toluene (20 mL) and acetic acid (100 L). The resulting mixture was refluxed for 6 hours followed by cooling to room temperature and concentrating under reduced pressure. Flash column chromatography (40% diethyl ether in hexanes) yielded 4 (0.94 g, 62.8 mmol) and less than 5% of its diastereomer. Lactone 4: M.p. 74-75° C.; [α]_{D}²³ −33.0 (c 1.0, MeOH); lit. (Fray, A. H., et al., J. Org. Chem. 51:4828-4833 (1986)) [α]_{D}²³ −33.8 (c 1.0, MeOH); ¹H-NMR (400 MHZ, CDCl₃) δ 4.50-4.44 (m, 2H), 3.84-3.82 (m, 1H), 2.50 (t, 2H, J=7.8 Hz), 2.22-2.10 (m, 2H), 1.64-1.31 (m, 3H), 1.41 (s, 9H), 0.91 (dd, 6H, J=2.2, 6.7 Hz); ¹³C-NMR (75 MHZ, CDCl₃) δ 177.2, 156.0, 82.5, 79.8, 51.0, 42.2, 28.6, 28.2, 24.7, 24.2, 23.0, 21.9; IR (neat) 2956, 2918, 2859, 1774, 1695, 1522, 1168 cm⁻¹; mass (EI) m/z 294 (M⁺+Na); HRMS: m/z (M+Na)⁺ calc'd for C₁₄H₂₅NO₄Na, 294.1681, found 294.1690.

### E. (3R,5S,1′S)-5-[1′-[(tert-Butoxycarbonyl)amino)]-3′-methylbutyl]-3-methyl-(3H)-dihydrofuran-2-one (5)

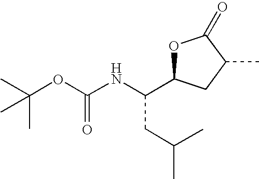

To a stirred solution of lactone 4 (451.8 mg, 1.67 mmol) in THF (8 mL) at −78° C. under a N₂ atmosphere, was added dropwise lithium hexamethyldisilazide (3.67 mL, 1.0 M in THF, 3.67 mmol). The resulting mixture was stirred at −78° C. for 30 minutes. Methyl iodide (MeI) (228 mL) was added dropwise and the resulting mixture was stirred at −78° C. for 20 minutes. The reaction was quenched with saturated aqueous NH₄Cl and allowed to warm to room temperature. The reaction mixture was concentrated under reduced pressure and the residue was extracted with EtOAc (three times). The combined organic layers were washed with brine, dried over Na₂SO₄ and concentrated under reduced pressure. Flash column chromatography (15% EtOAc in hexanes) yielded 5 (0.36 g, 76%). The stereochemistry of C₂-methyl group was assigned based upon NOESY and COSY experiments. Irradiation of the C₂-methyl group exhibited 6% NOE with the C₃ α-proton and 5% NOE with the C₄-proton. The α- and β-protons of C₃ were assigned by 2 D-NMR. [a]_{D}²³ −19.3 (c 0.5, CHCl₃); ¹H-NMR (300 MHZ, CDCl₃) δ 4.43 (t, 1H, J=6.3 Hz), 4.33 (d, 1H, J=9.6 Hz), 3.78 (m, 1H), 2.62 (m, 1H), 2.35 (m, 1H), 1.86 (m, 1H), 1.63-1.24 (m, 3H), 1.37 (s, 9H), 1.21 (d, 3H, J=7.5 Hz), 0.87 (dd, 6H, J=2.6, 6.7 Hz); ¹³C-NMR (75 MHZ, CDCl₃) δ 180.4, 156.0, 80.3, 79.8, 51.6, 41.9, 34.3, 32.5, 28.3, 24.7, 23.0, 21.8, 16.6; IR (neat) 2962, 2868, 1764, 1687, 1519, 1272, 1212, 1008 cm⁻¹; HRMS: m/z (M+Na)⁺ calc'd for C₁₅H₂₇NO₄Na, 308.1838, found 308.1828.

### F. (2R,4S,5S)-5-[(tert-Butoxycarbonyl)amino]-4-[(tert-butyldimethylsilyl)-oxy]-2,7-methyloctanoic acid (6)

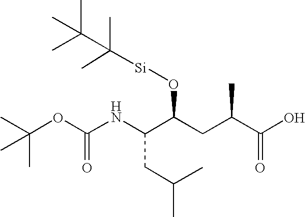

To a stirred solution of lactone 5 (0.33 g, 1.17 mmol) in a mixture of THF and water (5:1; 6 mL) was added LiOH.H₂O (0.073 g, 1.8 equiv). The resulting mixture was stirred at room temperature for 1 hour. The volatiles were removed under reduced pressure and the remaining solution was cooled to 0° C. and acidified with 25% aqueous citric acid to pH 3. The resulting acidic solution was extracted with EtOAc three times. The combined organic layers were washed with brine, dried over Na₂SO₄ and concentrated under reduced pressure to yield the corresponding hydroxy acid (330 mg) as a white foam. This hydroxy acid was used directly for the next reaction without further purification.

To the above hydroxy acid (330 mg, 1.1 mmol) in dimethylformamide (DMF) was added imidazole (1.59 g, 23.34 mmol) and tert-butyldimethylchlorosilane (1.76 g, 11.67 mmol). The resulting mixture was stirred at room temperature for 24 hours. MeOH (4 mL) was added and the mixture was stirred for an additional 1 hour. The mixture was acidified with 25% aqueous citric acid to pH 3 and was extracted with EtOAc three times. The combined extracts were washed with water, brine, dried over Na₂SO₄ and concentrated under reduced pressure. Flash column chromatography (35% EtOAc in hexanes) yielded 6 (0.44 g, 90%). M.p. 121-123° C.; [α]_{D}²³ −40.0 (c 0.13, CHCl₃); ¹H-NMR (400 MHZ, DMSO-d⁶, 343 K) δ 6.20 (br s, 1H), 3.68 (m, 1H), 3.51 (br s, 1H), 2.49-2.42 (m, 1H), 1.83 (t, 1H, J=10.1 Hz), 1.56 (m, 1H), 1.37 (s, 9H), 1.28-1.12 (m, 3H), 1.08 (d, 3H, J=7.1 Hz), 0.87 (d, 3H, J=6.1 Hz) 0.86 (s, 9H), 0.82 (d, 3H, J=6.5 Hz), 0.084 (s, 3), 0.052 (s, 3H); IR (neat) 3300-3000, 2955, 2932, 2859, 1711 cm⁻¹; HRMS: m/z (M+Na)⁺ calc'd for C₂₁H₄₃NO₅NaSi, 440.2808, found 440.2830.

II. Preparation of Other Isosteres Wherein P₁′ is an Alkyl Group

A. Isostere Used to Prepare MMI-133

The methyl diastereomers of the Leu-Ala isostere were synthesized using the minor product of the alkylation step (see Section I, step E).

Other isosteres with simple alkyl substituents in P₁′ (MMI-010, MMI-021, MMI-027-MMI-033, MMI-036, MMI-202, MMI-211, MMI-218) were produced following the general procedure for preparing the leucine-alanine isostere as set forth above except that a different alkylating agent was used in Section I, step E for alkylating the lactone. For example:

B. Leucine-Allyl Isostere Used to Prepare MMI-010 and MMI-021

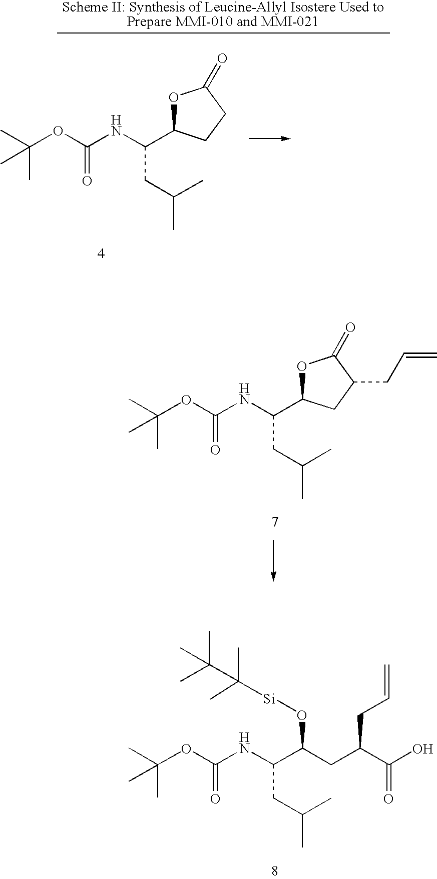

To a solution of 4 (2.41 g, 8.89 mmol) in THF (50 mL) was added lithium hexamethyldisilazane (1.0 M in THF, 19.56 mL, 19.56 mmol) dropwise at −78° C. The resulting mixture was stirred at −78° C. for 30 minutes. After this period, allyl iodide (0.89 mL, 9.78 mmol) was added dropwise at −78° C. and the resulting mixture was stirred at −78° C. for 15 minutes. The reaction mixture was poured into saturated aqueous NH₄Cl and extracted with EtOAc. The organic layer was washed with brine and dried over MgSO₄. Evaporation of the solvent under reduced pressure gave a residue which was purified by column chromatography (15% EtOAc in hexanes) to give 7 (1.94 g, 70%).

LiOH (66 mg, 1.58 mmol) was added to a solution of 7 (325 mg, 1.05 mmol) in dioxane/water (3:1, 4 mL) and stirred for 1 hour. The reaction mixture was acidified to pH 3 with 25% aqueous citric acid, extracted with EtOAc, dried over Na₂SO₄, and concentrated under reduced pressure to yield the corresponding hydroxyl acid (307 mg, 89%).

To a solution of the above hydroxyl acid (307 mg, 0.93 mmol) in DMF (8 mL) were added imidazole (1.07 g, 14.9 mmol) and TBSCl (1.12 g, 7.47 mmol). The reaction was stirred at room temperature for 15 hours. After this period, MeOH (4 mL) was added and the resulting mixture was stirred for 1 hour. The mixture was then diluted with 25% aqueous citric acid and extracted with EtOAc. The organic layer was washed with brine, dried with Na₂SO₄, and purified by column chromatography (10% EtOAc in hexanes) to yield 8 (383 mg, 93%).

C. Leucine-Homoserine Isostere Used to Prepare MMI-037:

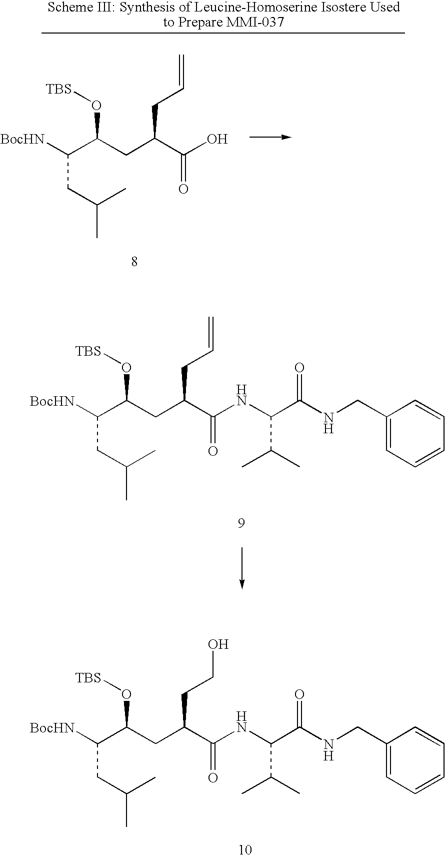

The isostere portion of MMI-037 is produced by coupling the above Leucine-Allyl isostere 8 with Valine-N-benzyl amide under standard EDCI/HOBt conditions (Section IV) to provide 9.

Ozone was bubbled through a solution of compound 9 in CH₂Cl₂/MeOH (1:1, 6 mL) at −78° C. until the blue color persisted (ca. 10 minutes). Oxygen was bubbled through the mixture until the blue color dissipated after which nitrogen was bubbled through the mixture for 10 minutes. Triphenylphosphine (124 mg, 0.47 mmol) was added at −78° C. and the mixture stirred and allowed to warm to room, temperature over 1 hour. The solvent was removed under reduced pressure and the residue was purified by column chromatography (30% EtOAc in hexanes) to yield the corresponding aldehyde (86 mg, 56%).

NaBH₄ (7.4 mg, 0.2 mmol) was added to a solution of the above aldehyde (86 mg, 0.13 mmol) in THF (3 mL) at 0° C. and stirred for 15 minutes. The reaction was quenched by addition of saturated aqueous NH₄Cl, extracted with EtOAc, dried with Na₂SO₄, and concentrated under reduced pressure. The resulting residue was purified by column chromatography (60% EtOAc in hexanes) to yield 10 (87%).

D. Leucine-Methionine Isostere Used to Prepare MMI-164:

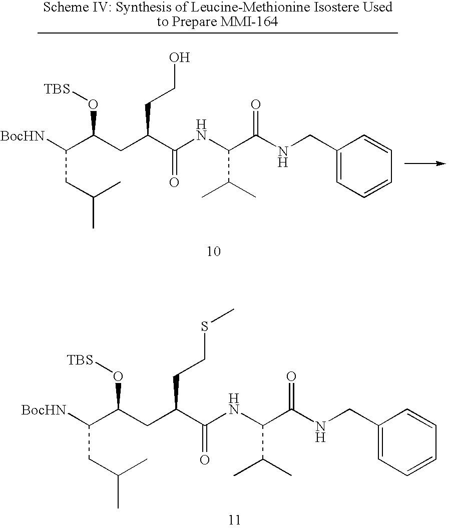

The isostere portion of MMI-164 is produced by treatment of a solution of 10 (70 mg, 0.11 mmol) in CH₂Cl₂ (2 mL) with Et₃N (0.03 mL, 0.22 mmol) and methane sulfonyl chloride (0.01 mL, 0.12 mmol) and stirred for 1 hour. The reaction mixture was diluted with CH₂Cl₂ and washed with saturated aqueous NH₄Cl. The organic layer was dried with Na₂SO₄ and concentrated under reduced pressure to yield the corresponding mesylate (67 mg).

To the above mesylate in DMF (2 mL) was added NaSMe (15 mg, 0.22 mmol) followed by heating to 70° C. for 1 hour. The reaction was cooled to room temperature, diluted with EtOAc, and washed with water. The organic layer was dried with Na₂SO₄, concentrated under reduced pressure and purified by column chromatography (20% EtOAc in hexanes) to yield 11 (65% for 2 steps).

E. Leucine-Asparagine Isostere Used to Prepare MMI-038:

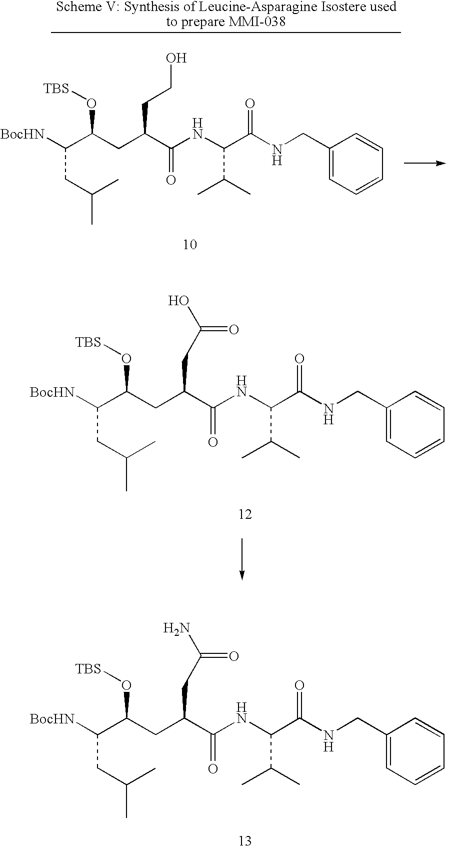

Pyridinium dichromate (302 mg, 0.81 mmol) was added to a solution of 10 (170 mg, 0.27 mmol) in DMF (2 mL) and stirred at room temperature for 12 hours. The reaction mixture was diluted with Et₂₀ and filtered through Celite®. The filtrate was concentrated under reduced pressure and purified by column chromatography (10% MeOH in CHCl₃) to afford 12 (130 mg, 77%).

HOBt (7.1 mg, 0.05 mmol) and EDCI (10 mg, 0.05 mmol) were added to 12 (30 mg, 0.04 mmol) in CH₂Cl₂ (2 mL). After stirring for 30 minutes at room temperature, the solution was added to liquid NH₃ in CH₂Cl₂ at −78° C. After stirring at −78° C. for 30 minutes, the reaction mixture was warmed to room temperature, diluted with CH₂Cl₂, and washed with water. The organic layer was dried with Na₂SO₄, concentrated under reduced pressure and purified by column chromatography (60% EtOAc in hexanes) to yield 13 (19 mg, 63%).

F. Leucine-Serine Isostere Used to Prepare MMI-078 and MMI-132:

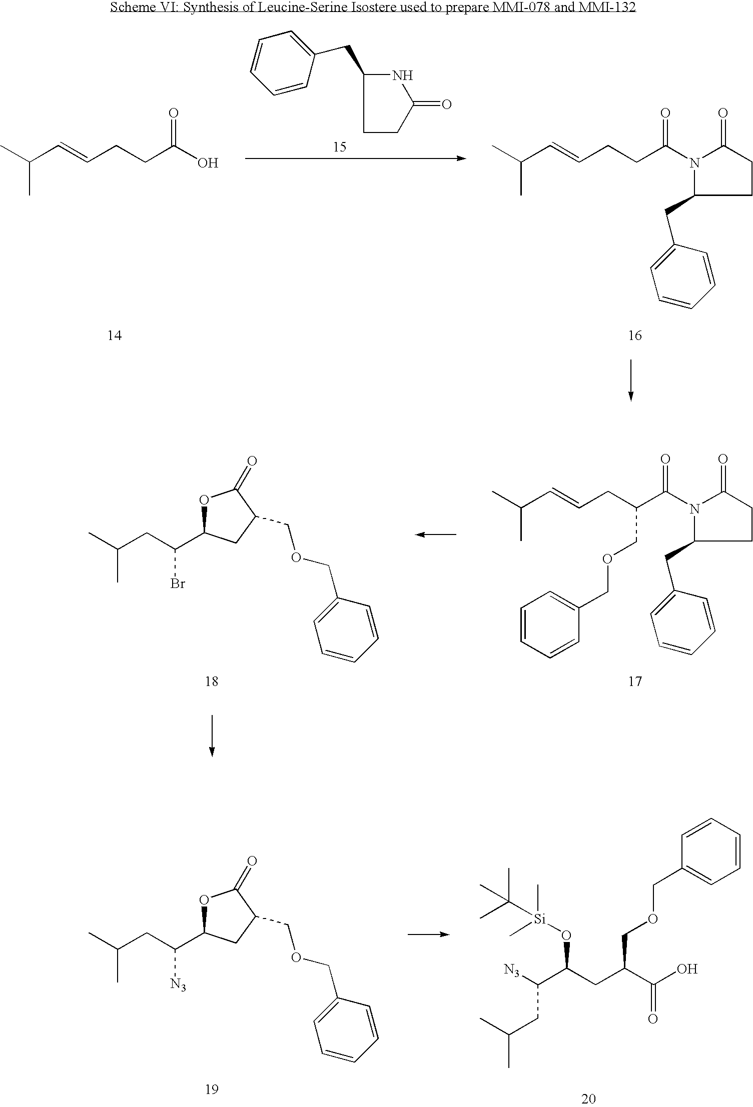

To a solution of known carboxylic acid 18 (Tetrahedron 1996, 8451) (1.05 g, 6.78 mmol) in THF (30 mL) at −20° C. was added Et₃N (1.2 mL, 8.82 mmol) dropwise followed by pivaloyl chloride (1.08 mL, 8.82 mmol). The mixture was stirred for 30 minutes at −20° C. followed by cooling to −78° C.

In a separate flask, oxazolidinone 15 (1.56 g, 8.82 mmol) was dissolved in THF (25 mL), cooled to −78° C. and BuLi (5.5 mL, 1.6 M in hexanes, 8.82 mmol) was added dropwise. After stirring for 30 minutes, the solution was transferred via cannula into the first flask containing the mixed anhydride at −78° C. The resulting mixture was stirred for 30 minutes and quenched with NaHSO₄ (5 g in 30 mL H₂O) at −78° C. and warmed to room temperature. The organic layer was dried with NaSO₄, concentrated under reduced pressure and purified by column chromatography (20% EtOAc in hexanes) to yield 16 (2.37 g, 71%).

To a solution of 16 (47 mg, 0.15 mmol) in THF (1 mL) was added lithium hexamethyldisilazane (1.0 M in THF, 0.19 mL, 0.19 mmol) dropwise at −78° C. The resulting mixture was stirred at −78° C. for 30 minutes. After this period, benzylchloromethyl ether (0.027 mL, 0.19 mmol) was added dropwise at −78° C. and the resulting mixture was stirred at −78° C. for 15 minutes. The reaction mixture was poured into saturated aqueous NH₄Cl and extracted with EtOAc. The organic layer was washed with brine and dried over MgSO₄. Evaporation of the solvent under reduced pressure gave a residue which was purified by column chromatography (10% EtOAc in hexanes) to give 17 (68%).

To a solution of 17 (157 mg, 0.36 mol) in DME/H2O (3:1, 8 mL) was added NBS (70.8 mg, 0.4 mmol) at 0° C. After stirring for 45 minutes at 0° C., the reaction was quenched by the addition of H₂O and extracted with EtOAc. The organic layer was washed with saturated aqueous NaHCO₃, brine and dried over MgSO₄. Evaporation of the solvent under reduced pressure gave a residue which was purified by column chromatography (10% EtOAc in hexanes) to give 18 (59%).

The reaction of 18 (73 mg, 0.21 mmol) with NaN₃ (27 mg, 0.42 mmol) in DMPU (1 mL) at room temperature for 3 days yielded 19 (66%) after column chromatography (15% EtOAc in hexanes).

Completion of the isostere synthesis was accomplished following procedures previously described (Section I, Step F) to afford 20 followed by coupling of Valine-N-benzyl amide under standard EDCI/HOBt coupling conditions (Section IV) and hydrogenation of the azide and benzyl protecting group following standard hydrogenation conditions to provide the corresponding aminoalcohol.

Standard hydrogenation procedure: A mixture of the alkene, benzyl-protected alcohol, or azide (135 mg, 0.4 mmol) and Pd(OH)/C (20%, 20 mg) in MeOH, EtOAc or a mixture thereof (5 mL) was stirred under an H₂ atmosphere for 5 hours. The catalyst was filtered off and the filtrate was concentrated under reduced pressure to yield the corresponding saturated compound, free alcohol, or free amine quantitatively.

G. Leucine-CH₂ Isostere Used to Prepare MMI-145:

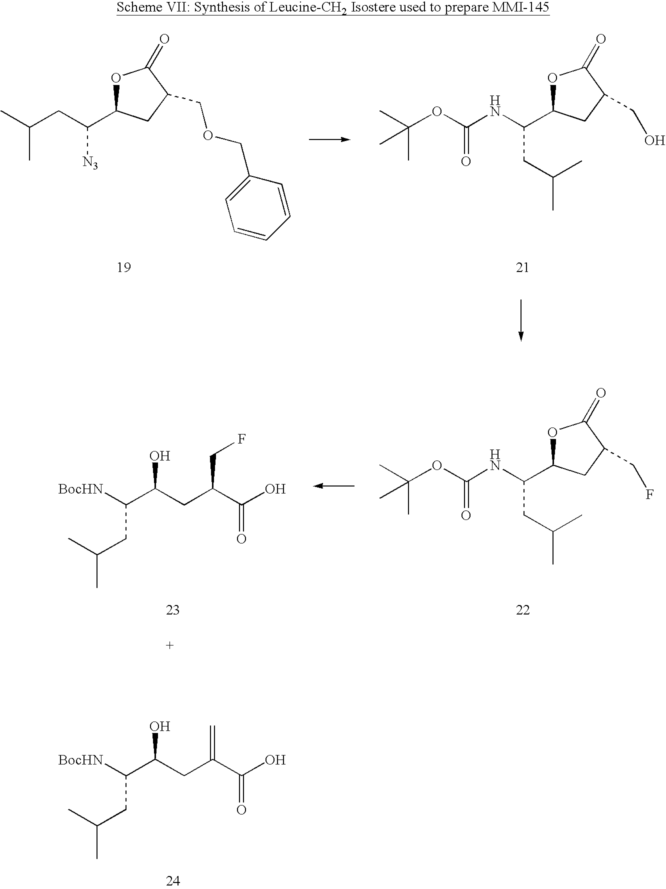

To a solution of 19 (35 mg, 0.11 mmol) in MeOH (2 mL) was added Boc₂O (0.038 mL, 0.16 mmol) and Pd(OH)₂/C (20% Pd, 5 mg). The mixture was placed under a hydrogen atmosphere and stirred for 12 hours at room temperature. The reaction was filtered through Celite®, the filtrate was concentrated under reduced pressure, and the residue was purified by column chromatography (50% EtOAc in hexanes) to yield 21 (44%).

To a solution of (diethylamino)sulfur trifluoride (0.0095 mL, 0.07 mmol) in CH₂Cl₂ (1 mL) at −78° C. was added dropwise a solution of 21 (20 mg, 0.06 mmol) in CH₂Cl₂ (1 mL). The reaction was warmed to room temperature and stirred for 12 hours. After this period, the reaction mixture was cooled to 0° C. and quenched with H₂O. The organic layer was dried with Na₂SO₄, concentrated under reduced pressure and purified by column chromatography (25% EtOAc in hexanes) to yield 22 (61%).

To a solution of 22 (46.5 mg, 0.15 mmol) in DME:H₂O (1:1, 3 mL) was added 1 N LiOH (0.46 mL) and stirred at room temperature for 2 hours followed by acidification with 1 N HCl to pH 3 and extraction with EtOAc. The organic layer was dried with Na₂SO₄, concentrated under reduced pressure and purified by column chromatography to yield a mixture of products. The mixture (44 mg, 0.13 mmol) was dissolved in DMF (1 mL) and imidazole (207 mg, 3.04 mmol) and TBSCl (209 mg, 1.38 mmol) was added and stirred for 12 hours. The reaction was quenched MeOH (1 mL), stirred for 1 hour, acidified with 5% citric acid to pH 3, extracted with EtOAc, dried with Na₂SO₄, concentrated under reduced pressure and purified by column chromatography to yield 23 (13.8 mg) and the isostere 24 (37.6 mg).

H. Leucine-Tyrosine Isostere Used to Prepare MMI-101 and MMI-102:

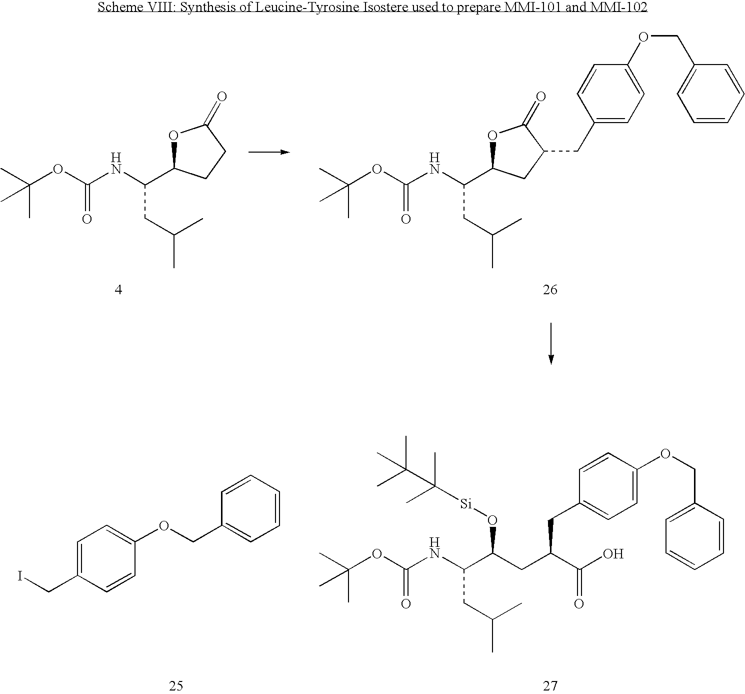

To a solution of 4 (220 mg, 0.81 mmol) in THF (50 mL) was added lithium hexamethyldisilazane (1.0 M in THF, 1.78 mL, 1.78 mmol) dropwise at −78° C. The resulting mixture was stirred at −78° C. for 30 minutes. After this period, iodide 25 (22 mg, 0.89 mmol) was added dropwise at −78° C. and the resulting mixture was stirred at −78° C. for 15 minutes. The reaction mixture was poured into saturated aqueous NH₄Cl and extracted with EtOAc. The organic layer was washed with brine and dried over MgSO₄. Evaporation of the solvent under reduced pressure gave a residue which was purified by column chromatography (10% EtOAc in hexanes) to give 26 (242 mg, 63%).

LiOH (32 mg, 0.77 mmol) was added to a solution of 26 (242 mg, 0.51 mmol) in dioxane/water (3:1, 4 mL) and stirred for 1 hour. The reaction mixture was acidified to pH 3 with 25% aqueous citric acid, extracted with EtOAc, dried over NaSO₄, and concentrated under reduced pressure to yield the corresponding hydroxy acid (240 mg, 96%).

To a solution of the above hydroxyl acid (240 mg, 0.49 mmol) in DMF (6 mL) were added imidazole (533 mg, 7.84 mmol) and TBSCl (588 mg, 3.92 mmol). The reaction was stirred at room temperature for 15 hours. After this period, MeOH (4 mL) was added and the resulting mixture was stirred for 1 hour. The mixture was then diluted with 25% aqueous citric acid and extracted with EtOAc. The organic layer was washed with brine, dried with Na₂SO₄, and purified by column chromatography (10% EtOAc in hexanes) to yield 27 (89%).

After the standard EDCI/HOBt couplings (Section IV) to produce MMI-101, the benzyl protecting group was removed by hydrogenation following the standard hydrogenation procedure described previously (Section II, Step F).

III. Other Isosteres

A. Isosteres Having an Inverted Hydroxyl Group (MMI-003, MMI-113, MMI-133)

The methyl/hydroxy diastereomer of the Leucine-Alanine isostere (MMI-133) was synthesized using the minor product from the ethylpropiolate addition step (Section I, Step C) following the regular sequence for the Leucine-Alanine isostere.

MMI-133 was produced from the above diastereomer by the following sequence to invert the methyl chiral center:

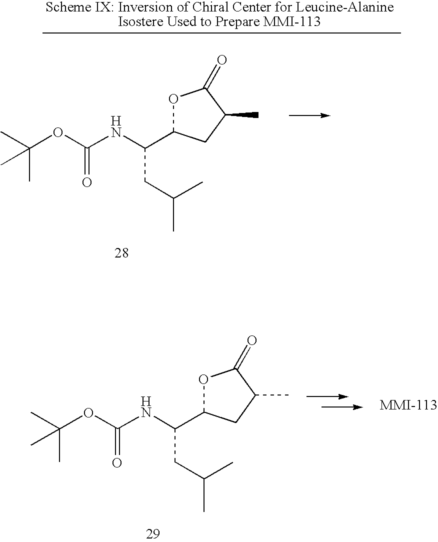

To a solution of iPr₂NEt (0.07 mL, 0.5 mmol) in THF (2 mL) at 0° C. was added BuLi (0.32 mL, 1.6 M in hexanes, 0.51 mmol) and stirred for 30 minutes. The above solution was cooled to −78° C. and 28 (28.3 mg, 0.1 mmol) in THF (1 mL) followed by HMPA (0.1 mL, 0.55 mmol) were added. After stirring for 1 hour at −78° C. and 1.5 hours at −42° C., the reaction was cooled to −78° C. and dimethylmalonate (0.11 mL, 1.0 mmol) was added. The reaction was allowed to warm to room temperature, diluted with EtOAc, washed with saturated aqueous NH₄Cl, brine, dried with NaSO₄, concentrated under reduced pressure and purified by column chromatography (15% EtOAc in hexanes) to yield 29 (86%). 29 was then carried through the normal procedures for the production of the isostere of MMI-133 (see Section I).

B. Hydroxyethylamine Isostere Used to Prepare MMI-061, MMI-062, MMI-092, MMI-106:

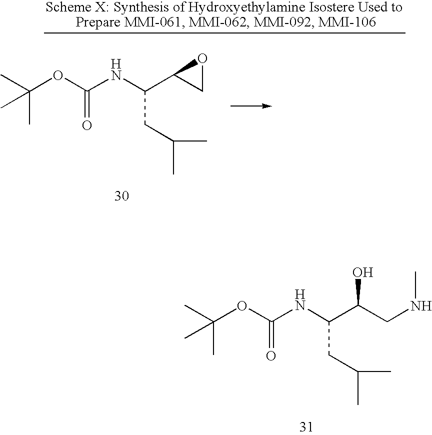

To a solution of known epoxide 30 (Tetrahedron Lett., 1995, 36, 2753-2756) (229 mg, 1.0 mmol) in MeOH (5 mL) was added methylamine (2.5 mL, 2.0 M solution in MeOH, 5.0 mmol) and the resulting mixture was stirred for 4-5 hours at room temperature. The solvent was removed under reduced pressure and the residue was purified by column chromatography (50% EtOAc in hexanes) to afford 31 (90% yield).

C. Isostere Wherein P₁′ and R₄ Form a Pyrrolidin-2-one Ring (MMI-181, MMI-185, MMI-187, MMI-191, MMI-192):

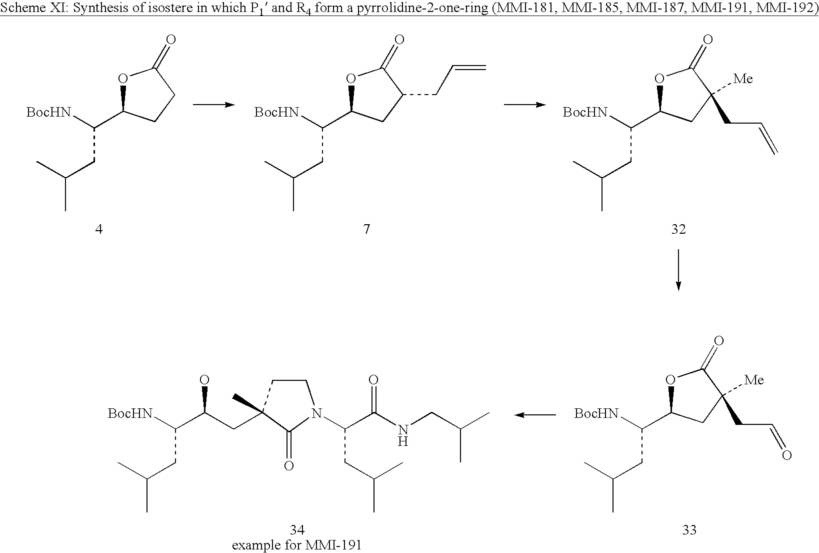

To a solution of 4 (2.41 g, 8.89 mmol) in THF (50 mL) was added lithium hexamethyldisilazane (1.0 M in THF, 19.56 mL, 19.56 mmol) dropwise at −78° C. The resulting mixture was stirred at −78° C. for 30 minutes. After this period, allyl iodide (0.89 mL, 9.78 mmol) was added dropwise at −78° C. and the resulting mixture was stirred at −78° C. for 15 minutes. The reaction mixture was poured into saturated aqueous NH₄Cl and extracted with EtOAc. The organic layer was washed with brine and dried over MgSO₄. Evaporation of the solvent under reduced pressure gave a residue which was purified by column chromatography (15% EtOAc in hexanes) to give 7 (1.94 g, 70%).

To a solution of 7 (1.5 g, 4.82 mmol) in THF (30 mL) was added lithium hexamethyldisilazane (1.0 M in THF, 10.60 mL, 10.60 mmol) dropwise at −78° C. The resulting mixture was stirred at −78° C. for 30 minutes. After this period, methyl iodide (0.39 mL, 6.26 mmol) was added dropwise at −78° C. and the resulting mixture was warmed to 0° C. for 1 hour. The reaction mixture was poured into saturated aqueous NH₄Cl and extracted with EtOAc. The organic layer was washed with saturated aqueous NaHCO₃, brine and dried over MgSO₄. Evaporation of the solvent under reduced pressure gave a residue which was purified by column chromatography (12% EtOAc in hexanes) to give 32 (0.684 g, 44%).

Ozone was bubbled through a solution of 32 (250 mg, 0.768 mmol) in CH₂Cl₂/MeOH (1:1, 20 mL) at −78° C. until the blue color persisted. The solution was then flushed with N₂ for 10 minutes. Me₂S was added (0.31 mL, 4.23 mmol) slowly and the reaction mixture was allowed to warm to room temperature. After being stirred for 12 hours, the solvent was removed under reduced pressure and the resulting residue was purified by column chromatography (30% EtOAc in hexanes) to give 33 (155 mg, 62%).

Incorporation of compound 34 into the isostere used to prepare MMI-191 is described as an example:

To a solution of the trifluoroacetic acid (TFA) salt of leucine N-ⁱbutyl amide (0.092 mmol) and sodium acetate (10 mg, 0.73 mmol) was added compound 34 (20 mg, 0.061 mmol) at room temperature. The mixture was stirred at room temperature for 15 minutes followed by addition of NaBH₃CN (5.4 mg, 0.086 mmol). The resulting mixture was stirred at room temperature for 24 h, poured into H₂O, and extracted with EtOAc. The organic layer was washed with brine and dried with MgSO₄. Concentration under reduced pressure afforded a residue which was chromatographed (40% EtOAc in hexanes) to give compound 13 (30 mg, 99%).

D. Phenylalanine-Methionine Isostere Used to Prepare MMI-201:

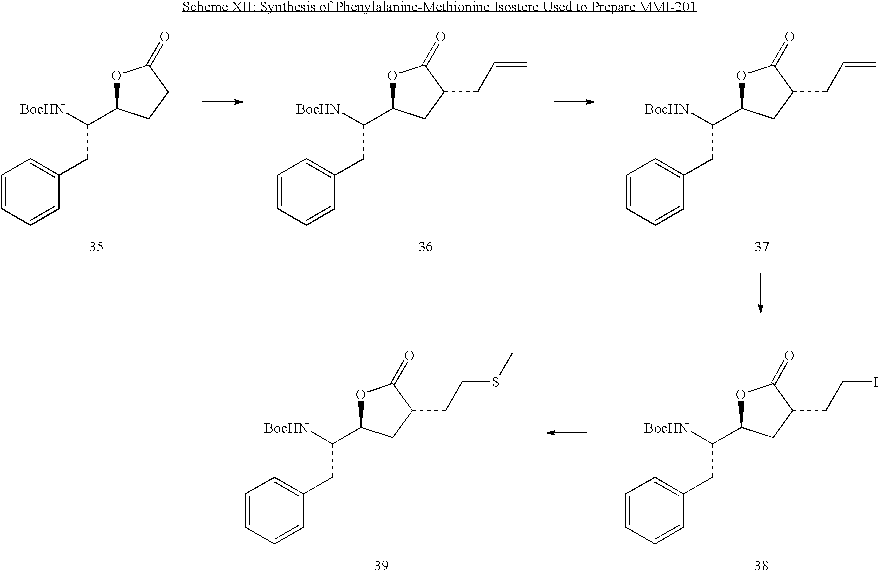

To a solution of 35 (635 mg, 2.08 mmol) in THF (15 mL) was added lithium hexamethyldisilazane (1.0 M in THF, 4.57 mL, 4.57 mmol) dropwise at −78° C. The resulting mixture was stirred at −78° C. for 30 minutes. After this period, allyl iodide (0.21 mL, 2.29 mmol) was added dropwise at −78° C. and the resulting mixture was stirred at −78° C. for 15 minutes. The reaction mixture was poured into saturated aqueous NH₄Cl and extracted with EtOAc. The organic layer was washed with brine and dried over MgSO₄. Evaporation of the solvent under reduced pressure gave a residue which was purified by column chromatography (15% EtOAc in hexanes) to give 36 (413 mg, 58%).

Ozone was bubbled through a solution of 36 (400 mg, 1.158 mmol) in CH₂Cl₂/MeOH (1:1, 30 mL) at −78° C. until the blue color persisted. The solution was then flushed with N2 for 10 minutes. Triphenylphosphine (334 mg, 1.274 mmol) was added slowly and the reaction mixture was allowed to warm to room temperature. After being stirred for 15 minutes, the solvent was removed under reduced pressure and the resulting residue was purified by column chromatography (40% EtOAc in hexanes) to give 37 (336 mg, 84%).

To a solution of aldehyde 37 (300 mg, 0.86 mmol) in MeOH (10 mL) was added NaBH₄ (49 mg, 1.3 mmol) at −78° C. The reaction was allowed to warm to 0° C. and was stirred at that temperature for 20 minutes. The reaction mixture was poured into saturated aqueous NH₄Cl and extracted with EtOAc. The organic layer was washed with brine and dried over MgSO₄. Concentration under reduced pressure afforded a residue that was chromatographed (60% EtOAc in hexanes) to yield the corresponding alcohol (200 mg, 66%).

To a solution of the above alcohol (170 mg, 0.49 mmol) in CH₂Cl₂ (5 mL) was added imidazole (83 mg, 1.22 mmol), Ph₃P (319 mg, 1.22 mmol) and iodine (247 mg, 0.97 mmol) at 0° C. The reaction was stirred for 15 minutes at 0° C., poured into saturated aqueous Na₂SO₄, and extracted with CH₂Cl₂. The organic layer was washed with brine, dried over MgSO₄, concentrated under reduced pressure, and chromatographed (30% EtOAc in hexanes) to give 38 (124 mg, 55%).

To a solution of iodide 38 (124 mg, 0.27 mmol) in DMF (5 mL) was added sodium thiomethoxide (23 mg, 0.32 mmol) at 0° C. The reaction was stirred for 10 minutes, poured into saturated aqueous NH₄Cl, and extracted with diethylether. The organic layer was washed with saturated aqueous NaHCO₃, brine and dried over MgSO₄. Concentration under reduced pressure gave a residue which was chromatographed (30% EtOAc in hexanes) to give 39 (66 mg, 64%). The lactone was then hydrolyzed with LiOH and the resulting free alcohol protected as in the synthesis of the Leucine-Alanine isostere.

E. Isostere Having Dimethyl Groups at the P₁′ Position (MMI-218):

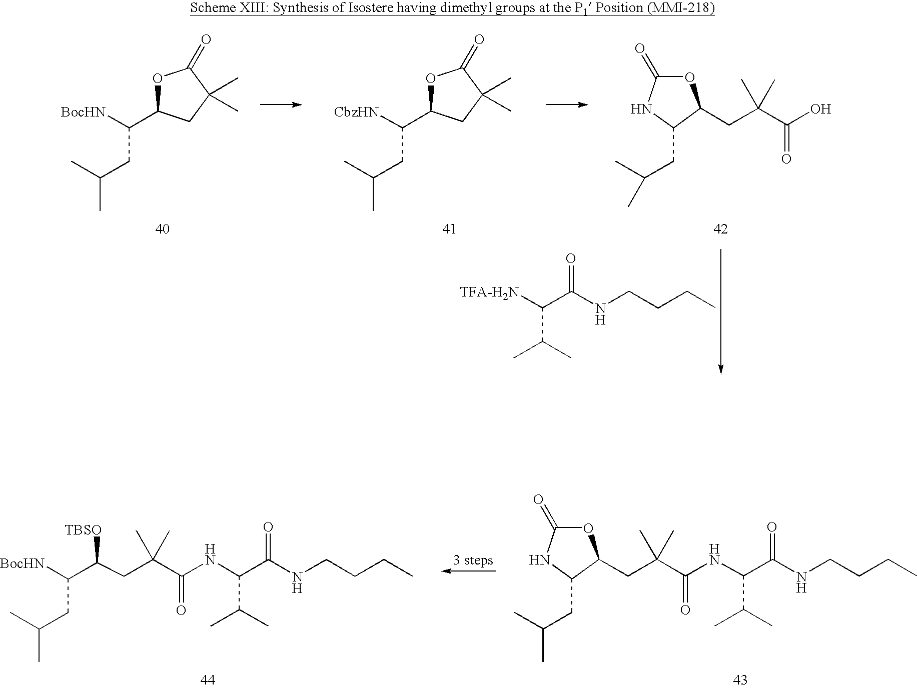

To a solution of 4 (625 mg, 2.19 mmol) in THF (20 mL) was added lithium hexamethyldisilazane (1.0 M in THF, 4.8 mL, 4.8 mmol) dropwise at −78° C. The resulting mixture was stirred at −78° C. for 1 hour. After this period, methyl iodide (0.15 mL, 2.41 mmol) was added dropwise at −78° C. and the resulting mixture was warmed to −45° C. for 1 hour. After this period, to the reaction mixture was added lithium hexamethyldisilazane (1.0 M in THF, 4.8 mL, 4.8 mmol) dropwise at −78° C. The resulting mixture was stirred at −78° C. for 1 hour. After this period, methyl iodide (0.15 mL, 2.41 mmol) was added dropwise at −78° C. and the resulting mixture was warmed to −45° C. for 1 hour. The reaction mixture was poured into saturated aqueous NH₄Cl and extracted with EtOAc. The organic layer was washed with brine and dried over MgSO₄. Evaporation of the solvent under reduced pressure gave a residue which was purified by column chromatography (15% EtOAc in hexanes) to give 40 (416 mg, 63%).

To a solution of 40 (416 mg, 1.389 mmol) in CH₂Cl₂ (8 mL) was added trifluoroacetic acid (2 mL) at 0° C. and the resulting mixture was stirred at 0° C. for 3.5 hours. After this time, the reaction was concentrated under reduced pressure to obtain the crude amine. To this crude amine in CH₂Cl₂ (15 mL) was added iPr₂NEt (0.8 mL, 4.58 mmol) and benzylchloroformate (0.22 mL, 1.53 mmol) at −78° C. The reaction was stirred for 1 hour at −78° C., poured into saturated aqueous NH₄Cl, and extracted with CH₂Cl₂. The organic layer was washed with brine and dried over MgSO₄. Evaporation of the solvent under reduced pressure gave a residue which was purified by column chromatography (20% EtOAc in hexanes) to give 41 (408 mg, 88%).

To a solution of 41 (408 mg, 1.22 mmol) in THF (15 mL) was added 1N aqueous LiOH solution (9.8 mL, 9.8 mmol) at room temperature. The resulting mixture was stirred at room temperature for 15 hours. After this period, the reaction was concentrated under reduced pressure and the remaining aqueous residue was cooled to 0° C. and acidified with 25% aqueous citric acid to pH 4. The resulting acidic solution was extracted with EtOAc. The organic layer was washed with brine and dried over MgSO₄. Evaporation of the solvent under reduced pressure gave a residue which was purified by column chromatography (70% EtOAc in hexanes) to give 42 (110 mg, 37%).

42 was coupled with Valine-N-nbutyl amide under standard EDCI/HOBt coupling conditions (Section IV) to afford 43.

To a solution of 43 (81 mg, 0.20 mmol) in THF (4 mL) was added Et₃N (0.032-mL, 0.224 mmol), Boc₂O (53 mg, 0.245 mmol), and dimethylaminopyridine (5 mg, 0.041 mmol) at 0° C. After being stirred at room temperature for 3 hours, the reaction mixture was poured into saturated aqueous NH₄Cl and extracted with EtOAc. The organic layer was washed with brine and dried over MgSO₄. Evaporation of the solvents under reduced pressure gave a residue which was purified by column chromatography (5% MeOH in CHCl₃) to give the corresponding Boc-protected oxazolidinone (99 mg, 98%).

To the above Boc-protected oxazolidinone (74 mg, 0.149 mmol) in MeOH (4 mL) was added Cs₂CO₃ (97 mg, 0.297 mmol) at room temperature. After stirring at room temperature for 20 hours, the reaction mixture was neutralized with 1 N aqueous HCl and extracted with EtOAc. The organic layer was washed with brine and dried over MgSO₄. Evaporation of the solvent under reduced pressure gave a residue which was purified by column chromatography (2% MeOH in CHCl₃) to give the corresponding amino alcohol (38 mg, 54%).

To the above amino alcohol (38 mg, 0.081 mmol) in CH₂Cl₂ (2 mL) were added t-butyldimethylsilyl trifluoromethanesulfonate (0.022 mL, 0.097 mmol) and iPr₂NEt (0.034 mL, 0.193 mmol) at −78° C. After being stirred at −78° C. for 15 minutes, the reaction mixture was poured into saturated aqueous NH₄Cl and extracted with CH₂Cl₂. The organic layer was washed with brine and dried over MgSO₄. Evaporation of the solvent under reduced pressure gave a residue which was purified by column chromatography (1.5% MeOH in CHCl₃) to give 44 (42 mg, 89%).

B. Isosteres Having P₁ Amino Acid Side Chains Other Than Leucine Side Chain

Inhibitors with a different amino acid-based side-chain in P₁ were produced by substitution the appropriate Boc-protected amino acids for N-(t-butyloxycarbonyl)-L Leucine (Boc-Phe: MMI-040, MMI-048, MMI-201; Boc-Ser: MMI-155) in Section I, Step A.

C. Isostere Having Non-Natural P₁ Amino Acid Side Chains (MMI-178, MMI-179, MMI-170, MMI-172)

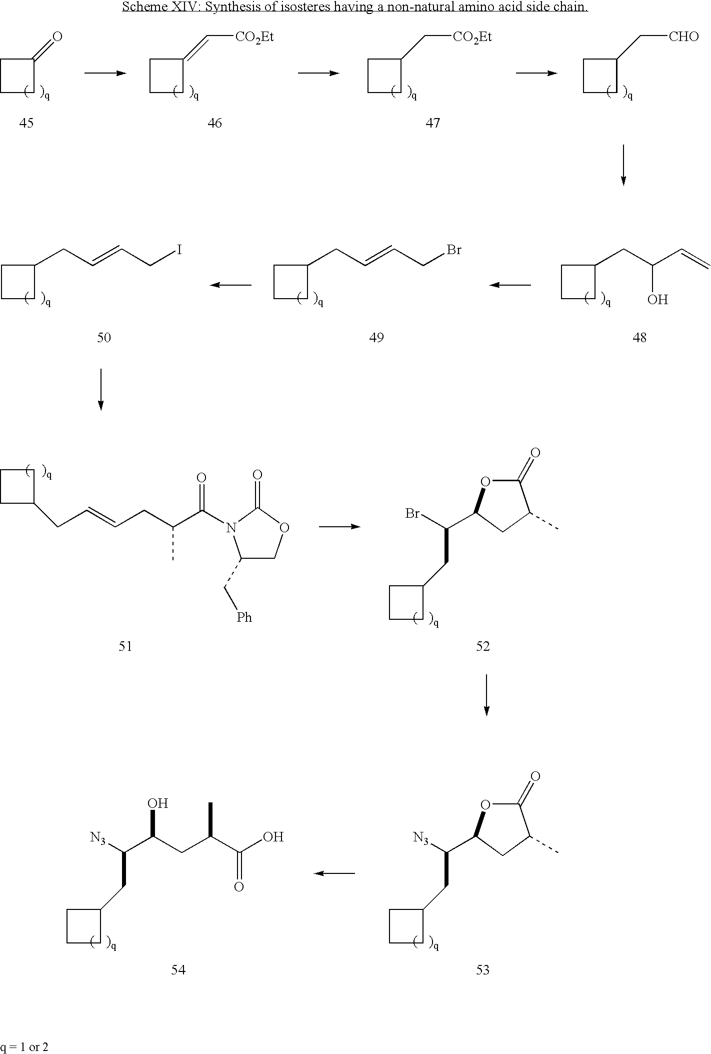

i) Preparation of Compound 46:

To a solution of NaH (4.8 g, 0.12 mol) in THF (150 mL) was added triethylphosphonoacetate (23.8 mL, 0.12 mol) dropwise at 0° C. for 10 minutes. To the stirred mixture was added cyclobutanone (7.5 mL, 0.10 mol) (for q=2, cyclopentanone was added instead of cyclobutanone). After 1 hour at room temperature, the reaction mixture was poured into saturated aqueous NH₄Cl and was extracted with EtOAc. The organic layer was washed with saturated aqueous NaHCO₃, brine and dried over MgSO₄. Evaporation of the solvent under reduced pressure gave a residue which was purified by column chromatography (5% EtOAc in hexanes) to give 46 (13.66 g, 96%).

ii) Preparation of Compound 47:

Compound 46 was hydrogenated at 40 psi with Pd/C in ethanol (EtOH) to afford compound 47 in 84% yield.

iii) Preparation of Compound 48:

Compound 47 was reduced to an aldehyde with diisobutylaluminum hydride (DIBAL-H) at −78° C. and the aldehyde was reacted with vinylmagnesium bromide at −20° C. to yield compound 48 (39% for two steps).

iv) Preparation of Compound 49:

To a solution of compound 48 (2.158 g, 17.1 mmol) and 1,5-hexadiene (1.52 mL, 12.83 mmol) in CH₂Cl₂ was added SOBr₂ (2.0 mL, 25.65 mmol) at 0° C. After the mixture was stirred at 0° C. for 45 min, the reaction was quenched by the addition of H₂O and stirred at 0° C. for 15 minutes. The mixture was extracted with CH₂Cl₂. The organic layer was washed with saturated aqueous NaHCO₃, brine and dried over MgSO₄. Evaporation of the solvent under reduced pressure gave a residue which was purified by column chromatography (hexanes) to give compound 49 (2.85 g, 88%).

v) Preparation of Compound 50:

To a solution of compound 49 (2.4 g, 12.69 mol) in acetone (40 mL) was added NaI (2.47 g, 16.50 mmol). After 1 hour at room temperature, the reaction was quenched by the addition of H₂O. The mixture was concentrated under reduced pressure and the remaining aqueous residue was extracted with EtOAc. The organic layer was washed with saturated aqueous Na₂S₂O₃, brine and dried over MgSO₄. Evaporation of the solvent under reduced pressure gave a residue which was purified by column chromatography (hexanes) to give compound 50 (2.43 g, 81%).

vi) Preparation of Compound 51:

Compound 30 was prepared from compound 50 in 71% yield following Evan's protocol (J. Med. Chem. 33:2335-2342 (1990)).

vii) Preparation of Compound 52:

To a solution of compound 52 (2.1 g, 6.15 mol) in ethylene glycol dimethyl ether (DME)/H₂O (1:1, 40 mL) was added N-bromosuccinamide (NBS) (1.2 g, 6.77 mmol) at 0° C. After stirring for 45 minutes at 0° C., the reaction was quenched by the addition of H₂O and extracted with EtOAc. The organic layer was washed with saturated aqueous NaHCO₃, brine and dried over MgSO₄. Evaporation of the solvent under reduced pressure gave a residue which was purified by column chromatography (10% EtOAc in hexanes) to give compound 52 (727 mg, 45%).

viii) Preparation of 53:

The reaction of compound 52 with NaN₃ in 1,3-dimethyl-3,4,5,6-tetrahydro-2(1H)-pyrimidinone (DMPU) at room temperature for 3 days yielded compound 32 (65%). Completion of the isostere synthesis was accomplished by hydrolysis of the lactone with LiOH, TBS protection of the resulting alcohol (see Part L step F), and hydrogentaion of the azide following the standard hydrogenation procedure described previously (Section II, Step F).

H. Isosteres in MMI-162, MMI-163, MMI-168, MMI-169 are Described in the Following Scheme:

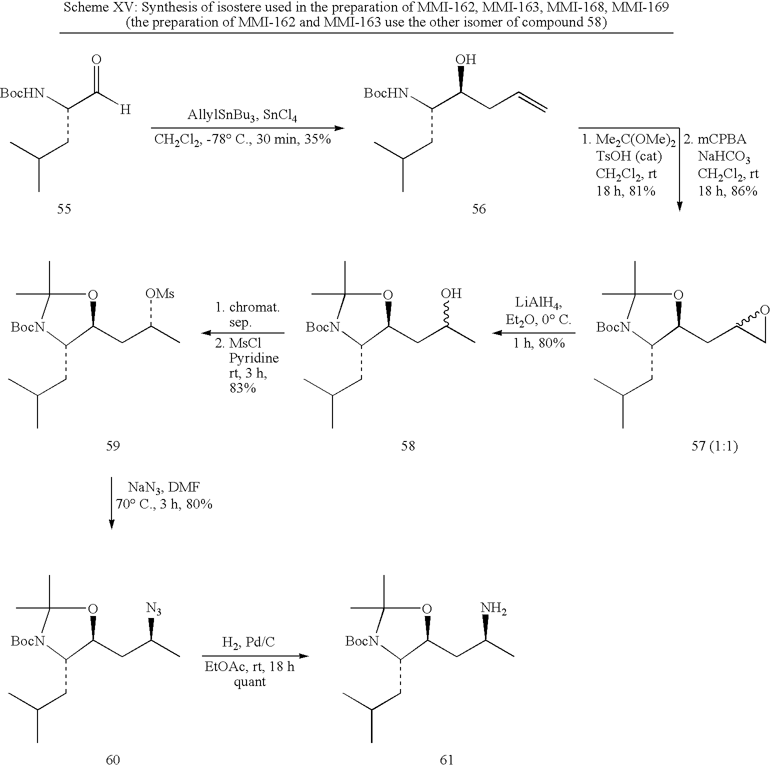

The synthesis of MMI-162 and MMI-163 used one isomer of compound 58, and the synthesis of MMI-168 and MMI-169 used the other isomer of compound 58.

IV. Amide Bond Formation

Amide bonds in inhibitors of the invention were generally created through 1-[3-(dimethylamino)propyl]-3-ethylcarbodiimide (EDCI) and 1-hydroxybenzotriazole (HOBt)-mediated coupling of the appropriate carboxylic acid and amine. An example is given below for the coupling of isostere 6 and amine-containing compound 62.

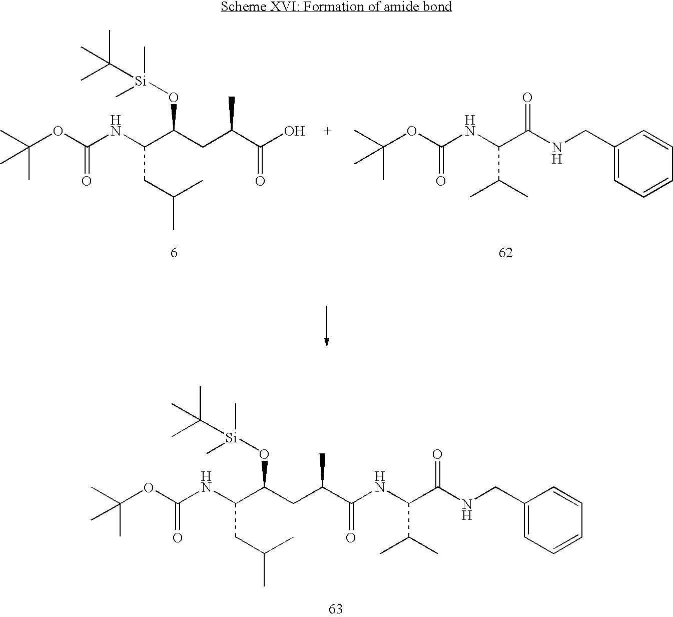

Boc-protected amine compound 62 (71 mg, 0.10 mmol) was dissolved in CH₂Cl, (3 mL) and TFA (0.75 mL) was added at room temperature. The reaction mixture was stirred for 30 minutes followed by concentrating under reduced pressure to provide the free amine (61 mg, quantitative). Leucine-alanine isostere 6 (42 mg, 0.1 mmol) was dissolved in dichloromethane (DCM) (2 mL). To this solution, HOBt (20 mg, 0.15 mmol) and EDCI (29 mg, 0.15 mmol) were added successively at room temperature and stirred for 5 minutes. To this solution was added dropwise a solution of the above free amine (41 mg, 0.2 mmol) and diisopropylethylamine (0.2 mL) and the resulting mixture was stirred overnight. The mixture was poured into H₂O and extracted with EtOAc, dried over Na₂SO₄ and concentrated under reduced pressure. Flash column chromatography (20% EtOAc in hexanes) yielded compound 63 (57 mg, 95%). ¹H-NMR (500 MHZ, CDCl₃) δ 0.09 (s, 3H), 0.10 (s, 3H), 0.91 (s, 9H), 0.92-0.98 (m, 12H), 1.10 (d, 3H, J=6.7 Hz), 1.25 (m 1H), 1.44 (m, 1H), 1.46 (s, 9H), 1.63 (m, 1H), 1.74 (br s, 1H), 1.80 (m, 1H), 2.18 (m, 1H), 2.56 (m, 1H), 3.62-3.78 (m, 2H), 4.13 (m, 1H), 4.48-4.56 (m, 3H), 6.35 (br d, 1H, J=8.5 Hz), 6.41 (br s, 1H), 7.26-7.40 (m, 5H).

V. Inhibitor Wherein R₁ is a Heteroazaaraloxy

A. General Synthetic Methods

Inhibitor MMI-138 (also referred to herein as MMI-138, OM-138, GT-138) was synthesized employing a N-(3,5-dimethylpyrazole-1-methoxy carbonyl)-L-methionine and Boc-Leu-Ψ-Ala-Val-NHCH₂Ph according to a procedure described by Ghosh, et al. 2001 (Ghosh, A. K., et al., J. Med. Chem. 44:2865-2868 (2001), the teachings of which are incorporated herein by reference in their entirety. N-(3,5-dimethylpyrazole-1-methoxy carbonyl)-L-methionine was prepared by alkoxycarbonylation of methionine methyl ester with commercially available 3,5-dimethylpyrazole-1-methanol (Aldrich Chemical) followed by saponification with aqueous lithium hydroxide (36% overall) as described by Ghosh, et al 1992 (Ghosh, A. K., et al., Tetrahedron Letter 22:781-84 (1992), the teachings of which are incorporated herein by reference in their entirety). Removal of the Boc (t-butoxycarbonyl) group of compound 43 shown below (Ghosh, A. K., et al., J. Med. Chem. 44:2865-2868 (2001), the teachings of which are incorporated herein by reference in their entirety) by treatment with trifluoroacetic acid in dichloromethane gave the corresponding amine which was reacted with N-(3,5-dimethylpyrazole-1-methoxy carbonyl)-L-methionine in the presence of N-ethyl-N′-(dimethylaminopropyl)-carbodiimide hydrochloride, diisopropylethylamine and 1-hydroxybenzotriazole hydrate in dichloromethane to compound MMI-138 in 50% yield.

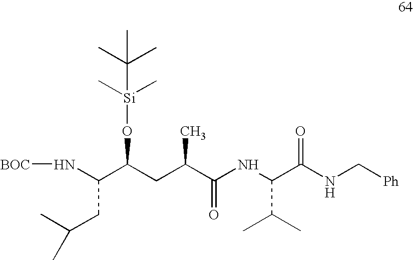

Other compounds of the invention in which R₁ or R₁₈ is a heteroazaaralkoxy group were prepared using the above-described method in which the various heteroazaaralkyl-alcohols in Table 4 were used instead of 3,5-dimethyl-pyrazole-1-methanol.

TABLE 4
STRUCTURES AND NAMES
OF HETEROAZAARALKYL-ALCOHOLS

| STRUCTURE | NAME |
|---|---|
| 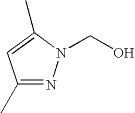 | (3,5-Dimethyl-pyrazol-1-yl)-methanol |
| 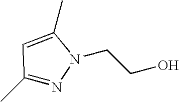 | 2-(3,5-Dimethyl-pyrazol-1-yl)- ethanol |
| 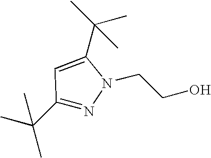 | 2-(3,5-Di-tert-butyl-pyrazol-1-yl)- ethanol |
| 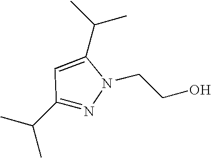 | 2-(3,5-Diisopropyl-pyrazol-1-yl)- ethanol |
| 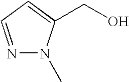 | (2-Methyl-2H-pyrazol-3-yl)-methanol |
| 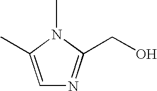 | (1,5-Dimethyl-1H-imidazol-2-yl)- methanol |
| 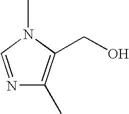 | (3,5-Dimethyl-3H-imidazol-4-yl)- methanol |
| 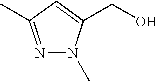 | (2,5-Dimethyl-2H-pyrazol-3-yl)- methanol |

In a typical procedure as outlined in Scheme XVII, L-methionine methyl ester hydrochloride in methylene chloride was added, in the presence of a tertiary base, to a solution of triphosgene in methylene chloride (molar ratio 1:0.37) over a period of 30 minutes using a syringe pump to form an isocyante intermediate. The alcohol component was then added to the above solution and stirred for 12 hours to provide the 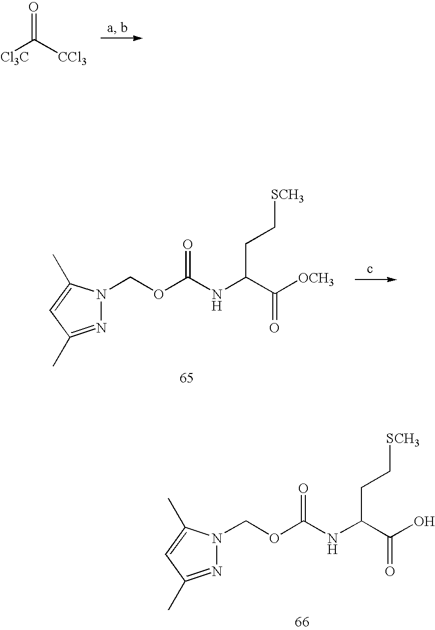
urethane-methyl ester which was hydrolyzed with LiOH in 10% aqueous THF to give the corresponding acid.

### Scheme XVII: Synthesis of N-(3,5-Dimethylpyrazole-1-Methoxy Carbonyl)-Methionine

(a) L-Methionine methylester hydrochloride, Et₃N, CH₂Cl₂, 23° C., 30 minutes;

(b) 3,5-Dimethylpyrazol-1-yl)-methanol, CH₂Cl₂, 23° C., 12 hours;

(c) LiOH, 10% aqueous THF, 23° C. 3 hours.

Other heteroaralkyl-alcohols that may be employed in the synthesizing shown in Scheme XVII are listed in Table 5.

TABLE 5
STRUCTURES AND NAMES OF HETEROARALKYL-ALCOHOLS

| STRUCTURE | NAME |
|---|---|
| 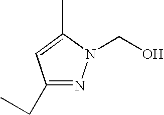 | (3-Ethyl-5-methyl-pyrazol-1-yl)- methanol |
| 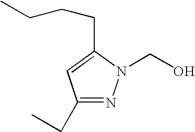 | (5-Butyl-3-ethyl-pyrazol-1-yl)-methanol |
| 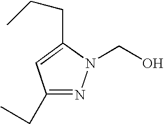 | (3-Ethyl-5-propyl pyrazol-1-yl)- methanol |
| 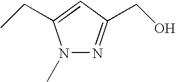 | (5-Ethyl-1-methyl-1H-pyrazol-3-yl)- methanol |
| 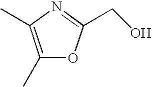 | (4,5-Dimethyl-oxazol-2-yl)-methanol |
| 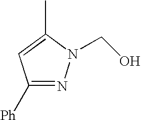 | (5, Methyl-3-phenyl-pyrazol-1-yl)- methanol |
| 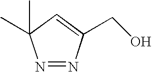 | (5,5-Dimethyl-5H-pyrazol-3-yl)- methanol |
| 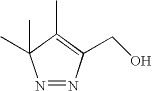 | (4,5,5-Trimethyl-5H-pyrazol-3-yl)- methanol |

Table 6 lists memapsin inhibitors of the invention that were prepared that have a heteroazaaralkoxy R₁ group. A representative example of the general synthesis of various inhibitors listed in Table 6 is outlined in Scheme XVIII.

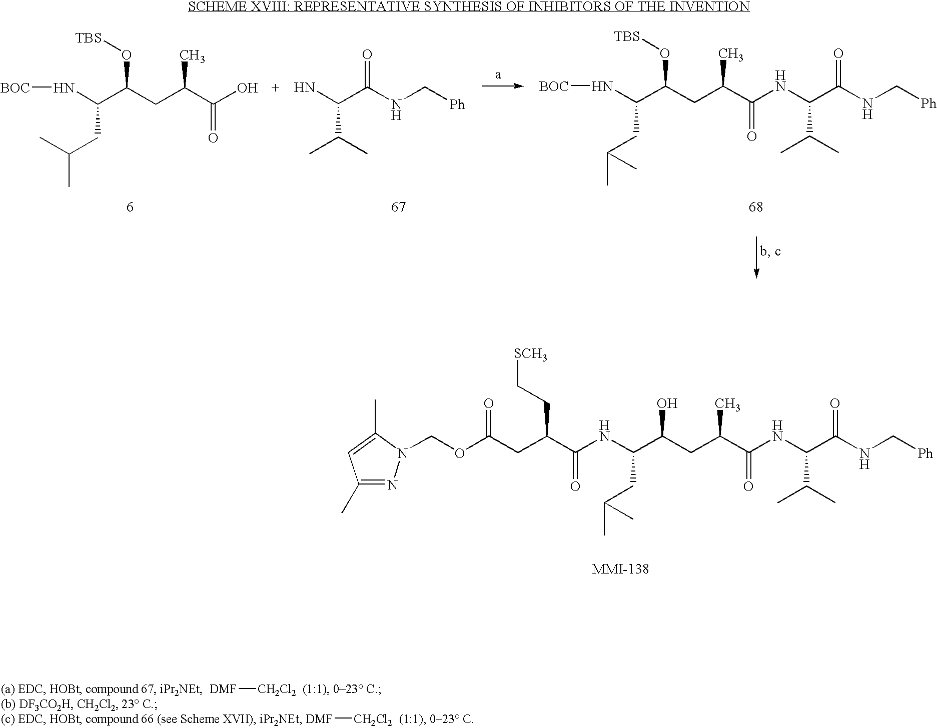

Thus, valine derivative 67 (Scheme XVIII) was reacted with the known dipeptide isostere 6 (see Part I, step F) in the presence of N-ethyl-N′-(dimethylaminopropyl)carbodiimide hydrochloride, diisopropylethylamine, and 1-hydroxybenzotriazole hydrate in a mixture of DMF and CH₂Cl₂ to generate amide derivative. Compound 68 was initially exposed to trifluoroacetic acid (TFA) in CH₂Cl₂ to remove the Boc and silyl groups. Coupling of the resulting aminol with the compound 66 generated inhibitor MMI-138. All the other inhibitors containing different R₁, P₂′, and R₃ groups were prepared following analogous procedures using the corresponding substituted heteroazaaralkoxy urethanes and valine (or leucine) derivatives.

TABLE 6
STRUCTURES OF MEMAPSIN INHIBITORS

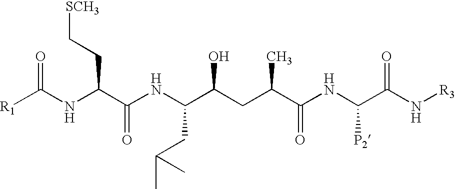

| Comp. | R₁ | P₂′ | R₃ |
|---|---|---|---|
| MMI-156 | 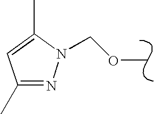 | —CH(CH₃)₂ | —CH₂CH(CH₃)₂ |
| MMI-165 | 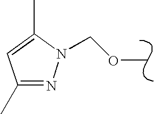 | —CH(CH₃)₂ | —CH(CH₃)₂ |
| MMI-166 | 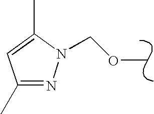 | —CH₂CH(CH₃)₂ | —CH₂CH(CH₃)₂ |
| MMI-167 | 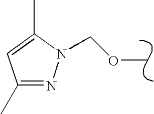 | —CH(CH₃)₂ | —(CH₂)₃CH₃ |
| MMI-176 | 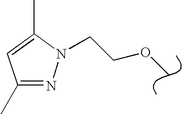 | —CH₂CH(CH₃)₂ | —CH₂CH(CH₃)₂ |
| MMI-177 | 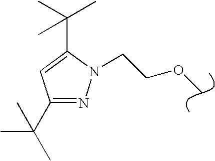 | —CH₂CH(CH₃)₂ | —CH₂CH(CH₃)₂ |
| MMI-180 | 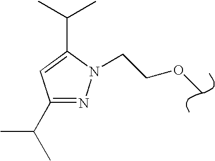 | —CH(CH₃)₂ | —CH₂CH(CH₃)₂ |
| MMI-186 | 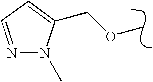 | —CH(CH₃)₂ | —CH₂CH(CH₃)₂ |
| MMI-188 | 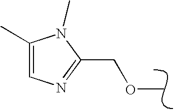 | —CH(CH₃)₂ | —CH₂CH(CH₃)₂ |
| MMI-189 | 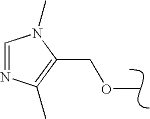 | —CH(CH₃)₂ | —CH₂CH(CH₃)₂ |
| MMI-193 | 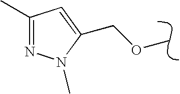 | —CH(CH₃)₂ | —CH₂CH(CH₃)₂ |

The inhibitor MMI-139 was synthesized by the oxidation of MMI-138 with OXONE® in a mixture (1:1) of methanol and water at 23° C. for 12 hours as depicted on Scheme XIX. 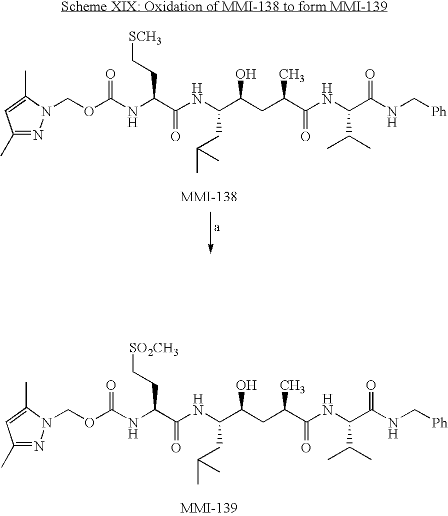
(a) OXONE®, NaHCO₃, MeOH—H₂O (1:1), 23° C., 12 hours.

A. Representative Synthesis of MMI-138 and MMI-139

### i) 2-(2-Metyhlsulfanyl-ethyl)-succinic acid 4-(3,5-dimethyl-pyrazol-1-ylmethyl)ester 1-methyl ester (65)

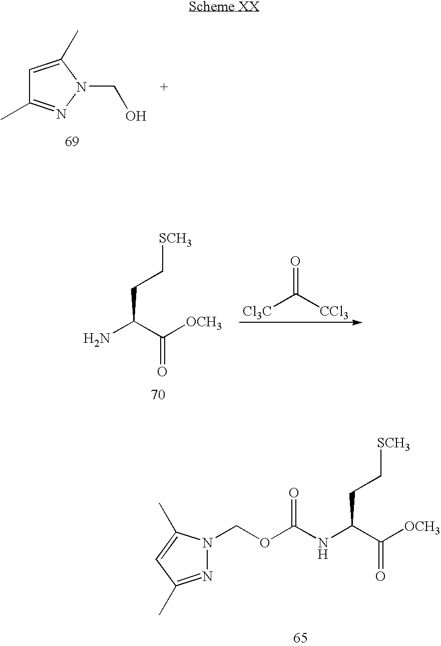

To a stirred solution of triphosgene (132 mg, 0.44 mmol) in methylene chloride (2 mL) at 23° C., a solution of L-methionine methyl ester hydrochloride 50 (242 mg, 1.21 mmol) and triethylamine (0.42 mL, 3.03 mmol) in methylene chloride (4 mL) was added slowly over a period of 30 minutes using a syringe pump. After further 5 minutes of stirring, a solution of (3,5-dimethyl-pyrazol-1-yl)-methanol 49 (152 mg, 1.21 mmol) in methylene chloride was added in one portion. The reaction mixture was stirred for 12 hours, diluted with ethyl acetate, washed with water, brine dried over NaSO₄ and concentrated under reduced pressure. The residue was purified by flash chromatography (50% EtOAC/Hexane) to give 143 mg (36%) of the compound 65. ¹H-NMR (300 MHZ, CDCl₃): δ 1.94-2.20 (2H, m), 2.0 (3H, s), 2.18 (3H, s), 2.26 (3H, s), 2.41 (2H, m), 3.74 (3H, s), 4.48 (1H, m), 5.50 (1H, br s), 5.82 (1H, s), 5.90 (2H, s).

In general, carbamates linkages of inhibitors of the invention were synthesized by the above method of coupling a compound having an alcohol group with a compound having an amine group using triphosgene. Urea linkages in inhibitors of the invention were formed by an analogous method in which triphosgene is used to couple two compound that have amine groups using the procedure described above.

### ii) 2-(2-Metyhlsulfanyl-ethyl)-succinc acid-4-(3,5-dimethyl-pyrazol-1-ylmethyl)ester (66)

To a stirred solution of above ester 65 (140 mg, 0.43 mmol) in a mixture of 10% aqueous THF (3 mL) was added LiOH (27 mg, 0.65 mmol). The mixture was stirred for 3 hours. After this period, solvents were removed and the residue was acidified with aqueous 1N HCl to pH˜4. The white solid was extracted twice with ethyl acetate and the combined extracts were dried over anhydrous sodium sulfate and concentrated under reduced pressure to provide compound 66 (134 mg, quantitative) which was carried on to the next step without further purification. ¹H-NMR (300 MHZ, CDCl₃): δ 1.94-2.20 (2H, m), 2.0 (3H, s), 2.18 (3H, s), 2.26 (3H, s), 2.48 (2H, m), 3.74 (3H, s), 4.40 (1H, m), 5.50 (1H, br s), 5.82 (1H, s), 5.90 (2H, s).

### iii) Compound 67

To a stirred solution of N-Boc-Valine (500 mg, 2.3 mmol) and benzylamine (0.50 mL, 4.60 mmol) in a mixture of CH₂Cl₂ (20 mL) and DMF (2 mL), HOBt (373 mg, 2.8 mmol), EDC (529 mg, 2.8 mmol) and diisopropylethylamine (2.4 mL, 13.8 mmol) were added successively at 0° C. After the addition, the reaction mixture was allowed to warm to 23° C. and it was stirred overnight. The mixture was poured into aqueous NaHCO₃ solution and the mixture was extracted with 30% EtOAc/hexane. The organic layer was washed with brine and dried over Na₂SO₄. Evaporation of the solvent under reduced pressure gave a residue which was purified by flash column chromatography (30% EtOAc/hexane) to give 442 mg (63%) of coupled product. The resulting amine was dissolved in CH₂Cl₂ (20 mL), and TFA (4 mL) was added at 23° C. The reaction mixture was stirred for 30 minutes and then it was concentrated under reduced pressure to provide the compound 67 (297 mg, quantitative). ¹H-NMR (500 MHZ, CDCl₃): δ 0.87 (3H, d, J=6.9 Hz), 1.02 (3H, d, J=6.9 Hz), 2.00 (2H, br s), 2.37 (1H, m), 3.36 (1H, br s), 4.43-4.52 (2H, m), 7.27-7.37 (5H, m), 7.70 (1H, brs).

iv) Compound 68:

Dipeptide isostere 6 (42 mg, 0.1 mmol) and compound 67 (41 mg, 0.2 mmol) were dissolved in DM (2 mL). To this solution, HOBt (20 mg, 0.15 mmol), EDC (29 mg, 0.15 mmol) and diisopropylethylamine (0.2 mL) were added successively at 0° C. After the addition, the reaction mixture was allowed to warm to 23° C. and it was stirred overnight. The mixture was poured into aqueous NaHCO₃ and it was extracted with 30% EtOAc/hexane. The organic layer was washed with brine and dried over anhydrous Na₂SO₄. Evaporation of the solvent under reduced pressure gave a residue which was purified by column chromatography (20% EtOAc/hexane) to give 55 mg (95%) of compound 68. ¹H-NMR (500 MHZ, CDCl₃) δ 0.09 (3H, s), 0.10 (3H, s), 0.91 (9H, s), 0.92-0.98 (12H, m), 1.10 (3H, d, J=6.7 Hz), 1.25 (1H, m), 1.44 (1H, m), 1.46 (9H, s), 1.63 (1H, m), 1.74 (1H, br s), 1.80 (1H, m), 2.18 (1H, m), 2.56 (1H, m), 3.62-3.78 (2H, m), 4.13 (1H, m), 4.48-4.56 (3H, m), 6.35 (1H, br d, J=8.5 Hz), 6.41 (1H, br s), 7.26-7.40 (5H, m).

### v) MMI-138

To a solution of 68 (37 mg, 0.06 mmol) in CH₂C₂ (1 mL) was added TFA (0.4 mL) at 23° C. The resulting mixture was stirred at 23° C. for 1 hour, the concentrated under reduced pressure and the residue was dissolved in DMF (2 mL). To this solution, compound 66 (18 mg, 0.06 mmol), HOBt (8 mg, 0.06 mmol), EDC (11 mg, 0.06 mmol) and diisopropylethylamine (0.2 mL) were added successively at 0° C. After the addition, the reaction mixture was allowed to warm to 23° C. and it was stirred overnight. The mixture was poured into aqueous NaHCO₃ and it was extracted with EtOAc. The organic layer was washed with brine and dried over anhydrous Na₂SO₄. Evaporation of the solvent under reduced pressure gave a residue which was purified by column chromatography (2% MeOH/CHCl₃) to provide the inhibitor MMI-138 (16 mg, 40%). ¹H-NMR (300 MHZ, CD₃OD): δ 0.80-0.97 (12H, m), 1.10 (3H, d, J=6.7 Hz), 1.20-2.38 (8H, m), 2.0 (3H, s), 2.18 (3H, s), 2.24 (3H, s), 2.41 (3H, t, J=6.4 Hz), 2.60 (1H, m), 3.41 (1H, m), 3.80 (1H, m), 4.15 (1H, m), 4.20-4.32 (3H, m), 5.80 (3H, s) 7.17-7.30 (5H, m).

vi) Preparation of inhibitor MMI-139:

To a solution of MMI-138 (10 mg, 0.015 mmol) in MeOH—H₂O (1:1) (2 mL), were added NaHCO₃ (11.6 mg, 0.12 mmol) and potassium peroxymonosulfate (OXONE®) (27 mg, 0.05 mmol) and stirred for 12 hours. The reaction was then diluted with ethyl acetate, washed with water and dried over anhydrous Na₂SO₄. Evaporation of the solvent under reduced pressure gave a residue which was purified by column chromatography (4% MeOH/CHCl₃) to provide the inhibitor MMI-139 (6.8 mg, 65%). ¹H-NMR (300 MHZ, CD₃OD): δ 0.72-0.92 (12H, m), 1.20 (3H, d, J=6.0 Hz), 1.15-2.06 (6H, m), 2.16 (3H, s), 2.24 (3H, s), 2.58 (3H, s), 2.82 (3H, s), 3.30 (2H, m), 3.60 (1H, m), 3.78 (1H, m), 4.0 (2H, m), 4.22 (1H, m), 4.34-4.38 (3H, m), 5.80 (3H, s), 7.18-7.36 (5H, m).

C. Other Inhibitors of the Invention

The following memapsin inhibitors of the invention were prepared via a method analogous to the method of preparing MMI-138 and MMI-139. The various R₁ groups of the inhibitors listed below were obtained by substituting the appropriate heteroazaaryalkyl-alcohol listed in Table 1 for 3,5-dimethylpyrazol-1-yl)-methanol in the method of preparing the urethane portion of the molecule (see Scheme 10). A leucine side chain was obtained at the P₂′ position in the inhibitors listed below by substituted N-Boc-leucine for N-Boc-valine in the method described in Section V-B(iii). Other natural and non-natural Boc-protected amino acids may be substituted for N-Boc-valine in the method described in Section V-B(iii) to obtain other P₂′ groups in the inhibitors of the invention. Inhibitors having 2-methylprop-1-yl or 1-methyleth-1-yl R₃ groups were obtained by substituting 2-methylpropyl amine or 1-methylethyl amine for benzylamine in the synthesis described Section V-B(iii). Other compounds containing amine groups may also be substituted for benzyl amine in the synthesis described in Section V-B(iii). For example, aliphatic amines, aryl amines, aralkyl amines, heterocycle amines, heterocycloalkyl amines, heteroaryl amines, heteroaralkyl amines, peptide or a carrier molecule containing amine groups may be used instead of benzylamine in the synthesis describe in Section V-B(iii). In addition, heterocycles or heteroaryl compounds that have secondary amines may be used instead of benzylamine in Section V-B(iii).

i) Inhibitor MMI-156:

¹H-NMR (300 MHZ, CD₃OD): δ 0.80-0.90 (18H, m), 1.20 (3H, d, J=6.6 Hz), 1.18-2.04 (8H, m), 2.0 (3H, s), 2.17 (3H, s), 2.24 (3H, s), 2.42 (3H, t, J=6.2 Hz), 2.50 (1H, m), 2.80-3.30 (m, 2H), 3.41 (1H, m), 3.78 (1H, m), 3.90 (1H, d, J=6.8 Hz), 4.18 (1H, t, J=6.3 Hz), 5.80 (3H, s).

ii) Inhibitor MMI-165:

¹H-NMR (300 MHZ, CD₃OD): δ 0.80-0.97 (12H, m), 1.40 (9H, m,), 1.18-2.20 (8H, m), 2.0 (3H, s), 2.18 (3H, s), 2.26 (3H, s), 2.50 (3H, m), 3.42 (1H, m), 3.80 (1H, m), 3.90 (2H, m), 4.20 (1H, m), 5.80 (3H, s).

iii) Inhibitor MMI-166:

¹H-NMR (300 MHZ, CD₃OD): δ 0.80-0.96 (18H, m), 1.20 (3H, d, J=6.7 Hz), 1.06-2.20 (8H, m), 2.0 (3H, s), 2.17 (3H, s), 2.23 (3H, s), 2.38-2.60 (3H, m), 3.0 (2H, m), 3.42 (1H, m), 3.78 (1H, m), 4.2 (3H, m), 4.38 (1H, s), 5.80 (3H, s).

iv) Inhibitor MMI-167:

¹H-NMR (300 MHZ, CD₃OD): δ 0.80-1.0 (19H, m), 1.10 (3H, d, J=6.2 Hz), 1.20-2.26 (8H, m), 2.0 (3H, s), 2.18 (3H, s), 2.3 (3H, s), 2.5 (2H, m), 2.6 (3H, m), 3.40 (1H, m), 4.10 (1H, m), 4.20 (1H, m), 4.44 (1H, s), 5.84 (3H, s).

v) Inhibitor MMI-176:

¹H-NMR (500 MHZ, CD₃OD): δ 0.78-0.85 (18H, m), 1.10 (3H, d, J=6.2 Hz), 1.20-2.0 (9H, m), 1.93 (3H, s), 2.11 (3H, s), 2.15 (3H, s), 2.42 (2H, t, J=5.1 Hz), 2.55 (1H, m), 2.80 (1H, m), 3.10 (1H, m), 3.40 (1H, m), 3.80 (2H, m) 3.90 (1H, m), 4.10 (2H, m), 4.2 (2H, m), 5.7 (1H, s).

vi) Inhibitor MMI-177:

¹H-NMR (500 MHZ, CD₃OD): δ 0.75-0.81 (18H, m), 1.0 (3H, d, J=6.8 Hz), 1.1 (9H, s), 1.2 (9H, s), 1.10-2.0 (9H, m), 1.90 (3H, s), 2.35 (2H, t, J=5.3 Hz), 2.60 (1H, m), 2.80 (1H, m), 2.90 (1H, m), 3.30 (1H, m), 3.60 (1H, m) 3.90 (1H, m), 4.10 (1H, m), 4.20 (2H, m), 5.70 (1H, s).

vii) Inhibitor MMI-180:

¹H-NMR (300 MHZ, CD₃OD): δ 0.82-1.15 (18H, m), 1.19 (3H, d, J=6.2 Hz), 1.21 (6H, s), 1.23 (6H, s), 1.22-2.60 (8H, m), 2.30 (3H, s), 2.54 (2H, t, J=5.0 Hz), 2.60 (1H, m), 2.82-3.18 (3H, m), 3.60 (1H, m), 3.82 (1H, m) 4.12 (1H, m), 4.2 (2H, m), 4.4 (2H, m), 5.82 (1H, s).

viii) Inhibitor MMI-186:

¹H-NMR (300 MHZ, CD₃OD): δ 0.77-0.85 (18H, m), 1.10 (3H, d, J=6.0 Hz), 1.16-2.0 (9H, m), 1.98 (3H, s), 2.42 (2H, t, J=5.6 Hz), 2.50 (1H, m), 2.84 (1H, m), 3.00 (1H, m), 3.40 (1H, m), 3.72 (1H, m), 3.78 (3H, s), 3.94 (1H, m) 4.18 (1H, m), 5.0 (2H, s), 6.20 (1H, s), 7.36 (1H, s).

ix) Inhibitor MMI-188:

¹H-NMR (300 MHZ, CD₃OD): δ 0.77-0.85 (18H, m), 1.20 (3H, d, J=6.4 Hz), 1.18-2.05 (9H, m), 2.03 (3H, s), 2.16 (3H, s), 2.4-2.6 (3H, m), 2.84-2.98 (2H, m), 3.44 (1H, m), 3.57 (3H, s), 3.80 (1H, s), 3.98 (1H, s), 4.20 (1H, s), 5.0 (2H, s), 7.31 (1H, s).

x) Inhibitor MMI-189:

¹H-NMR (300 MHZ, CD₃OD): δ 0.78-0.92 (18H, m), 1.05 (3H, d, J=5.8 Hz), 1.20-2.05 (9H, m), 2.03 (3H, s), 2.13 (3H, s), 2.4-2.6 (3H, m), 2.84-3.40 (2H, m), 3.44 (1H, m), 3.47 (3H, s), 3.78 (1H, m), 3.98 (1H, m), 4.20 (1H, m), 5.05 (2H, s), 6.65 (1H, s).

xi) Inhibitor MMI-193:

¹H-NMR (300 MHZ, CD₃OD): δ 0.78-0.92 (18H, m), 1.05 (3H, d, J=6.6 Hz), 1.18-2.02 (9H, m), 2.02 (3H, s), 2.15 (3H, s), 2.46 (2H, t, 3=5.8 Hz), 2.56 (1H, m), 2.84-2.96 (1H, m), 3.00 (1H, m), 3.44 (1H, m), 3.72 (3H, s), 3.78 (1H, m), 3.98 (1H, s) 4.22 (1H, m), 4.97 (2H, s), 5.99 (1H, s).

VI. Synthesis of Starting Materials

Synthesis of compounds used in the preparation of inhibitors of the invention that are not commercially available are described below.

A. Synthesis of Starting Material for Inhibitors Having Heteroazaaralkyl R₁ Groups

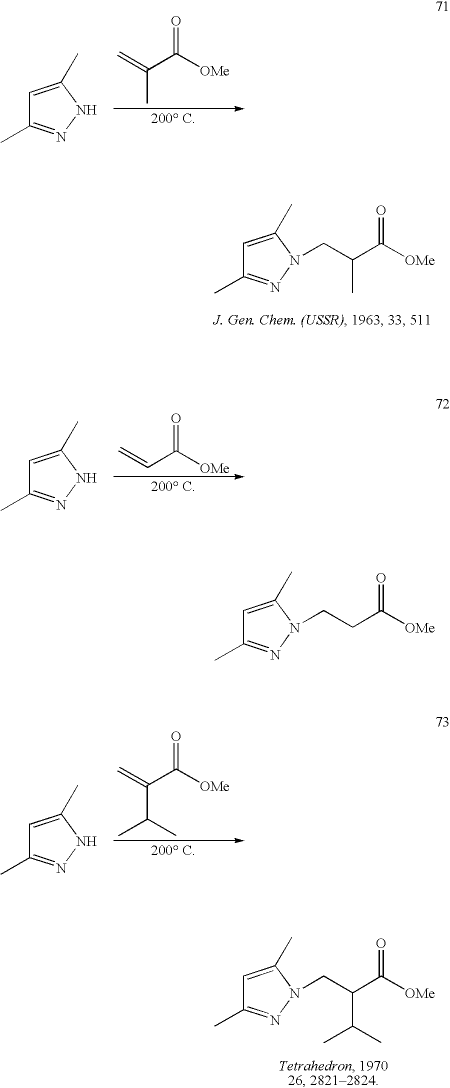

General procedure (J. Gen. Chem. (UUSR) 33:511 (1963)): A mixture of 1,3-dimethylpyrazole (395 mg, 4.11 mmol) and 2-methyl acrylic acid methyl ester (1.0 mL) were heated in a sealed tube at 200° C. for 4 hours. The reaction was cooled to room temperature, the solvent was removed under reduced pressure and the residue was chromatographed (35% EtOAc in hexanes) to afford 51 (470 mg, 58%) which was used to prepare inhibitors MMI-195, MMI-196, MMI-214, and MMI-226. Pyrazoles 72 and 73 were synthesized using analogous procedures. Compound 72 was used to prepare inhibitors MMI-194 and MMI-213, and compound 73 was used to prepare inhibitors MMI-204, MMI-225, MMI-228 and MMI-229. Hydrolysis of the methyl esters was accomplished by stirring the ester in a room temperature saturated solution of LiOH in 10% aqueous THF, for 3-48 hours.

B. Synthesis of Starting Material for Inhibitors Having Heteroazaaralkoxy R₁

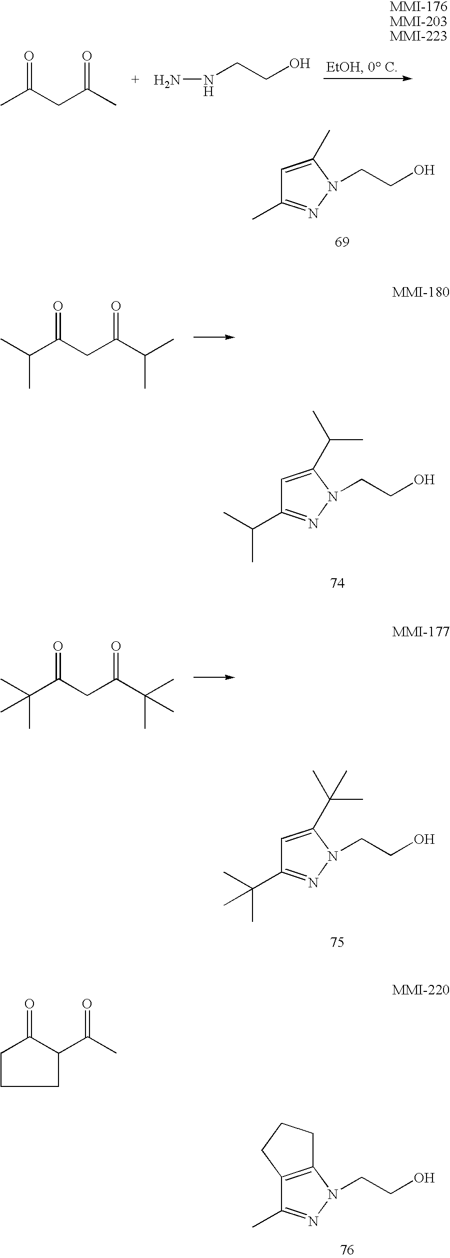

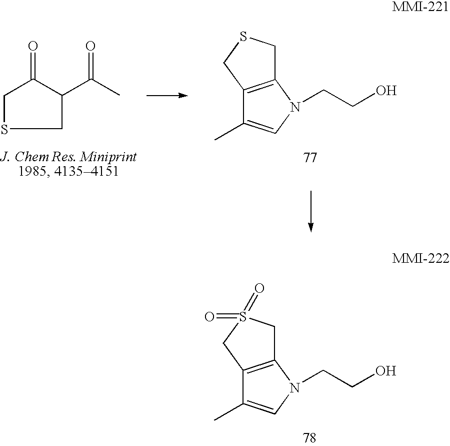

i) Compounds 69 and 74-77 were Prepared Using the Following General Procedure:

A solution of 2-hydroxyethylhydrazine (1.02 mmol) in absolute ethanol (1 mL) was added dropwise to a solution of the corresponding diketone (1.0 mmol) at 0° C. The mixture was warmed to room temperature and stirred for 1 hour. The solvent was removed under reduced pressure and the residue was dissolved in CH₂Cl₂ and washed with water. The organic layer was dried with Na₂SO₄, concentrated, and purified by flash chromatography (60% EtOAc in hexanes) to yield the product.

ii) Oxidation Procedure of Compound 77 to Yield Compound 78:

To a solution of compound 77 (184 mg, 1 mmol) in acetone/H₂O (3:1, 20 mL) was added N-methyl morpholine N-oxide (292 mg, 2.5 mmol) followed by OsO₄ (0.38 mL, 2 wt % in t-BuOH, 0.03 mmol) and stirred overnight. The solvent was removed under reduced pressure and the residue was dissolved in CH₂Cl₂ and washed with water. The organic layer was dried with Na₂SO₄, concentrated under reduced pressure and chromatographed (4% MeOH in CHCl₃) to yield compound 78 (110 mg, 51%).

iii) Preparation of 1-(3,5-dimethyl-pyrazol-1-yl)-2-methyl-propan-2-ol (60) Used to Prepare Inhibitor MMI-219:

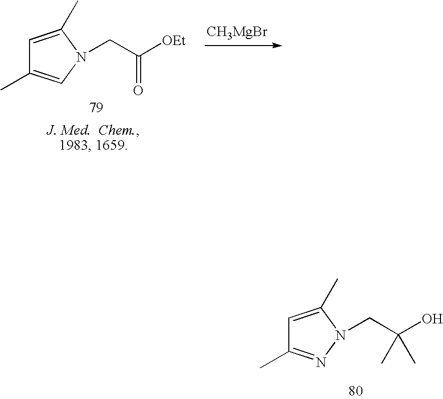

Methylmagnesium bromide (5.4 mL, 1.4 M in THF, 7.6 mmol) was added dropwise to a solution of (3,5-dimethylpyrazole-1-yl)-acetic acid ethyl ester (J. Med. Chem., p. 1659 (1983)) (compound 79, 554 mg, 3.04 mmol) in THF at 0° C. After 30 minutes the reaction was quenched with saturated aqueous NH₄Cl and extracted with EtOAc. The organic layer was dried with Na₂SO₄, concentrated, and purified by column chromatography (40% EtOAc in hexanes) to yield 276 mg (65%) of compound 80.

C. Synthesis of Additional Starting Materials for Inhibitors Having Heteroazaaralkoxy R₁ Groups

The following heteroazaaralkyl-alcohol starting materials were synthesized via the method described in the cited reference.

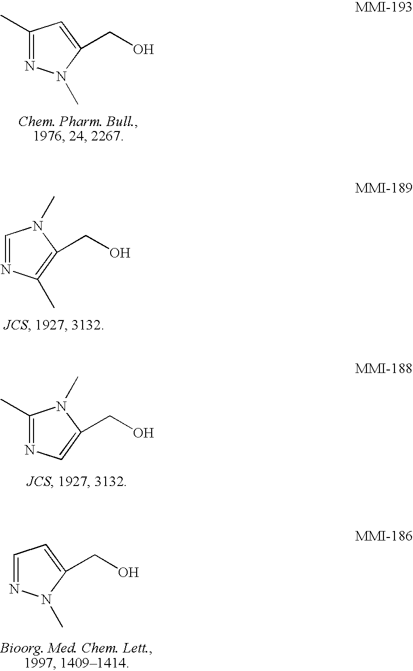

D. Synthesis of Boc-Protected Non-Natural Amino Acid Having a Tetrahydrofuranylmethyl Side Chain Used to Form P₁ Substituent of Inhibitors MMI-013, MMI-014, MMI-019, MMI-020, MMI-034, MMI-035, MMI-205 and MMI-215:

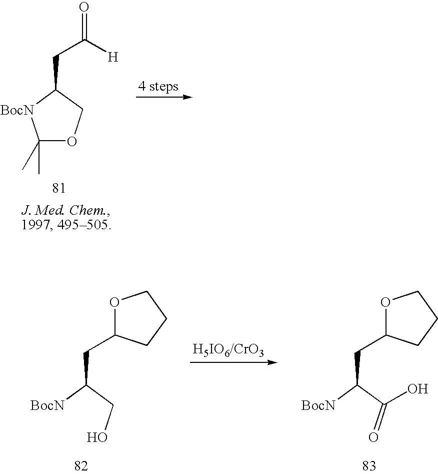

i) Step 1:

To a solution of compound 81 (J. Med. Chem., p. 495-505 (1997)) (1.17 g, 4.8 mmol) in diethylether (20 mL) at −78° C. was added dropwise allylmagnesium bromide (7.5 mL, 1.0 M in diethylether, 7.5 mmol). After stirring for 30 min, the reaction was quenched with saturated aqueous NH₄Cl at −78° C. The mixture was warmed to room temperature and the layers were separated. The organic layer was dried with Na₂SO₄ and concentrated under reduced pressure. The diastereomers were separated by flash column chromatography (25% EtOAc in hexanes) to yield 500 mg (37%) of the faster isomer and 630 mg (46%) of the slower isomer. The remainder of the synthesis was carried out on each of the isomers separately to prepare non-natural amino acid used to form inhibitors MMI-205 and MMI-215.

Non-natural amino acids used to prepare inhibitors MMI-013, MMI-014, MMI-019, MMI-020, MMI-034, MMI-035 were synthesized by the same protocol using the appropriate aldehyde with one less methylene (Bioorg. Med. Chem. Lett. 8:179-182 (1998)).

ii) Step 2 (Example with One Isomer Only):

9-borabicyclo[3.3.1]nonane (9-BBN) (3.86 mL, 0.5 M in THF, 1.93 mmol) was added to a solution of the product from Step 1 (500 mg, 1.75 mmol) in THF (5 mL) and stirred for 12 h, after which time the reaction mixture was cooled to −20° C. and MeOH (0.13 mL), 3 N NaOH (0.87 mL), and 30% H₂O₂ (0.87 mL) were added sequentially. The reaction mixture was warmed to 60° C. and stirred for 1 hour. The resulting clear solution was poured into brine (25 mL), extracted with diethylether, dried with Na₂SO₄, concentrated, and purified by flash column chromatography (70% EtOAc in hexanes) to yield 280 mg (53%) of the product.

iii) Step 3:

To a solution of the product from step 2 (112 mg, 0.34 mmol) in CH₂Cl₂ (3 mL) was added triethylamine (0.1 mL, 0.74 mmol), p-toluene sulfonyl chloride (78 mg, 0.41 mmol), dimethylaminopyridine (9 mg, 0.07 mmol) sequentially and the reaction was stirred at room temperature for 12 hours, after which it was diluted with CH₂Cl₂ and washed with saturated aqueous NH₄Cl, dried with Na₂SO₄, concentrated under reduced pressure and purified by column chromatography (20% EtOAc in hexanes) to yield 83 mg (86% of the corresponding tetrahydrofuran.

iv) Step 4:

To a stirred solution of the tetrahydrofuran prepared in step 3 in MeOH (3 mL) was added p-toluene sulfonic acid hydrate (13 mg, 0.07 mmol) and stirred at room temperature for 1 hour. The reaction was then quenched with saturated aqueous NaHCO₃ and extracted with EtOAc. The organic layer was dried with Na₂SO₄, concentrated and chromatographed (50% in EtOAc in hexanes) to yield compound 82 (55 mg, 65%).

v) Formation of the Carboxylic Acid:

The compound 82 was oxidized to the corresponding carboxylic acid using H₅IO₆/CrO₃ in wet CH₃CN via the following procedure (Tetrahedron Lett., p. 5323 (1998)): A stock solution of H₅IO₆/CrO₃ was prepared by dissolving H₅IO₆ (11.4 g, 50 mmol) and CrO₃ (23 mg, 1.2 mol %) in wet CH₃CN (0.75 v % water) to a volume of 114 mL (complete dissolution typically required 1-2 h). The H₅IO₆/CrO₃ solution (0.7 mL) was then added to a solution of compound 82 (30 mg, 0.12 mmol) in wet CH₃CN (1 mL) over a period of 30 minutes 0° C. The reaction was quenched by adding aqueous Na₂HPO₄; The mixture was extracted with diethylether and the organic layer was washed with brine, aqueous Na₂HPO₄, brine, dried with Na₂SO₄, and concentrated under reduced pressure. The crude yield of 83 was 22 mg (69%).

E. Synthesis of Cbz-Protected Non-Natural Amino Acid Having a Methoxymethoxyethyl Side Chain Used to Form P₂ Substituent of Inhibitor MMI-190:

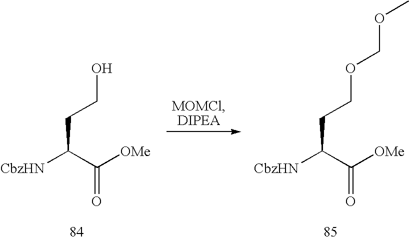

To a solution of Cbz-protected homoserine (J. Org., Chem., 5442 (1997)) (60, 140 mg, 0.52 mmol) in CH₂Cl₂ (3 mL) at 0° C. were added diisopropylethylamine (DIPEA) (0.28 mL, 1.6 mmol) and chloromethylmethylether (MOMCl) (0.05 mL, 0.62 mmol). After stirring for 3 h, the reaction was quenched with saturated aqueous NH₄Cl and extracted with diethylether. The organic layer was dried with Na₂SO₄, concentrated under reduced pressure and chromatographed (30% EtOAc in hexanes) to yield 116 mg (71%) of compound 85. Removal of the Cbz protecting group by hydrogenation provided the free amine for coupling.

F. Synthesis of Boc-Protected Non-Natural Amino Acid Having a Methoxyethyl Side Chain Used to Form P₂ Substituent of Inhibitors MMI-079, MMI-185, MMI-228:

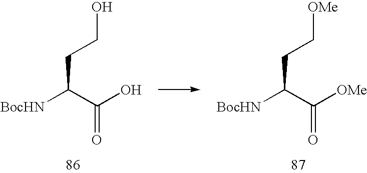

To a solution of Boc-protected homoserine (86, 400 mg, 1.83 mmol) in DMF (8 mL) at 0° C. were added NaH (60%, 155 mg, 4.02 mmol) followed by MeI (0.45 mL, 7.3 mmol). The reaction was stirred for 12 hours at room temperature. The DMF was removed under reduced pressure. The residue was dissolved in EtOAc and washed with saturated aqueous NH₄Cl, dried with Na₂SO₄, concentrated under reduced pressure and chromatographed (20% EtOAc in hexanes) and yielded 323 mg (72%) of compound 87. Compound 87 was hydrolyzed with LiOH (as described above) to quantitatively yield the free acid.

G. Synthesis of Starting Materials for Macrocyclic Inhibitors MMI-149, MMI-150, MMI-152, MMI-153, MMI-174, and MMI-175 and Macrocyclic Inhibitor Precursors MMI-148, MMI-151, and MMI-173

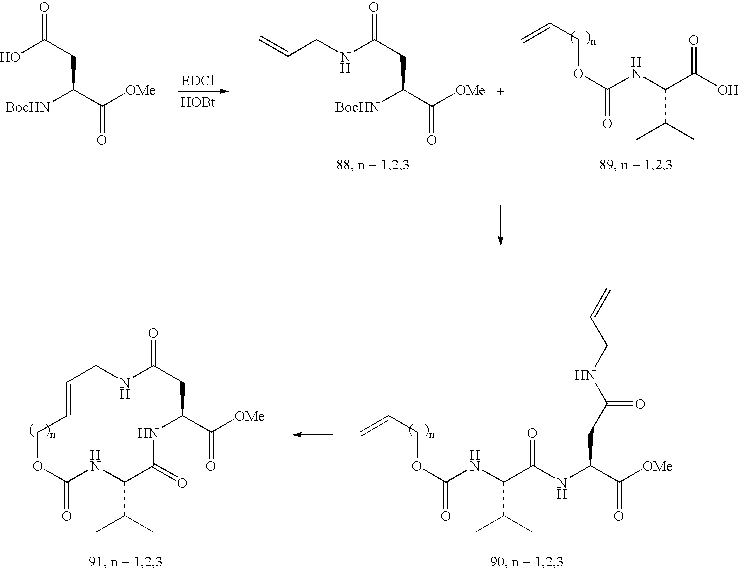

EDCI/HOBt coupling of Boc-Asp methyl ester with allyl amine (see Section IV) was followed by TFA removal of the Boc protecting group and coupling with various Val derivatives 89 (these carbamates were produced by triphosgene coupling of Val methyl ester with various alcohols—allyl alcohol, 4-butenol, and 5-pentenol) (see Section V-B(i). The compounds represented by structure 90 were incorporated into inhibitors MMI-148, MMI-151, and MMI-173 by hydrolysis followed by coupling of the free acid. The macrocycles were formed using ring-closing olefin metathesis to form the macrocyclic group in inhibitors MMI-149, MMI-150, MMI-152, MMI-153, MMI-174, and MMI-175. A representative procedure for the formation of the macrocyclic group follows:

To a 0.002 M solution of the diene (90) in CH₂Cl₂ was added Grubbs's catalyst (20 mol %). The flask was flushed with Argon and stirred at room temperature for 12 hours. The solvent was removed under reduced pressure and the residue was chromatographed (2% MeOH in CHCl₃) to yield approximately 75% of the desired macrocycle. This metathesis step was followed by LiOH hydrolysis and yielded the free acid for further coupling. This produced ligands for MMI-149, MMI-152, and MMI-174.

To prepare inhibitors MMI-150, MMI-153, and MMI-175 inhibitors MMI-149, MMI-152, and MMI-174, respectively, were hydrogenated following the standard hydrogenation procedure described previously (Section II, Step F).

H. Synthesis of 2-methyl-1-(tetrahydrofuran-2-yl)-propylamine and 2-methyl-1-(tetrahydro-pyran-2-yl)-propylamine Used to Form R₃ Substituent of Inhibitors MMI-154 and Its Pyran Derivative: 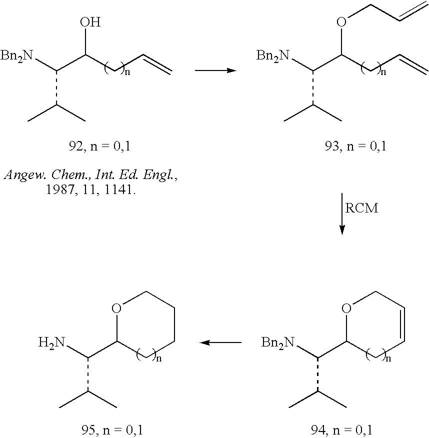
Representative Procedure:

i) Step 1:

To a solution of compound 92 (Angew. Chem., Int. Ed. Engl. 11:1141 (1988)) (530 mg, 1.64 mmol) in THF at 0° C. were added NaH (60%, 130 mg, 3.28 mmol) and allyl iodide (0.23 mL, 2.46 mmol) and stirred for 12 hours at room temperature. The reaction was quenched with saturated aqueous NH₄Cl, extracted with diethylether, dried with Na₂SO₄, concentrated under reduced pressure, and chromatographed (2% EtOAc in hexanes) to yield 530 mg (90%) of allyl ether 93.

ii) Step 2:

To a solution of compound 93 (200 mg, 0.55 mmol) in 100 mL of CH₂Cl₂ was added Grubbs's catalyst (20 mg, 5 mol %) and the mixture was refluxed under argon for 2 hours. The solvent was removed under reduced pressure and the residue was chromatographed (3% EtOAc in hexanes) to provide 171 mg (93%) of dihydropyran 94.

iii) Step 3:

A mixture of compound 94 (135 mg, 0.4 mmol) and Pd(OH)₂/C (20%, 20 mg) in MeOH was stirred under an H₂ atmosphere for 5 hours. The catalyst was filtered off and the filtrate was concentrated under reduced pressure to yield compound 95 quantitatively.

I. Synthesis of 3-(1-amino-2-methyl-propyl)-5-benzyl-cyclohexanone (100) and 1-(3-benzyl-cyclohexyl)-2-methyl-propylamine (101) Used to Form the R₃ Substituent of Inhibitors MMI-140, MMI-141, MMI-146, and MMI-147

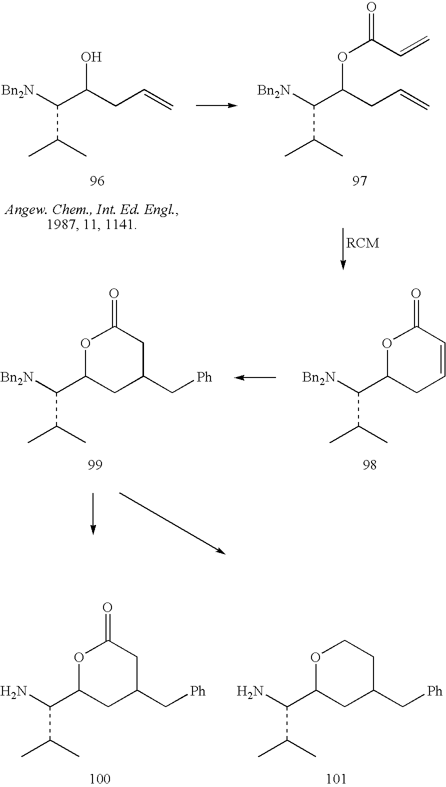

### i) Synthesis of 3-(1-amino-2-methyl-propyl)-5-benzyl-cyclohexanone (100) for Preparation of MMI-140 and MMI-141

a) Step 1:

To a solution of 96 (930 mg, 2.8 mmol) in CH₂Cl₂ (10 mL) at 0° C. were added Et₃N (1.2 mL, 8.64 mmol) and acryloyl chloride (0.3 mL, 3.74 mmol). The reaction was stirred at room temperature for 1 hour and quenched with saturated aqueous NH₄Cl. The aqueous layer was extracted with diethylether and the combined organic layers were dried with Na₂SO₄, concentrated under reduced pressure and chromatographed (4% EtOAc in hexanes) to yield lactone 97 (700 mg, 660%).

b) Step 2:

Ring-closing olefin metathesis following the same procedure as previously described in Section VI, Part G was performed and yielded compound 98 in 89% yield.

c) Step 3:

To a solution of compound 98 (75 mg, 0.2 mmol) in diethylether was added CuCN (2 mg, 10 mol %). The mixture was cooled to −78° C. and PhCH₂MgCl (0.24 mL, 1.0 M in diethylether, 0.24 mmol) was added dropwise. The reaction was allowed to warm to room temperature over a period of 1 hour and quenched, with saturated aqueous NH₄Cl, extracted with diethylether. The organic layer was dried with Na₂SO₄, concentrated under reduced pressure, and chromatographed (25% EtOAc in hexanes) to yield compound 98 (47 mg, 50%).

d) Step 4:

Hydrogentaion of compound 99 to remove the benzyl protecting groups as previously described (Section U, Step F) led to 3-(1-amino-2-methyl-propyl)-5-benzyl-cyclohexanone (100) which was used to prepare inhibitors MMI-140 and MMI-141.

### i) Synthesis of 1-(3-benzyl-cyclohexyl)-2-methyl-propylamine (101) for Preparation of MMI-146 and MMI-147

DIBAL-H (1.28 mmol, 1.0M in hexanes, 1.28 mmol) was added to a solution of compound 99 (225 mg, 0.57 mmol) in toluene (3 mL) at −78° C. and stirred for 30 minutes. The reaction was quenched with aqueous Na—K-tartrate, warmed to room temperature, and extracted with diethylether. The organic layer was dried with Na₂SO₄ and concentrated under reduced pressure to yield the crude lactol.

The crude lactol was dissolved in CH₂Cl₂ (5 mL), cooled to 0° C., and Et₃SiH (0.12 mL, 0.75 mmol) and BF₃.OEt₂ (0.07 mL, 0.55 mmol) were added successively. After 30 minutes, the reaction was quenched with saturated aqueous NaHCO₃ and extracted with EtOAc. The organic layer was dried with Na₂SO₄, concentrated under reduced pressure, and chromatographed to afford the corresponding tetrahydropyran (175 mg, 80%) which was hydrogenated to remove the benzyl protecting groups as previously described to afford compound 101. Compound 101 was used to prepare inhibitors MMI-146 and MMI-147.

J. Synthesis of 4-amino-6-methyl-1-phenyl-heptan-3-ol Used to Form the P₂′-P₃′ Substituents of Inhibitor MMI-091:

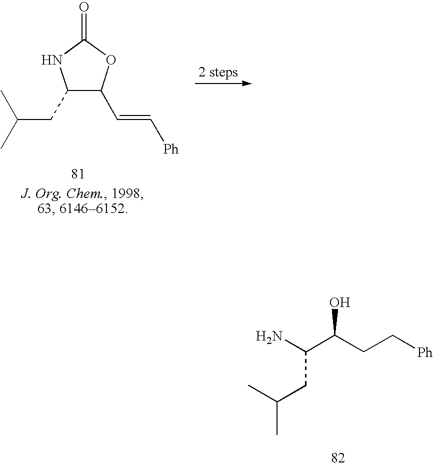

i) Step 1:

To a mixture of know oxazolidinone 81 (J. Org. Chem. 63:6146-6152 (1998)) (80 mg, 0.33 mmol) and 10% Pd/C (15 mg) in MeOH (4 mL) was stirred under an H₂ atmosphere for 1 hour. The catalyst was filtered off and the filtrate was concentrated under reduced pressure and chromatographed (40% EtOAc in hexanes) to yield 48 mg (61%) of the saturated product.

ii) Step 2:

To a solution of the product of step 1 (48 mg, 0.19 mmol) in, EtOH/H₂O (1:1, 4 mL) was added KOH (45 mg, 0.78 mmol) and stirred for 12 hours. The reaction was then acidified to pH 3 with 1 M HCl, extracted with CHCl₃, dried with Na₂SO₄, and concentrated under reduced pressure to yield 35 mg (83%) of 82.

K. Preparation of Sulfone Ligand of MMI-003, MMI-007, MMI-009, MMI-016, MMI-018, MMI-024, MMI-026, MMI-035, MMI-043, MMI-045, MMI-047, MMI-052, MMI-054, MMI-056, MMI-058, MMI-060, MMI-067, MMI-069, MMI-071, MMI-073, MMI-082, MMI-088, MMI-090, MMI-096, MMI-098, MMI-100, MMI-105, MMI-122, MMI-123, MMI-126, MMI-128, MMI-129, MMI-135, MMI-136, MMI-137, MMI-139:

Representative Procedure:

Inhibitor MMI-139: To a solution of MMI-138 (10 mg, 0.015 mmol) in MeOH—H₂O (1:1) (2 mL), were added NaHCO₃ (11.6 mg, 0.12 mmol) and Oxone® (potassium peroxymonosulfate) (27 mg, 0.05 mmol) and stirred for 12 hours. The reaction was diluted with ethyl acetate, washed with water and dried with Na₂SO₄. Evaporation of the solvent under reduced pressure gave a residue which was purified by column chromatography (4% MeOH in CHCl₃) to provide the inhibitor MMI-139 (6.8 mg, 65%). ¹H-NMR (300 MHZ, CD₃OD): δ 0.72-0.92 (12H, m), 1.20 (3H, d, J=6.0 Hz), 1.15-2.06 (6H, m), 2.16 (3H, s), 2.24 (3H, s), 2.58 (3H, s), 2.82 (3H, s), 3.30 (2H, m), 3.60 (1H, m), 3.78 (1H, m), 4.0 (2H, m), 4.22 (1H, m), 4.34-4.38 (3H, m), 5.80 (3H, s), 7.18-7.36 (5H, m).

L.

M. Literature References for Other Starting Materials:

The following starting materials were prepared as described in the cited references. The teachings of all of the references cited below are incorporated herein by reference.

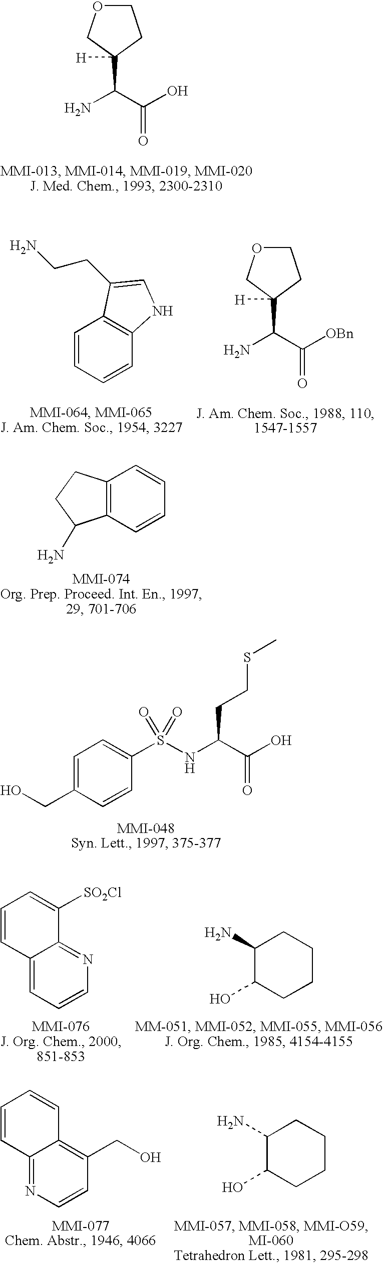

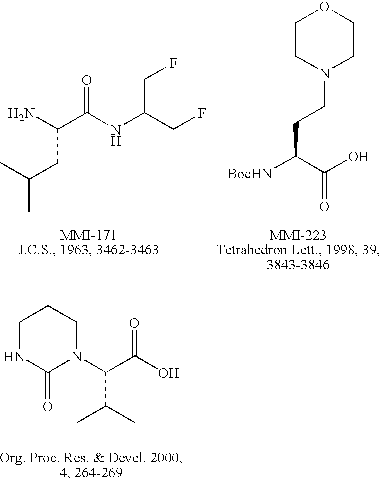

All other fragments needed for the synthesis of inhibitors of the invention are commercially available and were coupled using the appropriate procedures described above.

Determination of Kinetic Parameters

An aliquot of the inhibitor of known concentration in DMSO was diluted into 1.8 ml 0.1 M NaOAc, pH 4.0. DMSO and added to a final concentration of 10% (v/v), and memapsin 2 (final concentration of 80 nM), followed by a 20 minute equilibration at 37° C. Compounds were evaluated for the ability to inhibit memapsin 1 and memapsin 2 at concentrations between about 10 nM and about 10 μm of inhibitor. Proteolytic activity in presence of inhibitor was measured by addition of 20 μl of 300 μM substrate FS-2 dissolved in DMSO and increase in fluorescence intensity measured as previously described (Ermolieff, J., et al., Biochemistry 39:12450-12456 (2000), the teachings of which are incorporated herein by reference in their entirety).

The Kᵢₐₚₚ (apparent Kᵢ) values of inhibitors against memapsins 1 and 2 were determined employing previously described procedures (Ermolieff, J., et al., Biochemistry 39:12450-12456 (2000), the teachings of which are incorporated herein by reference in their entirety). The relationship of Kᵢ (independent of substrate concentration) to Kᵢₐₚₚ is a function of substrate concentration in the assay and the Kₘ for cleavage of the substrate by either memapsin 1 or memapsin 2 by the relationship: Kᵢₐₚₚ=Kᵢ (1+[S]/Kₘ).

Results and Discussion

Memapsin 1 is a protease that is closely homologous to memapsin 2 (also referred to herein as BACE, ASP2, β-secretase). Memapsin 2 catalyzes cleavage of β-amyloid precursor protein (APP) to produce β-amyloid (Aβ) peptide (also referred to herein as β-amyloid protein or β-amyloid peptide). Accumulation of Aβ peptide is associated with Alzheimer's disease. Memapsin 1 hydrolyzes the β-secretase site of APP, but is not significantly present in the brain. Further, there is no direct evidence the memapsin 1 activity is linked to Alzheimer's disease. The residue specificity of eight memapsin 1 subsite is: in positions P₄, P₃, P₂, P₁, P₁′, P₂′, P₃′ and P₄′ of the substrate, the most preferred residues are Glu, Leu, Asn, Phe, Met, Ile, Phe and Trp; while the second preferred residues are Gln, Ile, Asp, Leu, Leu, Val, Trp and Phe. Other less preferred residues can also be accommodated in these positions of the substrates. Some of the memapsin 1 residue preferences are similar to those of human memapsin 2, as described above. One embodiment of Applicants' invention is an N-terminal blocking group at P₃ of the inhibitor to attain the selectivity of the inhibitor for memapsin 2 activity over memapsin 1 activity. For example, compound MMI-138 with a dimethylpyrazole group at P₃ resulted in an inhibitor with a Kᵢ value about 60 times lower for memapsin 2 relative to memapsin 1 (see Table 1).

Determination of Side Chain Preference in Memapsin 1 Subsites

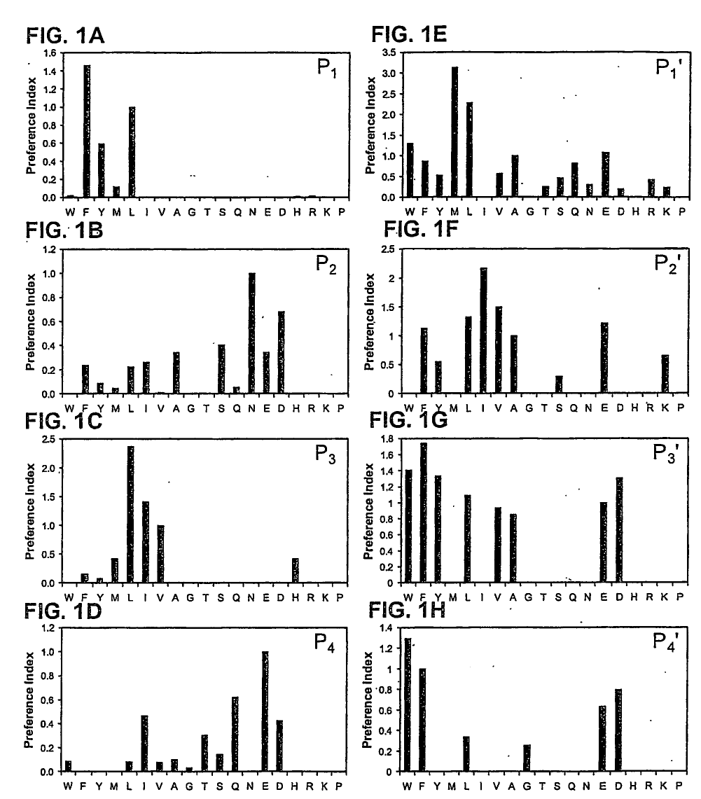

The relative hydrolytic preference of memapsin 1 at all eight positions of the peptide substrate is depicted in FIG. 1. Multiple substrate residues can be accommodated in each of the memapsin 1 subsites. The side chains on the P side are, in general, more stringent in specificity than those in the P′ side. P₁ is by far the most stringent position. Phe, Leu and Tyr have been found to be the most effective amino acid residues at P₁. All other position can accommodate more residues (FIG. 1). The most preferred residues are summarized in Table 4.

Farzan, et al. (Proc. Natl. Acad. Sci., USA 97:9712-9717 (2000), the teachings of which are incorporated herein by reference in their entirety) reported that memapsin 1 hydrolyzes APP preferentially at two sites in the sequence, between phe-phe and phe-ala in the sequence KLVFFAED (SEQ ID NO: 42). Based on specificity data described herein, either cleavage site has the most favored residue Phe at P₁ and medium or high ranking residues at P₂, P₁′, P₂′ and P₃′. P₂, P₄ and one of the P₄′ residues are clearly unfavorable (FIG. 1). These observations suggest that a memapsin 1 substrate can have some unfavorable residues and yet be a substrate for memapsin 1.

The screening of memapsin 1 binding to a combinatorial inhibitor library produced about 30 darkly stained beads. The sequences of fourteen of the darkest ones produced consensus residues in three of the four randomized positions on the substrate: P₃, Leu>Ile; P₂, Asp>Asn/Glu; P₂′ Val (Table 5). Side chain P₃′ did not produce clear consensus. Leu and Trp and Glu, which appeared more than once, are also preferred in substrate hydrolysis (FIG. 1). However, other residues unfavorable for substrates are also present. The lack of consensus at side chain P₃′ in the inhibitor library differs with substrate kinetic results, which clearly prefers Glu and Gln (Table 4). This discrepancy indicates that the nature of P₃′ residue is more important to effective substrate hydrolysis than to inhibitors binding.

Comparison on Subsite Preferences of Memapsin 1 and Memapsin 2

As discussed above, the overall substrate specificity of memapsin 1 subsites is similar to that for memapsin 2. As shown in Table 4, the top side chain preferences are either identical (for P₄) or differ only in the order of preference (for P₁, P₂, P₃ and P₂′). The two memapsins differ in residue preferences at the least specific P₃′ and P₄′ positions. The close similarity in consensus inhibitor residues at positions, P₃, P₂ and P₂′ are also seen from the inhibitor library (Table 7). In contrast to the preference of Glu and Gln in memapsin 2 sub-site S₃′, memapsin 1 failed to show a preference in this sub-site. The P₃′ side chain may interact poorly with memapsin 1 S₃′ site. Poor binding of both P₃′ and P₄′ has been observed for the binding of inhibitor OM99-2 to memapsin 2.

Implications on the Design of Selectivity for Memapsin 2 Inhibitors

β-secretase, also referred to herein as memapsin 2 or Asp 2, has been implicated in Alzheimer's disease since it cleaves the β-secretase site of β-amyloid precursor protein (APP) to generate β-amyloid (Aβ) protein which is localized in the brain. Memapsin 1 is a weak β-secretase enzyme compared to memapsin 2 and is not localized in the brain. Differences in the tissue distribution and β-secretase activity of memapsin 1 and memapsin 2 indicates they have different physiological functions.

The capping group (also referred herein as “blocking group”) at position P₃ in memapsin 2 inhibitors of the invention was evaluated to create selectivity of memapsin 2 inhibition. Small, yet potent, memapsin 2 inhibitors can be achieved by the elimination of the P₄ and the substitution of P₃ residue with a capping group as described above. New inhibitor MMI-138 (also referred to herein as “GT-1138”), which differs from inhibitor compound MMI-017 by a dimethylpyrazole group instead of a Boc at the N-terminus, produced a Kᵢ for memapsin 2 about 60 fold lower than the Kᵢ for memapsin 1 (Table 9, a blank space in the Table indicating that the value was not determined). Other inhibitors containing P₃ pyrazole capping group exhibited similar selectivity toward memapsin 2 (Table 9, % Inh (M1/M2)), whereas compounds with standard amino acid side chains in the P₃ position did not (Table 9).

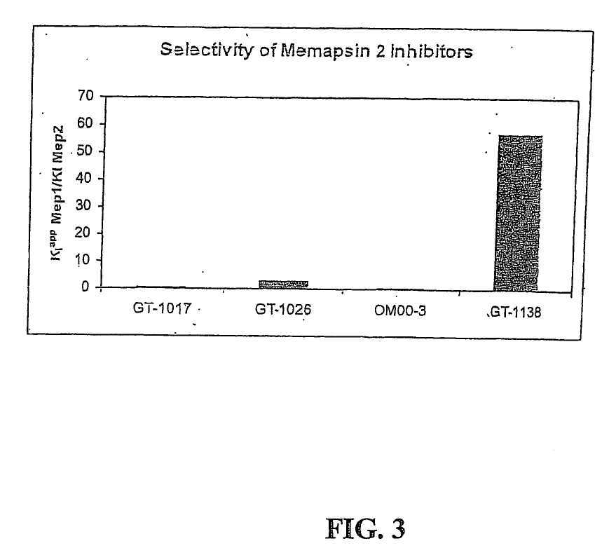

The data depicted in FIG. 3 was calculated from the Kᵢ apparent data depicted in Table 9. The inhibitors GT-1017 (also referred to herein as 017 and MMI-017), GT-1026 (also referred to herein as MMI-026) and OM00-3 have natural amino acids in the P₃ position, whereas inhibitor GT-1138 (also referred to herein as MMI-138) has a 3,5-dimethylphrazolyl derivative at the P₃ carbonyl. As shown in FIG. 3, the inhibitor MMI-138 resulted in about 60 fold selectivity of memapsin 1 relative to memapsin 2.

For effective penetration of the blood-brain barrier, memapsin 2 inhibitor drugs should be small in size. In view of the close similarity in inhibitor specificity of memapsin 1 and memapsin 2, a P₃ blocking group and other blocking groups to enhance binding and selectivity of memapsin 2 inhibitors were employed to design selective memapsin 2 inhibitors with desirable characteristics (e.g., appropriate size to penetrate the blood brain barrier, minimal peptide bonds, maximal hydrophobicity). The synthesis of inhibitors is described above and then Kᵢ, Kᵢₐₚₚ and relative selective inhibition are listed in Tables 1 and 9.

TABLE 7
PREFERRED AMINO ACID RESIDUES IN THE SUBSITES OF
MEMAPSINS 1 AND 2

| | Memapsin 1 | | | Memapsin 2^{b} | | |
|---|---|---|---|---|---|---|
| | Best | 2^{nd} | 3^{rd} and others | Best | 2^{nd} | 3^{rd} and others |
| P₁ | F | L | Y | L | F | M, Y, T |
| P₂ | N | D | S, A, E | D | N | M, F, Y, S |
| P₃ | L | I | V | I | V | L, E, H |
| P₄ | E | Q | D, I | E | Q | D, N, G |
| P₁′ | M | L | W, E, A, F | M | E | Q, A, D, S |
| P₂′ | I | V | L, E, F, A, K | V | I | T, L, F, M, Y |
| P₃′ | F | W, Y, D | L, V, A | W, V | I, T | D, E |
| P₄′ | W | F | D, E, L | D, E | W | F, Y, M |

^{a}Amino acid residues are shown in one-letter code
^{b}Memapsin 2 (amino acid residues 43-456 of SEQ ID NO: 8 (FIG. 11) and amino acid residues 45-456 of SEQ ID NO: 8 (FIG. 11)).

TABLE 8

OBSERVED RESIDUES AT THE P₃, P₂, P₂′ AND
P₃′ POSITIONS FROM BEADS WHICH STRONGLY
BOUND MEMAPSIN 1 SELECTED FROM A
COMBINATORIAL INHIBITOR LIBRARY

| | | | | |
|---|---|---|---|---|
| Bead No. | P₃ | P₂ | P₂′ | P₃′ |

| | | | | |
|---|---|---|---|---|
| 1 | Leu | Asp | Val | Met |
| 2 | Leu | ND^{b} | Ala | Leu |
| 3 | Leu | Glu | Val | Gln |
| 4 | Leu | Asp | Val | Trp |
| 5 | Ile | Asp | Val | Val |
| 6 | Ile | Phe | Val | Glu |
| 7 | Ile | Asp | Val | Asn |
| 8 | Ile | Asn | Val | Leu |
| 9 | Leu | Asp | Val | Lys |
| 10 | Leu | Asp | Val | Thr |
| 11 | Leu | Glu | Val | Trp |
| 12 | Leu | Gln | Val | Ile |
| 13 | Leu | Asn | Val | Glu |
| 14 | Leu | Asp | Val | Leu |
| Consensus | Leu > Ile | Asp > Asn/Glu | Val | None |
| Negative | | | | |
| controls^{c} | | | | |

^{a}Library template: Gly-P₃-P₂-Leu*Ala-P₂′-P₃′-Phe-Arg-Met-Gly-Gly-resin (SEQ ID NO: 27). The asterisk “*” denotes a hydroxyethylene
^{b}Not determinable
^{c}Negative controls are randomly selected beads with no memapsin 1 binding capacity

TABLE 9
SELECTIVITY OF INHIBITORS OF MEMAPSIN 2 AND MEMAPSIN 1

| | | | | Ki apparent | | | |
|---|---|---|---|---|---|---|---|
| | Compound | % Inhibition^{d} | | (nM) | | % Inh | Ki apparent |
| P3 Surrogate^{a} | Number | Mep2^{e} | Mep1^{f} | Mep2 | Mep1 | (M1^{e}/M2^{f})^{b} | (M1/M2)^{c} |
| heteroaralkoxy | 138 | 72 | 7 | 14.2 | 811.5 | 0.10 | 57.15 |
| heteroaralkoxy | 139 | 50 | 0 | | | 0 | |
| heteroaralkoxy | 156 | 68 | 32 | | | 0.47 | |
| heteroaralkoxy | 165 | 60 | 27 | 24.5 | | 0.45 | |
| heteroaralkoxy | 167 | 44 | 8 | | | 0.18 | |
| heteroaralkoxy | 171 | 45 | 8 | | | 0.18 | |
| heteroaralkoxy | 176 | 47 | 0 | | | 0 | |
| heteroaralkoxy | 181 | 47 | 9 | | | 0.19 | |
| heteroaralkoxy | 182 | 34 | 0 | | | 0 | |
| heteroaralkoxy | 180 | 26 | 0 | | | 0 | |
| heteroaralkyl | 196 | | | 28.8 | 351 | | 12.19 |
| heteroaralkyl | 204 | | | 27.4 | 1028 | | 37.56 |
| Valine | 116 | 56 | 85 | | | 1.52 | |
| Valine | 132 | 47 | 61 | | | 1.30 | |
| Leucine | 134 | 81 | 98 | | | 1.21 | |
| Valine | 78 | 37 | 28 | | | 0.76 | |
| Valine | 73 | 52 | 38 | | | 0.73 | |
| Valine | 17 | | | 3.9 | 1.2 | | 0.31 |
| Valine | 26 | | | 15.9 | 44.7 | | 2.80 |
| Leucine | OM00-3 | | | 0.31 | 0.18 | | .59 |

^{a}Heteroaralkyl or heteraaralkoxy derivative attached at P3 carbonyl, or standard P3 amino acid side chain
^{b}Values of 1 or less indicate inhibition which is greater towards memapsin 2 than memapsin 1
^{c}Larger values indicate inhibitors with greater affinity to memapsin 2 than to memapsin 1
^{d}Percentage of inhibition of proteolytic activity measured under conditions where the enzyme concentration equals the compound concentration
^{e}“Mep 2” and “M2” refer to memapsin 2
^{f}“Mep 1” and “M1” refer to memapsin 1

### Example 2

### Crystallization of Memapsin 2 Protein and Inhibitor of Memapsin 2

The hallmark of the Alzheimer's disease (AD) is a progressive degeneration of the brain caused by the accumulation of amyloid beta peptide, as referred to herein as β-amyloid protein (Selkoe, D. J., Physiol Rev 81:741-66 (2001)). The first step in the production of β-amyloid protein is the cleavage of a membrane protein called amyloid precursor protein (APP) by a protease known as the β-secretase, which has been identified as a membrane anchored aspartic protease termed memapsin 2 (or BACE or ASP-2). A first-generation inhibitor OM99-2 (Ghosh, A. K., et al., J. Am. Chem. Soc. 122:3522-3523 (2000)) was designed based on substrate information (Lin, X., et al., Proc Natl Acad Sci USA 97.1456-60 (2000), the teachings of which are incorporated herein by reference in their entirety) which is an eight-residue transition-state analogue, EVNL*AAEF (SEQ ID NO: 20) with Kᵢ near 1 nM (Ermolieff, J., et al., Biochemistry 39:12450-6 (2000)). A 1.9-Å crystal structure of the catalytic unit of memapsin 2 bound to OM99-2 (Hong, L., et al., Science 290:150-3 (2000), the teachings of which are incorporated herein by reference in their entirety) revealed that the conformation of the protease and the main features of its active site are those of the aspartic proteases of the pepsin family. All eight residues of OM99-2 were accommodated within the substrate-binding cleft of memapsin 2. The locations and structures of six memapsin 2 subsites for the binding of residues P₄ to P₂′ of OM99-2 were clearly defined in the structure (Hong, L., et al., Science 290:150-3 (2000), the teachings of which are incorporated herein by reference in their entirety). This part of the inhibitor assumed an essentially extended conformation with the active-site aspartyls positioned near the transition-state isostere between P₁ and P₁′. Unexpectedly, the backbone of the inhibitor turned at P₂′ Ala, departing from the extended conformation, to produce a kink. With less defined electron density, the side chains of P₃′ Glu and P₄′ Phe appeared to be located on the molecular surface and to have little interaction with the protease. These observations led to the idea that the S₃′ and S₄′ subsites in memapsin 2 were not well formed and perhaps contributed little to the interaction with substrates and inhibitors (Hong, L., et al., Science 290:150-3 (2000), the teachings of which are incorporated herein by reference in their entirety).

The detailed subsite preferences of memapsin 2 was determined as described above and by using preferred binding residues selected from a combinatorial inhibitor library, a second-generation inhibitor OM00-3, Glu-Leu-Asp-Leu*Ala-Val-Glu-Phe SEQ ID NO: 23 was designed and found to have a Ki of 0.3 nM as described below. The structure of the catalytic unit of memapsin 2 in complex with OM00-3 is described herein. The new structure defines the locations and structures of sub-sites S₃′ and S₄′, redefines subsite S₄ and provides new insight into their functions. Novel inhibitor/enzyme interactions were also observed in other sub-sites.

Methods to Generate Crystals of Protein and a Substrate Crystallization

Promemapsin 2-T1 (amino acid residues 1-456 of SEQ ID NO: 8 (FIG. 11)) was expressed in E. coli as an inclusion body and subsequently refolded and purified as previously described (Lin, X., et al., Proc Natl Acad Sci USA 97:1456-60 (2000); Ermolieff, J., et al., Biochemistry 39:12450-6 (2000), the teachings of all of which are incorporated herein by reference in their entirety). Crystallization of memapsin 2 amino acid residues 43-456 of SEQ ID NO: 8 (FIG. 11); amino acid residues 45-456 of SEQ ID NO: 8 (FIG. 11) complexed with OM99-2 and OM00-3 were carried out using established procedures (Hong, L., et al., Science 290:150-3 (2000), the teachings of which are incorporated herein by reference in their entirety) with minor modifications. For Memapsin2/OM99-2, the crystals were grown at 20° C. in 25% PEG (polyethylene glycol) 8000, 0.2 M (NH₄)₂SO₄ buffered with 0.1 M Na-Cacodylate at pH 6.5 using hanging drop vapor diffusion method with 1:1 volume ratio of well to sample solution. For OM00-3, 22.5% PEG 8000 was used at pH 6.2. Orthorhombic crystals were obtained under these conditions.

Data Collection and Processing

For data collection at 100° K, a crystal was first cryoprotected by transferring to well solution containing 20% (v/v) glycerol and then quickly frozen with liquid nitrogen. Diffraction data were collected on a Mar 345 image plate mounted on a Msc-Rigaku RU-300 X-ray generator with Osmic focusing mirrors. The data were processed using the HKL program package (Otwinowski, Z., et al., W. Methods in Enzymol. 276:307-326 (1997), the teachings of which are incorporated herein by reference in their entirety). Statistics are shown in Table 10.

Structure Determination and Refinement

Molecular replacement solutions were obtained for both crystals with the program AmoRe (Navaza, J., Acta Crystallogr D Biol Crystallogr 57:1367-72 (2001), the teachings of which are incorporated herein by reference in their entirety) using the previously determined memapsin 2 structure (Identifier Code: PDB ID 1FKN) as the search model. Translation search confirmed the two crystal forms are isomorphous in space group P2₁2₁2₁ (Table 7) with two memapsin 2/inhibitor complexes per crystallographic asymmetric unit. The refinement was completed with iterative cycles of manual model fitting using graphics program 0 (Jones, T. A., et al., Acta Crystallogr A 47:110-9 (1991), the teachings of which are incorporated herein by reference in their entirety) and model refinement using CNS (Brunger, A. T., et al., Acta Crystallogr D Biol Crystallogr 54:905-21 (1998), the teachings of which are incorporated herein by reference in their entirety). Water molecules were added at the later stages of refinement as identified in |Fo|−|Fc| maps contoured at 3 σ level. Ten percent of the diffraction data were excluded from the refinement at the beginning of the process to monitor the R values. The two memapsin 2/inhibitor complexes in the crystallographic asymmetric unit were found to be essentially identical. The coordinates for the structure reported here have been deposited in the Protein Data Bank (Accession Code 1M4H).

Kinetic Measurements

The measurement of relative k_{cat}/Kₘ values for the determination of residue preference at P₃′ and P₄′ were carried out as described above. Two template substrate sequences, WHDREVNLAAEF (SEQ ID NO: 28) and WHDREVNLAVEF (SEQ ID NO: 44) were used. The former had a P₃′ Ala and the latter a P₃′ Val. Four N-terminal residues, WHDR (SEQ ID NO: 29), were added to the substrate to facilitate the analysis using mass spectrometry. For each template, two each of peptide mixtures containing a total of 11 representative residues (in single letter code: A, D, E, F, L, M, R, T, V, W and Y) each at either P₃′ or P₄′ were designed and synthesized. The initial velocities for memapsin 2 hydrolysis of each peptides in the mixtures were determined in MALDI-TOF mass spectrometer as described above. The internal standards and the calculation of relative k_{cat}/Kₘ values were also as described above.

Results and Discussion

The crystal structure of OM99-2 bound to memapsin 2 is previously described in monoclinic space group P2₁ (Hong, L., et al., Science 290:150-3 (2000), the teachings of which are incorporated herein by reference in their entirety). In this study, the structures of OM99-2 and OM00-3/memapsin 2 complexes were solved and compared in the same space group—P2₁2₁2₁ (Table 10).

OM00-3 was designed based the crystal structure data of OM99-2 bound to memapsin 2 and the binding of memapsin 2 to a combinatorial inhibitor library as described above. Three amino acid residues are different in OM00-3 relative to OM99-2: P₃ Val to Leu, P₂ Asn to Asp, and P₂′ Ala to Val. These modifications improve the Kᵢ by 5.2 fold as shown above. The crystal structure of the OM00-3/memapsin 2 complex shows conformational changes for both the inhibitor and the enzyme. The most significant changes on the inhibitor can be observed at P₄ Glu.

In the OM99-2 structure, the P₄ Glu side chain carboxylate forms a strong hydrogen bond with the P₂ Asn side chain amide nitrogen (bond distance 2.9 Å). This conformation stabilizes the inhibitor N-terminus, but the P₄ side chain makes little contacts with the enzyme. The P₂ change from Asn to Asp in OM00-3 introduces electrostatic repulsion between the P₂ and P₄ side chains and eliminates the hydrogen bond between them. For the same reason, there is a rotation of the P₄ Glu main chain torsion of about 152 degrees, which places the P₄ side chain in a new binding pocket. At this position, the carboxylate oxygen atoms of P₄ Glu form several ionic bonds with the guanidinium nitrogen atoms of the Arg³⁰⁷ (SEQ ID NO: 9 (FIG. 12)) side chain. (References to the position of amino acid residues referred to in this example are to SEQ ID NO: 9 (FIG. 12)).

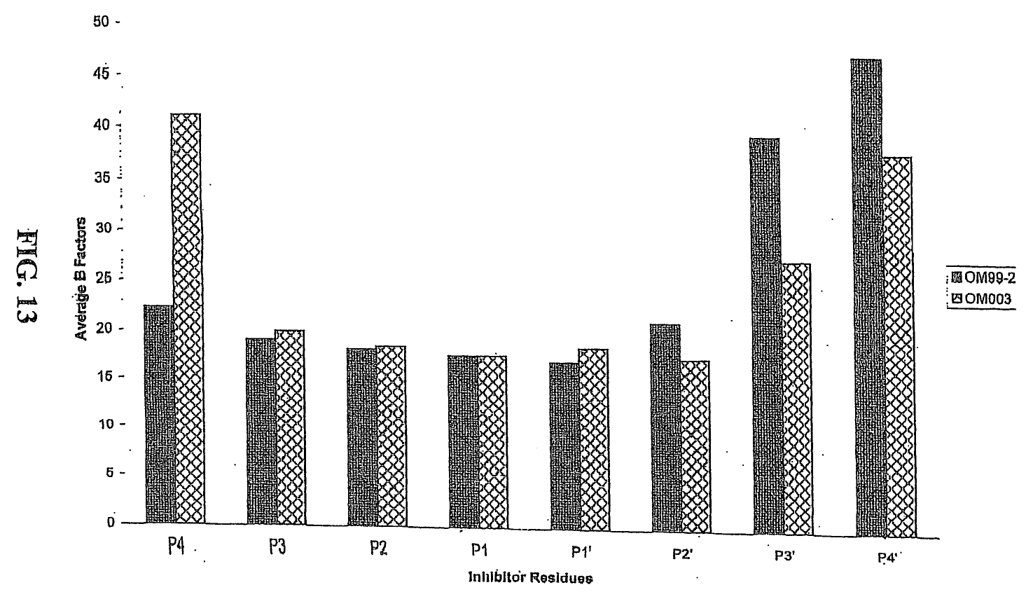

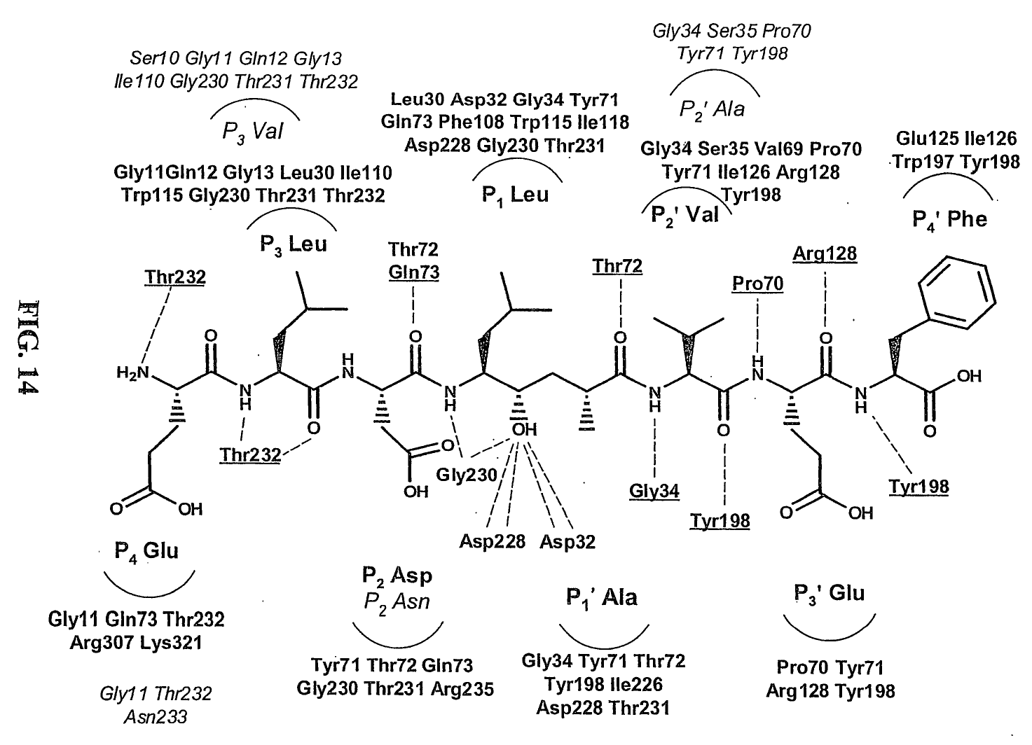

The memapsin 2 residues contacting the P₃ Leu, P₁ Leu, P₂′ Val, and P₄′ Phe (distance less than 4 Å) are shown in bold cased letters. The salt linkages (ion pairs) are likely to significantly increase the binding energy contributions of P₄ Glu to memapsin 2; yet, P₄ has increased mobility compared to that of the OM99-2 as indicated by their crystallographic B factors, whereas the average B factor differences between the two inhibitors from P₃ to P₂′ are insignificant (FIG. 13). This large difference is presumably due to the loss of the hydrogen bond to P₂ side chain. As a result of this Ψ rotation, the backbone nitrogen of P₄ is hydrogen bonded to Thr²³² (SEQ ID NO: 9 (FIG. 12)) side chain oxygen instead of to the Gly¹¹ (SEQ ID NO: 9 (FIG. 12)) main chain oxygen as observed in the OM99-2 structure.

OM99-2 was designed based on the Swedish Mutation of APP (SEVNLDAEFR; SEQ ID NO: 11) (Ghosh, A. K., et al., J Med Chem 44:2865-8 (2001), the teachings of which are incorporated herein by reference in their entirety). In its complex with memapsin 2, the side chains of P₂ Asn and Arg²³⁵ (SEQ ID NO: 9 (FIG. 12)) form hydrogen bonds, which may contribute to enhanced proteolysis and subsequently elevated Aβ production, leading to the early onset of Alzheimer's Disease (Hong, L., et al., Science 290:150-3 (2000), the teachings of which are incorporated herein by reference in their entirety). In the OM00-3 structure, the P₂ side chain is changed to Asp, and the Arg²³⁵ side chain adopts a new conformation, forming two salt linkages to the P₂ Asp side. These new ionic bonds make additional contributions to the inhibitor binding.

The effect of Val to Leu change at P₃ is subtle and involves adding and rearranging of hydrophobic interactions. The longer side chain of Leu at P₃ allows it to make van der Waals contacts with that of the P₁ Leu. The interactions between P₁ and P₃ side chains make them fit better into the corresponding hydrophobic binding pockets of the enzyme. Conformational changes are observed on the enzyme at Leu³⁰.

In the OM99-2 structure, the Leu³⁰ (SEQ ID NO: 9 (FIG. 12)) side chain does not contact the inhibitor but has extensive interactions with the Trp¹¹⁵ (SEQ ID NO: 9 (FIG. 12)) side chain and the main chain atoms of Glu¹² (SEQ ID NO: 9 (FIG. 12)) and Gly¹³ (SEQ ID NO: 9 (FIG. 12)). However, in the OM00-3 structure, the inhibitor side chain of P₁ Leu is extended and closer to that of Leu³⁰ (SEQ ID NO: 9 (FIG. 12)). In this case, the Leu³⁰ side chain makes a 60 degree rotation on the chi2 torsion angle. At this new position, the Leu³⁰ side chain has reduced interactions with Trp¹¹⁵ (SEQ ID NO: 9 (FIG. 12)), but makes van der Waals contacts to that of the P₃ Leu and P₁ Leu of the inhibitor as well as to the main chain atoms of Gly¹³ (SEQ ID NO: 9 (FIG. 12)) and Tyr¹⁴ (SEQ ID NO: 9 (FIG. 12)).

Structural flexibilities of the substrate binding sites of memapsin 2, such as the variations of side chain positions of Arg²³⁵ (SEQ ID NO: 9 (FIG. 12)) and Leu³⁰ (SEQ ID NO: 9 (FIG. 12)) upon binding to OM99-2 and OM00-3, were observed. The structural flexibility makes the enzyme bind to a broader range of substrates and/or inhibitors by improving the conformational complementarily between them.

The third residue change of OM00-3 from OM99-2 is at the P₂′ from Ala to Val. While the P₂′ is Ala in the pathogenic substrate APP, Val is a considerably better choice. The crystal structure indicates that the energetic benefit comes from the added van der Waals interactions in this hydrophobic pocket. The larger Val side chain has 5 more van der Waals contacts with the enzyme than the smaller Ala side chain (Table 11). There are 15 more van der Waals enzyme/inhibitor contacts in OM00-3 than that of OM99-2 because of the structure changes at P₃, P₁ and P₂′ (inter-atomic distances<4 Å).

TABLE 11

INTERACTIONS BETWEEN MEMAPSIN 2 AND COMPOUND
OM00-3 DETERMINED FROM THE CRYSTAL STRUCTURE
OF THEIR COMPLEX

| | | | |
|---|---|---|---|
| Residue on | Residue on | | |
| SEQ ID NO 9 | SEQ ID NO 8 | | |
| (FIG. 12) | (FIG. 11) | Interaction^{c} | OM00-3d |

| | |
|---|---|
| S₄ | Glu |

| | | | |
|---|---|---|---|
| Gly 11 bb | Gly 74 | Hbond | bb |
| Gln 73 sc | Gln 136 | Hbond | bb |
| Thr 232 sc | Thr 295 | Hbond | bb |
| Arg 307 sc | Arg 370 | Hbond, ionic | sc |
| Lys 321 Sc | Lys 384 | Hbond, ionic | sc |

| | |
|---|---|
| S₃ | Leu |

| | | | |
|---|---|---|---|
| Gly 11 bb | Gly 74 | Hbond | bb |
| Gln 12 bb | Gln 75 | Phobic | sc |
| Gly 13 bb | Gly 76 | Phobic | sc |
| Leu 30 sc | Leu 93 | Phobic | sc |
| Ile 110 sc | Ile 173 | Phobic | sc |
| Trp 115 sc | Trp 178 | Phobic | sc |
| Gly 230 bb | Gly 293 | Phobic | sc |
| Thr 231 bb | Thr 294 | Phobic | bb |
| Thr 232 bb | Thr 295 | Hbond | bb |

| | |
|---|---|
| S₂ | Asp |

| | | | |
|---|---|---|---|
| Tyr 71 bb | Tyr 134 | Phobic | bb |
| Thr 72 bb | Thr 135 | Phobic | sc |
| Gln 73 bb,sc | Gln 136 | Phobic, Hbond | sc |
| Gly 230 bb | Gly 293 | Hbond | bb |
| Thr 231 sc | Thr 294 | Phobic, Hbond | sc |
| Arg 235 sc | Arg 298 | Ionic | sc |

| | |
|---|---|
| S₁ | Leu |

| | | | |
|---|---|---|---|
| Leu 30 sc | Leu 93 | Phobic | sc |
| Asp 32 sc | Asp 95 | Hbond | bb |
| Gly 34 bb | Gly 97 | Phobic | bb |
| Tyr 71 sc | Tyr 134 | Phobic | sc |
| Gln 73 bb | Gln 136 | Phobic | sc |
| Phe 108 sc | Phe 171 | Phobic | sc |
| Trp 115 sc | Trp 178 | Phobic | sc |
| Ile 118 sc | Ile 181 | Phobic | sc |
| Asp 228 sc | Asp 291 | Hbond | bb |
| Gly 230 bb | Gly 293 | Phobic, Hbond | bb |
| Thr 231 sc | Thr 294 | Hbond | bb |

| | |
|---|---|
| S₁′ | Ala |

| | | | |
|---|---|---|---|
| Gly 34 bb | Gly 97 | Phobic | bb |
| Tyr 71 sc | Tyr 134 | Phobic | sc |
| Thr 72 sc | Tyr 135 | Phobic | sc |
| Tyr 198 sc | Tyr 261 | Phobic | sc |
| Ile 226 sc | Ile 289 | Phobic | sc |
| Asp 228 sc | Asp 291 | Hbond | bb |
| Thr 231 sc | Thr 294 | Phobic | bb |

| | |
|---|---|
| S₂′ | Val |

| | | | |
|---|---|---|---|
| Gly 34 bb | Gly 97 | Hbond | bb |
| Ser 35 sc | Ser 98 | Phobic | sc |
| Val 69 sc | Val 132 | Phobic | sc |
| Pro 70 bb | Pro 133 | Phobic | sc |
| Tyr 71 sc | Tyr 134 | Phobic | sc |
| Ile 126 sc | Ile 189 | Phobic | sc |
| Arg 128 sc | Arg 191 | Phobic | sc |
| Tyr 198 sc | Tyr 261 | Phobic | sc |

| | |
|---|---|
| S₃′ | Glu |

| | | | |
|---|---|---|---|
| Pro 70 sc | Pro 133 | Phobic | sc |
| Tyr 71 sc | Tyr 134 | Phobic | sc |
| Arg 128 sc | Arg 191 | Hbond | bb |
| Tyr 199 sc | Tyr 261 | Phobic | bb |

| | |
|---|---|
| S₄′ | Phe |

| | | | |
|---|---|---|---|
| Glu 125 sc | Glu 188 | Phobic | sc |
| Ile 126 sc | Ile 189 | Phobic | sc |
| Trp 197 sc | Trp 260 | Phobic | sc |
| Tyr 198 sc | Tyr 261 | Phobic | sc |

note:
^{a}Residue describes the amino acid residue and number of memapsin 2 (according to SEQ ID 9 (FIG. 12)) and whether side chain (sc) or backbone (bb) atoms of memapsin 2 are described in the interaction. Residues are grouped by subsite. Amino acid of compound OM00-3 appears in bold next to the subsite with which it is interacting.
^{b}Residue describes the amino acid residue and number of memapsin 2 (according to SEQ ID 8 (FIG. 11)).
^{c}Type of interaction: Phobic, hydrophobic or van der Waal contact; Hbond, hydrogen bond; ionic, ion pair between ionizable functional groups of opposite charges.
^{d}Interacting group of the compound OM00-3 is either side chain (sc) or backbone atoms (bb).

Glu and Phe comprise P₃′ and P₄′ for both of the inhibitors. Unlike the results obtained in space group P2₁ (Hong, L., et al., Science 290:150-3 (2000), the teachings of which are incorporated herein by reference in their entirety), the positions of P₃′ and P₄′ are better defined by electron density in space group P2₁2₁2₁. However, FIG. 13 shows that the average B factors for the P₃′ and P₄′ residues in the OM00-3 structure are considerably lower than that of the OM99-2 (the values are 27.1 and 37.6 for the former and 39.2 and 47.0 for the latter, respectively). Since the conformations of the inhibitor and the enzyme are nearly identical at these positions, an improved structure stability in OM00-3 at these two C-terminal residues, evidenced by lower average B factors, benefits inhibitor binding energetically, which consists of van der Waals contacts at P₃′ and P₄′. Considering the conformational and chemical resemblance at P₃′ and P₄′ between OM99-2 and OM00-3, it is considered that the large differences in B factors are caused by the Ala to Val change at P₂′. As discussed, Val improves the fit between the inhibitor and the enzyme at this position. The enhanced binding at P₂′ may stabilize the relative mobile P₃′ and P₄′.

The crystal structure of the memapsin 2/OM99-2 indicates of an S₅ substrate binding site on the enzyme. The N-terminal nitrogen of OM99-2 points to a hydrophilic opening on memapsin 2, which comprises Lys⁹, Ser¹⁰, Gly¹¹, Gln¹², Pro¹⁶⁰, and Pro³⁰⁸ (SEQ ID NO: 9 (FIG. 12)), and can potentially be used as a substrate or inhibitor binding pocket beyond S₄. The N-terminal nitrogen of OM00-3 points to the inside of the enzyme and does not likely mimic the extending N-terminal position of a protein substrate. On the contrary, the orientations of C-terminal carboxyl groups of both inhibitors indicate that the next residue would be pointing away from the enzyme surface and no additional binding sites can be found beyond S₄′.

The crystal structure of memapsin 2 and the compound, OM00-3, was compared with a crystal structure of memapsin 2 and the inhibitor compound OM99-2 in the same space group. New enzyme/inhibitor interactions have been identified in several binding pockets. These include both electrostatic and van der Waals contacts. A possible substrate binding site beyond S₄ was also identified.

The structure of the catalytic domain of human memapsin 2 bound to an inhibitor OM00-3 (ELDL*AVEF; SEQ ID NO: 23, Kᵢ=0.3 nM, * denotes hydroxyethylene transition-state isostere) has been determined at 2.1 Å resolution.

Uniquely defined in the structure are the locations of S₃′ and S₄′ sub-sites, which were not identified in the previous structure of memapsin 2 in complex with inhibitor OM99-2 (EVNL*AAEF; SEQ ID NO: 20 Kᵢ=1 nM). Different binding modes for P₂ and P₄ side chains are also identified. The structural and kinetic data demonstrate that the replacement of the P₂′ alanine in OM99-2 with a valine in OM00-3 stabilizes the binding of P₃′ and P₄′.

TABLE 10

| | OM99-2/Memapsin 2 | OM003/Memapsin 2 |
|---|---|---|
| | Data statistics | |
| Space group | P 2₁ 2₁ 2₁ | P 2₁ 2₁ 2₁ |
| Unit cell a, c, and c (D) | 86.1, 88.1, 130.8 | 86.5, 88.8, 131.0 |
| Resolution (D) | 25.0-2.0 | 25.0-2.1 |
| Number of observed | 348,996 | 190,727 |
| reflections | | |
| Number of unique | 65,542 | 58,864 |
| reflections | | |
| R_{merge} | 0.075 | 0.119 |
| Data completeness (%) | 96.6 (25.0-2.0 D) | 98.8 (25.0-2.1 D) |
| | 94.3 (2.07-2.00 D) | 97.1 (2.18-2.10 D) |
| I/F(I) | 20.0 (25.0-2.0 D) | 7.3 (25.0-2.1 D) |
| | 5.4 (2.07-2.00 D) | 2.2 (2.18-2.10 D) |
| | Refinement statistics | |
| R_{working} | 0.192 | 0.216 |
| R_{free} | 0.233 | 0.271 |
| RMS deviation from ideal | | |
| values | | |
| Bond length (D) | 0.008 | 0.009 |
| Bond angle (degrees) | 1.5 | 1.6 |
| Number of water molecules | 544 | 450 |
| Average B factors (D²) | | |
| Protein | 23.1 | 22.8 |
| Solvent | 28.9 | 26.1 |

Structure and Inhibitor Binding

The structure of the OM00-3/memapsin 2 complex in space group P2₁2₁2₁ was determined at 2.1 Å using the molecular replacement method. The structure of the enzyme, the interactions of the P₁/P₁′ (Leu*Ala) region of OM00-3 with the substrate binding cleft of memapsin 2 and the backbone conformation of the inhibitor from P₃ to P₂′ are essentially the same as in the structure of the OM99-2/memapsin 2 complex. However, the current structure shows different side-chain configurations within the S₄, S₃ and S₂ sub-sites when compared to those of the OM99-2 structure (Hong, L., et al, Science 290:150-3 (2000), the teachings of which are incorporated herein by reference in their entirety). In addition, the locations and nature of S₃′ and S₄′ binding pockets are defined.

S₄, S₃ and S₂ Subsites

The new S₄ pocket in the current structure involves memapsin 2 residues Gly¹¹, Gln⁷³, Thr²³², Arg³⁰⁷ and Lys³²¹. The Arg³⁰⁷ and Lys³²¹ (SEQ ID NO: 9 (FIG. 12)) form several ionic bonds to the carboxylate oxygen atoms of inhibitor P₄ Glu. In the previous OM99-2/memapsin 2 structure, the main-chain torsion angle, Ψ, of the P₄ Glu is different from the current one by 152°. Thus, in OM99-2 structure, there is a hydrogen bond between the side chains of P₄ Glu and P₂ Asn, and the P₄ Glu side chain in that structure has little interaction with the protease. In OM00-3 structure, however, P₂ is an Asp, and thus its interaction with P₄ Glu is unfavorable. Although the average B factor of P₄ Glu is somewhat higher (42 Å²) than those of the interior residues of P₃ to P₂′ (17-20 Åhu 2), its multiple interactions with the protease residues suggest that the newly observed S₄ pocket contributes significantly to the inhibitor binding.

In the OM00-3 structure, Leu³⁰ (SEQ ID NO: 9 (FIG. 12)) in S₃ of the protease has contacts with the leucines of the inhibitor at P₃ and P₁. These two side chains also contact each other, contributing to the further stabilization of the inhibitor conformation. These productive interactions are not present in the OM99-2 structure where P₃ is Val rather than Leu and the conformation of Leu³⁰ is different as a result of a 60 degree rotation around χ2.

In the S₂ pocket, the P₂ Asp of OM00-3 forms two ionic bridges to the Arg²³⁵ (SEQ ID NO: 9 (FIG. 12)) side-chain. The conformation of Arg²³⁵ (SEQ ID NO: 9 (FIG. 12)) is different from that in the OM99-2 structure where the P₂ residue is Asn. Flexibility within the S₂ pocket allows interaction with either Asp or Asn at P₂ and is consistent with the observation that these two residues are the most preferred substrate and inhibitor residues for this subsite (Table 7 and FIG. 2).

S₃′ and S₄′ Subsites

In contrast to the OM99-2/memapsin 2 structure, the conformation of the P₃′ and P₄′ side chains is well defined by electron density in the OM00-3/memapsin 2 structure. The backbone at P₃′ and P₄′ of OM00-3 assumes an extended conformation which is stabilized by a hydrogen bond from P₃′ backbone carbonyl to Arg¹²⁸ (SEQ ID NO: 9 (FIG. 12)). A very weak hydrogen bond from P₄′ backbone nitrogen to Tyr¹⁹⁸ may make small contributions to the binding. The S₃′ and S₄′ subsites are defined by several direct van der Waals interactions (<4.5 Å (Table 11)). By virtue of their location at the C-terminus of the inhibitor, both P₃′ and P₄′ residues have somewhat higher average B factor values (28 Å² and 37 Å², respectively) than those of the residues in the region from P₃ to P₂′ In the presence of easily interpretable electron density, these higher temperature factors do not compromise the validity of the structural information and the analysis of the interactions for sub-sites S₃′ and S₄′.

Contribution of P₂′ to the Binding of P₃′ and P₄′

Inhibitors OM99-2 and OM00-3 have identical P₃′ and P₄′ residues. It was therefore unexpected that the P₃′ and P₄′ are better defined for the latter structure. Kinetics studies have shown that, compared to the other subsites, subsites that bind P₃′ and P₄′ have a considerably broader range of amino acid preference (FIG. 2). Because the P₂′ Val in OM00-3 has several more contacts with the enzyme than the Ala in OM99-2 (Hong, L., et al., Science 290:150-3 (2000), the teachings of which are incorporated herein by reference in their entirety), it was reasoned that a better binding of P₂′ Val may contribute to the stability of P₃′ and P₄′ residues in OM00-3. P₂′ Val may shift residue preference at P₃′ and P₄′ toward Glu and Phe, respectively. Thus, the relative residue preference at P₃′ and P₄′ positions for two sets of substrates, EVNLAAEF (SEQ D NO: 15) and EVNLAVEF (SEQ ID NO: 45), which differed only in Ala or Val at P₂′, was measured.

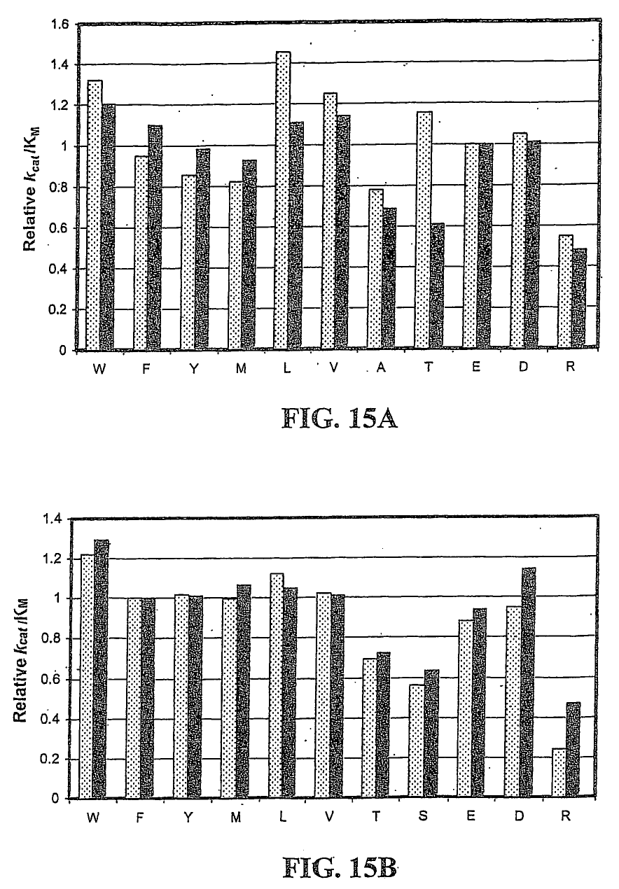

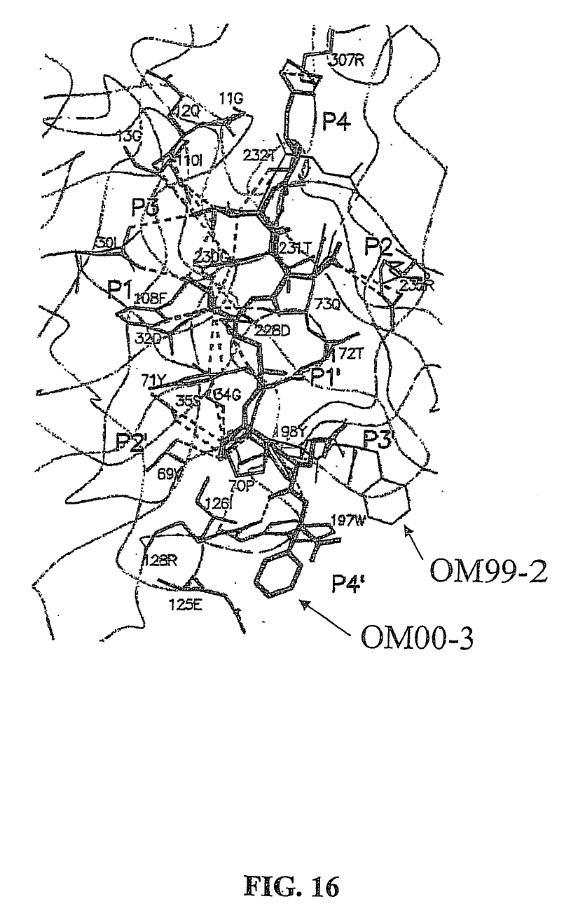

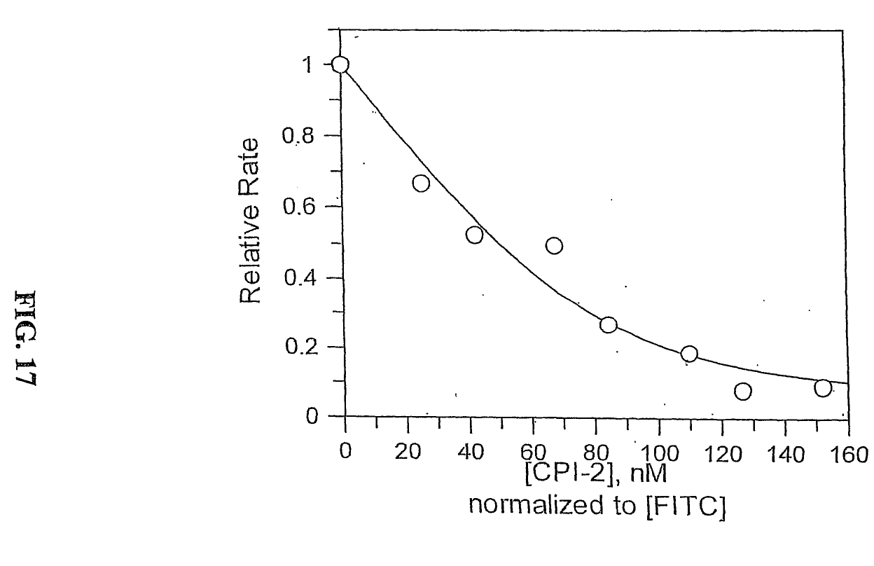

Ten representative residues were chosen for each of the P₃′ and P₄′ positions in addition to the native residue. The relative k_{cat}/Kₘ values of these eleven substrates in a single mixture were determined by their relative initial hydrolytic rate using a mass spectrometric method as described above. The results show that the differences in residue preferences at subsites that bind P₃′ (FIG. 15A) and P₄′ (FIG. 15B) side chains for two sets of substrates with P₂′ Ala and P₂′ Val are small.

The template sequence EVNLAAEF (SEQ ID NO: 15) employed to discern the amino acid residue preference (FIGS. 15A and 15B) included P₄ to P₄′. The data depict the relative k_{cat}/Kₘ compared to the substrate with a glutamic acid (E) at P₃′ and a phenylalanine (F) at P₄′ which are assigned a value of 1. k_{cat}/Kₘ values were normalized for substrates containing an alanine at the P₂′ and a valine at the P₂′ positions of both substrate mixtures.

A number of interactions are noted between the inhibitor compounds of the invention and memapsin 2. As shown in Table 11, there are hydrophobic contacts between the side chains of P₃, P₁ and P₂′. In addition, salt bridges and hydrogen bonds from the P₄ and P₂ side chains and the P₃′ and P₄′ backbone of the inhibitor are also observed.

There is no shift of preference at P₃′ and P₄′ side chains toward Glu and Phe, respectively, when P₂′ is Val; yet, peptide substrates with Val at P₂′ have on average about 30% higher k_{cat}/Kₘ values than their counterparts with Ala at P₂′. To determine which kinetic parameter contributes to this difference, the individual kcat and Km values for two substrates differing at only P₂′ by Val or Ala was measured. Substrate EVNLAVEFWHDR (SEQ ID NO: 30) produced a Kₘ of 83±8.9 mM and a k_{cat} of 1,007±106 s−1 (n=3) while substrate EVNLAAEFWHDR (SEQ ID NO: 31) had a Kₘ of 125±11 mM and a k_{cat} of 274±23 s−1 (n=2). The differences in kinetic parameters between P₂′ Val and P₂′ Ala substrates are much greater in k_{cat} (˜4 fold) than in Kₘ (˜1.5 fold). Thus, compared with P₂′ Ala, P₂′ Val primarily improves the transition-state binding of P₃′ and P₄′ residues, but does not alter their specificity.

New Subsites in Inhibitor Design

The first structure of memapsin 2 catalytic domain complexed to inhibitor OM99-3 (Hong, L., et al., Science 290:150-3 (2000), the teachings of which are incorporated herein by reference in their entirety) has been shown to be useful in the structural based design of smaller and potent memapsin 2 inhibitors (Table 1). The new structure described here provides improved versatility for inhibitor design. Memapsin 2 inhibitors with clinical potentials should be potent, selective and small enough to penetrate the blood-brain barrier. It is known that HIV protease inhibitor drug indinavir, 614 Da, can cross the blood-brain barrier (Martin, et al., Aids 13:1227-32 (1999), the teachings of which are incorporated herein by reference in their entirety). A memapsin 2 inhibitor of similar size would bind to about five sub-sites consecutively. Inhibitors with Kᵢ at low nM range can be designed without evoking binding at the P₃′ and P₄′ subsites (Table 1). The new binding modes at P₄ and P₂ can be utilized for the design of inhibitors of this type. The new sub-site structures of S₃′ and S₄′ described above can be incorporated in the design of inhibitors with P₃′ and P₄′ but without P₄ and P₃ residues. Such a design is predicted to have a strong binding side chain, such as Val, at P₂′.

### Example 3

### Crystal Structure of Compound MMI-138 Complexed to Memapsin 2

Compound MMI-138 selectively inhibits memapsin 2 over memapsin 1, evident as the Kᵢ value for the former are 60-fold lower than that of the latter. Moreover, other compounds that have a functional group containing pyrazole as the R₁ group of formula II likewise demonstrate selectivity based upon their relative Vi/Vo measurements (Table 9). To determine the structural features of MMI-138 that contribute to the selectivity of the inhibitor, a crystal structure of memapsin 2 in complex with MMI-138 was determined. The structure reveals the pyrazole group was bound to the enzyme in the S₃ subsite, forming hydrogen bonds. A peptide bond in memapsin 2 was flipped relative to its orientation in the crystal structures of complexes between memapsin 2 and either OM99-2 (Hong, L., Turner, R. T., 3rd, Koelsch, G., Shin, D., Ghosh, A. K., Tang, J., “Crystal structure of memapsin 2 (beta-secretase) in complex with an inhibitor OM00-3,” Biochemistry 41:10963-10967 (2002); and Hong, L., Koelsch, G., Lin, X., Wu, S., Terzyan, S., Ghosh, A. K., Zhang, X. C., Tang, J., “Structure of the protease domain of memapsin 2 (beta-secretase) complexed with inhibitor,” Science 290:150-153 (2000)) or OM00-3 (Hong, L., Turner, R. T., 3rd, Koelsch, G., Shin, D., Ghosh, A. K., Tang, J., “Crystal structure of memapsin 2 (beta-secretase) in complex with an inhibitor OM00-3,” Biochemistry 41:10963-10967 (2002)). Modeling of the memapsin 1 structure in the vicinity of the pyrazole binding region suggests that such an orientation is unfavorable for memapsin 1. The possibility of other energetic or structural features that impart selectivity are not excluded by the model.

Experimental Procedure

Enzyme Preparation

Promemapsin 2-T1 was expressed as outlined in Example 1 and purified. The memapsin 2 used in the crystallization procedure was obtained by activation of promemapsin 2-T1 (SEQ ID NO 8 as shown in FIG. 11) with clostripain and purification by anion exchange FPLC (Ermolieff et al., Biochemistry 39: 12450-12456 (2000)). The activated memapsin 2 corresponded to amino acids 60-456 of SEQ ID NO 8 (as shown in FIG. 11).

Crystallization

The memapsin 2/MMI-138 crystals were obtained by a replacement or “soaking” procedure (Munshi, S., Chen, Z., Li, Y., Olsen, D. B., Fraley, M. E., Hungate, R. W. and Kuo, L. C., “Rapid X-ray diffraction analysis of HIV-1 protease-inhibitor complexes: inhibitor exchange in single crystals of the bound enzyme,” Acta Cryst. D54:1053-1060 (1998); see procedure below). In this procedure, a complex is obtained between the protein and a compound of affinity less than the compound of interest (in this case MMI-138). This crystal is then placed in a solution of the compound of interest (i.e., “soaked”) to allow the compound of interest to diffuse and exchange with the compound of weaker affinity present in the proteins of the crystal. Therefore, for crystals of memapsin 2 in complex with MMI-138, crystals first had to be obtained with a complex of memapsin 2 and a compound of weaker affinity. The compound of weaker affinity used in the procedure was designated OM01-1 (Ki=126 nM): 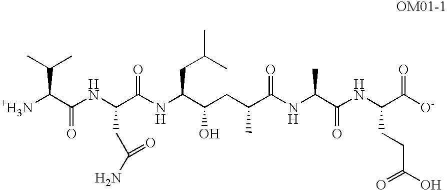
OM01-1 was dissolved in dimethyl sulfoxide (DMSO) to a concentration of 25 mg/ml. Memapsin 2 (amino acids 60-456 of SEQ ID NO: 8 (FIG. 11)) was produced as described above. The purified memapsin 2 protein was concentrated to 40 mg/ml and was mixed with the 25 mg/ml OM01-1 solution, such that the final concentration of DMSO was 10%, and the final concentration of OM01-1 was 2.5 mg/ml. Crystallization buffer (20% PEG 8000, 0.2 M (NH₄)₂SO₄, and 0.1 M sodium cacodylate at pH 6.5) was combined with the memapsin 2/OM01-1 complex mixture, and mixed 1:1 (vol:vol) with the crystallization buffer (well solution), and allowed to equilibrate with the well solution at 20° C. according to the established hanging drop procedure.
Soaking Procedure for Compound Exchange

To obtain crystals of a complex between memapsin 2 and compound MMI-138, a replacement or “soaking” procedure was followed (Munshi, et al. 1998). OM01-1 synthesized using standard solid-phase peptide synthesis using an FMOC-protected hydroxyethylene isostere established by our lab (Ghosh, et al. 2000). Crystals of memapsin 2 in the presence of OM01-1 were obtained by the above mentioned crystallization procedure and were transferred to a 10 μl volume of a solution of the crystallization buffer containing 10% DMSO and 2 mg/ml of compound MMI-138, as well as memapsin 2 protein, present at a concentration of no more than one-half the molar concentration of MMI-138, but preferably one-fifth the molar amount of MMI-138, for the purpose of stabilizing the crystal during the soaking procedure. The solution was incubated at 20° C. for 48 hours to allow the compound OM01-1 present in the crystal to equilibrate with the compound MMI-138, resulting in an exchange between OM01-1 in complex with memapsin 2 in the crystals for compound MMI-138.

X-Ray Diffraction, Data Collection, and Analysis

Crystals of memapsin 2 in complex with MMI-138, obtained by the above procedure were incubated in cryo-protectant buffer (crystallization buffer containing 20% glycerol) for 1-2 minutes, followed by flash-freezing in a liquid nitrogen stream. Diffraction data was collected on a Rigaku RU-300 X-ray generator with a M345 image plate at 100° K. Data was indexed and reduced with the HKL program package (Otwinowski, Z., and Minor, W., Methods in Enzymol. 276:307-326 (1997)). Molecular replacement method was used to solve the structure with the memapsin 2/OM99-2 crystal structure as the initial model. Molecular replacement solutions were obtained with the program AmoRe (Navaza, J., Acta Crystallogr D Biol Crystallogr 57:1367-72 (2001)). The refinement was completed with iterative cycles of manual model fitting using graphics program O (Jones, T. A., Zou, J. Y., Cowan, S. W., and Kjeldgaard, Acta Crystallogr A 47:110-9 (1991)) and model refinement using CNS (Brunger, A. T., Adams, P. D., Clore, G. M., DeLano, W. L., Gros, P., Grosse-Kunstleve, R W., Jiang, J. S., Kuszewski, J., Nilges, M., Pannu, N. S., Read, R. J., Rice, L. M., Simonson, T., and Warren, G. L., Acta Crystallogr D Biol Crystallogr 54:905-21 (1998)). The data obtained is shown in Table 12.

TABLE 12
Data Collection and Refinement Statistics for MMI-138
complexed to memapsin 2.

| | MMI-138/Memapsin 2 |
|---|---|
| Space group | P2₁ |
| Unit cell a, c, and c (Å) | 86.3, 87.9, 131.0 |
| Unit cell α, β, and γ (degrees) | 90.0, 89.97, 90.0 |
| Resolution (Å) | 25.0-2.1 |
| Number of observed reflections | 322,438 |
| Number of unique reflections | 106,913 |
| ^{a}R_{merge} | 0.080 |
| Data completeness (%) | 92.7 (50.0-2.1 Å) |
| | 71.9 (2.18-2.10 Å) |
| I/σ(I) | 12.7 (50.0-2.1 Å) |
| | 2.4 (2.18-2.10 Å) |
| ^{b}R_{work} | 0.247 |
| ^{b}R_{free} | 0.291 |
| RMS deviation from ideal values | |
| Bond length (Å) | 0.011 |
| Bond angle (degrees) | 1.7 |
| Number of water molecules | 518 |
| Average B factors (Å²) | |
| Protein | 30.3 |
| Solvent | 30.3 |

^{a}R_{merge} = Σₕₖ₁Σᵢ|Iₕₖ₁, ᵢ − <Iₕₖ₁> Σ/Σₕₖ₁ <Iₕₖ₁>, where Iₕₖ₁, ᵢ is the intensity of the i^{th measurement and <I}ₕₖ₁> is the weighted mean of all measurements of Iₕₖ₁.
^{b}R_{work} (free) = Σ||Fₒ| − |F_{c}||/Σ|Fₒ|, where Fₒ and F_{c} are the observed and calculated structure factors. Numbers in parentheses are the corresponding numbers for the highest resolution shell (2.18-2.1 Å). Reflections with Fₒ/σ(Fₒ) >= 0.0 are included in the refinement and R factor calculation.

Results and Discussion

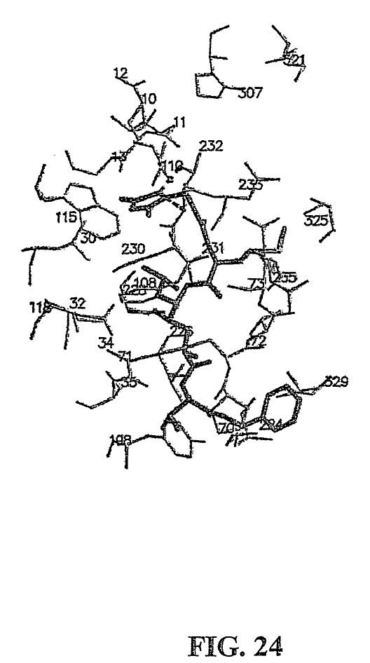

The dimethylpyrazole group at the N-terminus (e.g., the R₁ group of Formula II) of the compounds of the invention provides inhibition selectivity for memapsin 2 over memapsin 1 (see FIG. 24). FIG. 24 shows the active site region of the crystal structure of MMI-138 (shown as the darker bonds) complexed to memapsin 2 (shown as lighter bonds). The dimethylpyrazole moiety is pictured with hydrogen bonds (dashed lines) between N11 of the pyrazole ring of MMI-138 and Thr²³² backbone and side chain atoms of memapsin 2. Throughout the discussion in this section, amino acid residues are numbered according to SEQ ID NO: 9 (FIG. 12). The Kᵢ for memapsin 2 of MMI-138 is about 60 times lower (more potent) than that of memapsin 1. The crystal structure shown in FIG. 24 shows that the pyrazole group binds to the S₃ pocket of memapsin 2. It resides in a much deeper position in the pocket than of the P₃ amino acid side chains such as Val and Leu in OM99-2 and OM00-3, respectively. The contacting residues of memapsin 2 to the pyrazole group consist of Gly¹¹, Gln¹², Gly¹³, Gly²³⁰, Thr²³¹, and Thr^{232 9}. The pyrazole derivative is further makes hydrophobic contacts with Leu³⁰, Ile¹¹⁰, and Trp¹¹⁵.

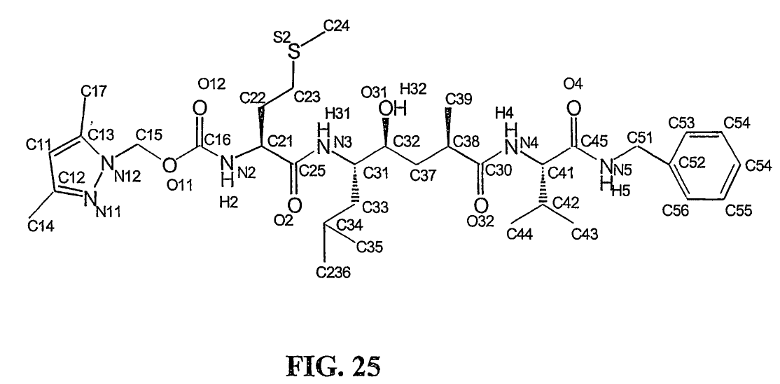

FIG. 25 is a structural schematic of MMI-138. The atoms of MMI-138 are numbered to correspond to the atoms named in the atomic coordinates of the crystal structure of the complex between MMI-138 and memapsin 2. As discussed above, the nitrogen atom N11 of the pyrazole ring forms two hydrogen bonds with the Thr²³² backbone nitrogen and side chain oxygen atoms. In this position, the N11 would be very close to the carbonyl oxygen of Ser¹⁰ (˜2.4 Ångstroms), as it exists in the structure of the complex between OM99-2 and LRL-memapsin 2 (Hong, et al. 2000) and in the structure of the complex between OM00-3 and LRL-memapsin 2 (Hong, et al. 2002). The close contact of the two electronegative atoms would be very energetically costly. To allow the binding of the pyrazole ring and avoid the close contact with N11, the backbone carbonyl oxygen of Ser¹⁰ reorients such that the peptide bond is flipped. Crystal structure of LRL-memapsin 2/MMI-138 complex clearly shows a 180 degree flip of Ser¹⁰ backbone oxygen in comparison with the structure of other memapsin 2/inhibitor compound complexes. This conformation change is required to accommodate the pyrazole ring in the S₃ pocket.

However, the crystal structure likewise indicates the flip of the carbonyl oxygen of Ser¹⁰ is unfeasible for memapsin 1. The Lys⁹ in memapsin 2 is an Asp in memapsin 1. According to our modeled memapsin 1 structure, the Asp side chain would form a hydrogen bond with the backbone nitrogen of Arg¹² (2.9 Ångstroms). This hydrogen bond and position of Asp⁹ side chain should stabilize the hairpin loop from Ser⁹ to Arg¹² and prevent the peptide flip as required for the pyrazole group binding. The flip would position the Ser¹⁰ carbonyl oxygen in close proximity (˜2.3 Å) to the negatively charged Asp⁹ side chain and/or distort the hydrogen bond, which is not energetically favorable. It is also possible that in memapsin 1, the main chain conformation is different from that of the memapsin 2 around Ser¹⁰, and the peptide flip would cause the main chain torsion angles to have disfavored ψ and Φ combinations.

### Example 4

### Inhibition of β-Amyloid Protein Production in a Mammal Following Administration of a Compound Which Inhibits Memapsin 2 Activity

Preparation of the Carrier Peptide-Inhibitor (CPI) Conjugates

The carrier molecule peptide employed in these experiments was a peptide derived from a segment of the HIV tat protein (amino acid residues 47-57) (Schwarze, S. R., et al., Science 285:1569-1572 (1999), the teachings of which are incorporated herein in their entirety) or has an amino acid sequence Tyr-Gly-Arg-Lys-Lys-Arg-Arg-Gln-Arg-Arg-Arg (SEQ ID NO: 32) and an oligo-D-arginine residue (R-R-R-R-R-R-R-R-R (SEQ ID NO: 33)) (Wender, P. A., et al. Proc. Natl. Acad. Science USA 97:664-668 (2000), the teachings of which are incorporated herein in their entirety).

Carrier Peptide-Inhibitor conjugates are referred to herein by the designation “CPI” followed by a number, e.g., CPI-1, CPI-2 and CPI-3. CPI-1 is the OM99-2 inhibitor complexed to a carrier peptide. CPI-2 is the OM00-3 inhibitor complexed to a carrier peptide.

The structure of the carrier peptide inhibitor conjugates employed in the experiments was:

(SEQ ID NO: 34)

CPI-1: FAM-Ahx-(EVNL*AAEF)-G-(YGRKKRRQRRR)

(SEQ ID NO: 35)

CPI-2: FAM-Ahx-(ELDL*AVEF)-GG-(RRRRRRRRR)

Where G is glycine; Y, R, K, Q, E, V, N, L, A, F and D are L-amino acids tyrosine, arginine, lysine, glutamine, glutarmic acid, valine, asparagine, leucine, alanine, phenylalanine and aspartic acid, respectively. Italic R represents D-arginine. 5-(and 6-) carboxyfluorescein (FAM), is linked to the amino group of the 6-aminohexanoic acid (Ahx) group. The carboxyl group of Ahx is linked by an amide bond to amino group of the first amino acid in the inhibitor moiety.

Ahx and glycine residues were employed as spacers in the complex. The square brackets enclose the carrier peptides, which are tat residues 47-57 in CPI-1 and nine D-arginine residues (Wender, P. A., et al., Proc. Natl. Acad. Sci. USA 97:13003-13008 (2000), the teachings of which are incorporated hereby in their entirety) in CPI-2, respectively. The asterisks in the inhibitor sequences represent the transition-state isostere, hydroxyethylene (Ghosh, A. K., et al., J. Am. Chem. Soc. 122:3522-3523 (2000), the teachings of which are incorporated hereby in their entirety).

The Carrier Peptide is referred to herein a “CP,” followed by a number. A fluorescein-labeled carrier peptide, CP-1, excluding a conjugated inhibitor moiety, was also designed for control experiments. The structure of CP-1 is as follows:

| | |
|---|---|
| CP-1: FAM-Ahx-GGG-(YGRKKRRQRRR) | (SEQ ID NO: 36) |

The peptide portions of CPI-1, CPI-2 and CP-1 were synthesized using solid-phase peptide synthesis and purified by reversed phase HPLC. Protected Leu*Ala diisostere derivative was used at a single step in the synthesis of CPI-1 and CPI-2 (Ghosh, A. K., et al., J. Am. Chem. Soc. 122:3522-3523 (2000) the teachings of which are incorporated hereby in their entirety). FAM attachment was facilitated by active ester chemistry according to procedures of the supplier (Molecular Probes).

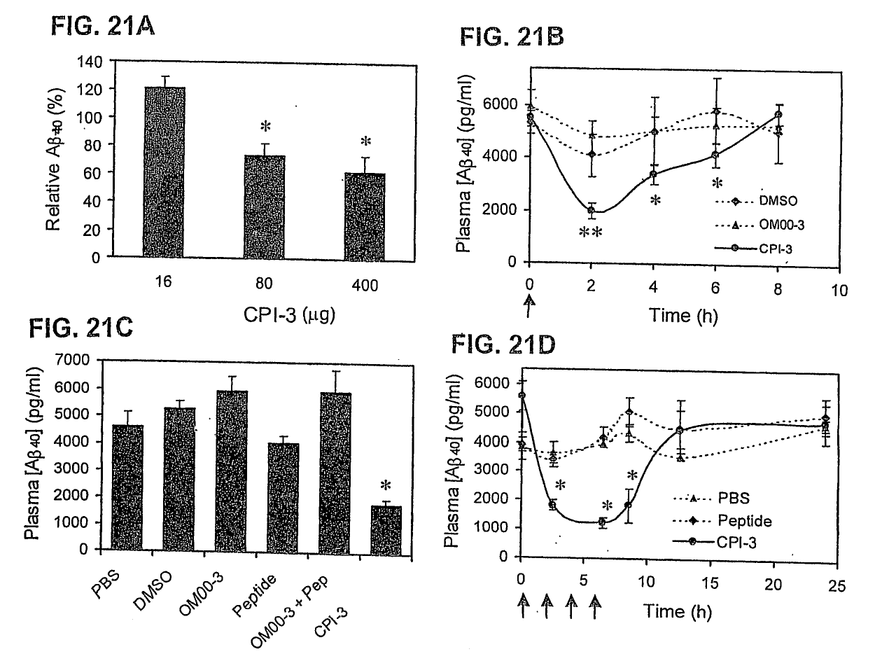

Kinetic inhibition experiments (FIG. 21), using a procedure as described in Ermolieff, et al. (Biochemistry 39:12450-12456 (2000), the teachings of which are incorporated hereby in their entirety), showed that the conjugated inhibitors CPI-1 and CPI-2 had similar inhibition potencies as their inhibitors, OM99-2 and OM00-3 with Ki apparent values of 39 and 58 nM, respectively (Lin, X., et al., Proc. Natl. Acad. Sci. USA 97:1456-1460 (2000); Ermolieff, J., et al., Biochemistry 39:12450-12456 (2000), the teachings of all of which are incorporated hereby in their entirety).

The concentrations of the conjugates and control were normalized to peptide concentration either from amino acid analysis or by fluorescence values using a fluorescence spectrophotometer AMINCO-Bowman Series 2. An excitation wavelength of 492 nm and an emission wavelength of 516 nm were used to monitor the amount of fluorescence from the conjugated fluorescein.

Transport of Conjugated Inhibitors to Mouse Brain Experimental Procedure

Two- to four-month-old Cd72c mice were injected intraperitoneally (i.p.) with 0.3 to 10 nmoles of the conjugates (CPI-1 or CPI-2) or with control fluorescein, in 200 μl of PBS. Whole blood cells (with EDTA as anti-coagulant in the syringe or in the capillary tube) were isolated from anesthetized animals from the orbital artery or by heart puncture and diluted 1:10 in PBS. Prior to the harvest of other tissue samples, animals were anesthetized and perfused with 150 ml of neutral-buffered 10% formalin. Spleens were harvested intact. Brains were harvested and cerebral hemispheres separated, one for sectioning by cryostat, the other for single cell isolation for flow cytometry.

Sections of the brain hemispheres were obtained by soaking in OCT/PBS at 4° C. for overnight, recovered and frozen in Histo Prep Media. Sections (10 μm) were cut on a cryostat, fixed in 0.25% of formalin for 15 min, and histologically stained with three antibodies: (1) Alexa Fluor 488 conjugated anti-fluorescein (Molecular Probes; (2) Polyclonal goat anti-human-pro-memapsin 2 antibody; (3) followed by Cy3 conjugated anti-goat IgG antibody (Sigma, St. Louis, Mo.); and (4) Biotin-conjugated anti-bovine α-tubulin followed by Alexa Fluor 350™ conjugated to neutravidin (Molecular Probes). After mounting with anti-fade solution with a cover slip, the sections were analyzed by fluorescence confocal microscopy.

To collect single cell suspensions, spleens and brain hemispheres were homogenized through a 30 μm screen and directly analyzed by flow cytometric analysis. An alternative means to staining brain cells was first to permeabilize them in 0.2% Tween 20 in PBS, blocked with 1% normal rabbit serum, incubated with 1:50 diluted Alexa Fluor 488™ conjugated anti-fluorescein (1 mg/ml; Molecular Probes, Eugene, Oreg.) for 30 minutes, then analyzed by flow cytometric analysis.

Fluorescein was conjugated to the amino terminus of OM00-3 by incubation with NHS-fluorescein (Pierce, Rockford, Ill.) and purified to >90% by reversed-phase HPLC and dissolved in DMSO to 50 mg/ml.

Fluorescently labelled inhibitors or fluorescein (Fs) as a control were incubated with suspended cells for time intervals ranging from 10 to 30 minutes. Cells were fixed with paraformaldehyde and permeabilized in 0.2% Tween-20 in PBS for 6 minutes and incubated with anti-fluorescein-Alexa™ 488 antibody (Molecular Probes, Eugene, Oreg.) in order to enhance detection of intracellular inhibitor present from penetration. Flow cytometry (FACSCalibur™) and confocal fluorescent microscopy (Leica TCS NT™) were performed at the Flow and Image Cytometry Lab, OUHSC.

Results

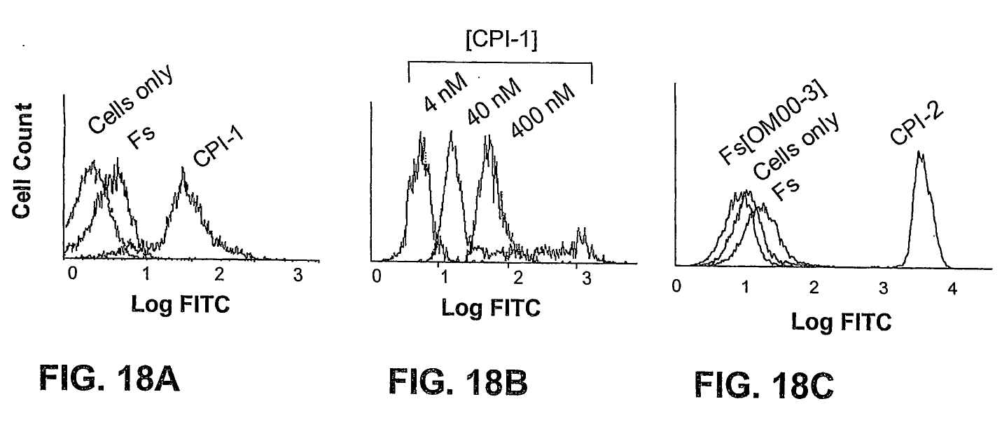

The conjugated inhibitors, CPI-1 and CPI-2, readily penetrated cultured cells within minutes, as indicated by intracellular fluorescence of FAM group (FIGS. 18A, 18B and 18C).

Incubation of HEK293 cells with Fs[OM99-2]tat resulted in an increase of fluorescence relative to cells incubated with fluorescein alone, as demonstrated by flow cytometry (FIG. 18A). Furthermore, the fluorescence intensity of the incubated cells correlated with the inhibitor concentration in the range of 4 nM to 400 nM (FIG. 18B). CPI-2 likewise penetrated cells, whereas Fs[OM00-3], without the CP moiety (oligo-D-arginine), did not (FIG. 18C “peptide”), demonstrating that the CP was necessary for transporting the inhibitor across the plasma membrane. The transport of conjugated inhibitors was observed in several cell lines including HeLa cells and M17 cells, the latter being a neuronal cell line.

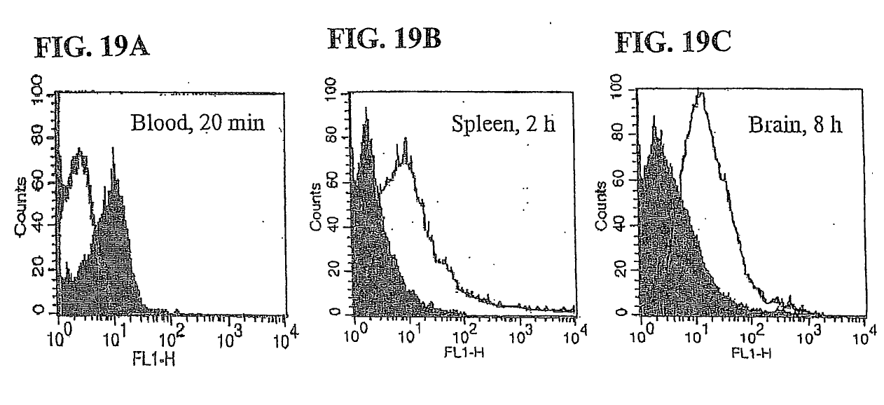

Entry of the CPI-1 and CPI-2 conjugates into the mammalian brain was determined. Mice, strain Cd27c, were injected i.p. with 0.3 nmol of either CPI-1, CPI-2 or CP-1 and cells and organs monitored for fluorescence due to the FAM group in the injected compounds. Flow cytometric analysis of whole blood isolated 20 minutes after i.p. injection with CPI-1 revealed a strong fluorescence signal in approximately 100% of blood cells (FIG. 19A). Blood cells from mice injected with fluorescein as a control showed a small constant increase in background fluorescence that was likely due to uptake of the compound from the peritoneum by the lymphatic system and adsorbed onto the cell surface.

Splenocytes were analyzed for the presence of CPI-1, CPI-2 and CP-1 by performing a splenectomy 2 hours after i.p. injection of the mice and isolating the splenocytes. Flow cytometric analysis revealed translocation of conjugates into all splenocytes, including T cells, B cells, and macrophages, resulting in a fluorescence peak shift in almost 100% of cells (FIG. 19B). Like blood cells, control i.p. injection of equimolar amounts of free fluorescein showed only a minor increase in fluorescence above background levels. The injected conjugate was rapidly transduced into blood and splenic cells in the mouse, within approximately 20 minutes and 2 hours, respectively.

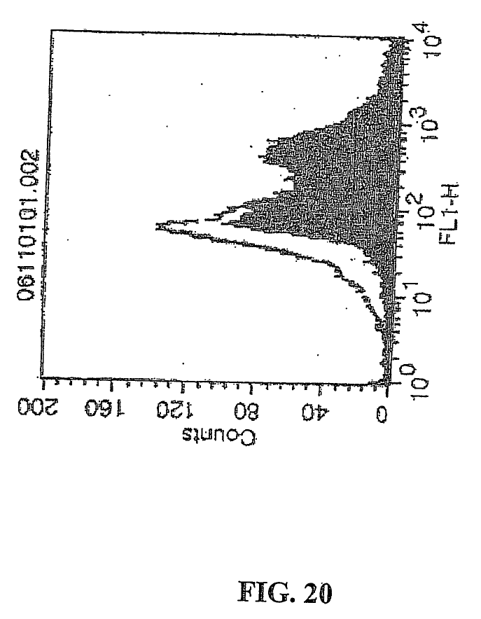

The uptake of the CPI-1 into brain tissue was determined. Whole brains were dissected from perfused mice 8 hours after i.p. injection of the conjugate or fluorescein as a control. Hemispheres were separated and either frozen for cryostat sectioning or for isolation of cells by homogenization on nylon mesh. Flow cytometric analysis revealed penetration of the fluorescent conjugate into all brain cells, resulting in a fluorescence peak shift (FIG. 19C). A two-peak intermediate stage showing brain cells being gradually transformed from a basal level population to a level containing higher fluorescence intensity was observed (FIG. 20).

Fluorescence confocal microscopy analysis of 10 μm hemispheric sagittal brain sections revealed a strong signal in all areas of the brain from mice injected with CPI-2, while the signal in fluorescein control injected mice remained at background levels. Eight hours after i.p. injection, the confocal microscopy result showed that fluorescein localized primarily to the nuclei of cell bodies throughout the brain section.

Inhibition of Aβ Secretion from Cultured Cells by Conjugated Inhibitors

Observations described above established that the two conjugated inhibitors, CPI-1 and CPI-2, were able to penetrate the plasma membrane of cells in vitro or the blood brain barrier (BBB) or in vivo. Inhibition of the activity of memapsin 2 in cultured cells by a conjugated inhibitor was determined. Since the hydrolysis of APP by memapsin 2 leads to the formation of Aβ and its secretion to the culture medium, the effect of conjugated inhibitor CPI-2 on APP cleavage was determined by measuring the secreted Aβ in the culture medium.

Experimental Procedure

Cultured cells, including human embryonic kidney (HEK293) cells, HeLa, and neuroblastoma line M17, purchased from American Type Culture Collection (ATCC), were stably transfected with two nucleic acid constructs that encode human APP Swedish mutant (APPsw; SEVNLDAEFR (SEQ ID NO: 11)); and human memapsin 2 (amino acid residues 14-501 of SEQ ID NO: 6 (FIG. 9)), which included leader peptide from PSEC-tag genes. Cells were maintained in Dulbecco's modified Eagle's medium supplemented with 10% (v/v) fetal calf serum and 1% penicillin/streptomycin. Two antibiotics, Zeocin (1 μg/ml) and G418 (250 μg/ml) were included in the media for maintenance of the stably transfected lines.

Either the parental lines (293, HeLa, or M17) or the stably transfected lines (293-D, HeLa-D, or M17-D) were plated on 6-well plates and grown in a 37° C., 5% CO₂ incubator until 90% confluent. Cells were then treated with or without 10 pmole of CPI-2 overnight then labeled by using [³⁵S]TransLabel Protein Labeling Mix (100 μCi/ml) (ICN) in methionine- and cysteine-free DMEM for an additional 18 hours. For treatment of cells with CPI-2, 10 pmole of the inhibitor conjugate was dispensed to cells 20 minutes prior to labeling, and likewise into labeling media.

Cells were lysed in 1 ml of RIPA buffer (10 mM Tris, pH 7.6, 50 mM NaCl, 30 mM sodiumpyrophosphate, 50 mM NaF, and 1% NP-40) supplemented with 1 mM PMSF, 10 μg/ml leupeptin, 2.5 mM EDTA, 1 M pepstatin, and 0.23 U/ml aprotonin. The total cell lysates were subject to immunoprecipitation by the addition of 1 μl of 1 mg/ml of monoclonal antibody raised specifically against human Aβ₁₇₋₂₄ (MAB 1561, Chemicon) with 20 μl of protein G-sepharose beads. Immunoprecipitated proteins were denatured in Tricine-SDS sample buffer with 2.5% β-mercaptoethanol by boiling for 5 minutes. Immunoprecipitated proteins were analyzed by using 10-20% gradient SDS-PAGE (NOVEX) and radiolabeled proteins were visualized by autoradiography. Quantitative results were obtained using the STORMY phosphorimaging system (Amersham).

Results

Immunoprecipitation of Aβ from HEK 293 cells transfected with sw (Swedish mutation) APP and memapsin 2 (amino acid residues 14-501 of SEQ ID NO: 6 (FIG. 9)) (293-D cells) revealed a clear Aβ band in SDS-PAGE at the position of 4.5 kDa as compared to the same treatment of native HEK 293 cells. Following treatment of cells with CPI-2, the amount of Aβ produced by the stably transfected cell line was markedly reduced, whereas no effect was seen in control HEK 293 cells. Quantification of ³⁵S intensity of the bands by phosphorimaging indicated over 95% inhibition of Aβ by the conjugated inhibitor CPI-2.

Preparation of Additional Carrier Peptide-Inhibitor Conjugates

The structure of the carrier peptide inhibitor conjugate CPI-3 was designed as follows:

(SEQ ID NO: 35)

CPI-2: FAM-Ahx-(ELDL*AVEF)-GG-(RRRRRRRRR)

(SEQ ID NO: 37)

CPI-3: (ELDL*AVEF)-GG-(RRRRRRRRR)

Where G is glyine; Y, R, K, Q, E, V, N, L, A, F and D are L-amino acids tyrosine, arginine, lysine, glutamine, glutarmic acid, valine, asparagines, leucine, alainine, phenylalanine and aspartic acid, respectively. Italic R represents D-arginine. The preparation of CPI-2 is described above. CPI-3 was synthesized employing a similar procedure. CPI-3 has the same amino acid sequence as CPI-2, but lacks the fluorescent FAM tag. The amino terminus of CPI-3 is a free primary amine and is not linked either to aminohexyl or to the FAM group. The asterisks in the inhibitor sequences represent the transition-state isostere, hydroxyethylene.

The peptide portion of CPI-3 was synthesized using solid-phase peptide synthesis and purified by reversed phase HPLC. Protected Leu*Ala diisostere derivative, described previously (Ghosh, A. K., et al., J. Am. Chem. Soc. 122:3522-3523 (2000), the teachings of which are incorporated hereby in their entirety), was used at a single step in the synthesis of CPI-3 as described in Ghosh, et al. (J. Am. Chem. Soc. 122:3522-3523 (2000), the teachings of which are incorporated hereby in their entirety).

Kinetic inhibition experiments using a procedure as described in Ermolieff, et al. (Biochemistry 39:12450-12456 (2000), the teachings of which are incorporated hereby in their entirety) showed that the conjugated inhibitors CPI-3 had similar inhibition potencies as the parent inhibitor, OM00-3, with a Kᵢ of 35 nM.

Inhibition of Aβ Production in Transgenic Mice

Experimental Procedure

Six-month-old tg2576 mice (n=21) were injected intraperitoneally (i.p.) 200 μg of conjugate CPI-3 or with control DMSO, in 200 μl of PBS. Plasma were collected from anesthetized animals by orbital bleed or sephaneous vein into heparinized capillary tubes and clarified by centrifugation. Plasma A≢ 1-40 levels were determined by capture ELISA (BioSource International, Camarillo, Calif.). The peptide analogue of CPI-3, with an amide group instead of the hydroxyethylene isostere was synthesized by SynPep (Camarillo, Calif.).

Results

The conjugated inhibitor CPI-2 readily penetrated cultured cells within minutes, and penetrated into the brain and other tissue within hours, as indicated by intracellular fluorescence of FAM group, as discussed above.

Since the conjugate inhibitors can cross the blood brain barrier in vivo, enter cells both in vitro and in vivo, and inhibit Aβ production in vitro, inhibition of Aβ production in vivo was determined. Tg2576 mice, expressing the Swedish mutation of the human amyloid precursor protein (including SEQ ID NO: 11) (Hsiao, K., et al., Science 274:99-102 (1996), the teachings of which are incorporated hereby in their entirety) were injected with CPI-3, which is identical in amino acid sequence to CPI-2 and lacks the amino-terminal fluorescein (FAM) derivative. Blood was collected from tg2576 animals at time intervals following injection of 400 μg of CPI-3.

At ages up to 9 months, plasma AD in the tg2576 mice serves as a reliable marker for brain Aβ production as a result of memapsin 2 activity (Kawarabayashi, T., et al., J. Neurosci. 21:372-381 (2001)). Nine tg2576 mice were injected intraperitoneally with various doses of inhibitor CPI-3. Two hours following the injection of CPI-3, plasma Aβ₄₀ showed a significant dose-dependent reduction relative to Aβ₄₀ from control mice injected with PBS (FIG. 21A).

To study the duration of inhibition, eight tg2576 mice were injected intraperitoneally with inhibitor CPI-3. The plasma Aβ₄₀ level dropped to about one third of the initial value at 2 hours following injection (FIG. 21B), consistent with presence of CPI-3 in the brain in the same range of time verified by confocal microscopy. The inhibition had a relatively short half-life of 3 hours, with the plasma Aβ₄₀ level then gradually returning to the initial value by 8 hours (FIG. 21B), consistent with the observed disappearance of fluorescent inhibitor CPI-3 from brains of mice at 8 hours post-injection, observed by confocal microscopy. Injecting either the unconjugated inhibitor OM00-3 or the peptide analogue of CPI-3 without the transition-state isostere (FIG. 21C “peptide”) did not reduce plasma Aβ₄₀ levels. The latter established that the carrier molecule was not responsible for the observed inhibition, nor did it facilitate a general permeabilization of the blood brain barrier, as simultaneous injection with the peptide analogue of CPI-3 and the inhibitor OM00-3 did not decrease plasma Aβ₄₀ (FIG. 21 COM00-3+peptide). The percentage of Aβ₄₀ relative to total Aβ was constant at 73±8% and 75±5% for Aβ levels ranging from about 1000 to about 5000 pg/ml in treated and untreated animals, respectively. These observations established that the measured Aβ₄₀ changes may be taken as the change of total Aβ in the observed range of inhibition.

Since the observed duration of inhibition had been relatively short, the maximal inhibition level of this inhibitor by repeated injections was determined. Experiments with four injections at 2 hour intervals significantly reduced the Aβ level to an average low of 29% (ranging 22% to 33%) of the average initial value (FIG. 21D). The difference in Aβ values of the experimental group and the control group receiving PBS or the peptide analogue of CPI-3 were statistically significant at time points 2 hours following a given injection. The observed reduction of plasma Aβ in these AD mice represents largely the inhibition of Aβ produced almost entirely in the brain, because Aβ has been demonstrated to rapidly exit from the brain to the plasma (Ghersi-Egea, J. F. et al. J. Neurochem. 67:880-883 (1996)). Thus the inhibition of about 80% of plasma Aβ must involve the reduction of Aβ output from the brain.

Carrier molecules had previously been shown to facilitate the transport of natural macromolecules such as protein and DNA across the cell membrane. The demonstration here that carrier molecules assist the transport of synthetic inhibitors containing non-peptidic bonds across the cell membrane and the blood brain barrier (BBB) raises the possibility that carrier molecules can be employed for the delivery of Alzheimer's diseasse therapeutics and others targeted to the central nervous system or other tissues or organelles. The advantage of such an approach is that the parental inhibitors need not be small enough for BBB penetration so the drug can be selected from a wider repertoire of candidate compounds based on potency, selectivity and other drug properties. Drug delivery employing carriers could be considered for those targets of the for which drugs with properties suitable for cell membrane penetration are difficult to attain.

Inhibition of Aβ Production in Transgenic Mouse Model of Alzheimer's Disease

Although many of the compounds of the invention demonstrate strong inhibition of memapsin 2 (amino acid residues 43-456 of SEQ ID NO: 8 (FIG. 11) and amino acid residues 45-456 of SEQ ID NO: 8 (FIG. 11)) in the in vitro fluorogenic assay, it was unknown whether any of these compounds could inhibit Aβ (also referred to herein as β-amyloid protein) production in vivo. Generally, the molecular size of the compounds would be considered too large to permit crossing of the blood brain barrier. Restrictions of about 500 g/mole or less have been reported (Brightman, M. W., et al., Curr. Top. Microbiol. Immunol. 202:63-78 (1995); Zolkovic, B., Neurobiol. Dis. 4:23-26 (1997); Egleton, R. D., et al., Peptides 18:1431-1439 (1997); van de Waterbeemd, H., et al., J. Drug Target 6:151-165 (1998), the teachings of all of which are incorporated hereby in their entirety). A critical feature required for the action of a compound to block Aβ production is that the compound can penetrate the blood brain barrier. The brain is an important site of action in the treatment of Alzheimer's disease since the brain mediates memory and cognition.

The tg2576 transgenic mouse expresses the human Swedish amyloid precursor protein (APP) under control of the prion promoter to direct expression mainly in the brain (Hsiao, K., et al., Science 274:99-102 (1996), the teachings of which are incorporated hereby in their entirety). The Aβ peptide produced in the brain can be detected in plasma of these transgenic animals from ages 3-12 months (Kawarabayashi, T., et al., J. Neurosci. 21:372-381 (2001), the teachings of which are incorporated hereby in their entirety) and results from its efflux from the brain, known to occur within minutes (Ghersi-Egea, et al., J. Neurochem. 67:880-883 (1996), the teachings of which are incorporated hereby in their entirety). Thus, monitoring the plasma Aβ provides a useful continuous measurement of effective inhibition of Aβ production in the brain.

Reduction of Aβ levels in the plasma, following adminstration of a memapsin 2 inhibitor, is an indication that the compound inhibited Aβ production in the brain by crossing the blood brain barrier. Fluorescently-labeled memapsin 2 inhibitor conjugated to a carrier peptide (CPI-2) was shown to cross the blood brain barrier, and inhibit Aβ production, as discussed above. Employing the same experimental protocol described above, it was demonstrated that three of the inhibitor compounds of the invention, MMI-138, MMI-165, and MMI-185 penetrated the blood brain barrier in transgenic mice (strain tg2576), resulting in reduction of Aβ production.

Materials and Methods

Compounds

Compounds MMI-138, MMI-165, and MMI-185 were synthesized as described above. Compounds were dissolved in 1 ml of dimethyl sulfoxide (DMSO) to a final concentration of about 1 mg/ml for MMI-165 and MMI-185, and about 10 mg/ml for MMI-138. Inhibitor OM00-3 was synthesized as described above and dissolved in DMSO to about 10 mg/ml. Inhibitors were diluted into PBS or H₂O immediately prior to injection, as described below. Inhibition constants were determined by methods described by Ermolieff, et al (Biochem. 39:12450-12456 (2000), the teachings of which are incorporated hereby in their entirety).

Animal Models, Treatment and Sampling Protocol

The tg2576 strain of mice was obtained from Taconic (Germantown, N.Y.). The APP/F strain of mice were obtained by mating the tg2576 mice with the FVB/N strain. To determine presence of the Swedish APP gene in APP/F mice, the DNA from mice was isolated according to the Qiagen Dneasy™ Tissue Kit. PCR (Qiagen kit and protocol) was used to amplify the fragment of DNA corresponding to the human Swedish APP gene. The following primers were used:

| | | |
|---|---|---|
| Beta actin XAHR17 | 5′ - CGG AAC CGC TCA TTG CC | (SEQ ID NO: 38) |
| Beta actin XAHR 20 | 5′ - ACC CAC ACT GTG CCC ATC TA | (SEQ ID NO: 39) |
| 1503 APP | 5′ - CTG ACC ACT CGA CCA GGT TCT GGG T | (SEQ ID NO: 40) |
| 1502 APP | 5′ - GTG GAT AAC CCC TCC CCC AGC CTA GAC CA | (SEQ ID NO: 41) |

Beta actin primers were used as a positive control. After PCR was performed, the samples were analyzed on a 1% agarose gel containing 0.5 μg/ml EtBr in a 1×TAE (Tris-Acetate-EDTA) running buffer.

At the age of three months, animals of the Alzheimer's disease mouse model APP/F were injected intraperitoneally (i.p.) with about 163 nanomoles of either compounds MMI-138 (molecular weight 674 g/mole; 110 μg per animal, n=2), MMI-165 (molecular weight 626 g/mole; 102 μg per animal, n—2), or MMI-185 (molecular weight 686; 112 μg per animal, n—2). Control animals were injected with either DMSO alone (100 μl diluted into 100 μl of PBS) or 163 nmoles of inhibitor OM00-3 (Table 3) to 10 mg/ml stock in DMSO diluted into PBS, final volume 200 μl.

Heparinized capillary tubes collected blood samples from anesthetized animals from either the retro-orbital sinus or from the saphenous vein at specified intervals following injection. The blood samples were transferred to sterile 1.5 mL microcentrifuge tubes, centrifuged at 5,100 RPM for 10 minutes to recover the plasma (supernatant), and stored at −70° C. until analysis for the Aβ₄₀ by Enzyme Linked-Immuno-Sorbent Assay (ELISA).

A sandwich ELISA (BioSource International, Camarillo, Calif.) was used to determine the levels of Aβ₄₀ in plasma samples. The ELISA utilizes a primary antibody specific for human Aβ for the immobilization of the amino-terminus and a detection antibody specific for the carboxy-terminal amino acids of Aβ₄₀. A conjugated secondary antibody was used to detect the ternary complex, using a stabilized chromogen substrate, quantifiable following addition of 1 M HCl, with the optical densities measured at 450 nm. The procedures were followed according to the BioSource protocol. Optical densities were converted to pg/ml quantities of Aβ₄₀ using a linear regression of the optical densities of standards obtained from the commercial kit, to their known concentrations.

Results and Discussion

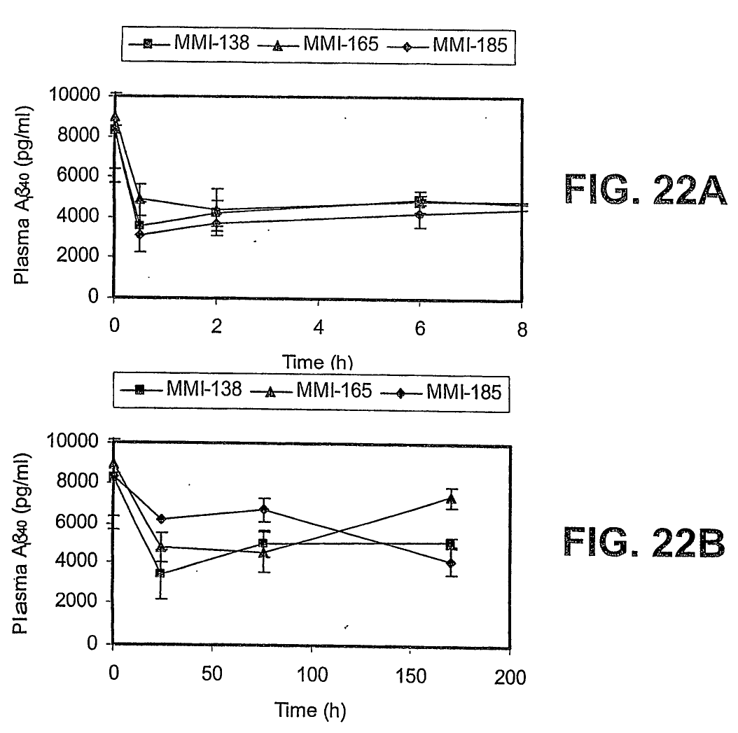

Six APP/F animals were injected intraperitoneally with one of three different memapsin 2 inhibitors, MMI-138, MMI-165, or MMI-185. Following injection, blood samples were removed at various times by bleeding the saphenous vein, and analyzed for amount of Aβ₄₀. FIGS. 22A and 22B show data indicating a precipitous decline in Aβ₄₀ within 30 minutes following injection, for all compounds tested. The decrease in Aβ₄₀ was lowest for MMI-185, dropping by 63%, whereas MMI-138 and MMI-165 both revealed reduction of Aβ₄₀ by 57% and 46%, respectively.

Transgenic mice were injected with a single injection of 163 nM of MMI-138, MMI-165 or MMI-185 and blood collected prior to the administration of the inhibitor compound (0 hours) and 2, 4, 6 and 8 hours following the administration of the inhibitor compound. Plasma β-amyloid protein (Aβ₄₀) was determined. Data expresses the mean±the standard error of the mean. Two animals were used in each treatment group. As shown in FIG. 22A, β-amyloid levels in the plasma decreased in less than about an hour following the administration of the compound. As shown in FIG. 22B, plasma β-amyloid protein levels were decreased beyond 150 hours following administration of the inhibitor compound. These data show a long term effect on inhibiting memapsin 2 activity which subsequently inhibits the production of β-amyloid protein following administration of the inhibitor compounds.

Control animals treated with DMSO or inhibitor OM00-3 revealed a decrease of only 21% and 16%, respectively, at 2 hours following injection (FIG. 21C). The inhibition remained nearly constant over a 24-h period for animals treated with either MMI-138 or MMI-165, whereas plasma Aβ₄₀ levels appeared to return to initial level in animals treated with MMI-185 (FIG. 22B). Generally, the Aβ₄₀ levels returned somewhat to their initial levels for all treated animals observed over a 170 hour period following treatment, although it was persistently lower, indicating a long-term level of inhibition of Aβ₄₀ production from the brains of these transgenic animals.

The extent of inhibition observed at 30 minutes (FIG. 22A) mirrored the Kᵢ values of the compounds (Ki=4.5, 8.8, 15.3 nM for MMI-185, MMI-138, and MMI-165, respectively). This observation shows the relevance of in vitro determinations of inhibition potency (Table 3) for ascertaining the degree of successful inhibition of Aβ₄₀ production in mammals. The compounds were related to their sustainment of inhibition. MMI-138 and MMI-165 are closely related and both bear the selective dimethylpyrazole group (Table 3), MMI-185, which is structurally less similar, did not sustain inhibition over the same extent of time. Nonetheless, all compounds tested capably inhibited Aβ₄₀ production. That reduction of Aβ₄₀ was observed is indicative that the compounds successfully crossed the blood-brain barrier to inhibit memapsin 2 activity in vivo, even though the size of these compounds is greater than the 500 g/mole size limit for exclusion from the blood-brain barrier, especially as inhibitor OM00-3 (MW 935 g/mole) failed to inhibit Aβ₄₀ production (FIG. 21C). The inhibition of Aβ₄₀ production in vivo could not have been predicted from the compounds themselves, nor from their in vitro measures of potency. Moreover, this is the first demonstration of in vivo memapsin 2 inhibition in mammals, resulting in reduction of Aβ₄₀ production from the brain, by administration of compounds of this kind.

### Example 5

### Novel Upstream Substrate Specificity Determination with Memapsin 2

Memapsin 2 has been identified and experimentally supported as the β-secretase enzyme involved in the pathogenesis of Alzheimer's disease, and has further been characterized as a novel membrane bound aspartic protease. As such, memapsin 2 has many of the observed characteristics of the aspartic protease family. These characteristics include: an acidic pH optimum, the conserved D T/S G catalytic aspartic acid motif, an observed large substrate binding cleft, and an extended peptide substrate specificity. These last two characteristics of the aspartic protease family have been analyzed in a number of experimental studies and across a variety of species. The consensus of these studies is that the extended substrate binding cleft facilitates the interaction of eight amino acid residues of the substrate peptide, four on either side of the scissile bond. Here, we report the observation of a catalytic effect resulting from four distal amino acid residues of its substrate, namely in positions P₅, P₆, P₇, and P₈, which are N-terminal (upstream) to the traditional catalytic binding sequence. We have further conducted a specificity analysis of these positions to determine the optimal amino acid composition for catalysis.

Experimental Procedure

Design of Defined Substrate Templates and Upstream Analysis Peptides

The peptide sequence EVNLAAEF (described in Example 1), successfully utilized in the memapsin 2 residue preference analysis for memapsin 2 was used as the base template peptide to analyze the extended upstream interaction. For the initial series of analyses, three peptides were created using solid phase peptide synthesis (Research Genetics, Invitrogen, Huntsville, Ala.). These peptides, EVNLAAEFWHDR (SEQ ID NO: 16) (designated WHDR), RWHHEVNLAAEF (SEQ ID NO: 17) (designated RWHH), and EEISEVNLAAEF (SEQ ID NO: 46) (designated EEIS) (asterisk denotes the cleavage site in each peptide) were created to examine the downstream, upstream, and native APP sequence extensions, respectively. Additionally, four peptide mixtures were synthesized based on the extended native APP sequence (P5: RTEEIxEVNLAAEF (SEQ ID NO: 47); P6: RTEExSEVNLAAEF (SEQ ID NO: 48); P7: RTExISEVNLAAEF (SEQ ID NO: 49); P8: RTxEISEVNLAAEF (SEQ ID NO: 50); where x denotes a mixture of nine amino acid residues at that position) to examine the residue preference of the four upstream amino acids. To facilitate MALDI-TOF detection, an arginine was added to the N-terminus of the peptides. These peptides were created through solid phase peptide synthesis with equimolar amounts of a mixture of nine amino acids added at the appropriate cycle of the synthesis. The resulting mixture of nine peptides differed by only one amino acid at a single subsite. The amino acid corresponding to the native APP sequence substrate was included in each mixture to serve as an internal standard.

MALDI-TOF/MS Kinetic Analysis

Substrate mixtures were prepared following the method of Example 1 to obtain an incubation mixture with memapsin 2 (SEQ ID NO: 9 (FIG. 12)) and peptide in the micromolar range at pH 4.0. The reactions were allowed to proceed for 60 minutes with aliquots removed periodically. Aliquots were mixed with an equal volume of MALDI matrix (α-hydroxycinnamic acid in acetone), and immediately spotted on a 96 dual-well Teflon coated analysis plate. The MALDI data collection and analysis was performed on a PE Biosystems Voyager DE instrument. Data were analyzed using the Voyager Data Explorer module to obtain ion intensity data. Relative product created per unit time was obtained from nonlinear regression analysis of the data representing the initial 15% of product formation and this data was used to determine the relative k_{cat}/Kₘ values.

Results and Discussion

Observation of Kinetic Effect

The crystal structure of memapsin 2 bound to inhibitor OM00-3 shows eight amino acid side chains accommodated within the substrate binding cleft of the enzyme (Lin, 2000). MALDI-TOF analysis was utilized in this initial study to determine this primary specificity. For this analysis, two template peptide sequences were designed to facilitate the examination of both the upstream and downstream interacting residues. These templates, RWHHEVNLAAEF (SEQ ID NO: 17) (designated RWHH) and EVNLAAEFWHDR (SEQ ID NO: 16) (designated WHDR), utilized an asymmetric design to allow the separation of the common product of catalysis from the unique catalytic products, dramatically enhancing the sensitivity of the assay system. While this design allowed an extremely sensitive analysis of the specificity for the observed binding sites, a very interesting and dramatic difference was observed in the rate of catalysis between all substrate mixtures of the P side relative to the P′ side, which might have resulted from simply extending the substrate with either RWHH (SEQ ID NO: 53) upstream of the template sequence, or WHDR (SEQ ID NO: 29) downstream. This change in the rate of catalysis due to changes in the peptide sequence outside of the traditional interacting residues is a novel observation for aspartic proteases in general (Davies, 1990). Whereas this initial observation was made with independent assays, it was sought to confirm and directly measure this effect by competitive cleavage assays of a mixture of the two peptides. These data supported the initial observation revealed a 60-fold decrease in the rate of catalysis for the upstream RWHH (SEQ ID NO: 53) sequence addition when compared to the downstream WHDR (SEQ ID NO: 29) sequence addition.

Analysis of the Observed Effect on Catalytic Efficiency

An analysis of the crystal structure of memapsin 2 (Lin, 2000) and specifically of the positioning of bound inhibitor, suggests that the downstream WHDR (SEQ ID NO: 29) sequence would be sufficiently distant from the enzyme to have no effect on catalysis. The upstream RWHH (SEQ ID NO: 53) sequence addition, however, does not extend beyond the outer peptide loop insertions near the enzyme cleft and could potentially interact with two of the sequence insertions of memapsin 2. A comparison of the crystal structures of pepsin and memapsin 2 indicates these observed structural differences identified on the upstream side of the binding cleft and could therefore be supportive of a distal upstream substrate interaction. Moreover, the presence of structural features coupled with the observation of a catalytic rate difference permits a hypothesis of a distal substrate binding cleft, previously unobserved for aspartic proteases. Presence of a binding cleft implies the possibility of substrate selectivity. Based on these observations, we examined whether the observed kinetic interaction resulted from the RWHH (SEQ ID NO: 53) N-terminal sequence addition specifically, indicating a selective extended binding cleft, or whether this interaction would result from any extended upstream sequence. To this end, a third peptide was synthesized using the same eight residue template sequence, EVNLAAEF (SEQ ID NO: 51), and extending it upstream with amino acids EEIS (SEQ ID NO: 52), the native sequence from human APP. Competitive cleavage analysis of a mixture of these three peptides resulted in statistically identical rates of catalysis for the upstream EEIS (SEQ ID NO: 52) and the downstream WHDR (SEQ ID NO: 29) sequence additions, while the RWHH (SEQ ID NO: 53) sequence addition still demonstrated a 60-fold decrease in catalytic rate. This result confirmed that the change in catalytic efficiency resulted from an interaction with the upstream residues of the peptide, with particular amino acid sequence RWHH (SEQ ID NO: 53) having a negative effect. Furthermore, that the N-terminal amino acid composition altered the rate of catalysis directly, an analysis of the possibility of a residue preference in these four distal positions became the next experimental objective.

Determination of Substrate Side Chain Specificity for the Upstream Binding Interaction

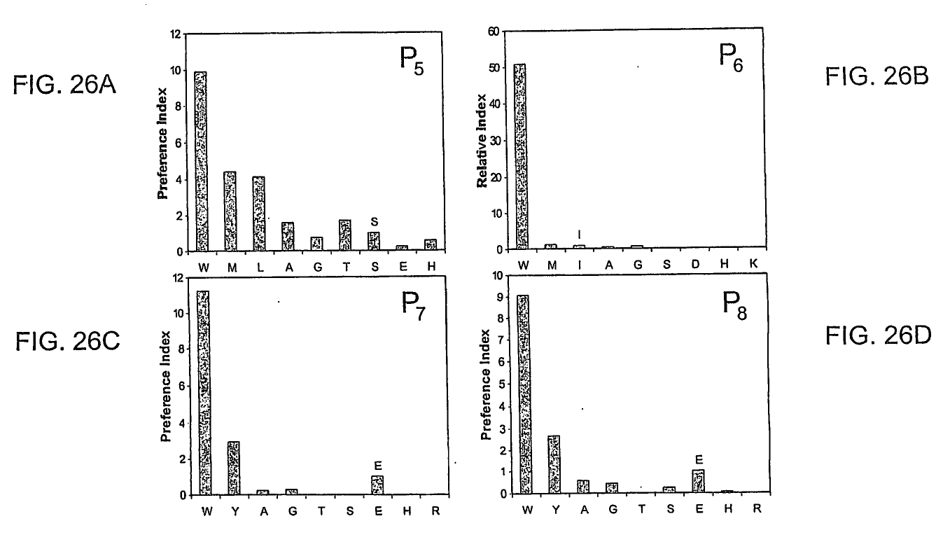

The observation that a negative effect on the catalytic efficiency was due to the specific upstream sequence extension of RWHH (SEQ ID NO: 53) suggests that a binding interaction is occurring. To further characterize this interaction, an analysis of the amino acid specificity for this change in enzyme efficiency was performed. This analysis was conducted using the MALDI-TOF/MS quantitation method as previously discussed in Example 1, utilizing a synthesized substrate mixture library to explore the distal upstream positions P₅, P₆, P₇, and P₈. The resulting substrate side-chain preferences, reported as the preference index, for these four positions are presented in FIGS. 26A, 26B, 26C and 26D. Interestingly, Trp is the most preferred residue in all four sites, with Tyr and Met also demonstrating improved catalytic efficiencies. The position with the greatest observed effect is clearly P⁶ with Trp having a 50-fold increase over the native APP Ile and with the RWHH (SEQ ID NO: 53) His residue having no detectable product in the incubation. The strong preference for hydrophobic residues suggests that there is a hydrophobic interaction resulting in the improved catalytic efficiency.

Summary of Sequences

Table 13 is a summary of the nucleic acid and amino acid sequences described herein.

TABLE 13

| | | | |
|---|---|---|---|
| SEQ | | | |
| ID | FIGURE NO. | TYPE OF | |
| NO: | OR SEQUENCE | SEQUENCE | COMMENT |

| | | | |
|---|---|---|---|
| 1 | 4 | nucleic acid | GenBank sequence of |
| | | | memapsin 1 |
| 2 | 5 | amino acid | Deduced sequence of |
| | | | memapsin 1 |
| 3 | 6 | nucleic acid | Promemapsin 1-T1 |
| 4 | 7 | amino acid | Deduced sequence of |
| | | | promemapsin 1-T1 |
| 5 | 8 | nucleic acid | GenBank memapsin 2 |
| 6 | 9 | amino acid | Deduced sequence of |
| | | | memapsin 2 |
| 7 | 10 | nucleic acid | Promemapsin 2 |
| 8 | 11 | amino acid | Deduced sequence of |
| | | | promemapsin 2 |
| 9 | 12 | amino acid | Portion of promemapsin |
| | | | 2 used in crystal |
| | | | structures |
| 10 | 23 | amino acid | GenBank amyloid |
| | | | precursor protein (APP) |
| 11 | SEVNLDAEFR | amino acid | Swedish mutation of |
| | | | APP β-secretase |
| | | | cleavage site |
| 12 | SEVKMDAEFR | amino acid | Native APP β-secretase |
| | | | cleavage site |
| 13 | YGRKKRRQRRR | amino acid | tat-peptide |
| 14 | RRRRRRRRR | amino acid | nine arginine carrier |
| | | | molecule |
| 15 | EVNLAAEF | amino acid | |
| 16 | EVNLAAEFWHDR | amino acid | |
| 17 | RWHHEVNLAAEF | amino acid | |
| 18 | EVNLXAEFWHDR | amino acid | |
| 19 | XAEFWHDR | amino acid | |
| 20 | EVNL*AAEF | amino acid | |
| 21 | Gly-Xx1-Xx2- | amino acid | Unspecified amino acids |
| | Leu*Ala-Xx3-Xx4- | | Xx1, Xx2, Xx3 and Xx4 |
| | Phe-Arg-Met-Gly- | | are equivalent to |
| | Gly-resin | | unspecified amino acids |
| | | | P3, P2, P2′ and P3′, |
| | | | respectively, in SEQ ID |
| | | | NO 27 |
| 22 | Xx3-Xx4-Phe-Arg- | amino acid | |
| | Met-Gly-Gly-resin | | |
| 23 | ELDL*AVEF | amino acid | |
| 24 | EVNLΨAAEF | amino acid | |
| 25 | Gly-Xx1-Xx2- | amino acid | Same as SEQ ID NO 21 |
| | LeuΨAla-Xx3-Xx4- | | except “Ψ” is used to |
| | Phe-Arg-Met-Gly- | | denote hydroxyethylene |
| | Gly-resin | | linkage instead of “*” |
| 27 | Gly-P₃-P₂-Leu*Ala- | amino acid | (see note for SEQ ID NO |
| | P₂′-P₃′-Phe-Arg-Met- | | 21) |
| | Gly-Gly-resin | | |
| 28 | WHDREVNLAAEF | amino acid | |
| 29 | WHDR | amino acid | |
| 30 | EVNLAVEFWHDR | amino acid | |
| 31 | EVNLAAEFWHDR | amino acid | |
| 32 | YGRKKRRQRRR | amino acid | tat-peptide |
| 33 | RRRRRRRRR | amino acid | (D-Arg)₉ carrier molecule |
| 34 | FAM-Ahx- | amino acid | CPI-1 carrier peptide |
| | (EVNL*AAEF)-G- | | inhibitor conjugate |
| | (YGRKKRRQRRR) | | |
| 35 | FAM-Ahx- | amino acid | CPI-2 carrier peptide |
| | (ELDL*AVEF)-GG- | | inhibitor conjugate |
| | (RRRRRRRRR) | | |
| 36 | FAM-Ahx-GGG- | amino acid | CP-1 fluorescein-labeled |
| | (YGRKKRRQRRR) | | carrier peptide |
| 37 | (ELDL*AVEF)-GG- | amino acid | CPI-3 carrier peptide |
| | (RRRRRRRRR) | | inhibitor conjugate |
| 38 | CGGAACCGCTCAT | nucleic acid | primer |
| | TGCC | | |
| 39 | ACCCACACTGTGC | nucleic acid | primer |
| | CCATCTA | | |
| 40 | CTGACCACTCGAC | nucleic acid | primer |
| | CAGGTTCTGGGT | | |
| 41 | GTGGATAACCCCT | nucleic acid | primer |
| | CCCCCAGCCTAGA | | |
| | CCA | | |
| 42 | KLVFFAED | amino acid | |
| 44 | WHDREVNLAVEF | amino acid | |
| 45 | EVNLAVEF | amino acid | |
| 46 | EEISEVNLAAEF | amino acid | |
| 47 | RTEEIxEVNLAAEF | amino acid | |
| 48 | RTEExSEVNLAAEF | amino acid | |
| 49 | RTExISEVNLAAEF | amino acid | |
| 50 | RTxEISEVNLAAEF | amino acid | |
| 51 | EVNLAAEF | amino acid | |
| 52 | EEIS | amino acid | |
| 53 | RWHH | amino acid | |

What is claimed is:

1. A compound represented by the following structural formula: 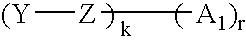
wherein:
Y is a carrier molecule;
Z is a covalent bond, —OP(O)⁻₂O—, —C(O)OR₃₃—, —C(O)NHR₃₃— or an amino acid sequence cleavable by a hydrolase;
R₃₃ is a bond or an alkylene;
k is 0 or an integer from 1 to about 100;
r is an integer from 1 to about 100; and
A₁ for each occurrence is a compound represented by the following structural formula: 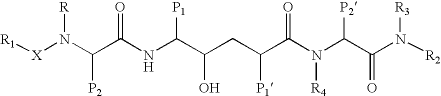
or optical isomers, diastereomers, or pharmaceutically acceptable salts thereof, wherein:
X is C═O or S(O)ₙ;
n is 1 or 2;
P₁ is an aliphatic group, a hydroxyalkyl, an aryl, an aralkyl, a heterocycloalkyl, or an alkylsulfanylalkyl;
P₂, P₁′, and P₂′ are each, independently, a substituted or unsubstituted aliphatic group, a substituted or unsubstituted heteroalkyl, a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl;
R is —H;
R₁ is a substituted or unsubstituted aliphatic group, a substituted or unsubstituted alkoxy, a substituted or unsubstituted aryl, a substituted or unsubstituted aralkyl, a substituted or unsubstituted heterocycle, a substituted or unsubstituted heterocycloalkyl, a substituted or unsubstituted heterocyclooxy, a substituted or unsubstituted heterocycloalkoxy, a substituted or unsubstituted heteroaryl, a substituted or unsubstituted heteroaralkyl, a substituted or unsubstituted heteroaralkoxy, or —NR₅R₆; or R₁, together with X, is a peptide or Y-Z-;
R₄ is H; or R₄ and P₁′, together with the atoms connecting R₄ and P₁′, form a five or six membered heterocycle;
R₂ and R₃ are each, independently, selected from the group consisting of H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl, and a substituted or unsubstituted heteroaralkyl; or one of R₂ and R₃, together with the nitrogen to which it is attached, is a peptide or a Y-Z-; or R₂ and R₃, together with the nitrogen to which they are attached, form a substituted or unsubstituted heterocycle or a substituted or unsubstituted heteroaryl; and
R₅ and R₆ are each, independently, H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl or a substituted or unsubstituted heteroaralkyl; or
R and one of R₅ or R₆, together with X and the nitrogen atoms to which they are attached, form a 5-, 6-, or 7-membered substituted or unsubstituted heterocycle or substituted or unsubstituted heteroaryl ring,
provided that the compound is not one of the following compounds:

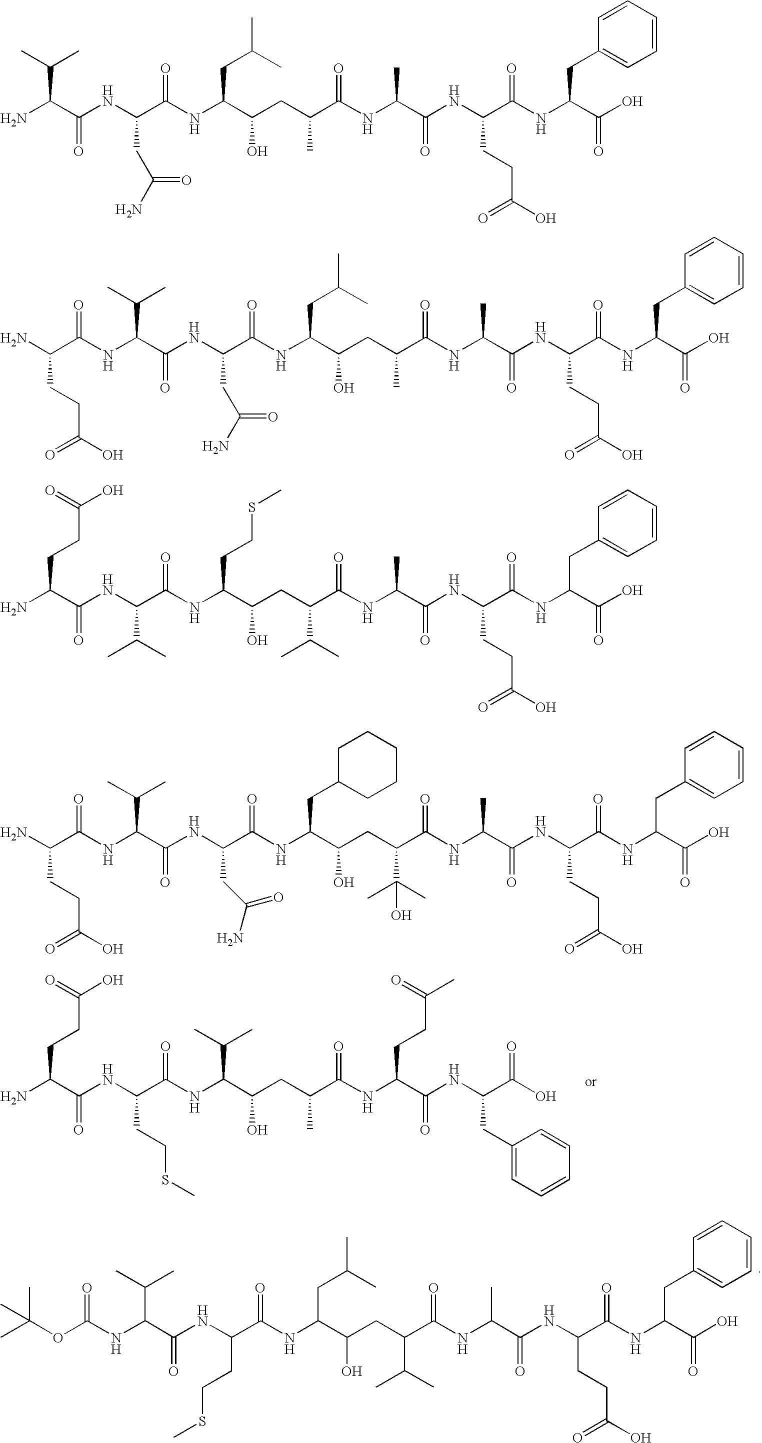

2. The compound of claim 1, wherein R₁ is —OR₁₅ or —NR₁₅R₁₆, wherein:
R₁₅ and R₁₆ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more substituents selected from the group consisting of an aliphatic group, hydroxy, —OR₉, a halogen, a cyano, a nitro, —NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀, an aryl, a heteroaryl, a heteroaralkyl, and a heterocycle, and wherein:
R₉ and R₁₀ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl heteroaryl or heteroaralkyl are optionally substituted with one or more aliphatic groups; and
p is 0, 1, or 2.

3. The compound of claim 1, wherein R₁ is a substituted aliphatic group.

4. The compound of claim 3, wherein R₁ is an aliphatic group that is substituted with one or more substituents selected from the group consisting of —NR₁₅C(O)₂R₁₆, —NR₁₅C(O)R₁₆, and —NR₁₅S(O)₂R₁₆, wherein:
R₁₅ and R₁₆ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more substituents selected from the group consisting of an aliphatic group, hydroxy, —OR₉, a halogen, a cyano, a nitro, —NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀, an aryl, a heteroaryl, a heteroaralkyl, and a heterocycle; and
p is 0, 1, or 2.

5. The compound of claim 4, wherein the compound is represented by the following structural formula: 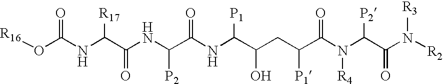
wherein R₁₇ is a substituted or unsubstituted aliphatic group.

6. The compound of claim 1, wherein R₁ together with X is a peptide represented by the following structural formula: 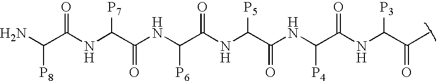
wherein:
P₃ and P₄ are each, independently, an amino acid side chain;
P₅ is an amino acid side chain selected from the group consisting of tryptophan side chain, methionine side chain, and leucine side chain;
P₆ is tryptophan side chain;
P₇ is an amino acid side chain selected from the group consisting of tryptophan side chain, tyrosine side chain; and glutamate side chain; and
P₈ is an amino acid side chain selected from the group consisting of tryptophan side chain, tyrosine side chain; and glutamate side chain.

7. The compound of claim 6, wherein P₅, P₆, P₇, and P₈ are each a tryptophan side chain.

8. The compound of claim 4, wherein P₁ is an aliphatic group.

9. The compound of claim 4, wherein P₁ is selected from the group consisting of isobutyl, hydroxymethyl, cyclopropylmethyl, cyclobutylmethyl, phenylmethyl, cyclopentylmethyl, and heterocycloalkyl.

10. The compound of claim 4, wherein P₂′ is a hydrophobic group.

11. The compound of claim 10, wherein P₂′ is isopropyl or isobutyl.

12. The compound of claim 4, wherein P₂ is a hydrophobic group.

13. The compound of claim 4, wherein P₂ is —R₁₁SR₁₂, —R₁₁S(O)R₁₂, —R₁₁S(O)₂R₁₂, —R₁₁C(O)NR₁₂R₁₃, —R₁₁OR₁₂, —R₁₁OR₁₄OR₁₃, or a hetercycloalkyl, wherein:
the heterocycloalkyl is optionally substituted with one or more alkyl groups;
R₁₁ and R₁₄ are each, independently, an alkylene; and
R₁₂ and R₁₃ are each, independently, H, an aliphatic group, an aryl, an arakyl, a heterocycle, a heterocyclalkyl, a heteroaryl, or a heteroaralkyl.

14. The compound of claim 13, wherein P₂ is —CH₂CH₂SCH₃, —CH₂CH₂S(O)CH₃, —CH₂CH₂S(O)₂CH₃, —CH₂C(O)NH₂, —CH₂C(O)NHCH₂CH═CH₂, tetrahydrofuran-2-yl, tetrahydrofuran-2-yl-methyl, tetrahydrofuran-3-yl, tetrahydrofuran-3-yl-methyl, pyrrolidin-2-yl-methyl, pyrrolidin-3-yl-methyl, or —CH₂CH₂OCH₂OCH₃.

15. The compound of claim 4, wherein R₂ is H and R₃ together with the nitrogen to which it is attached is a peptide.

16. The compound of claim 4, wherein R₂ is H and R₃ is selected from the group consisting of 2-furanylmethyl, phenylmethyl, indan-2-yl, n-butyl, isopropyl, isobutyl, 1-fluoromethyl-2-fluoroethyl, indol-3-yl, and 3-pyridylmethyl.

17. The compound of claim 4, wherein R₂ and R₃ together with the nitrogen to which they are attached form morpholino, piperazinyl or piperidinyl, wherein the morpholino, piperazinyl and piperidinyl are optionally substituted with one or more aliphatic groups.

18. The compound of claim 4, wherein k is 0 and r is 1.

19. The compound of claim 4, wherein k is 1 and r is 1.

20. The compound of claim 19, wherein Y is a peptide.

21. The compound of claim 19, wherein Y is selected from the group consisting of tat-peptide and polyarginine.

22. The compound of claim 20, wherein Z is selected from the group consisting of —OP(O)⁻₂O—, Phe-Phe, Phe-Leu, and Phe-Try.

23. A compound represented by the following structural formula: 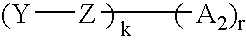
wherein:
Y is a carrier molecule;
Z is a bond, —OP(O)⁻₂O—, —C(O)OR₃₃—, —C(O)NHR₃₃— or an amino acid sequence cleavable by a hydrolase;
R₃₃ is a bond or an alkylene;
k is 0 or an integer from 1 to about 100;
r is an integer from 1 to about 100; and
A₂ for each occurrence is a compound represented by the following structural formula: 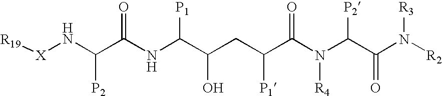
or optical isomers, diastereomers, or pharmaceutically acceptable salts thereof, wherein:
X is C═O or S(O)ₙ;
n is 1 or 2;
P₁ is an aliphatic group, a hydroxyalkyl, an aryl, an aralkyl, a heterocycloalkyl, or an alkylsulfanylalkyl;
P₂, P₁′, and P₂′ are each, independently, a substituted or unsubstituted aliphatic group, a substituted or unsubstituted heteroalkyl, a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl;
R₄ is H; or R₄ and P₁′, together with the atoms connecting R₄ and P₁′, form a five or six membered heterocycle;
R₂ and R₃ are each, independently, selected from the group consisting of H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl, and a substituted or unsubstituted heteroaralkyl; or one of R₂ and R_{3,} together with the nitrogen to which they are attached, is a peptide or Y-Z-; or R₂ and R₃ together with the nitrogen to which they are attached form a substituted or unsubstituted heterocycle or a substituted or unsubstituted heteroaryl; and
R₁₉ is an aliphatic group substituted with one or more substituents, wherein at least one substituent is a substituent selected from the group consisting of —NR₁₅C(O)R₁₆, —NR₁₅C(O)₂R₁₆ and —NR₁₅S(O)₂R₁₆, wherein:
R₁₅ and R₁₆ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more substituents selected from the group consisting of an aliphatic group, hydroxy, —OR₉, a halogen, a cyano, a nitro, —NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀, an aryl, a heteroaryl, a heteroaralkyl, and a heterocycle; and
p is 0, 1, or 2,
provided that when R₁₉ is substituted with —NR₁₅C(O)R₁₆ or —NR₁₅C(O)₂R₁₆, —NR₂R₃ is not a group having the following structural formula:

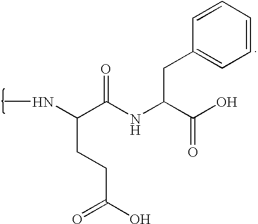

24. The compound of claim 23, wherein the compound is represented by the following structural formula: 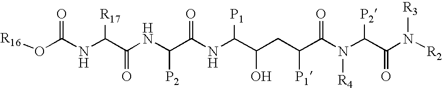
wherein R₁₇ is a substituted or unsubstituted aliphatic group.

25. The compound of claim 23, wherein P₁ is an aliphatic group.

26. The compound of claim 23, wherein P₁ is selected from the group consisting of isobutyl, hydroxymethyl, cyclopropylmethyl, cyclobutylmethyl, phenylmethyl, cyclopentylmethyl, and heterocycloalkyl.

27. The compound of claim 23, wherein P₂′ is a hydrophobic group.

28. The compound of claim 27, wherein P₂′ is isopropyl or isobutyl.

29. The compound of claim 23, wherein P₂ is a hydrophobic group.

30. The compound of claim 23, wherein P₂ is —R₁₁SR₁₂, —R₁₁S(O)R₁₂, —R₁₁S(O)₂R₁₂, —R₁₁C(O)NR₁₂R₁₃, —R₁₁OR₁₂, —R₁₁OR₁₄OR₁₃, or a hetercycloalkyl, wherein:
the heterocycloalkyl is optionally substituted with one or more alkyl groups;
R₁₁ and R₁₄ are each, independently, an alkylene; and
R₁₂ and R₁₃ are each, independently, H, an aliphatic group, an aryl, an arakyl, a heterocycle, a heterocycloalkyl, a heteroaryl, or a heteroaralkyl.

31. The compound of claim 30, wherein P₂ is —CH₂CH₂SCH₃, —CH₂CH₂S(O)CH₃, —CH₂CH₂S(O)₂CH₃, —CH₂C(O)NH₂, —CH₂C(O)NHCH₂CH═CH₂, tetrahydrofuran-2-yl, tetrahydrofuran-2-yl-methyl, tetrahydrofuran-3-yl, tetrahydrofuran-3-yl-methyl, pyrrolidin-2-yl-methyl, pyrrolidin-3-yl-methyl, or —CH₂CH₂OCH₂OCH₃.

32. The compound of claim 23, wherein R₂ is H and R₃ together with the nitrogen to which it is attached is a peptide.

33. The compound of claim 23, wherein R₂ is H and R₃ is selected from the group consisting of 2-furanylmethyl, phenylmethyl, indan-2-yl, n-butyl, isopropyl, isobutyl, 1-fluoromethyl-2-fluoroethyl, indol-3-yl, and 3-pyridylmethyl.

34. The compound of claim 23, wherein R₂ and R₃ together with the nitrogen to which they are attached form morpholino, piperazinyl or piperidinyl, wherein the morpholino, piperazinyl and piperidinyl are optionally substituted with one or more aliphatic groups.

35. The compound of claim 23, wherein k is 0 and r is 1.

36. The compound of claim 23, wherein k is 1 and r is 1.

37. The compound of claim 36, wherein Y is a peptide.

38. The compound of claim 36, wherein Y is selected from the group consisting of tat-peptide and polyarginine.

39. The compound of claim 37, wherein Z is selected from the group consisting of —OP(O)⁻₂O—, Phe-Phe, Phe-Leu, and Phe-Try.

40. A compound represented by the following structural formula: 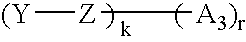
wherein:
Y is a carrier molecule;
Z is a bond, —OP(O)⁻₂O—, —C(O)OR₃₃—, —C(O)NHR₃₃— or an amino acid sequence cleavable by a hydrolase;
R₃₃ is a bond or an alkylene;
k is 0 or an integer from 1 to about 100;
r is an integer from 1 to about 100; and
A₃ for each occurrence is a compound represented by the following structural formula: 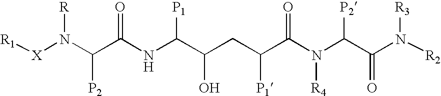
or optical isomers, diastereomers, or pharmaceutically acceptable salts thereof, wherein:
X is C═O or S(O)ₙ;
n is 1 or 2;
P₁ is an aliphatic group, a hydroxyalkyl, an aryl, an aralkyl, a heterocycloalkyl, or an alkylsulfanylalkyl;
P₂, P₁′, and P₂′ are each, independently, a substituted or unsubstituted aliphatic group, a substituted or unsubstituted heteroalkyl, a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl;
R is —H;
R₁ is a substituted or unsubstituted aliphatic group, a substituted or unsubstituted alkoxy, a substituted or unsubstituted aryl, a substituted or unsubstituted aralkyl, a substituted or unsubstituted heterocycle, a substituted or unsubstituted heterocycloalkyl, a substituted or unsubstituted heterocyclooxy, a substituted or unsubstituted heterocycloalkoxy, a substituted or unsubstituted heteroaryl, a substituted or unsubstituted heteroaralkyl, a substituted or unsubstituted heteroaralkoxy, or —NR₅R₆; R₁, together with X, is a peptide or Y-Z-;
R₄ is H; or R₄ and P₁′, together with the atoms connecting R₄ and P₁′, form a five or six membered heterocycle;
R₂ and R₃ are each, independently, selected from the group consisting of H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl, and a substituted or unsubstituted heteroaralkyl; or one of R₂ and R₃, together with the nitrogen to which they are attached, is a peptide or Y-Z-; or R₂ and R₃, together with the nitrogen to which they are attached, form a substituted or unsubstituted heterocycle or a substituted or unsubstituted heteroaryl; and
R₅ and R₆ are each, independently, H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl or a substituted or unsubstituted heteroaralkyl; or
R and one of R₅ or R₆, together with X and the nitrogen atoms to which they are attached, form a 5-, 6-, or 7-membered substituted or unsubstituted heterocycle or substituted or unsubstituted heteroaryl ring, wherein the compound selectively inhibits hydrolysis of a memapsin 2 β-secretase site relative to a memapsin 1 β-secretase site.

41. The compound of claim 40, wherein R₁ together with X is a peptide represented by the following structural formula: 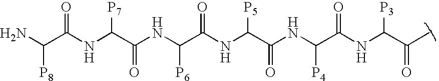
wherein:
P₃ and P₄ are each, independently, an amino acid side chain;
P₅ is an amino acid side chain selected from the group consisting of tryptophan side chain, methionine side chain, and leucine side chain;
P₆ is tryptophan side chain;
P₇ is an amino acid side chain selected from the group consisting of tryptophan side chain, tyrosine side chain; and glutamate side chain; and
P₈ is an amino acid side chain selected from the group consisting of tryptophan side chain, tyrosine side chain; and glutamate side chain.

42. The compound of claim 41, wherein P₅, P₆, P₇, and P₈ are each a tryptophan side chain.

43. The compound of claim 40, wherein R₁ is a substituted or unsubstituted heteroaralkoxy or a substituted or unsubstituted heteroaralkyl.

44. The compound of claim 43, wherein the heteroaryl group of the heteroaralkoxy or heteroaralkyl is selected from the group consisting of substituted or unsubstituted pyrazolyl, substituted or unsubstituted furanyl, substituted or unsubstituted imidazolyl, substituted or unsubstituted isoxazolyl, substituted or unsubstituted oxadiazolyl, substituted or unsubstituted oxazolyl, substituted or unsubstituted pyrrolyl, substituted or unsubstituted pyridyl, substituted or unsubstituted pyrimidyl, substituted or unsubstituted pyridazinyl, substituted or unsubstituted thiazolyl, substituted or unsubstituted triazolyl, substituted or unsubstituted thienyl, substituted or unsubstituted 4,6-dihydro-thieno[3,4-c]pyrazolyl, substituted or unsubstituted 5,5-dioxide-4,6-dihydrothieno[3,4-c]pyrazolyl, substituted or unsubstituted thianaphthenyl, substituted or unsubstituted carbazolyl, substituted or unsubstituted benzimidazolyl, substituted or unsubstituted benzothienyl, substituted or unsubstituted benzofuranyl, substituted or unsubstituted indolyl, substituted or unsubstituted quinolinyl, substituted or unsubstituted benzotriazolyl, substituted or unsubstituted benzothiazolyl, substituted or unsubstituted benzooxazolyl, substituted or unsubstituted benzimidazolyl, substituted or unsubstituted isoquinolinyl, substituted or unsubstituted isoindolyl, substituted or unsubstituted acridinyl, and substituted or unsubstituted benzoisazolyl.

45. The compound of claim 44, wherein the heteroaryl group is a heteroazaaryl.

46. The compound of claim 45, wherein the heteroazaaryl is selected from the group consisting of substituted or unsubstituted pyrazolyl, substituted or unsubstituted imidazolyl, substituted or unsubstituted isoxazolyl, substituted or unsubstituted oxadiazolyl, substituted or unsubstituted oxazolyl, substituted or unsubstituted pyrrolyl, substituted or unsubstituted pyridyl, substituted or unsubstituted pyrimidyl, substituted or unsubstituted pyridazinyl, substituted or unsubstituted thiazolyl, substituted or unsubstituted triazolyl, substituted or unsubstituted benzimidazolyl, substituted or unsubstituted quinolinyl, substituted or unsubstituted benzotriazolyl, substituted or unsubstituted benzooxazolyl, substituted or unsubstituted benzimidazolyl, substituted or unsubstituted isoquinolinyl, substituted or unsubstituted indolyl, substituted or unsubstituted isoindolyl, and substituted or unsubstituted benzoisazolyl.

47. The compounds of claim 46, wherein the compound has the following structural formula: 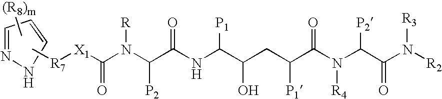
wherein:
X₁ is —O—, —NR₂₂— or a covalent bond;
R₇ is a substituted or unsubstituted alkylene;
m is 0, 1, 2, or 3;
R₈ is a substituted or unsubstituted aliphatic group, —OR₉, —R₂₃—O—R₉, a halogen, a cyano, a nitro, NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀ a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl;
p is 0, 1 or 2; and
R₉ and R₁₀ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more aliphatic groups;
R₂₃ is a substituted or unsubstituted alkylene; and
R₂₂ is —H; or
R and R₂₂, together with X and the nitrogen atoms to which they are attached, form a 5-, 6-, or 7-membered substituted or unsubstituted heterocycle or substituted or unsubstituted heteroaryl ring.

48. The compound of claim 47, wherein P₁ is an aliphatic group.

49. The compound of claim 47, wherein P₁ is selected from the group consisting of isobutyl, hydroxymethyl, cyclopropylmethyl, cyclobutylmethyl, phenylmethyl, cyclopentylmethyl, and heterocycloalkyl.

50. The compound of claim 47, wherein P₂′ is a hydrophobic group.

51. The compound of claim 47, wherein P₂′ is isopropyl or isobutyl.

52. The compound of claim 47, wherein P₂ is a hydrophobic group.

53. The compound of claim 47, wherein P₂ is —R₁₁SR₁₂, —R₁₁S(O)R₁₂, —R₁₁S(O)₂R₁₂, —R₁₁C(O)NR₁₂R₁₃, —R₁₁OR₁₂, —R₁₁OR₁₄OR₁₃, or a hetercycloalkyl, wherein:
the heterocycloalkyl is optionally substituted with one or more alkyl groups;
R₁₁ and R₁₄ are each, independently, an alkylene; and
R₁₂ and R₁₃ are each, independently, H, an aliphatic group, an aryl, an arakyl, a heterocycle, a heterocyclalkyl, a heteroaryl, or a heteroaralkyl.

54. The compound of claim 53, wherein P₂ is —CH₂CH₂SCH₃, —CH₂CH₂S(O)CH₃, —CH₂CH₂S(O)₂CH₃, —CH₂C(O)NH₂, —CH₂C(O)NHCH₂CH═CH₂, tetrahydrofuran-2-yl, tetrahydrofuran-2-yl-methyl, tetrahydrofuran-3-yl, tetrahydrofuran-3-yl-methyl, pyrrolidin-2-yl-methyl, pyrrolidin-3-yl-methyl, or —CH₂CH₂OCH₂OCH₃.

55. The compound of claim 47, wherein R₂ is H and R₃ together with the nitrogen to which it is attached is a peptide.

56. The compound of claim 47, wherein R₂ is H and R₃ is selected from the group consisting of 2-furanylmethyl, phenylmethyl indan-2-yl, n-butyl isopropyl, isobutyl, 1-fluoromethyl-2-fluoroethyl, indol-3-yl, and 3-pyridylmethyl.

57. The compound of claim 47, wherein R₂ and R₃ together with the nitrogen to which they are attached form morpholino, piperazinyl or piperidinyl, wherein the morpholino, piperazinyl and piperidinyl are optionally substituted with one or more aliphatic groups.

58. The compound of claim 46, wherein k is 0 and r is 1.

59. The compound of claim 46, wherein k is 1 and r is 1.

60. The compound of claim 59, wherein Y is a peptide.

61. The compound of claim 59, wherein Y is selected from the group consisting of tat-peptide and polyarginine.

62. The compound of claim 60, wherein Z is selected from the group consisting of —OP(O)⁻₂O—, Phe-Phe, Phe-Leu, and Phe-Try.

63. A compound represented by the following structural formula: 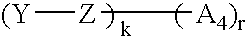
wherein:
Y is a carrier molecule;
Z is a bond, —OP(O)⁻₂O—, —C(O)OR₃₃—, —C(O)NHR₃₃— or an amino acid sequence cleavable by a hydrolase;
R₃₃ is a bond or an alkylene;
k is 0 or an integer from 1 to about 100;
r is an integer from 1 to about 100; and
A₄ for each occurrence is a compound represented by the following structural formula: 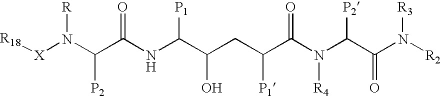
or optical isomers, diastereomers, or pharmaceutically acceptable salts thereof, wherein:
X is C═O or S(O)ₙ;
n is 1 or 2;
P₁ is an aliphatic group, a hydroxyalkyl, an aryl, an aralkyl, a heterocycloalkyl, or an alkylsulfanylalkyl;
P₂, P₁′, and P₂′ are each, independently, a substituted or unsubstituted aliphatic group, a substituted or unsubstituted heteroalkyl, a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl;
R is —H;
R₄ is H; or R₄ and P₁′, together with the atoms connecting R₄ and P₁′, form a five or six membered heterocycle;
R₂ and R₃ are each, independently, selected from the group consisting of H, a substituted or unsubstituted aliphatic group, a substituted or unsubstituted aryl, a substituted or unsubstituted aralkyl, a substituted or unsubstituted heterocycle, a substituted or unsubstituted heterocycloalkyl, a substituted or unsubstituted heteroaryl, and a substituted or unsubstituted heteroaralkyl; or one of R₂ or R₃, together with the nitrogen to which they are attached, is a peptide or Y-Z-; or R₂ and R₃ together with the nitrogen to which they are attached form a substituted or unsubstituted heterocycle or a substituted or unsubstituted heteroaryl;
R₁₈ is a substituted or unsubstituted heteroaralkoxy, a substituted or unsubstituted heteroaralkyl, or —NR₂₀R₂₁; and
R₂₀ and R₂, are each, independently, —H or a substituted or unsubstituted heteroaralkyl; or
R and one of R₂₀ or R₂₁, together with X and the nitrogen atoms to which they are attached, form a 5-, 6-, or 7-membered substituted or unsubstituted heterocycle or substituted or unsubstituted heteroaryl ring.

64. The compound of claim 63, wherein the heteroaryl group of the heteroaralkoxy or heteroarakyl is selected from the group consisting of substituted or unsubstituted pyrazolyl, substituted or unsubstituted furanyl, substituted or unsubstituted imidazolyl, substituted or unsubstituted isoxazolyl, substituted or unsubstituted oxadiazolyl, substituted or unsubstituted oxazolyl, substituted or unsubstituted pyrrolyl, substituted or unsubstituted pyridyl, substituted or unsubstituted pyrimidyl, substituted or unsubstituted pyridazinyl, substituted or unsubstituted thiazolyl, substituted or unsubstituted triazolyl, substituted or unsubstituted thienyl, substituted or unsubstituted 4,6-dihydro-thieno[3,4-c]pyrazolyl, substituted or unsubstituted 5,5-dioxide-4,6-dihydrothieno[3,4-c]pyrazolyl, substituted or unsubstituted thianaphthenyl, substituted or unsubstituted carbazolyl, substituted or unsubstituted benzimidazolyl, substituted or unsubstituted benzothienyl, substituted or unsubstituted benzofuranyl, substituted or unsubstituted indolyl, substituted or unsubstituted quinolinyl, substituted or unsubstituted benzotriazolyl, substituted or unsubstituted benzothiazolyl, substituted or unsubstituted benzooxazolyl, substituted or unsubstituted benzimidazolyl, substituted or unsubstituted isoquinolinyl, substituted or unsubstituted isoindolyl, substituted or unsubstituted acridinyl, and substituted or unsubstituted benzoisazolyl.

65. The compound of claim 64, wherein the heteroaryl group is a heteroazaaryl.

66. The compound of claim 65, wherein the heteroazaaryl is selected from the group consisting of substituted or unsubstituted pyrazolyl, substituted or unsubstituted imidazolyl, substituted or unsubstituted isoxazolyl, substituted or unsubstituted oxadiazolyl, substituted or unsubstituted oxazolyl, substituted or unsubstituted pyrrolyl, substituted or unsubstituted pyridyl, substituted or unsubstituted pyrimidyl, substituted or unsubstituted pyridazinyl, substituted or unsubstituted thiazolyl, substituted or unsubstituted triazolyl, substituted or unsubstituted benzimidazolyl, substituted or unsubstituted quinolinyl, substituted or unsubstituted benzotriazolyl, substituted or unsubstituted benzooxazolyl, substituted or unsubstituted benzimidazolyl, substituted or unsubstituted isoquinolinyl, substituted or unsubstituted indolyl, substituted or unsubstituted isoindolyl, and substituted or unsubstituted benzoisazolyl.

67. The compounds of claim 66, wherein the compound has the following structural formula: 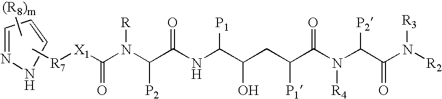
wherein:
X₁ is —O—, —NR₂₂—, or a covalent bond;
R₇ is a substituted or unsubstituted alkylene;
m is 0, 1, 2, or 3;
R₈ is a substituted or unsubstituted aliphatic group, —OR₉, —R₂₃—O—R₉, a halogen, a cyano, a nitro, NR₉R₁₀, guanidino, —OPO₃⁻², —PO₃⁻², —OSO₃⁻, —S(O)ₚR₉, —OC(O)R₉, —C(O)R₉, —C(O)₂R₉, —NR₉C(O)R₁₀, —C(O)NR₉R₁₀, —OC(O)NR₉R₁₀, —NR₉C(O)₂R₁₀ a substituted or unsubstituted aryl, a substituted or unsubstituted aralkyl, a substituted or unsubstituted heteroaryl, a substituted or unsubstituted heteroaralkyl, a substituted or unsubstituted heterocycle, or a substituted or unsubstituted heterocycloalkyl;
p is 0, 1 or 2; and
R₉ and R₁₀ are each, independently, H, an aliphatic group, an aryl, an aralkyl, a heterocycle, a heterocycloalkyl, a heteroaryl or a heteroaralkyl, wherein the aliphatic group, aryl, aralkyl, heterocycle, heterocyclalkyl, heteroaryl or heteroaralkyl are optionally substituted with one or more aliphatic groups;
R₂₃ is a substituted or unsubstituted alkylene; and
R₂₂ is —H; or
R and R₂₂, together with X and the nitrogen atoms to which they are attached, form a 5-, 6-, or 7-membered substituted or unsubstituted heterocycle or substituted or unsubstituted heteroaryl ring.

68. The compound of claim 67, wherein P₁ is an aliphatic group.

69. The compound of claim 67, wherein P₁ is selected from the group consisting of isobutyl, hydroxymethyl cyclopropylmethyl, cyclobutylmethyl, phenylmethyl, cyclopentylmethyl, and heterocycloalkyl.

70. The compound of claim 67, wherein P₂′ is a hydrophobic group.

71. The compound of claim 67, wherein P₂′ is isopropyl or isobutyl.

72. The compound of claim 67, wherein P₂ is a hydrophobic group.

73. The compound of claim 67, wherein P₂ is —R₁₁SR₁₂, —R₁₁S(O)R₁₂, —R₁₁S(O)₂R₁₂, —R₁₁C(O)NR₁₂R₁₃, —R₁₁OR₁₂, —R₁₁OR₁₄OR₁₃, or a hetercycloalkyl, wherein:
the heterocycloalkyl is optionally substituted with one or more alkyl groups;
R₁₁ and R₁₄ are each, independently, an alkylene; and
R₁₂ and R₁₃ are each, independently, H, an aliphatic group, an aryl, an arakyl, a heterocycle, a heterocyclalkyl, a heteroaryl, or a heteroaralkyl.

74. The compound of claim 73, wherein P₂ is —CH₂CH₂SCH₃, —CH₂CH₂S(O)CH₃, —CH₂CH₂S(O)₂CH₃, —CH₂C(O)NH₂, —CH₂C(O)NHCH₂CH═CH₂, tetrahydrofuran-2-yl, tetrahydrofuran-2-yl-methyl, tetrahydrofuran-3-yl, tetrahydrofuran-3-yl-methyl, pyrrolidin-2-yl-methyl, pyrrolidin-3-yl-methyl, or —CH₂CH₂OCH₂OCH₃.

75. The compound of claim 67, wherein R₂ is H and R₃ together with the nitrogen to which it is attached is a peptide.

76. The compound of claim 67, wherein R₂ is H and R₃ is selected from the group consisting of 2-furanylmethyl, phenylmethyl, indan-2-yl, n-butyl, isopropyl, isobutyl, 1-fluoromethyl-2-fluoroethyl, indol-3-yl, and 3-pyridylmethyl.

77. The compound of claim 67, wherein R₂ and R₃ together with the nitrogen to which they are attached form morpholino, piperazinyl or piperidinyl, wherein the morpholino, piperazinyl and piperidinyl are optionally substituted with one or more aliphatic groups.

78. The compound of claim 66, wherein k is 0 and r is 1.

79. The compound of claim 66, wherein k is 1 and r is 1.

80. The compound of claim 79, wherein Y is a peptide.

81. The compound of claim 79, wherein Y is selected from the group consisting of tat-peptide and polyarginine.

82. The compound of claim 80, wherein Z is selected from the group consisting of —OP(O)⁻₂O—, Phe-Phe, Phe-Leu, and Phe-Try.

83. A compound represented by the following structural formula: 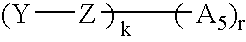
wherein:
Y is a carrier molecule;
Z is a bond, —OP(O)⁻₂O—, —C(O)OR₃₃—, —C(O)NHR₃₃— or an amino acid sequence cleavable by a hydrolase;
R₃₃ is a bond or an alkylene;
k is 0 or an integer from 1 to about 100;
r is an integer from 1 to about 100; and
A₅ for each occurrence is, independently, a compound selected from the group consisting of: 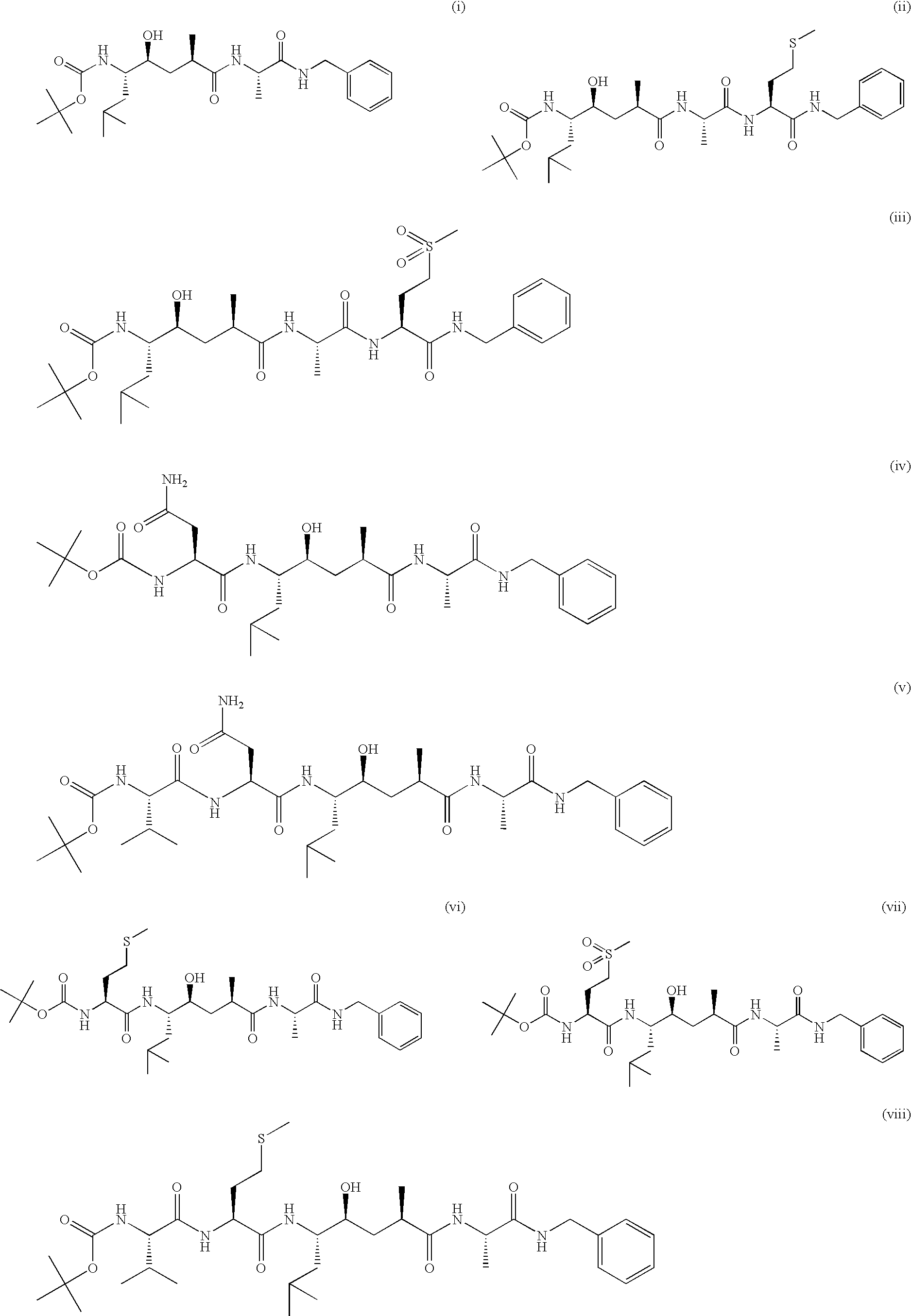 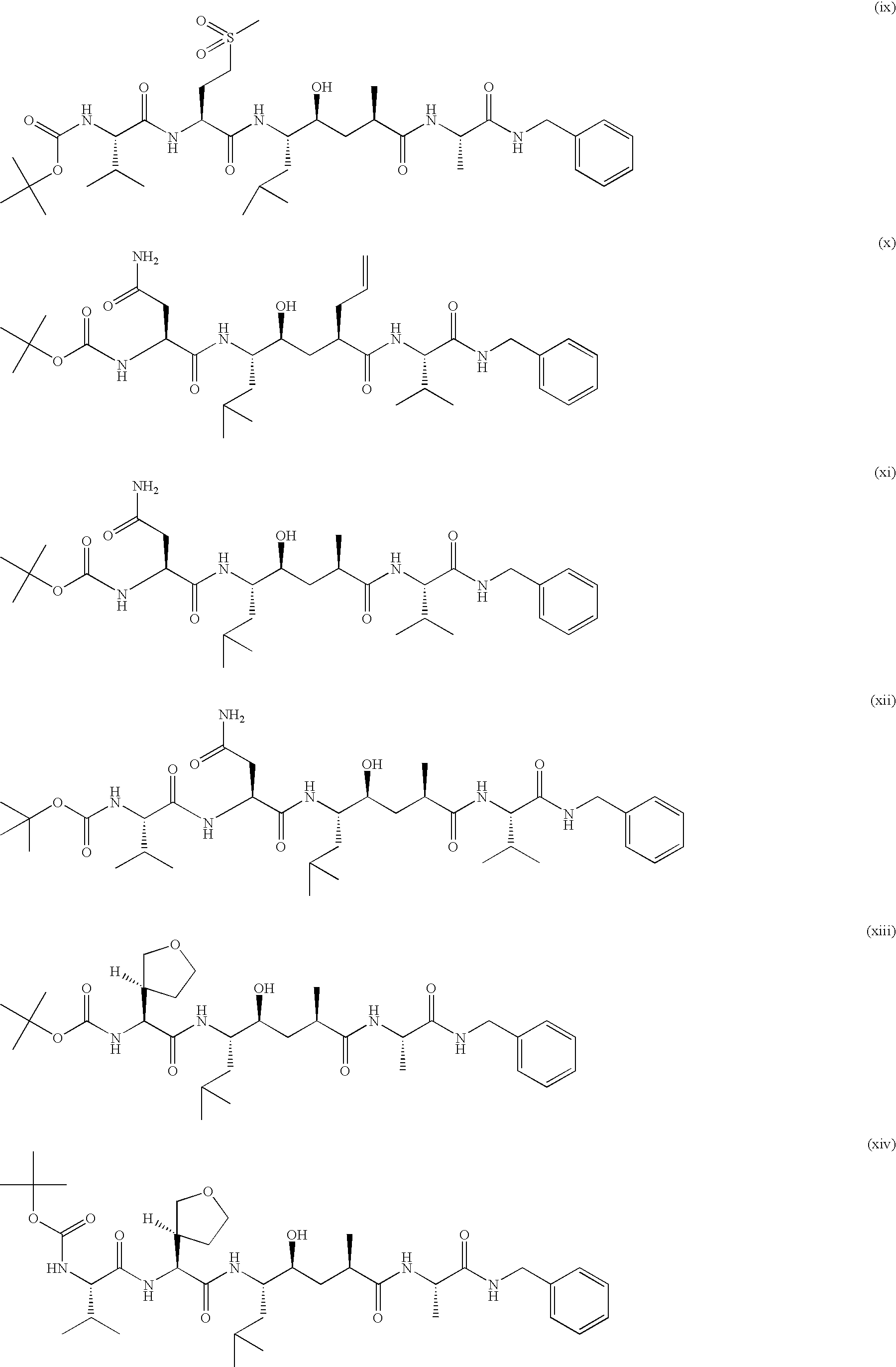 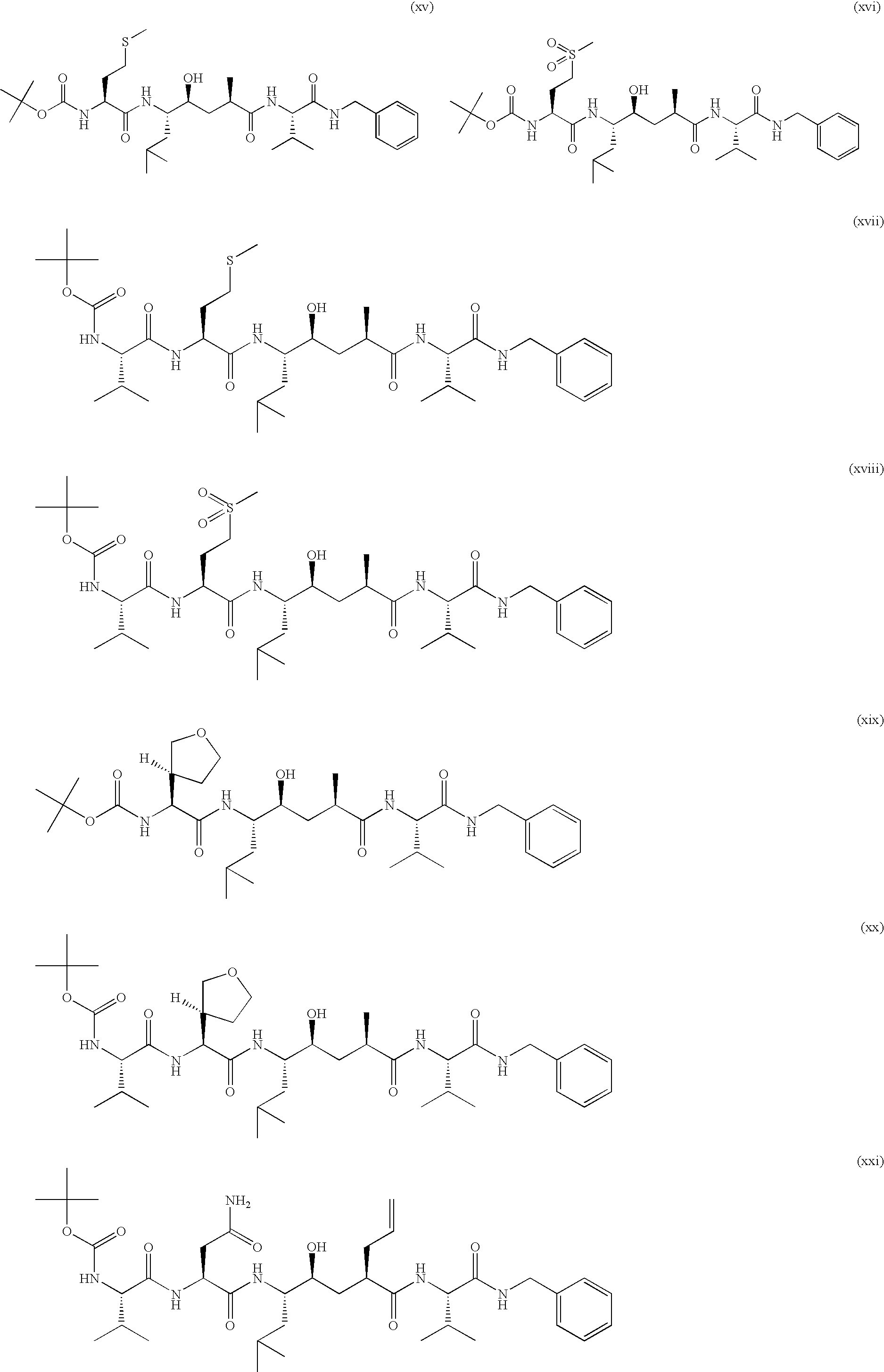 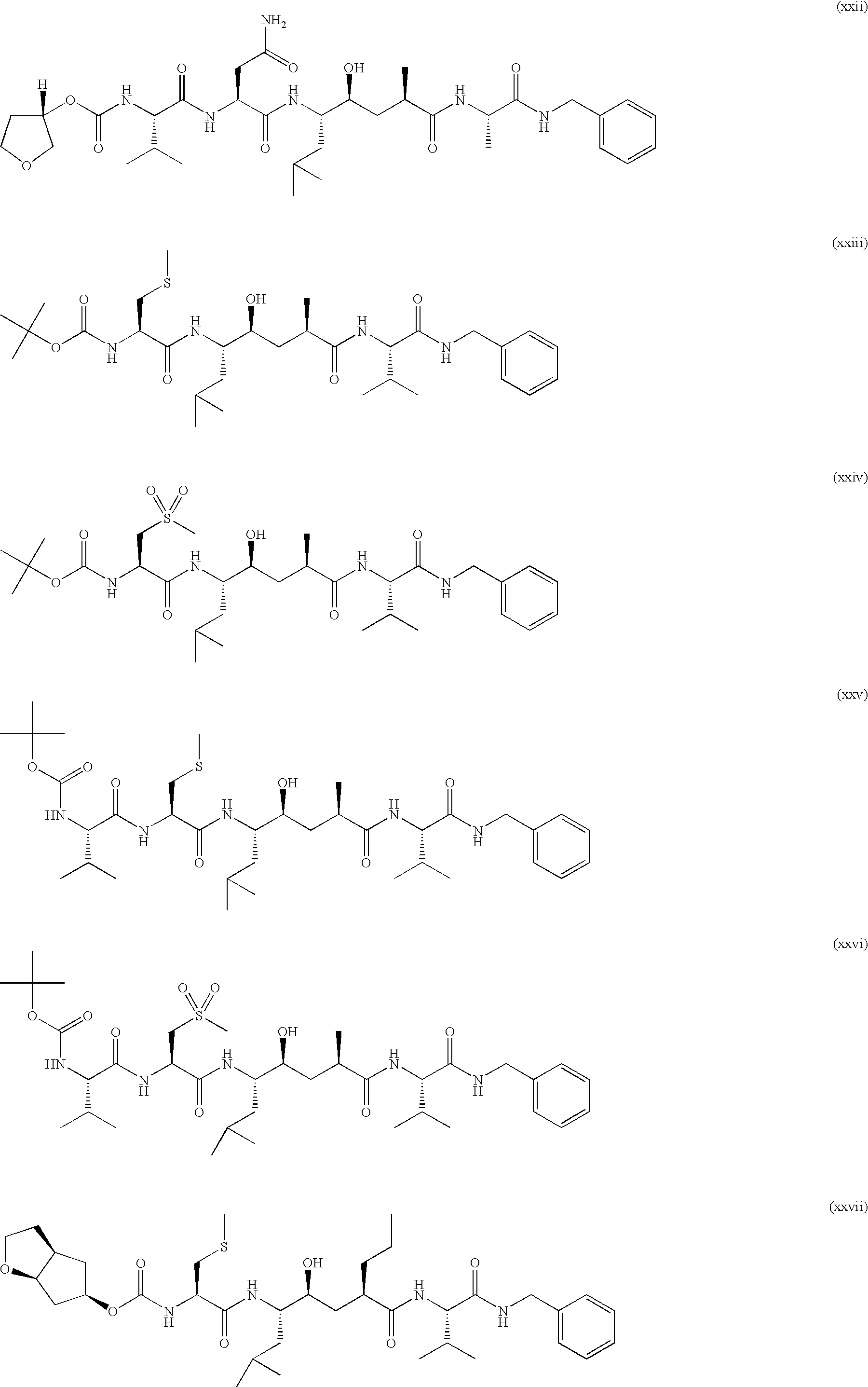 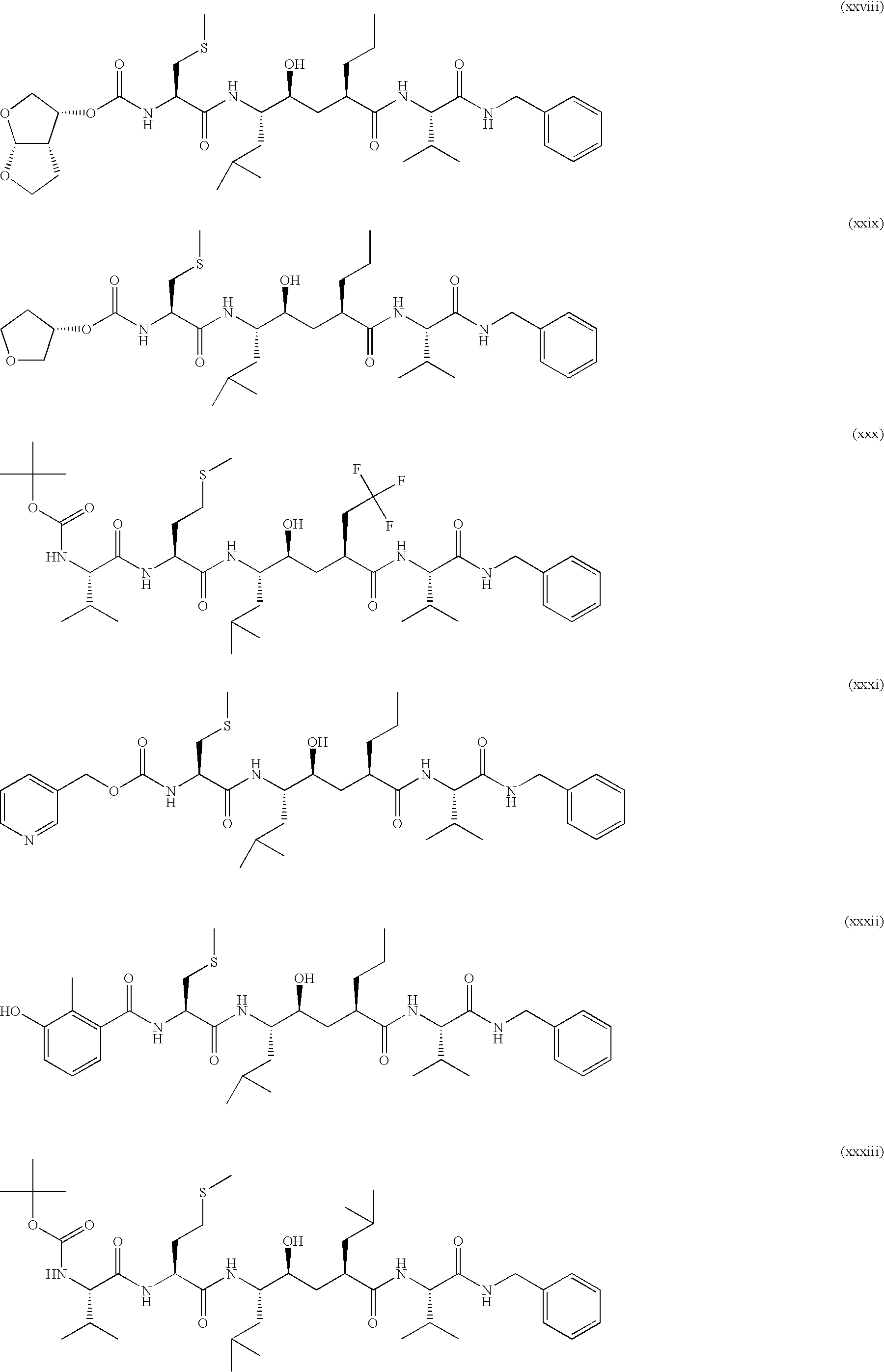 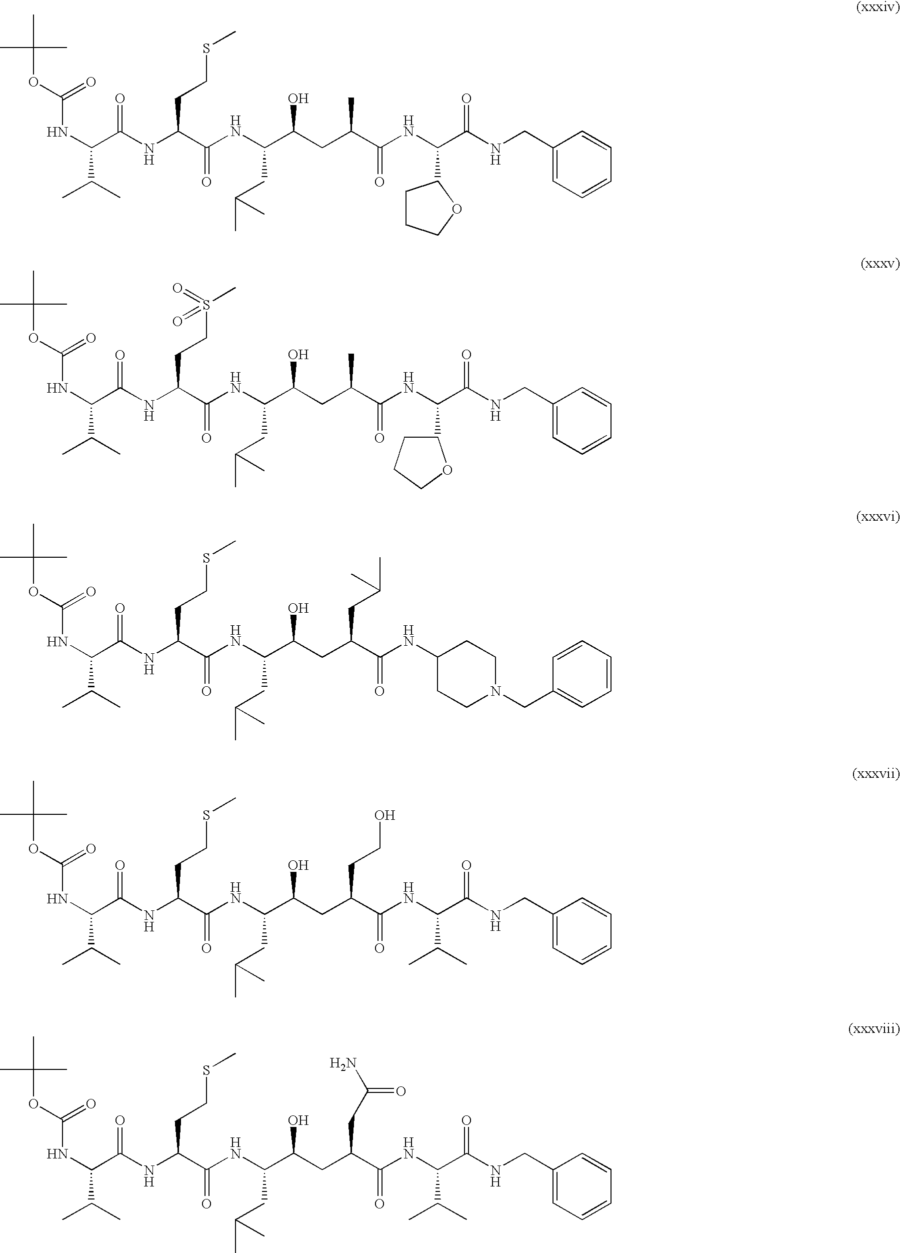 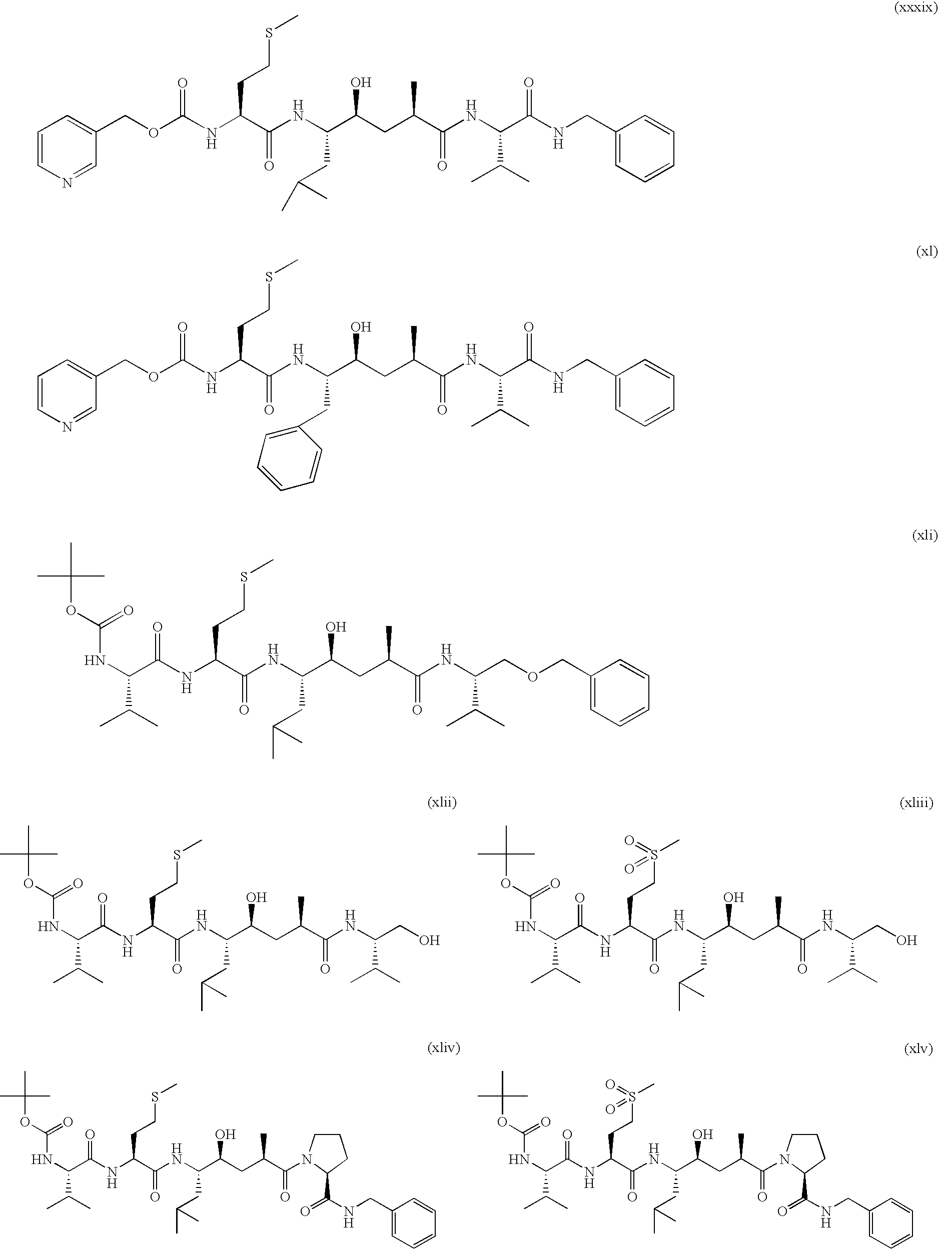 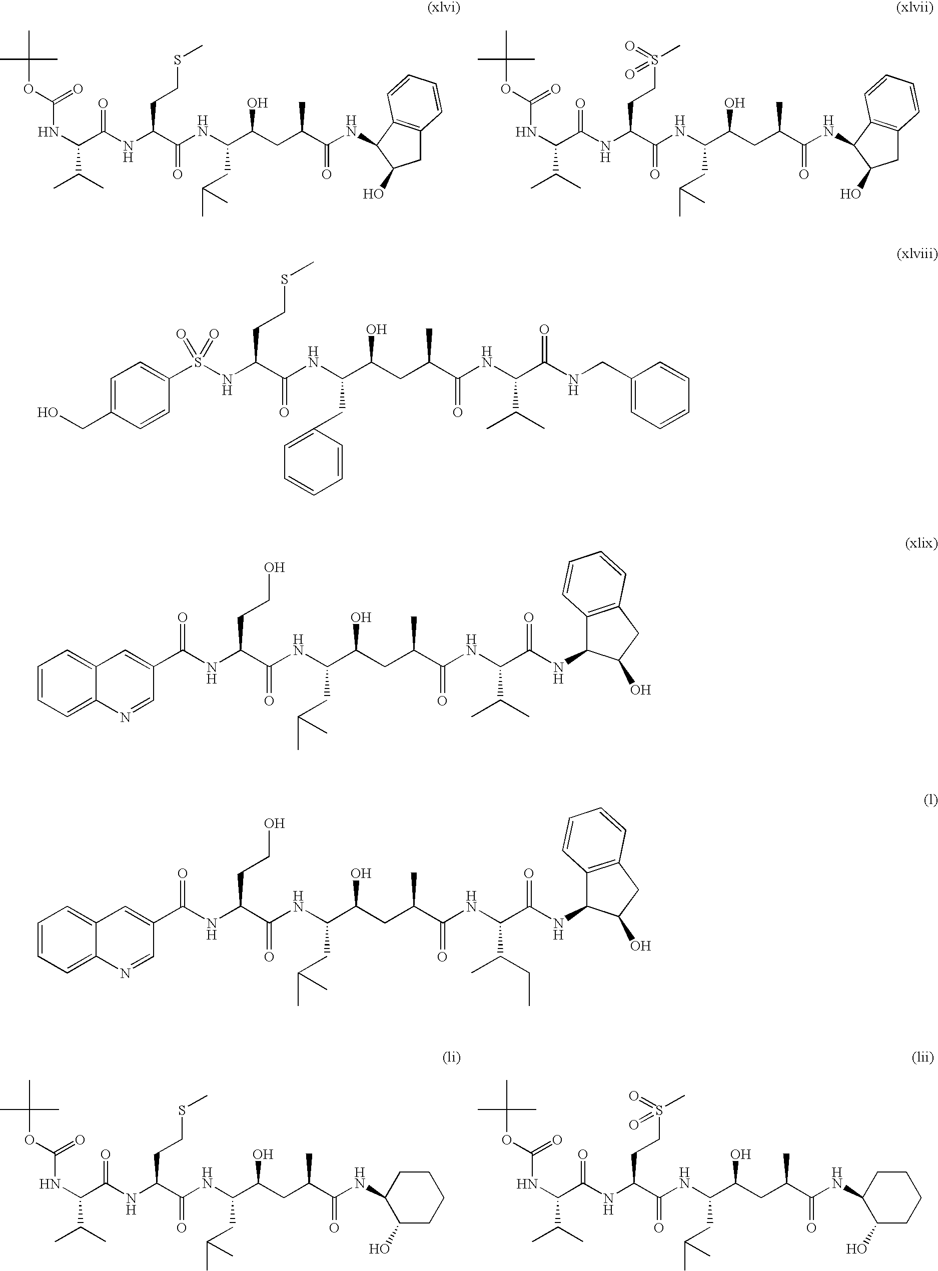 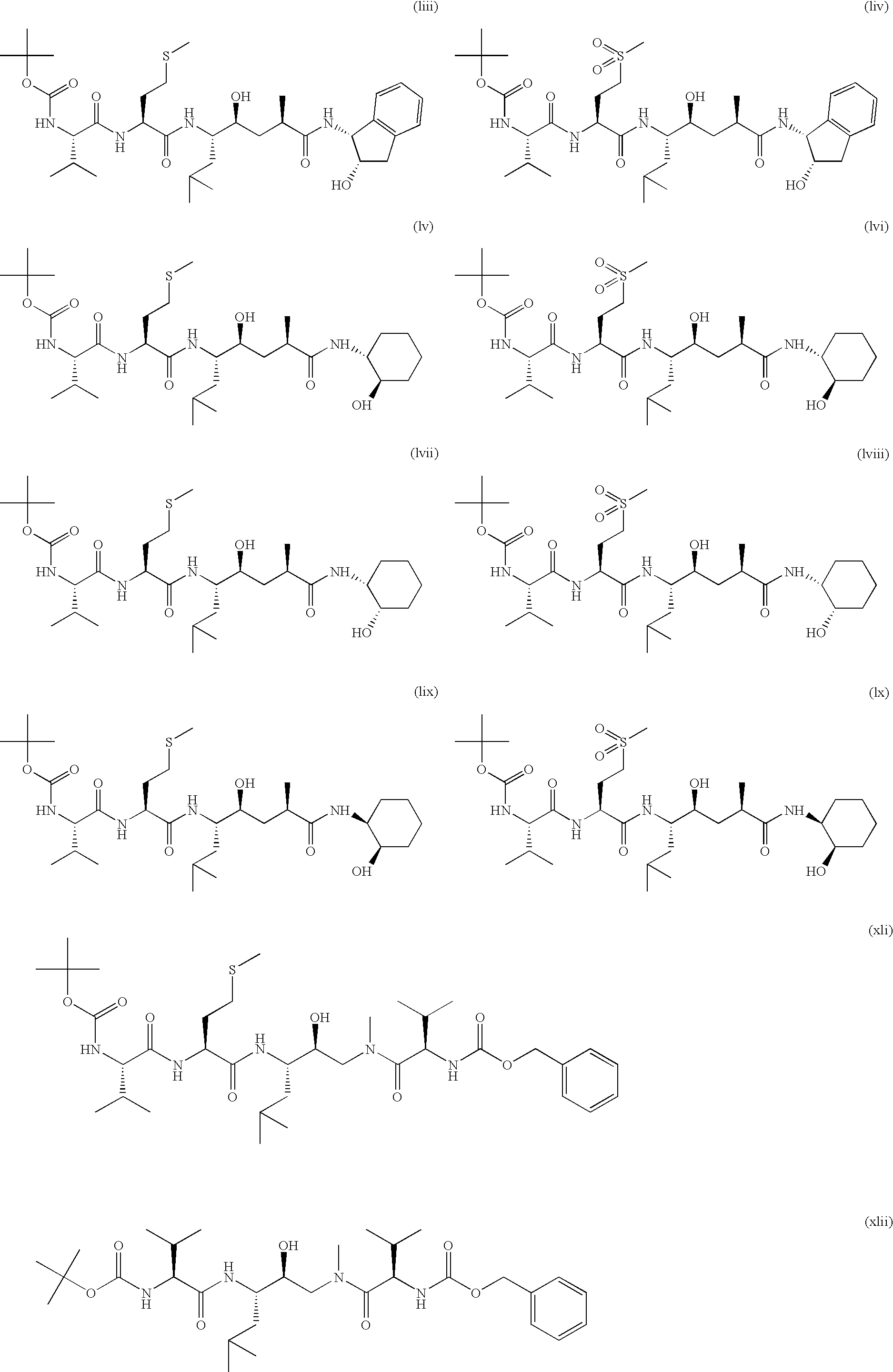 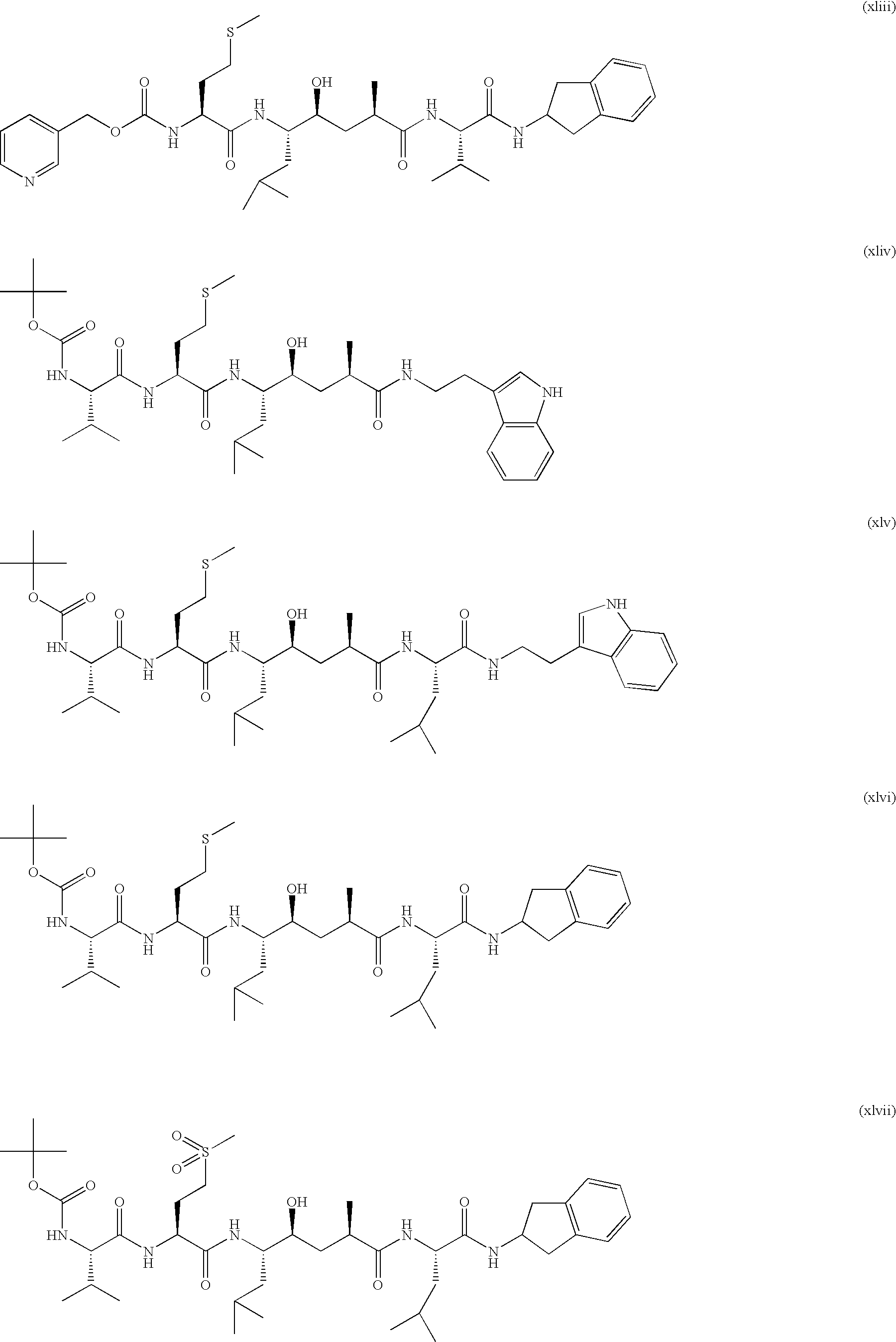 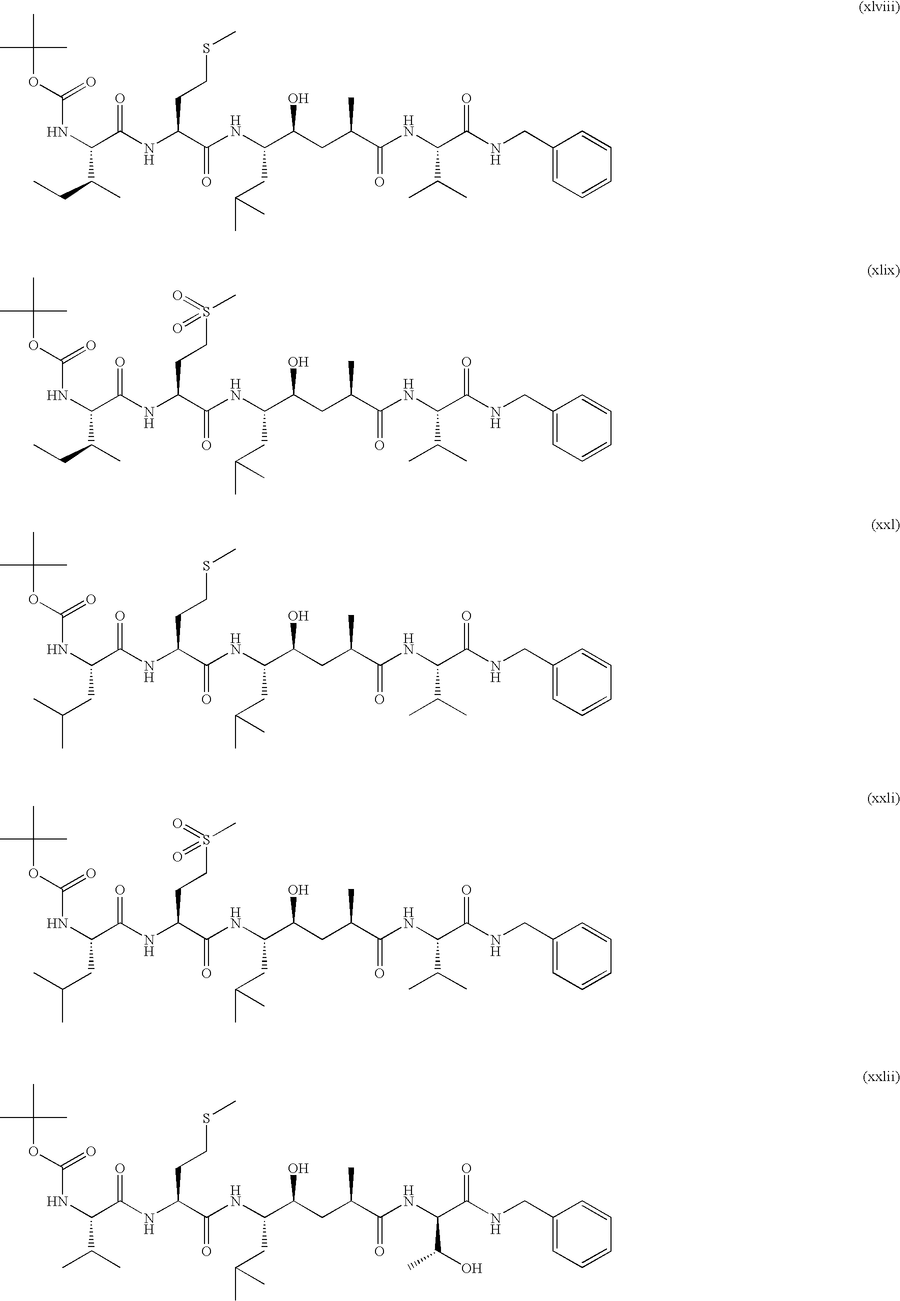 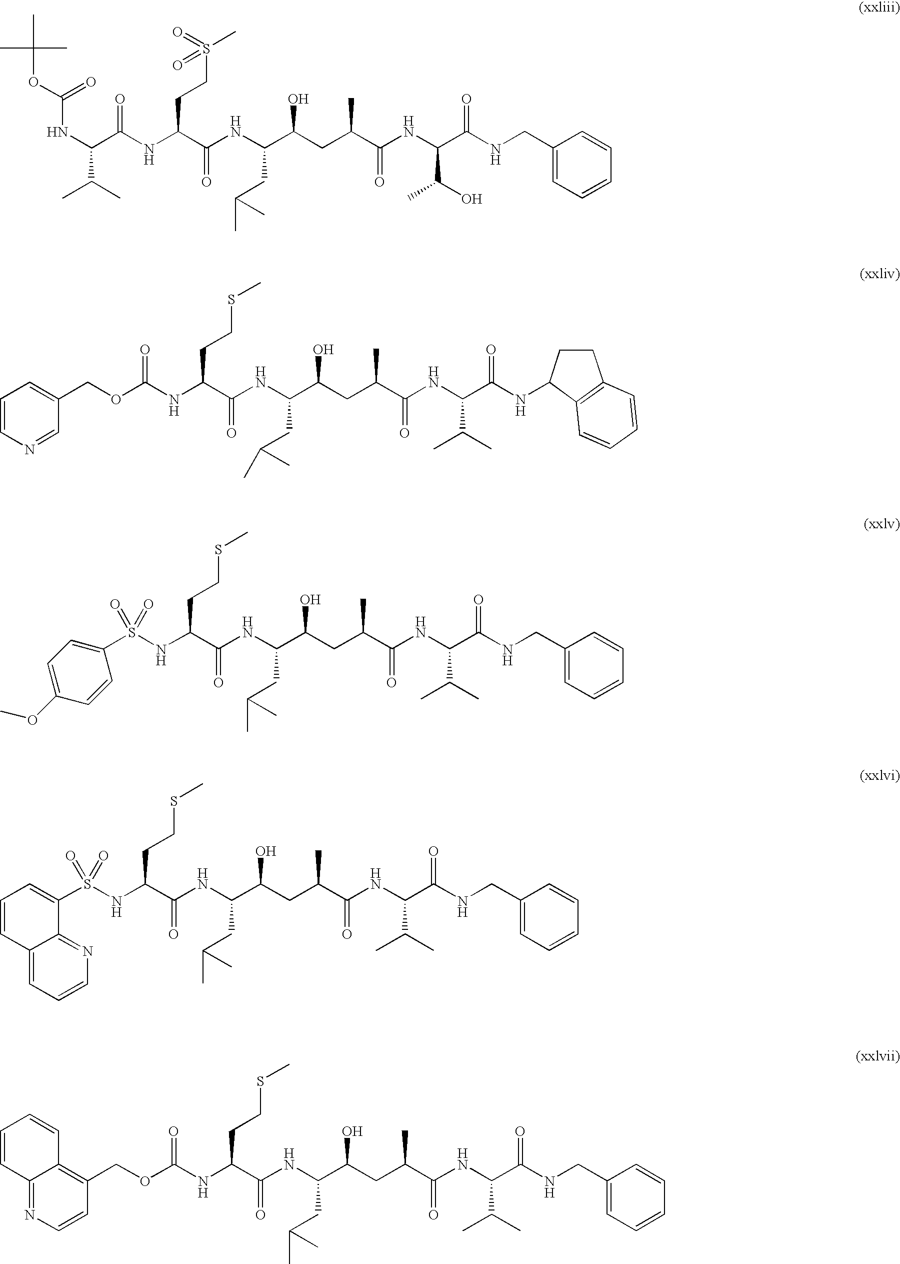 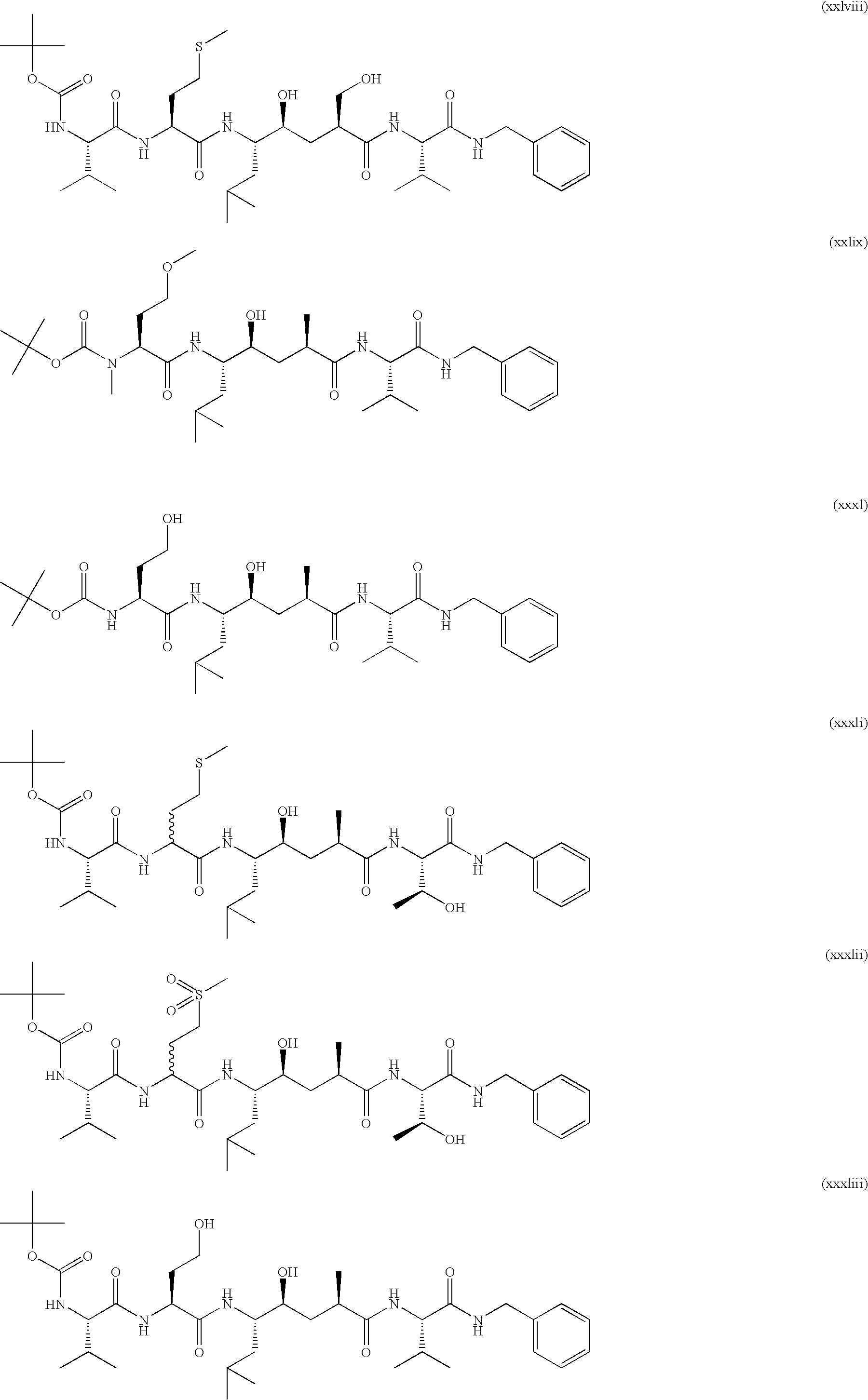 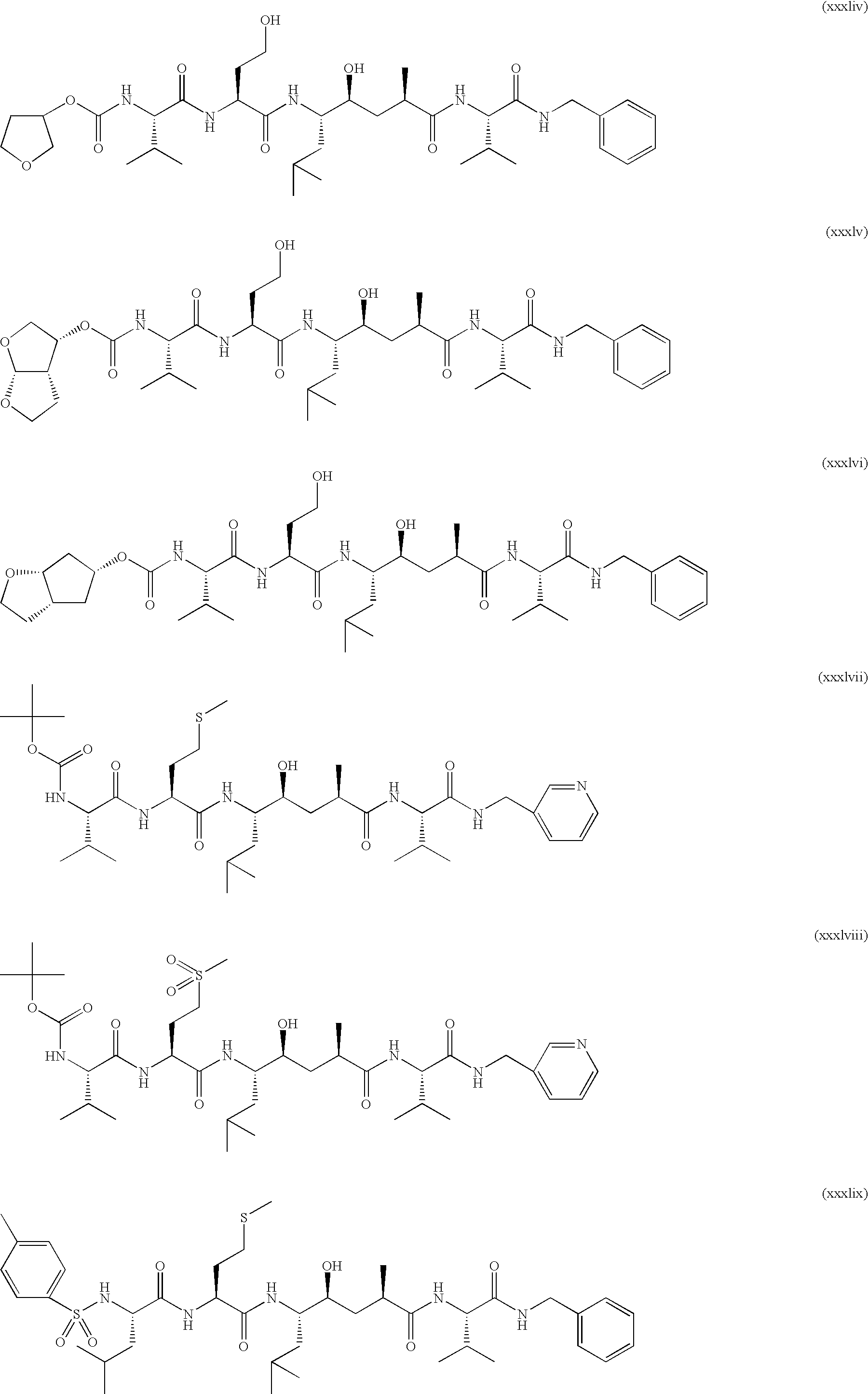 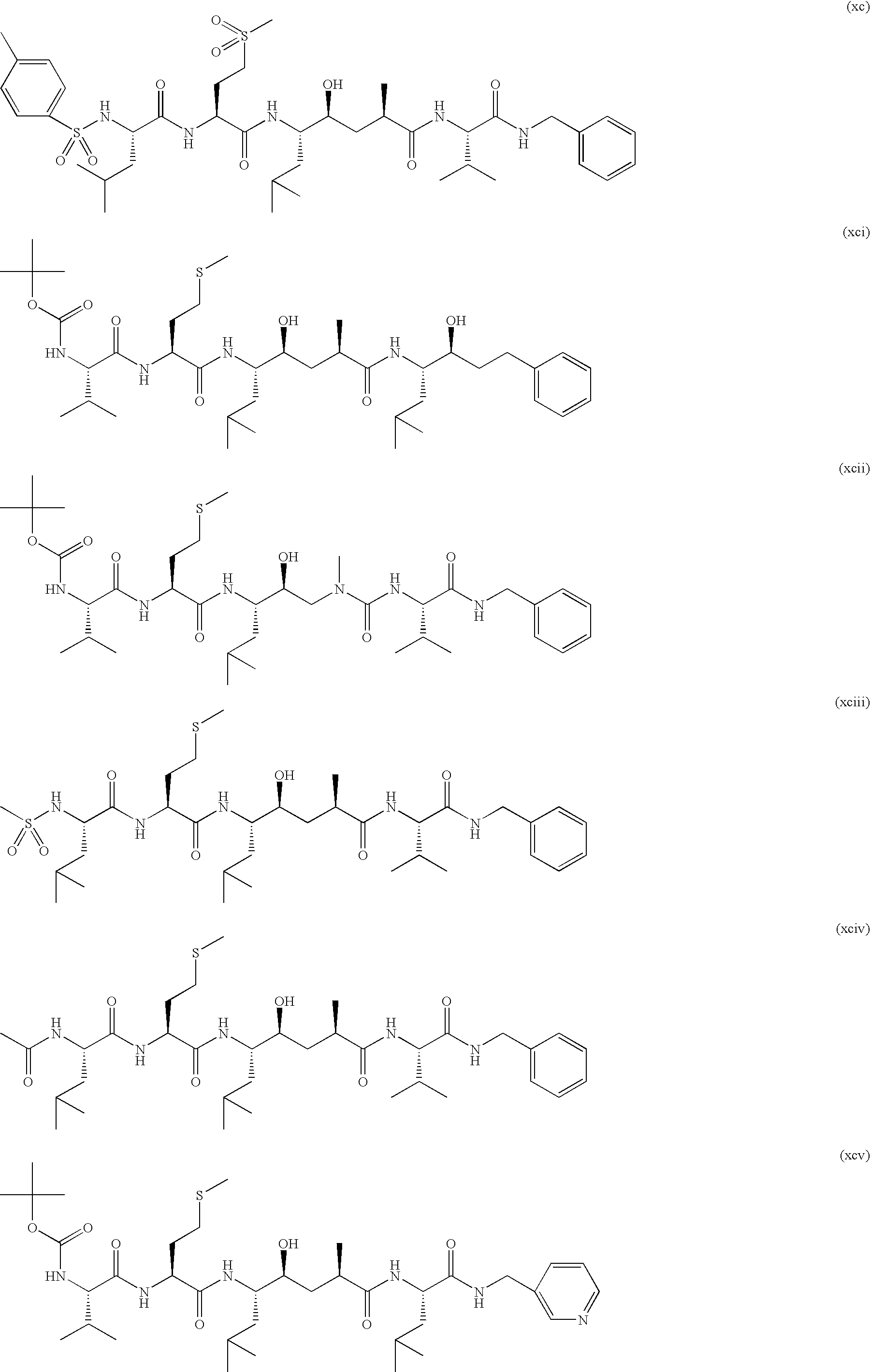 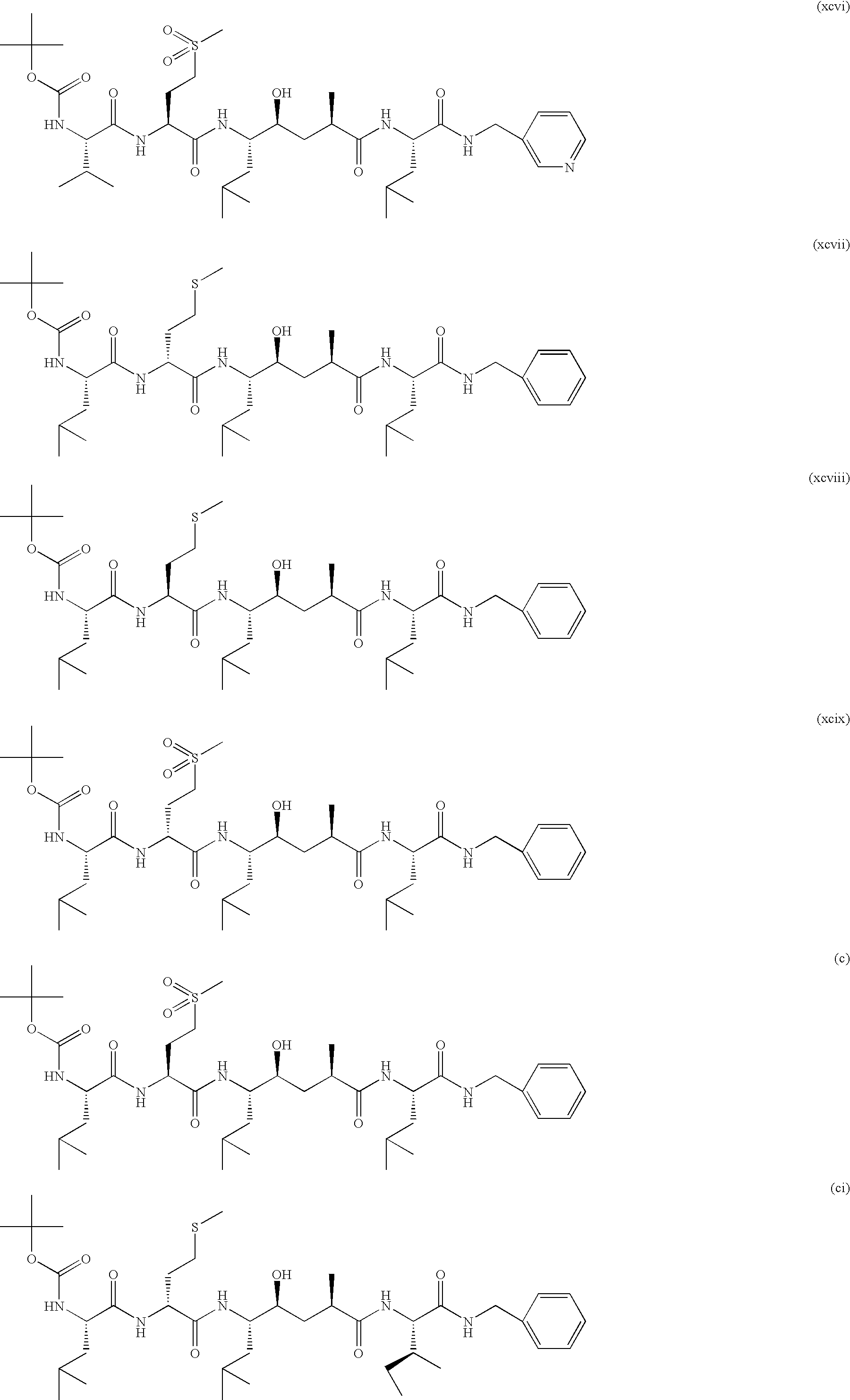 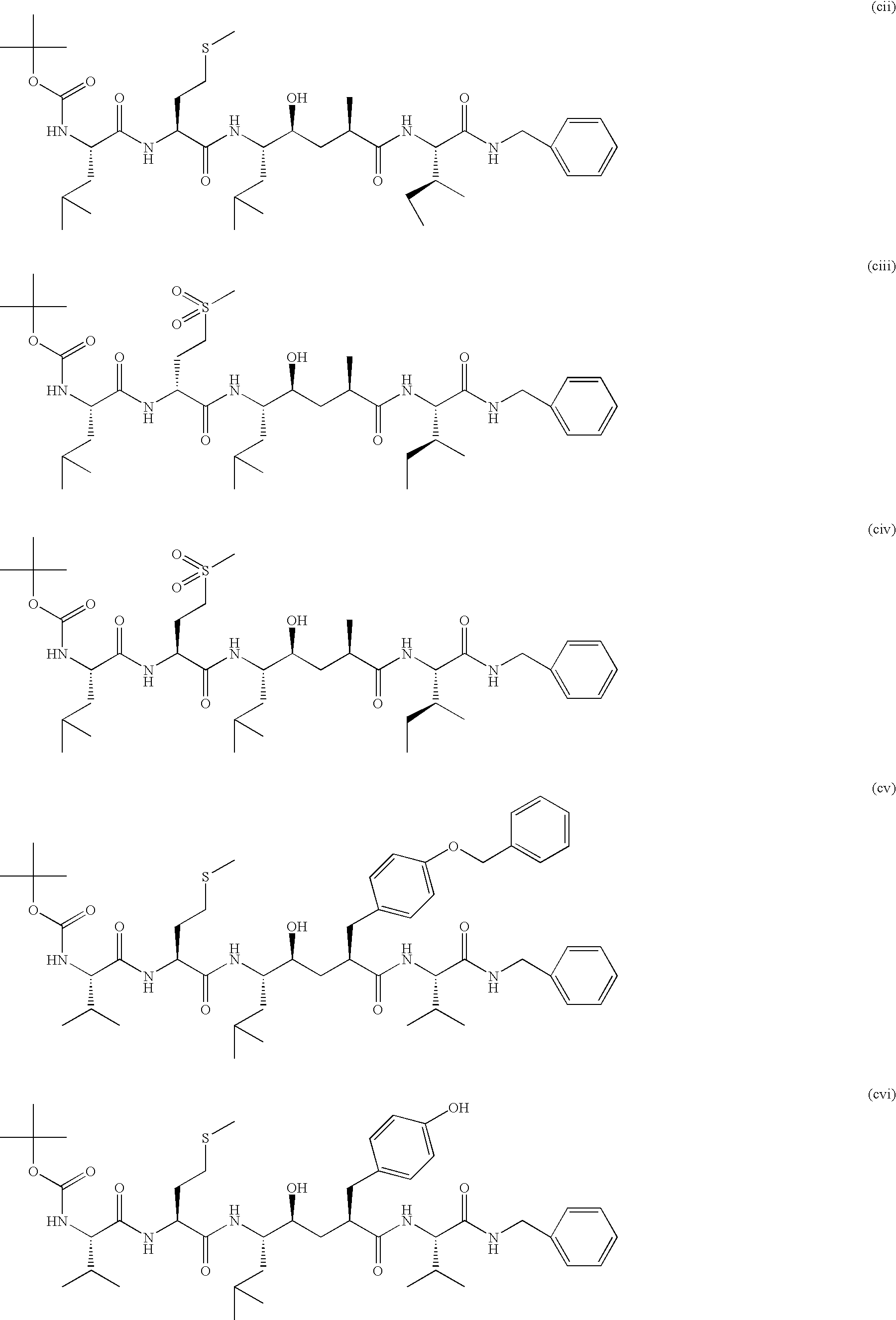 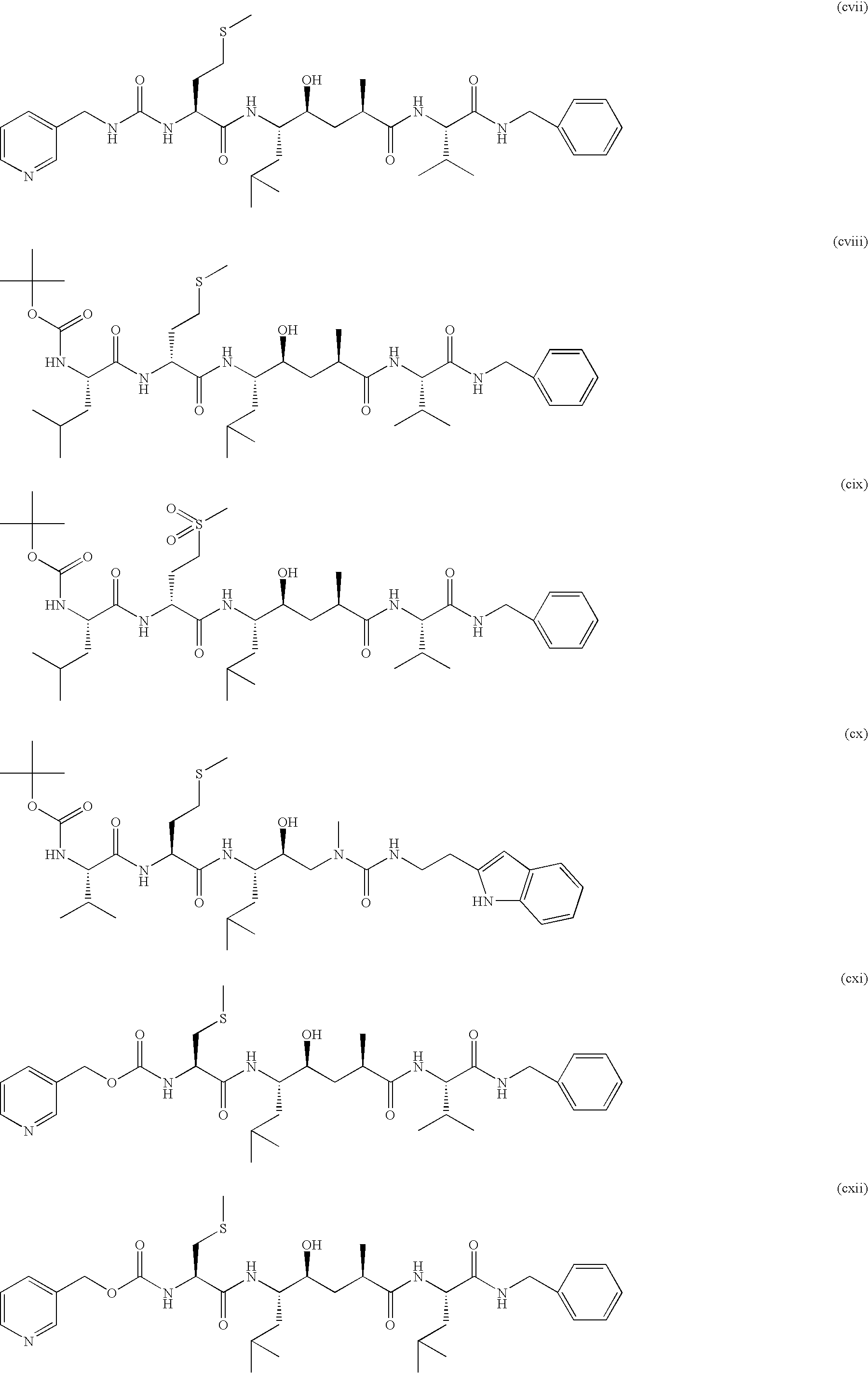 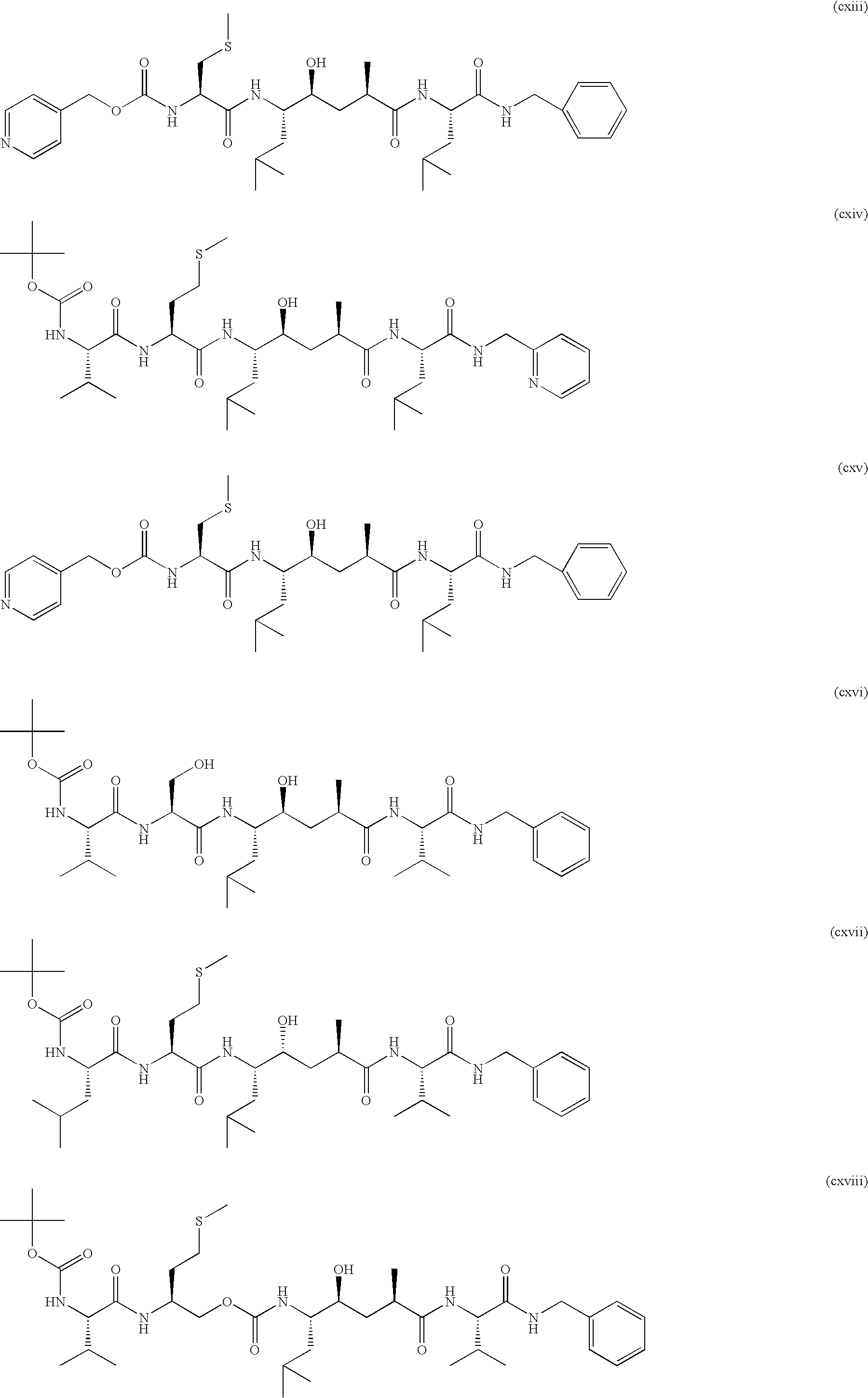 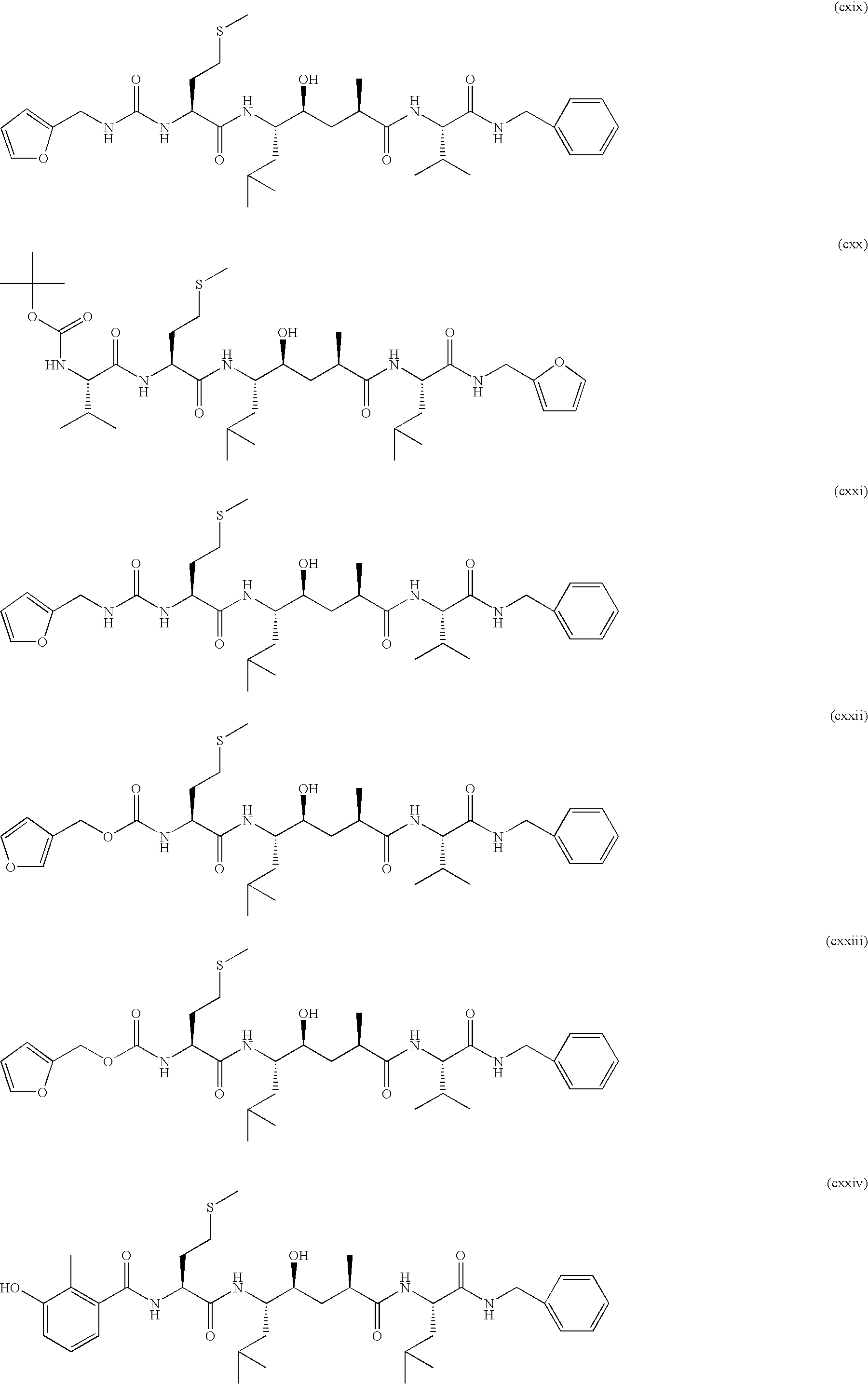 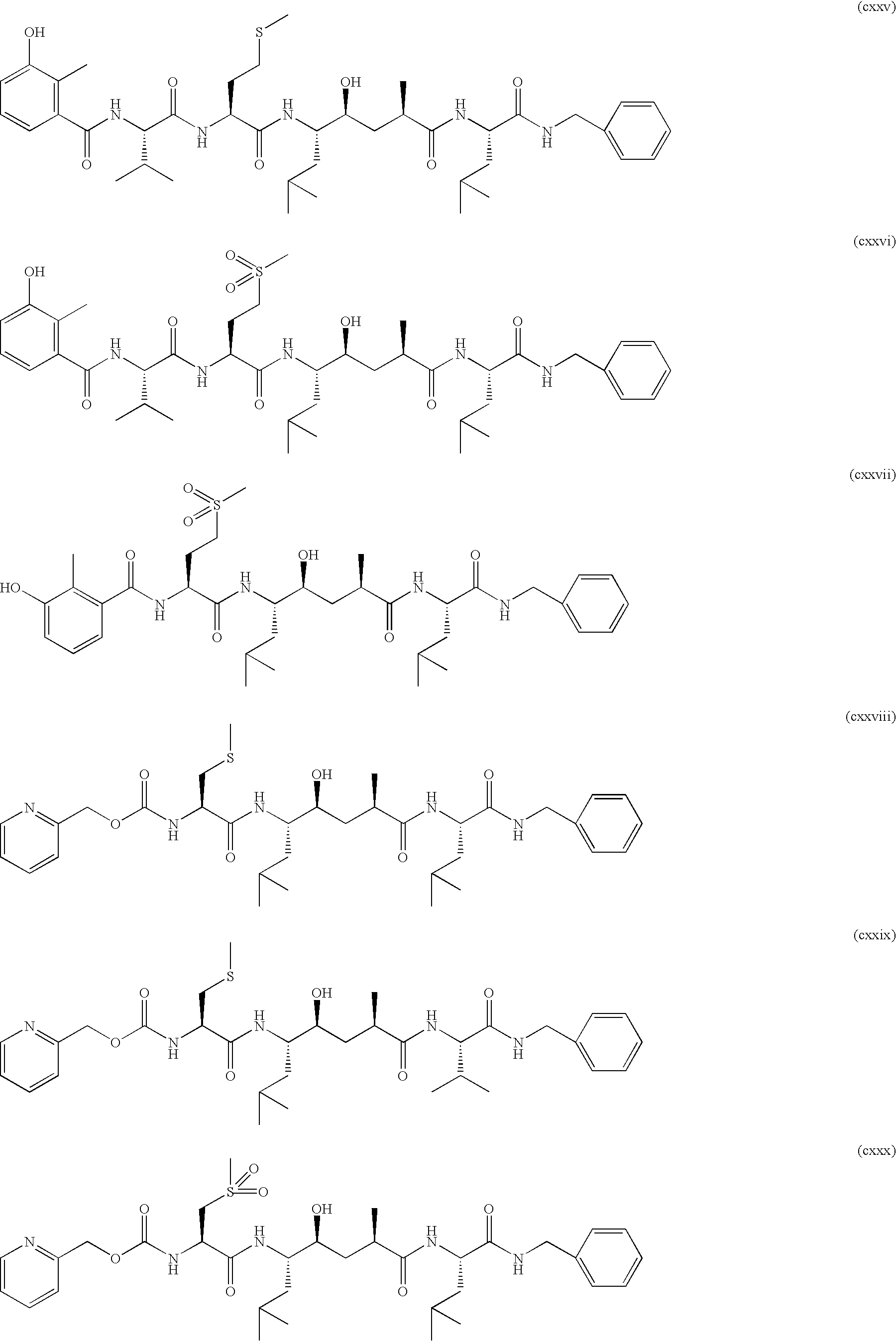 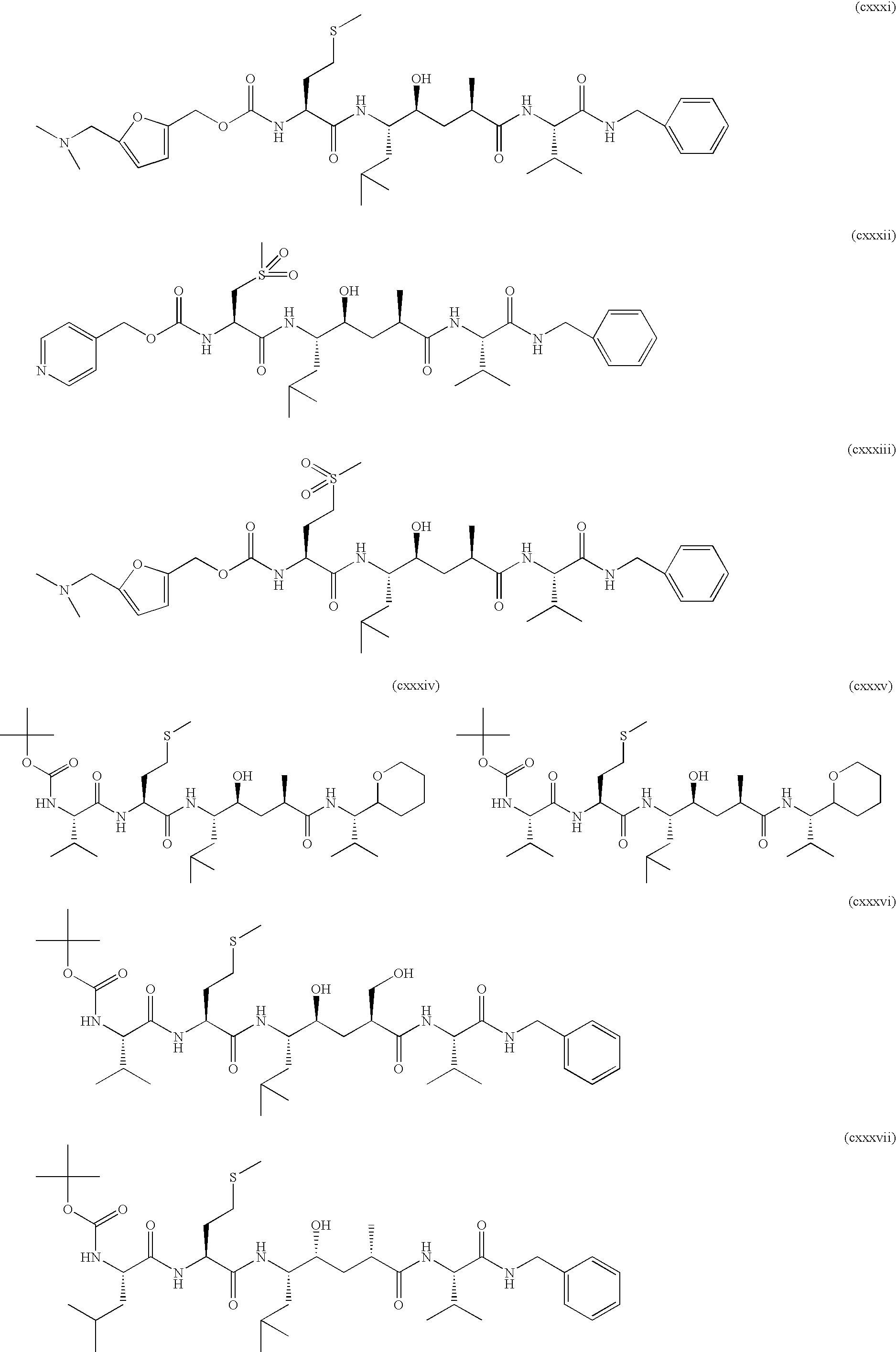 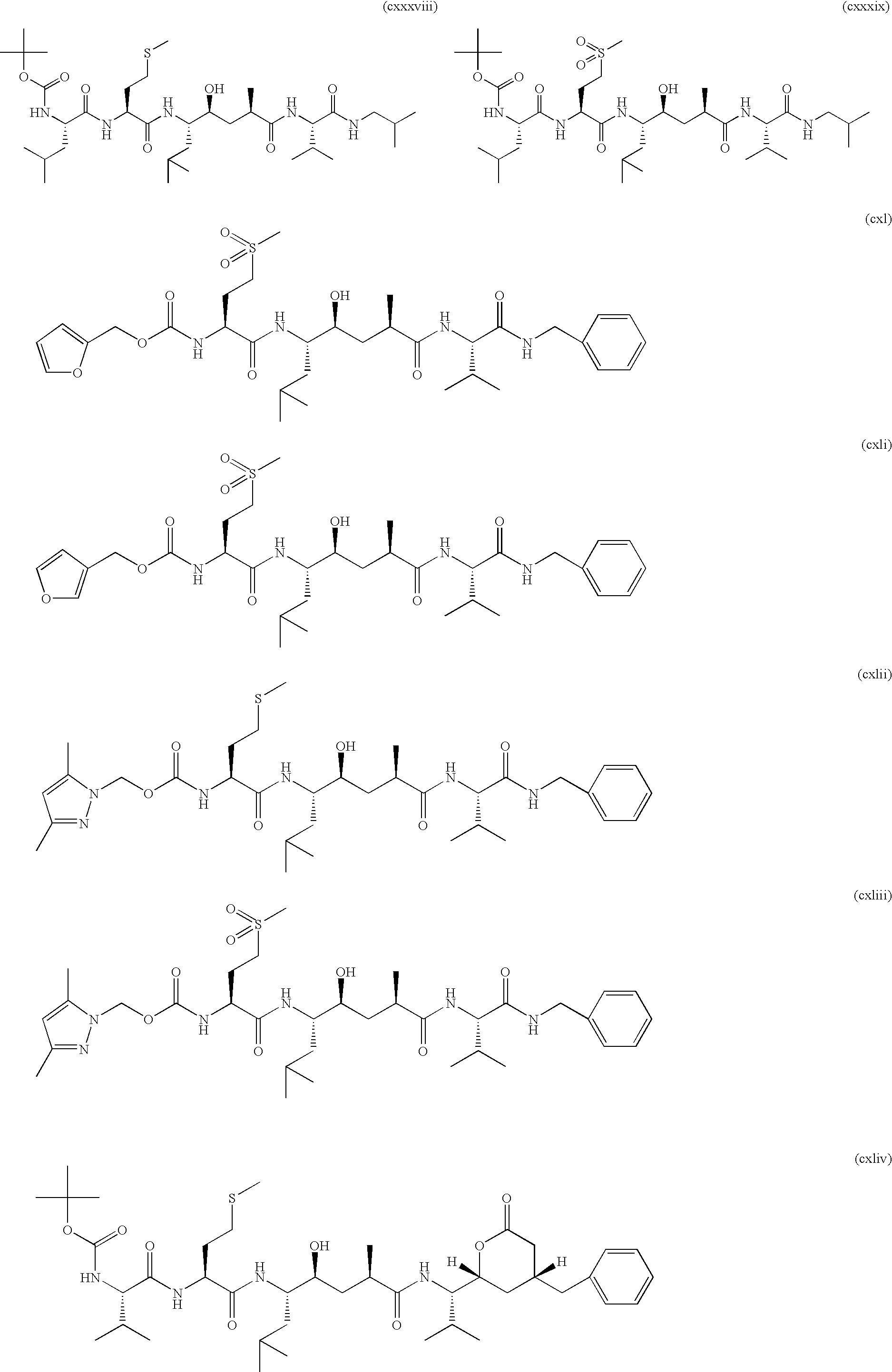 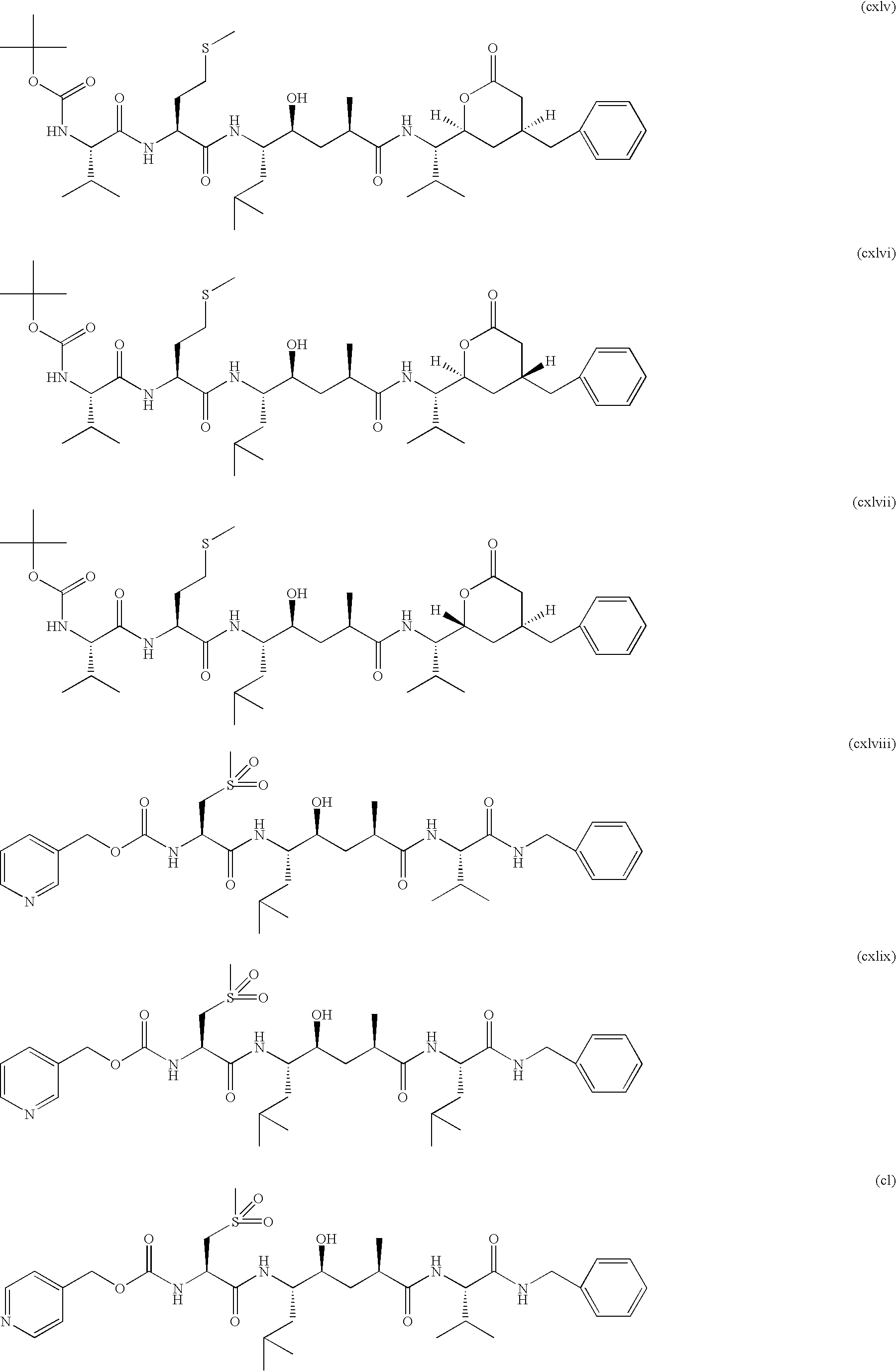 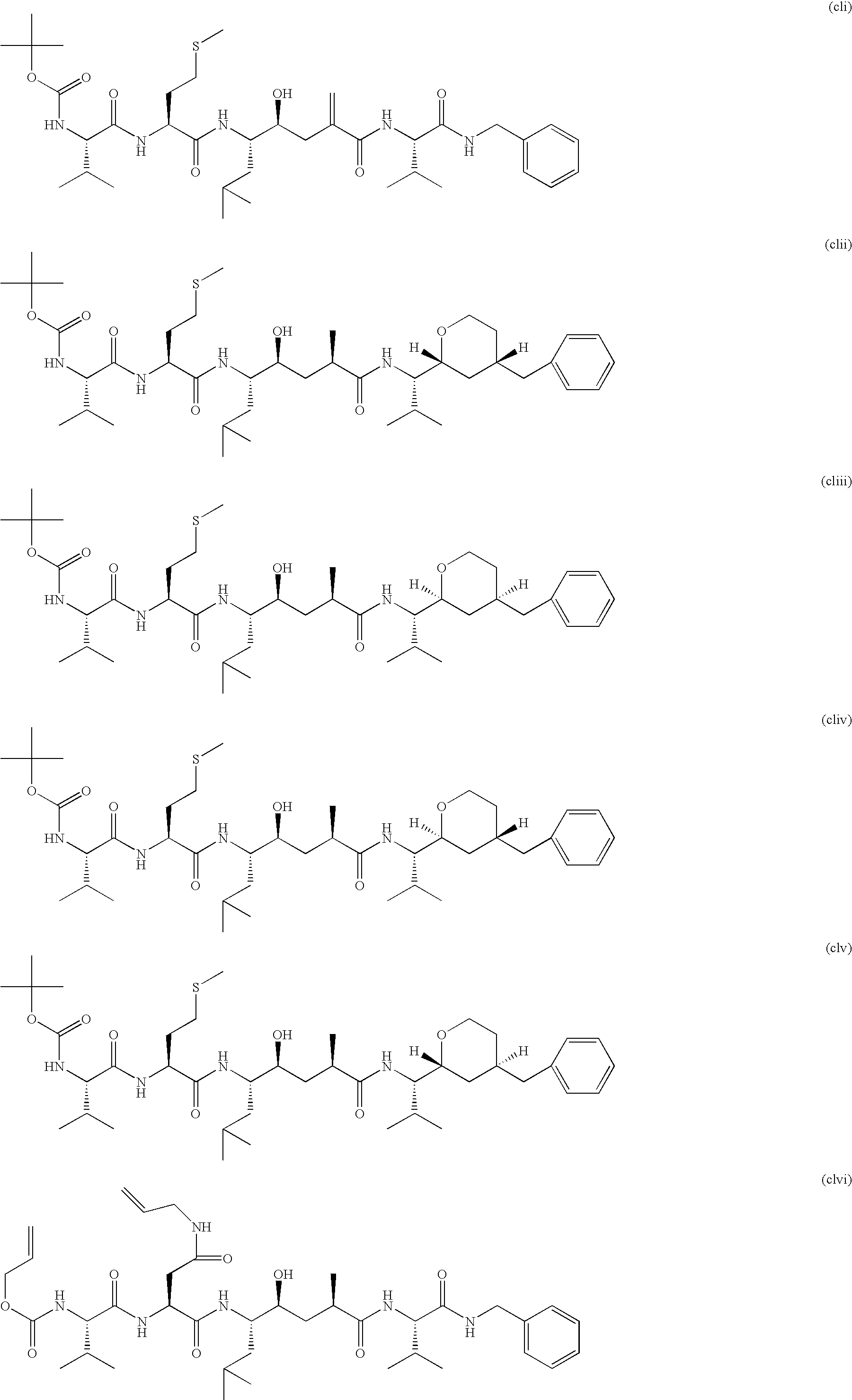 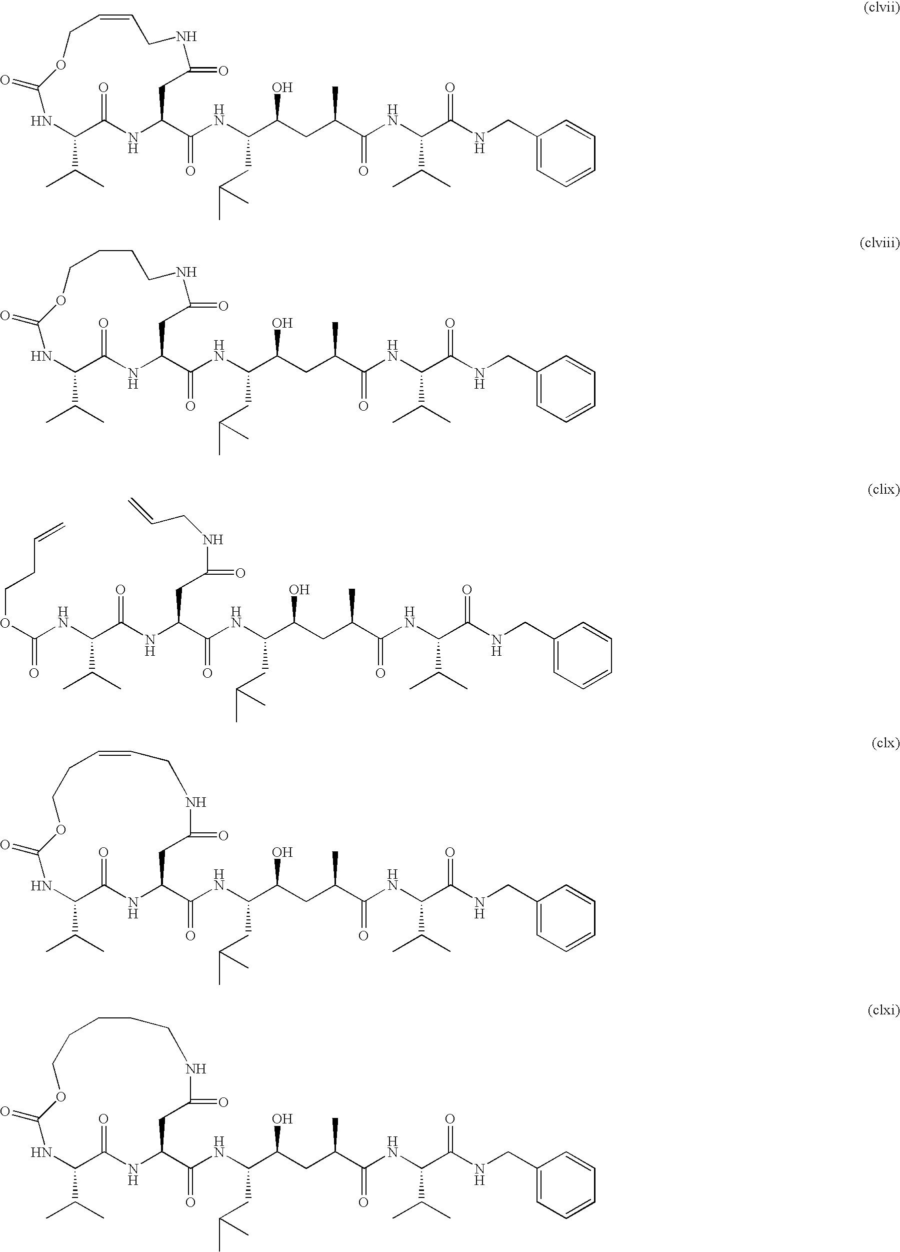 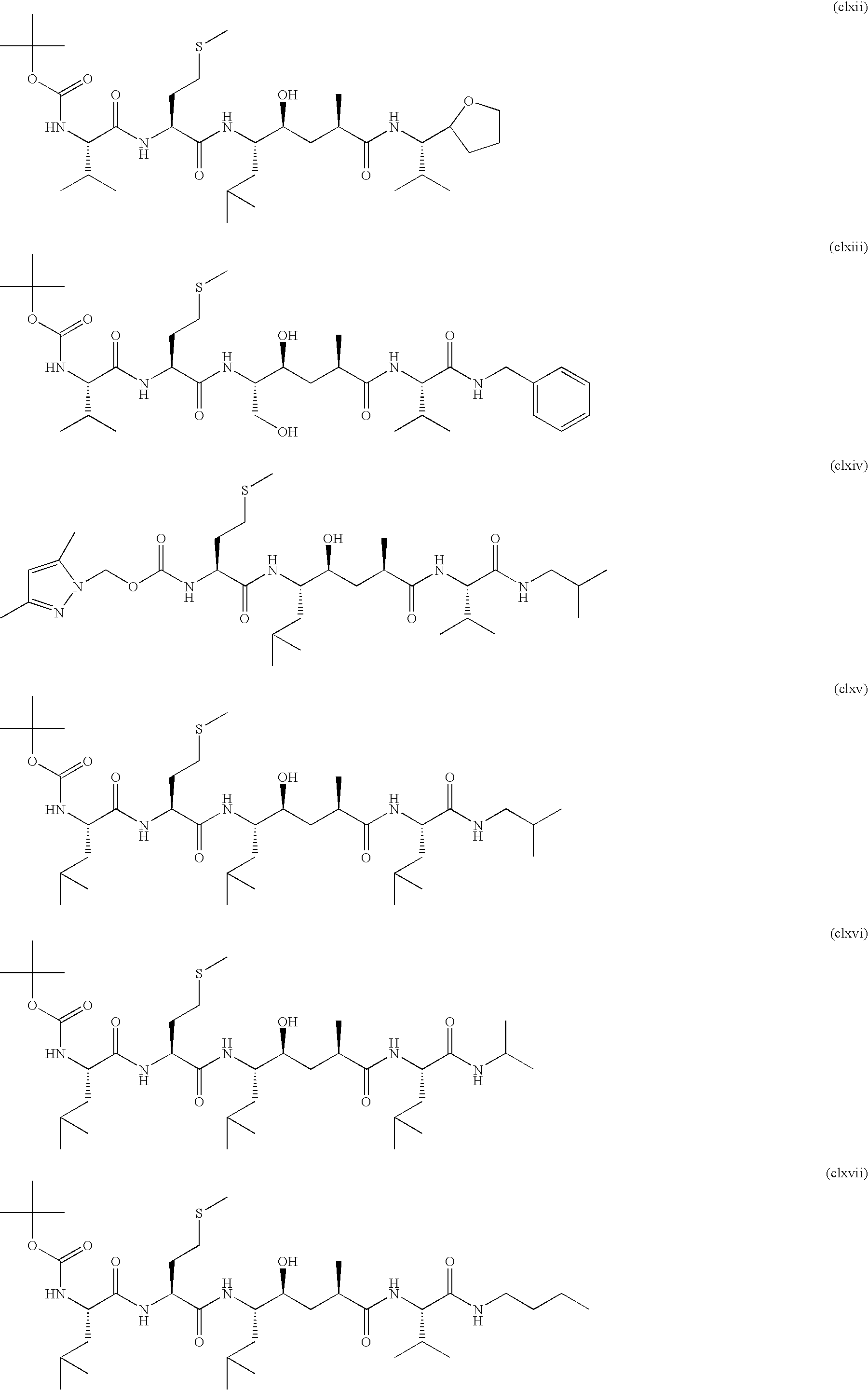 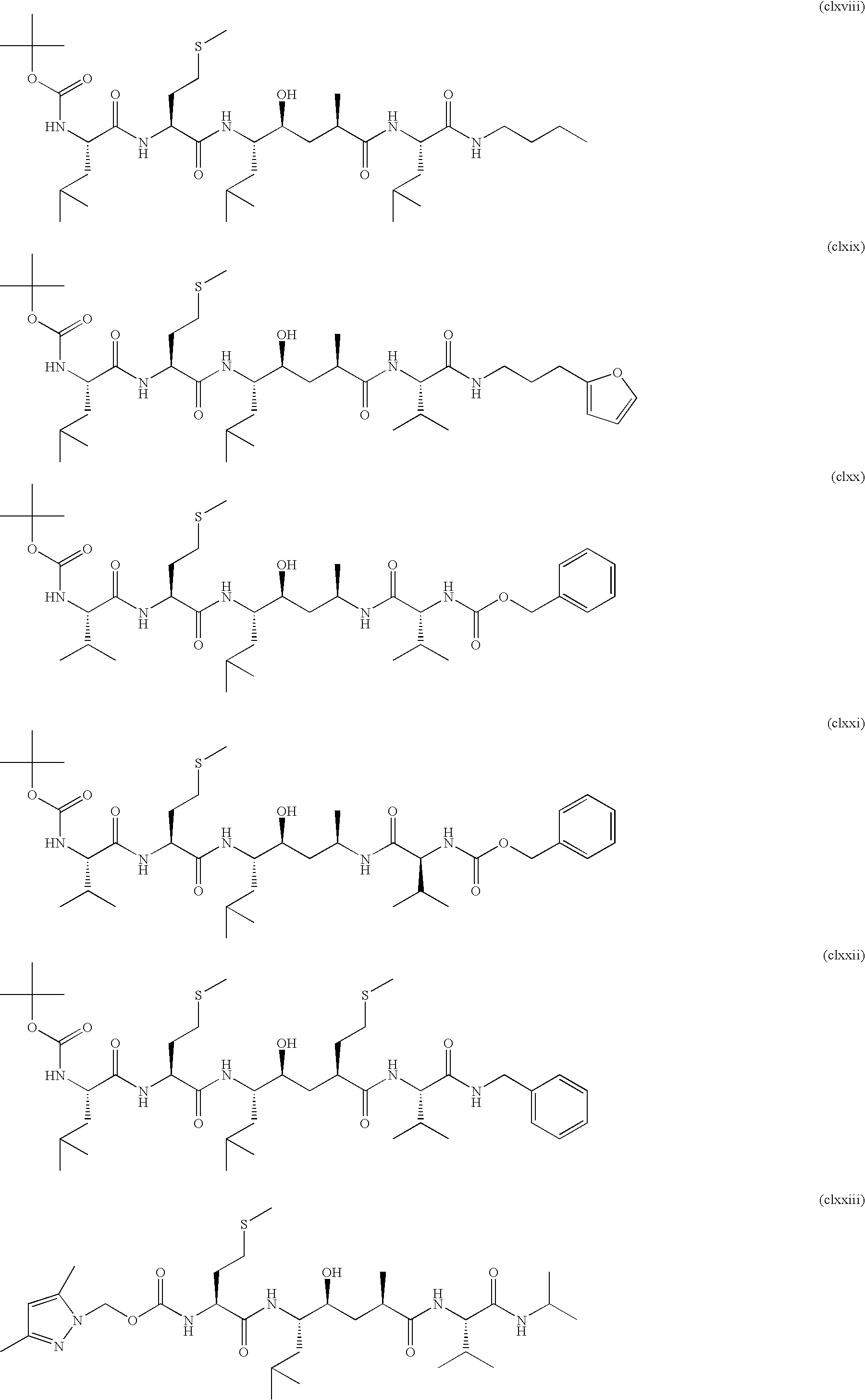 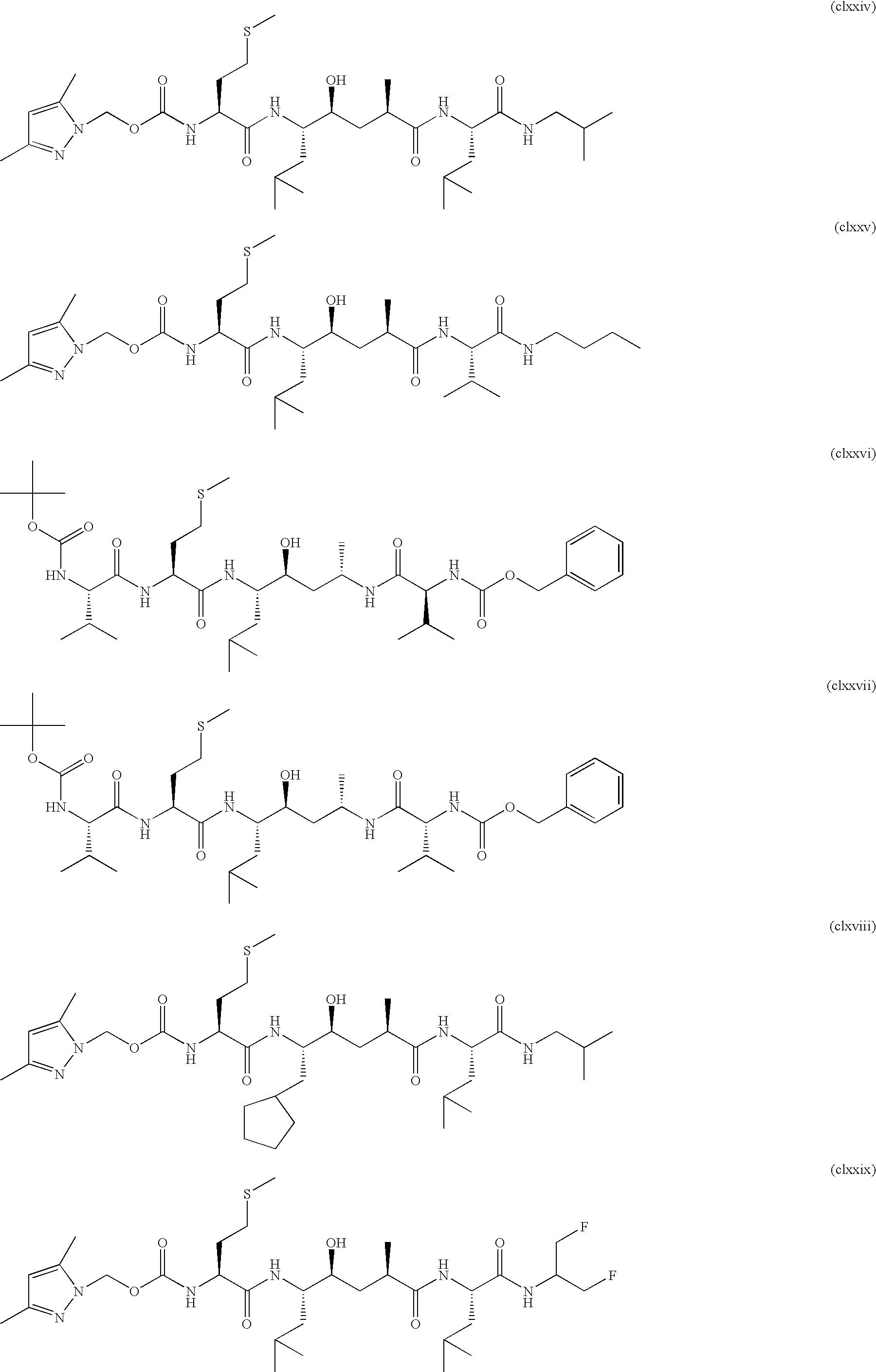 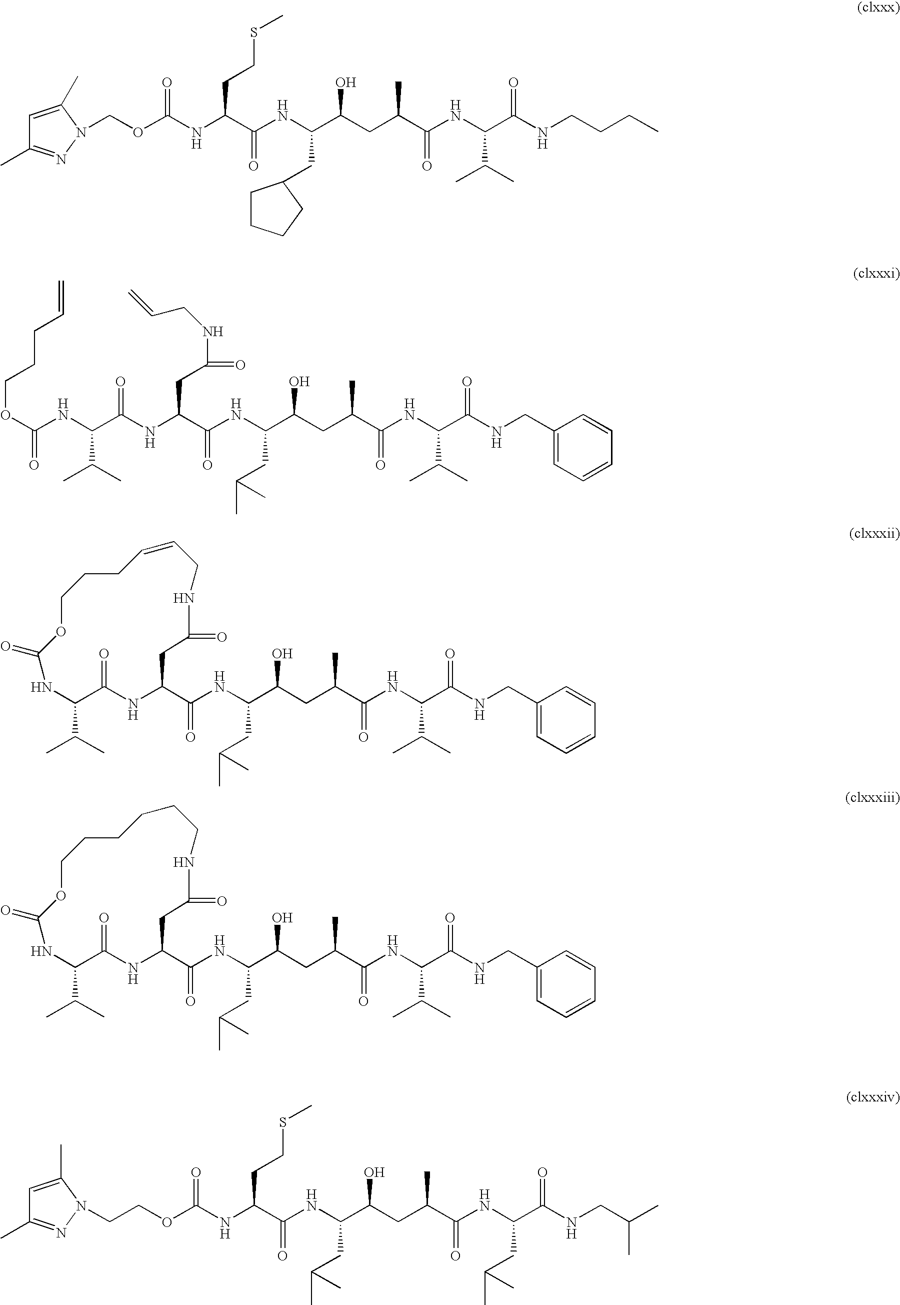 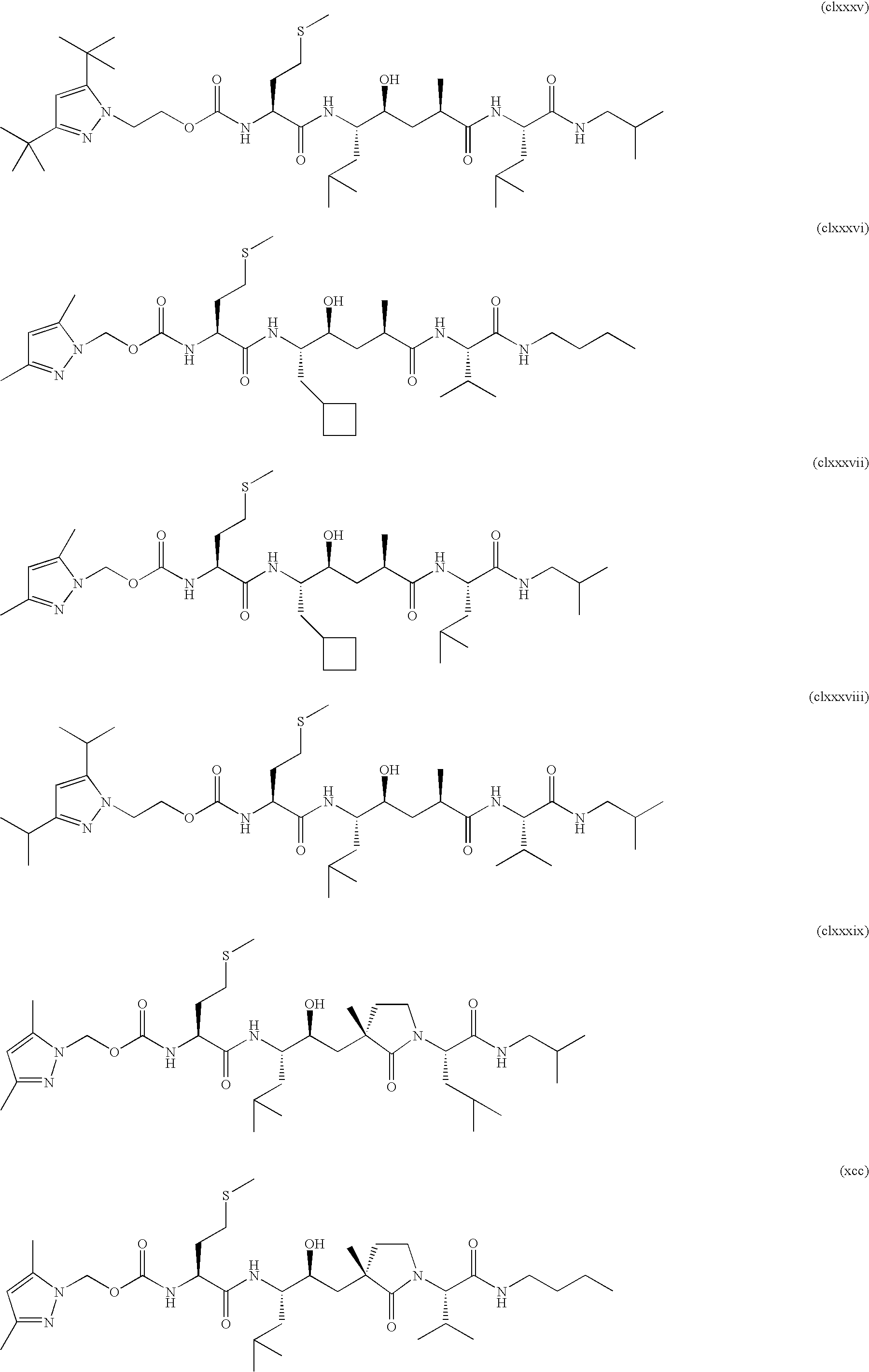 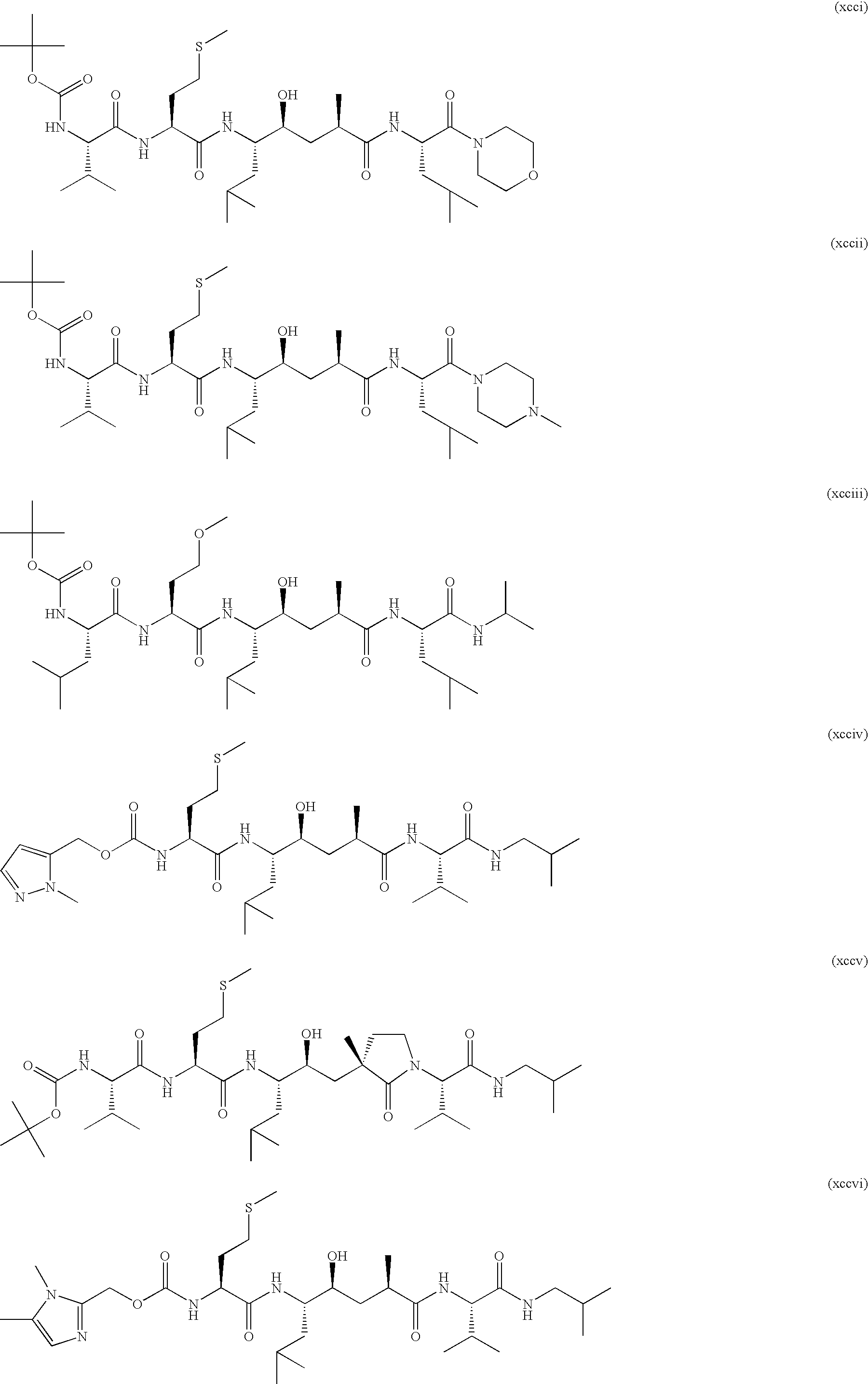 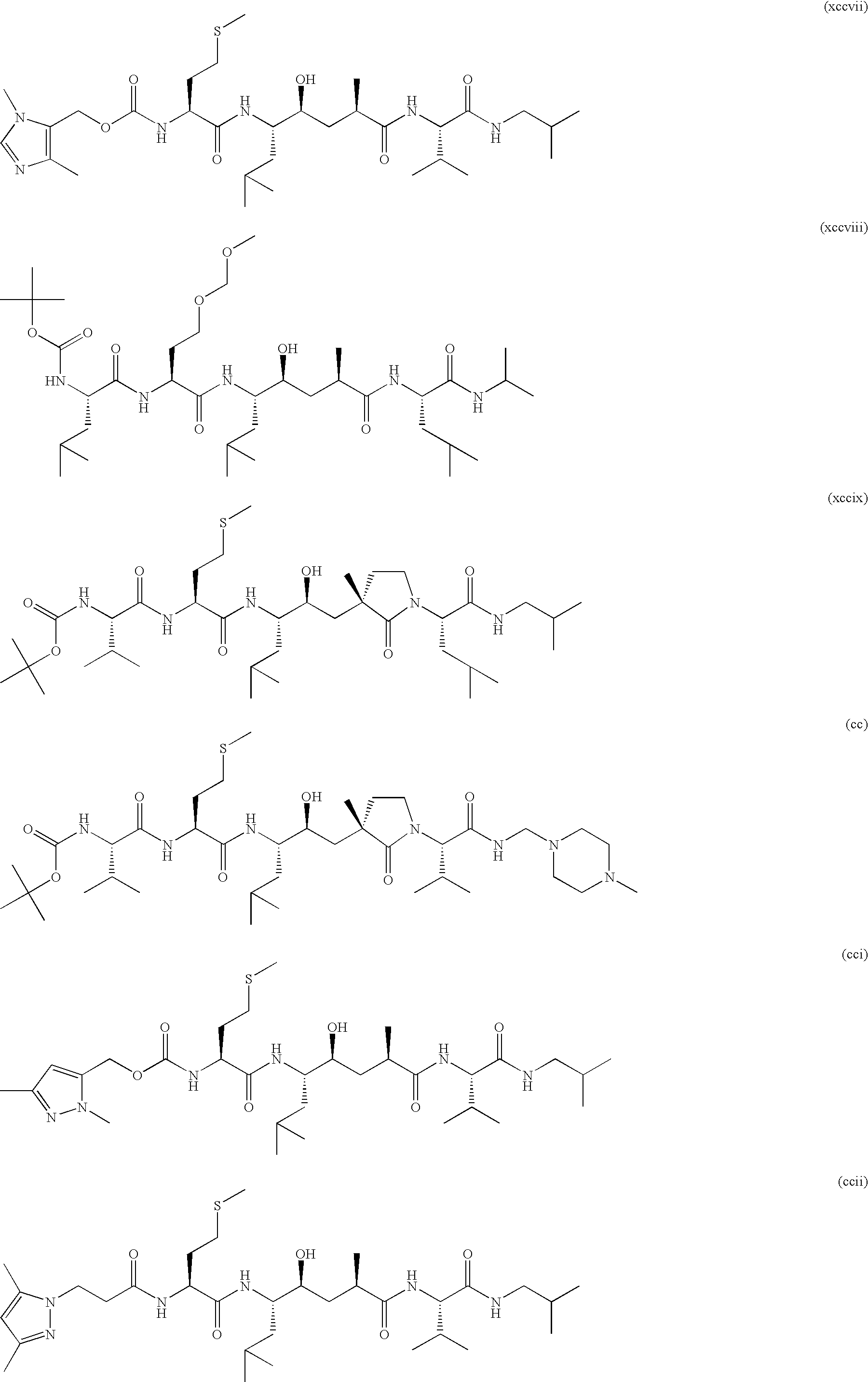 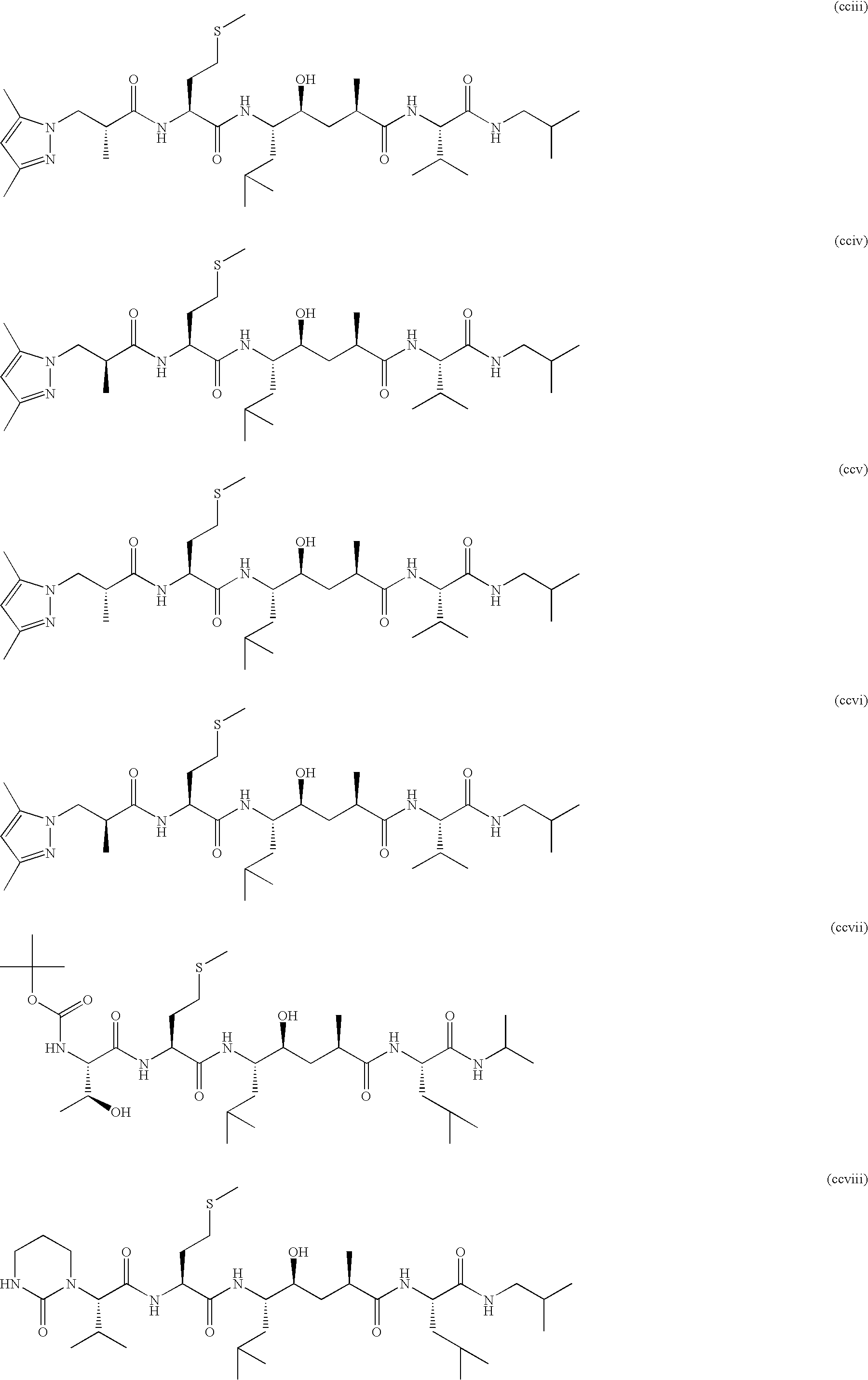 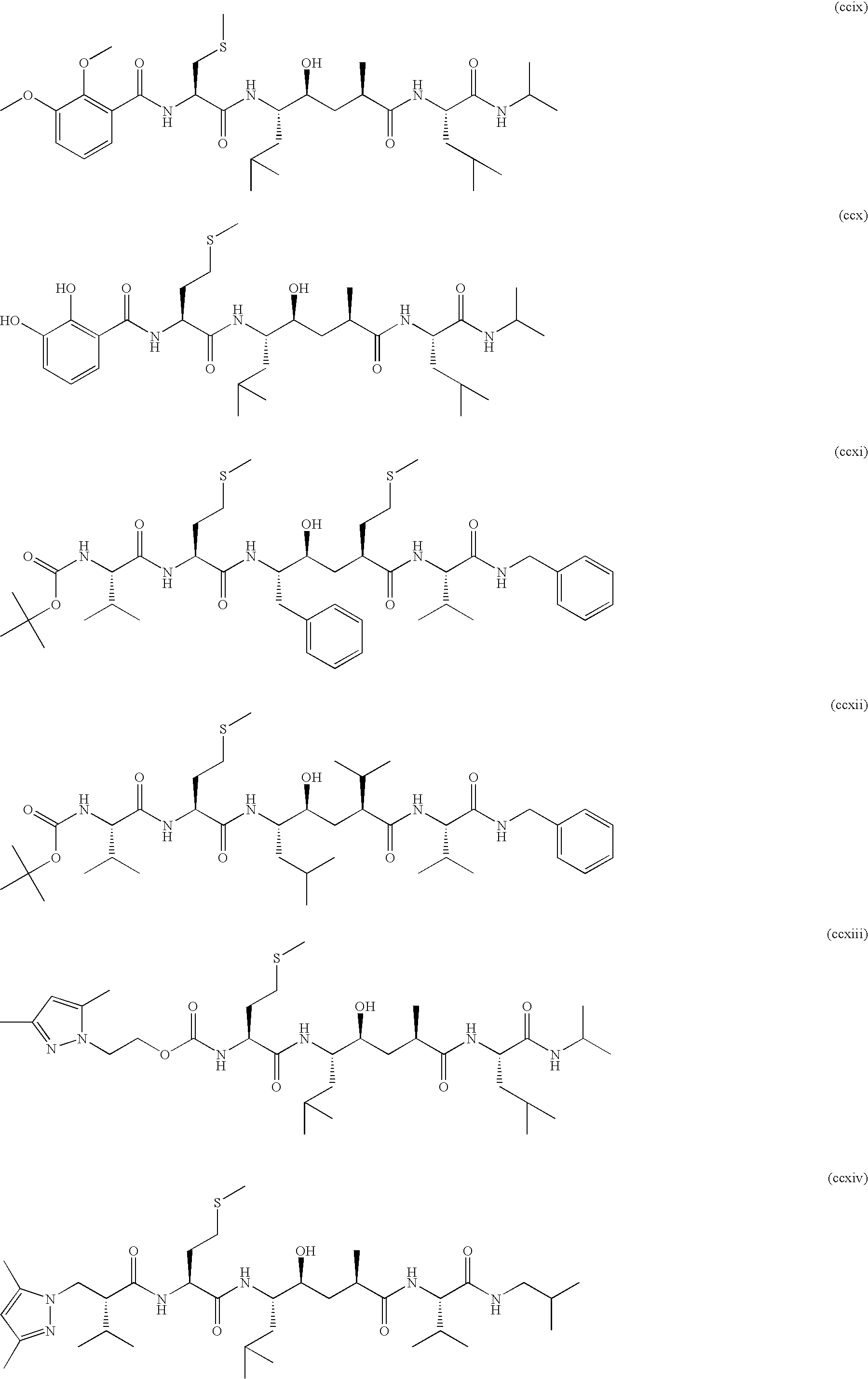 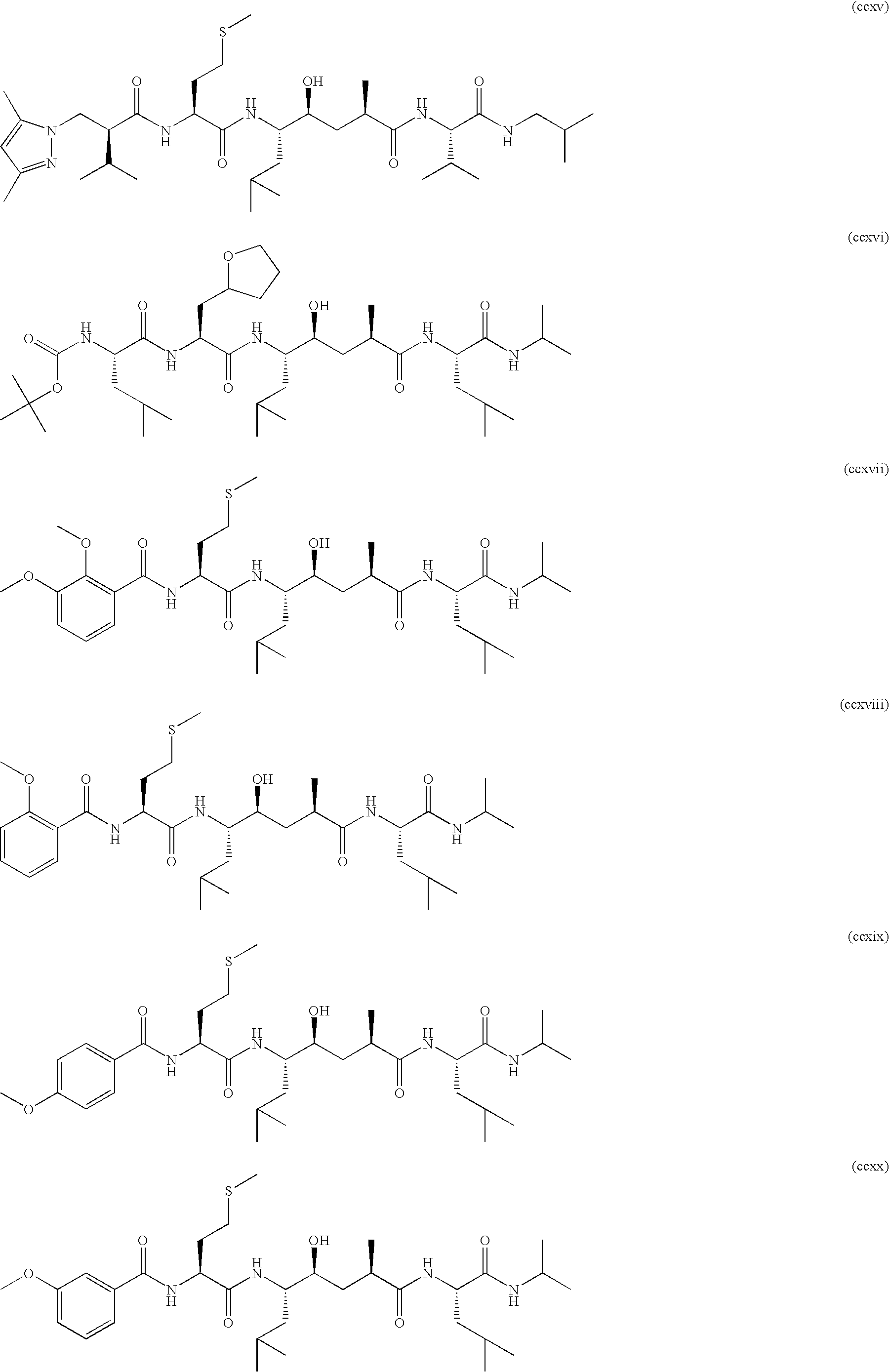 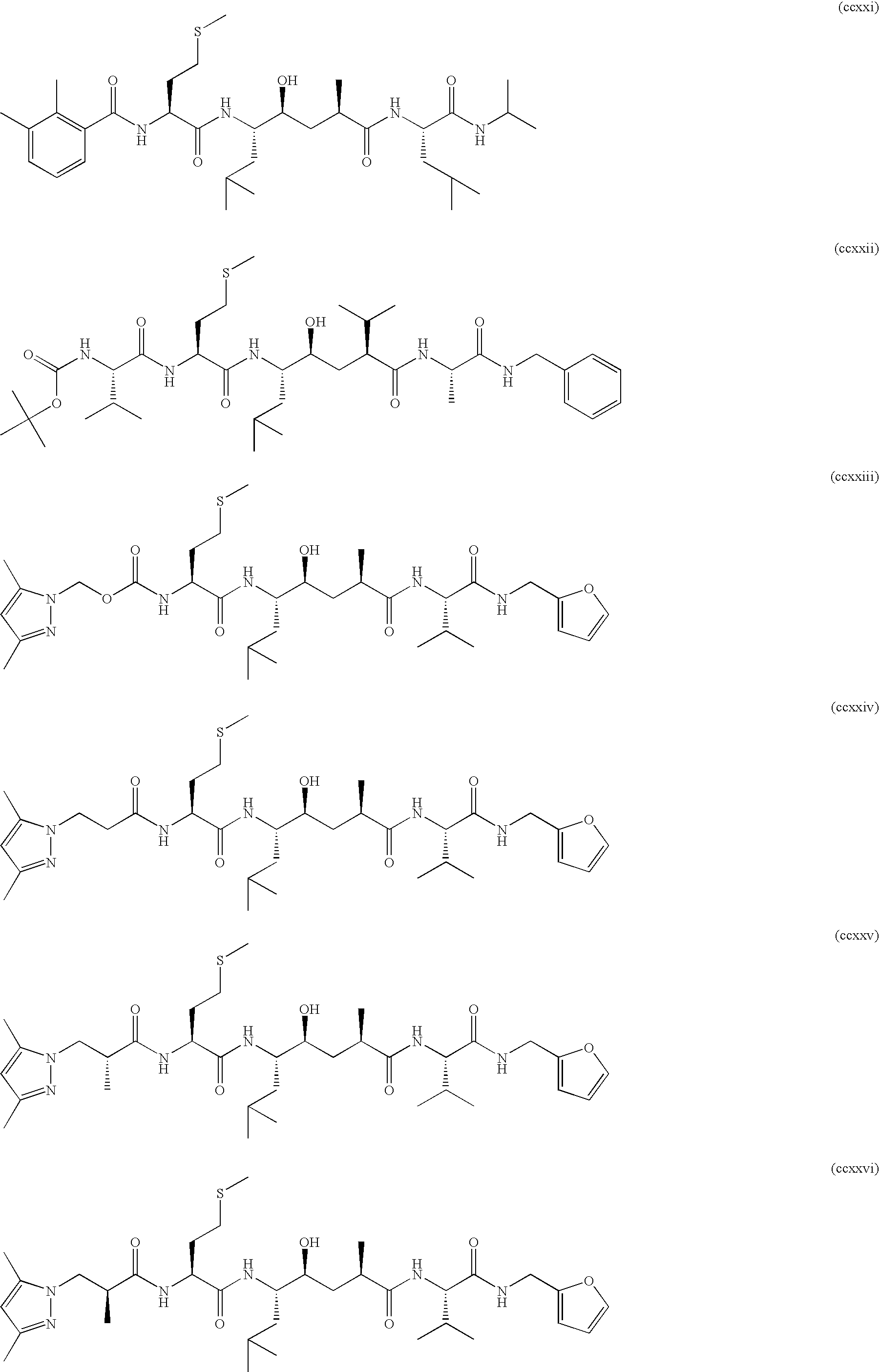 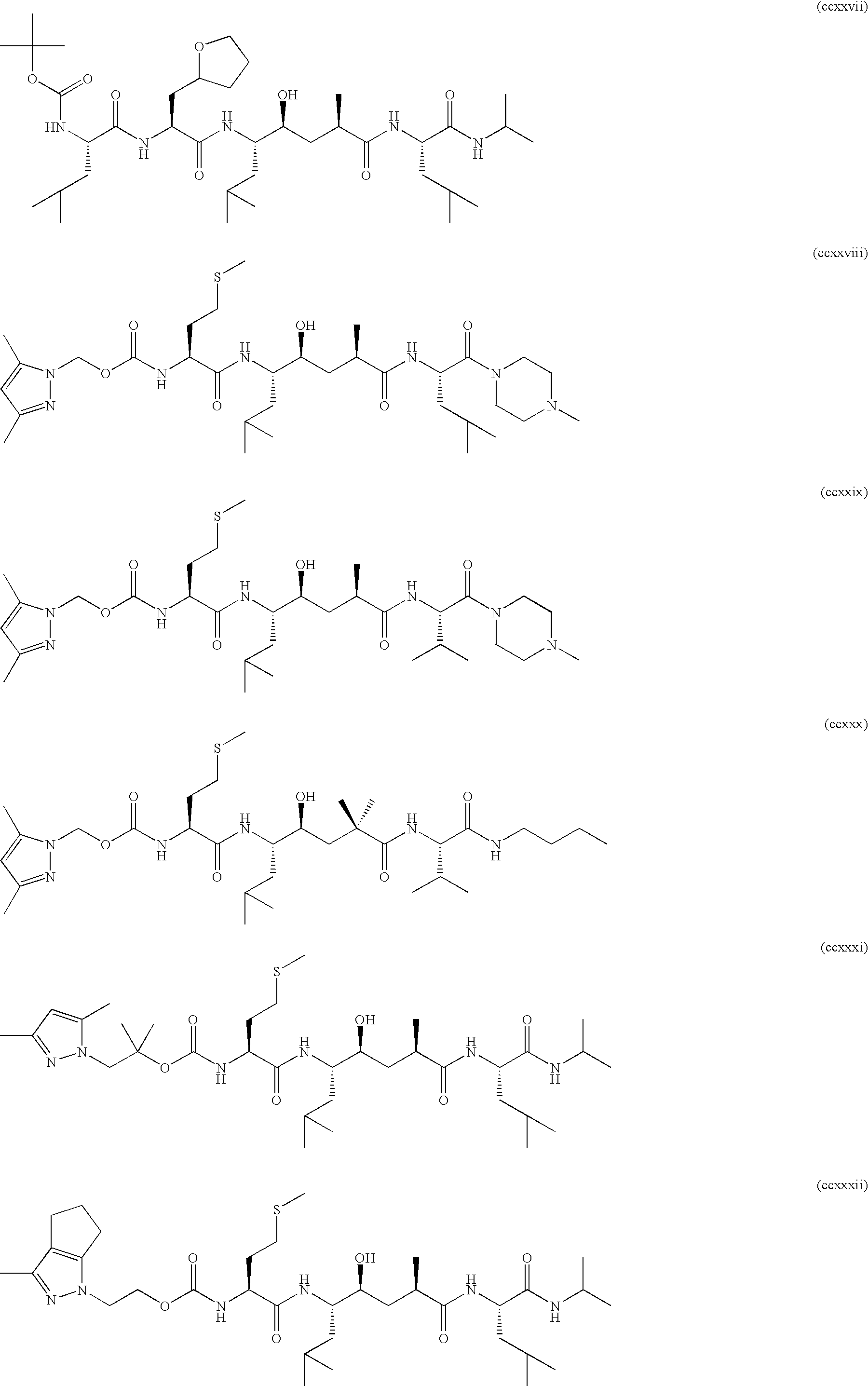 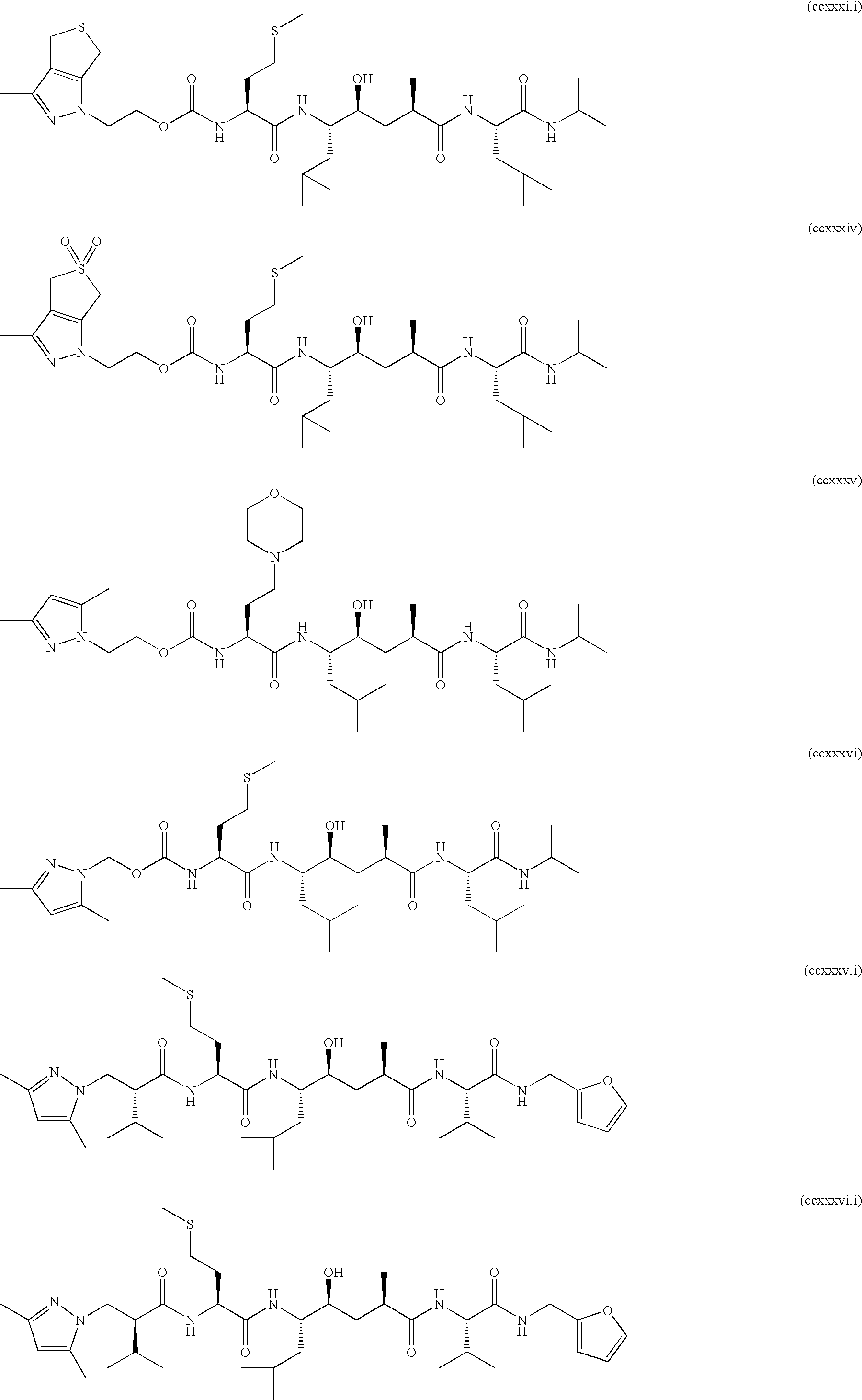 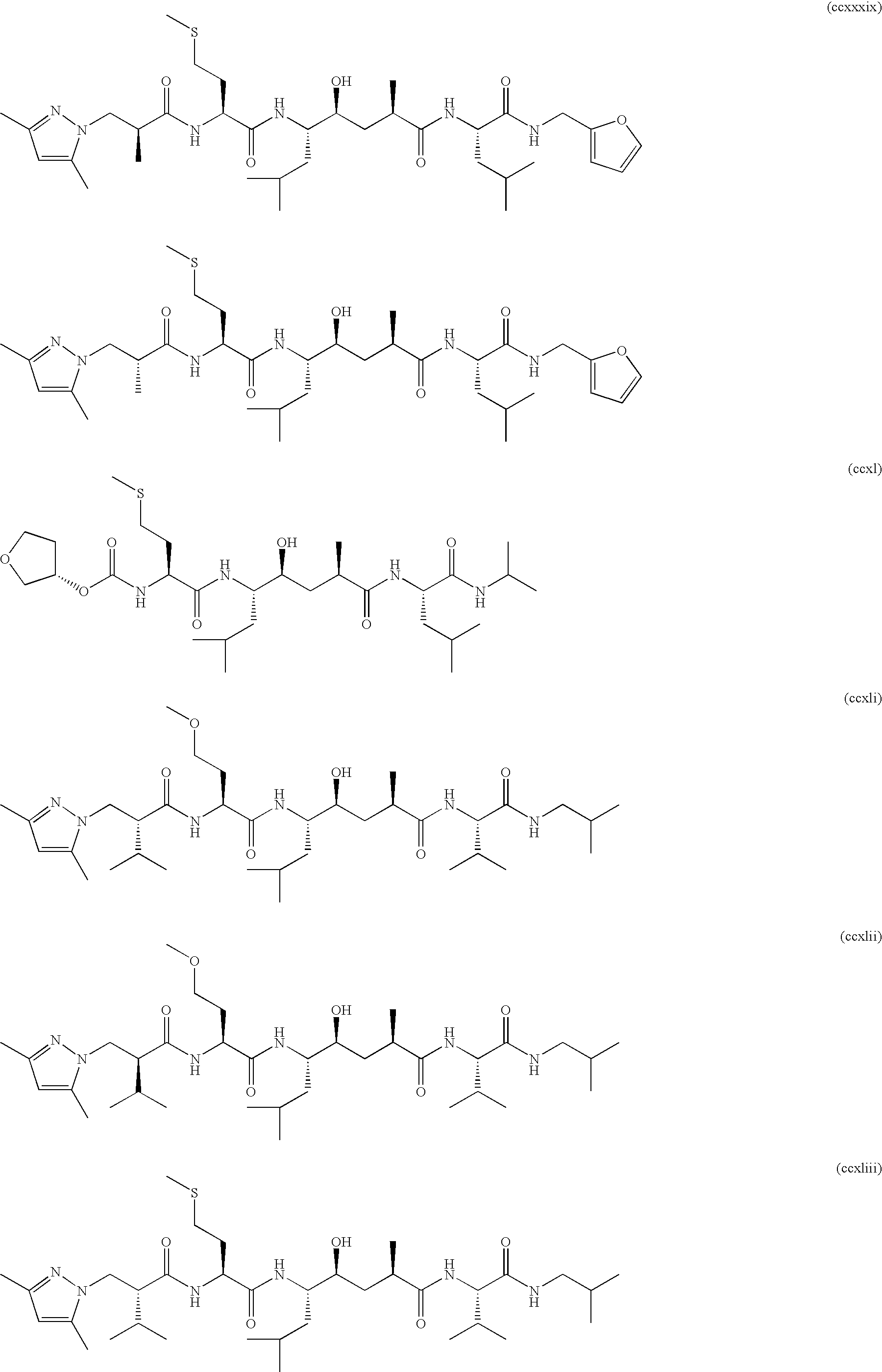 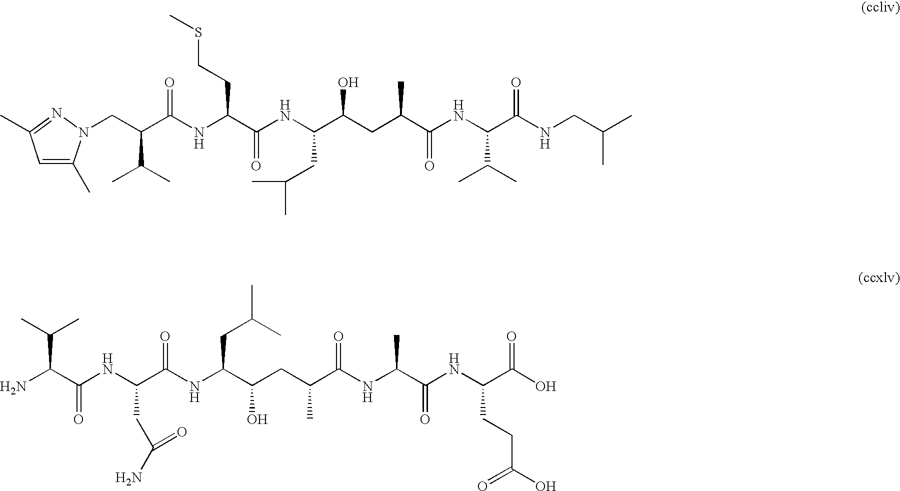
or pharmaceutically acceptable salts thereof.

84. The compound of claim 83, wherein the compound is selected from the group consisting of: 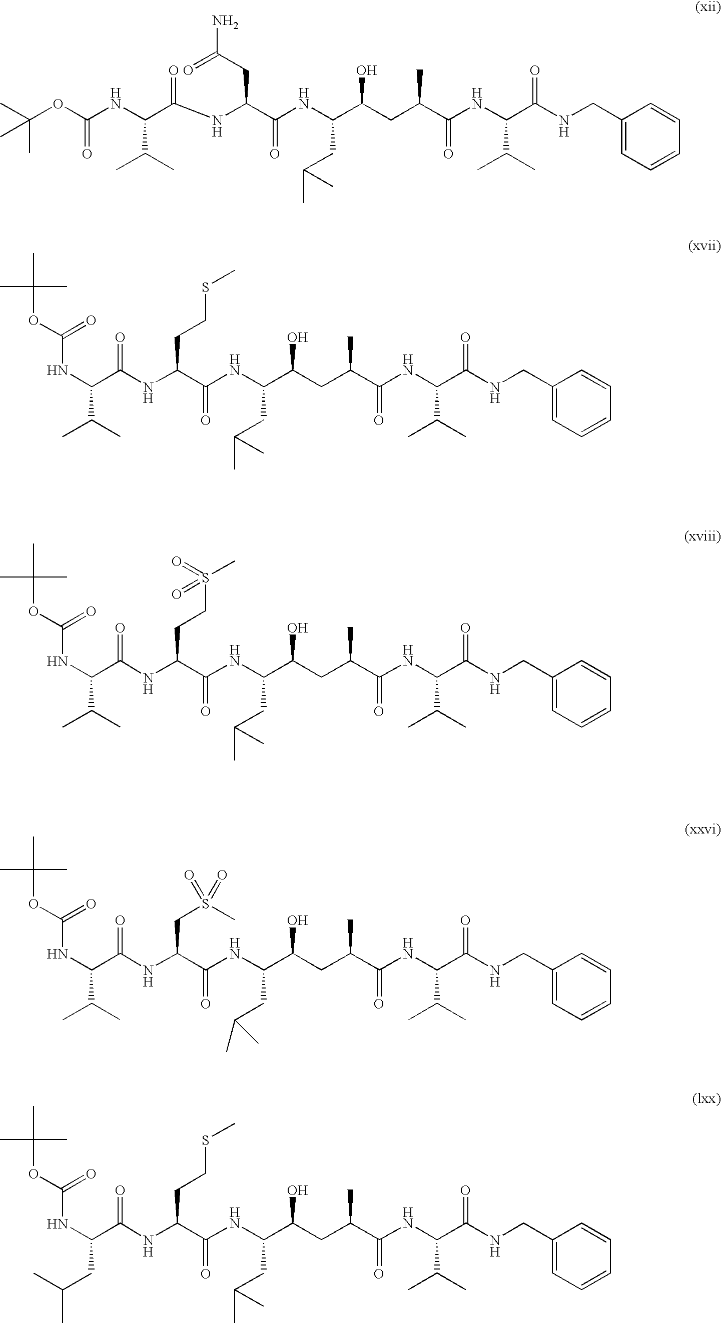 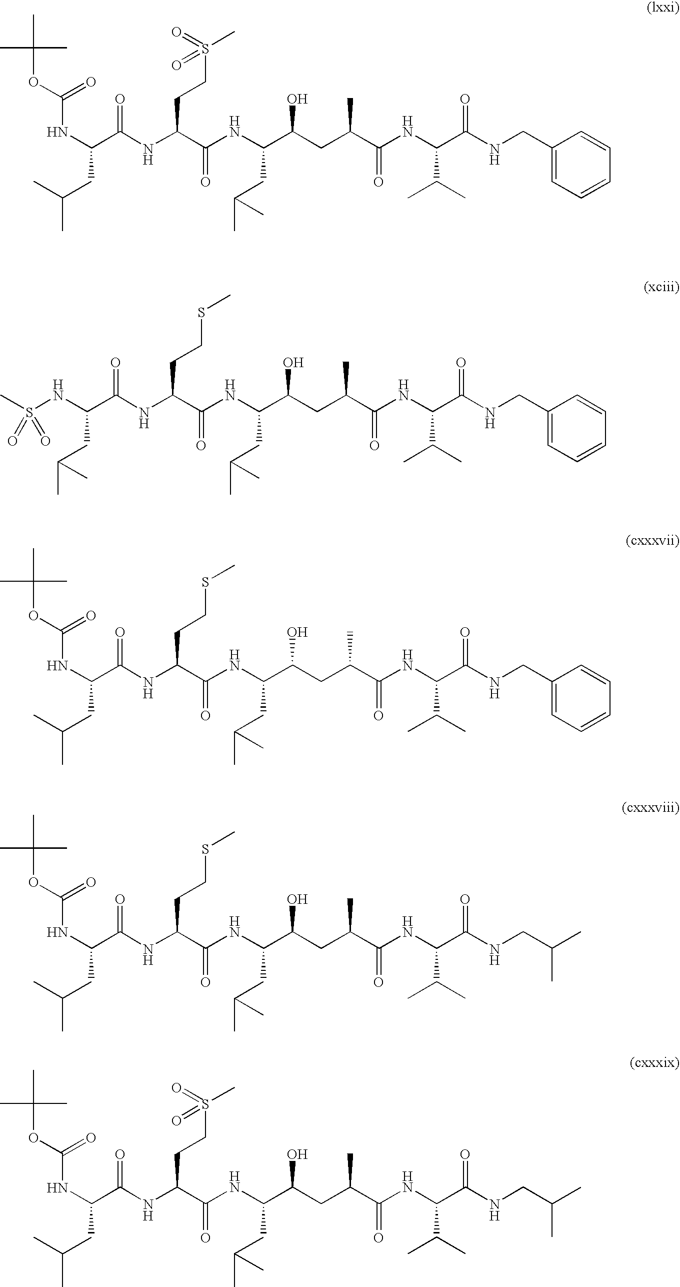 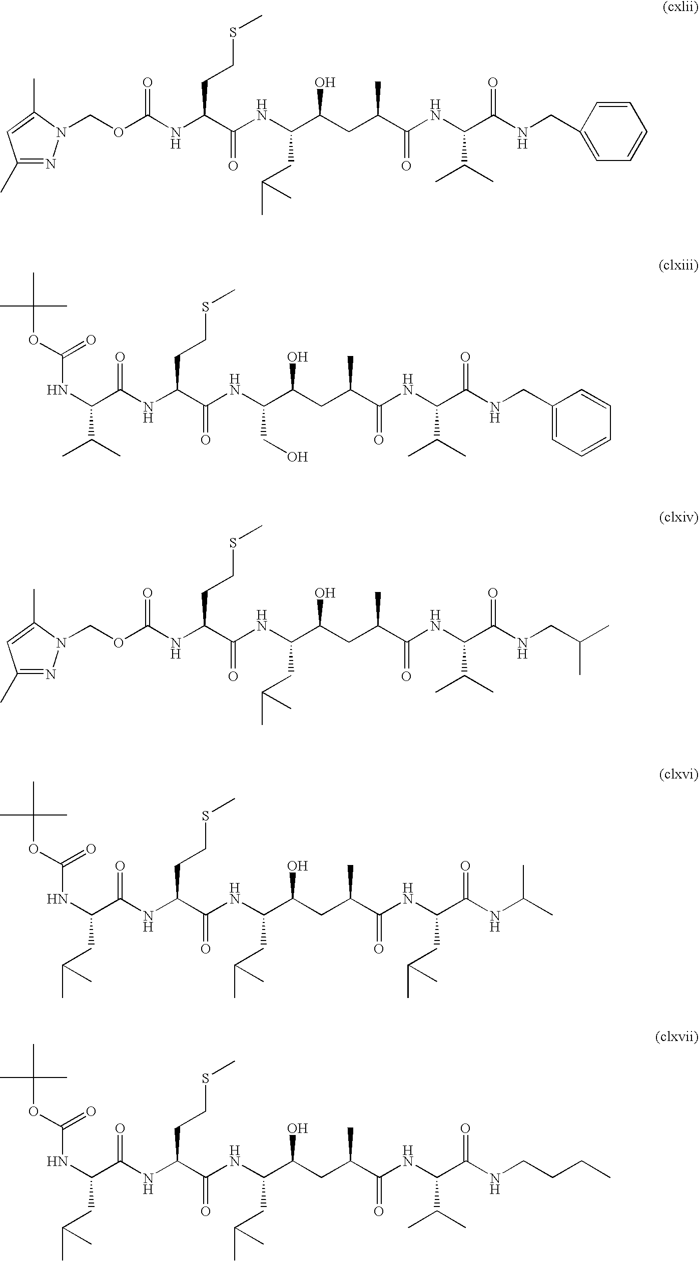 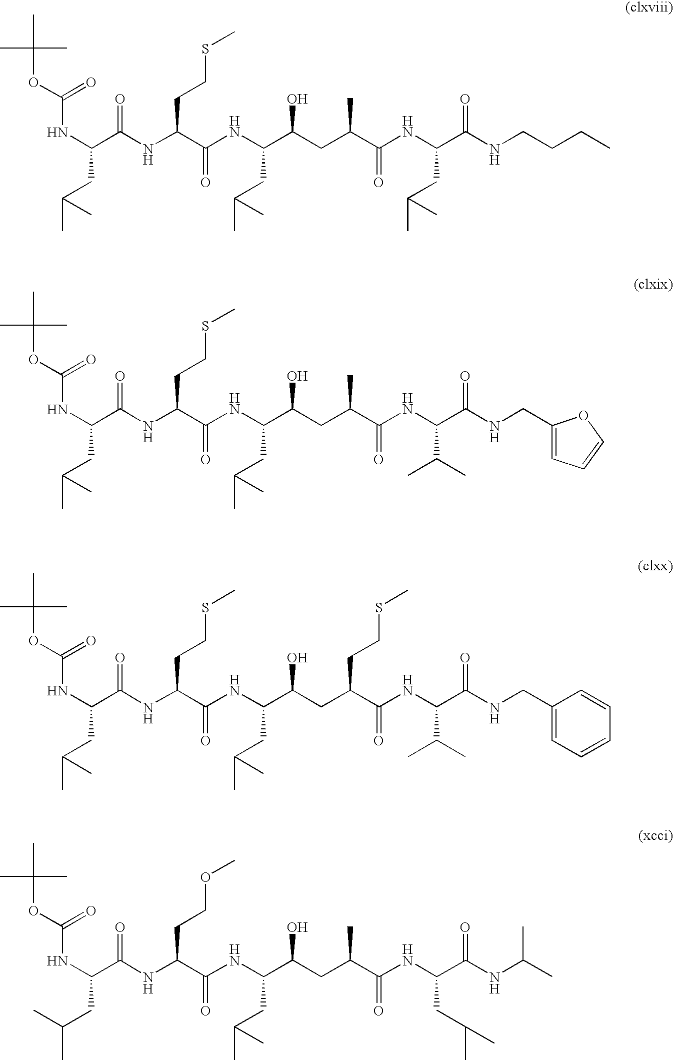
and pharmaceutically acceptable salts thereof.

85. A compound of claim 83, wherein the compound is selected from the group consisting of: 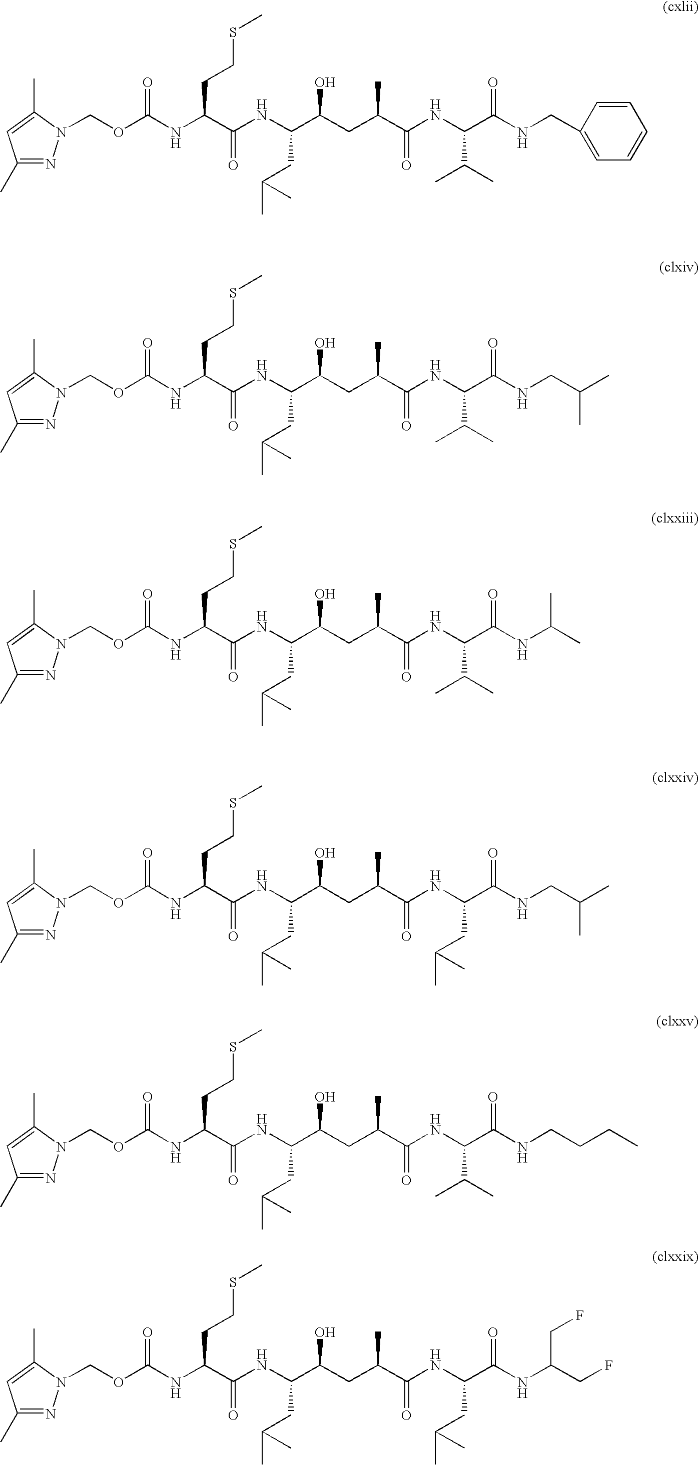 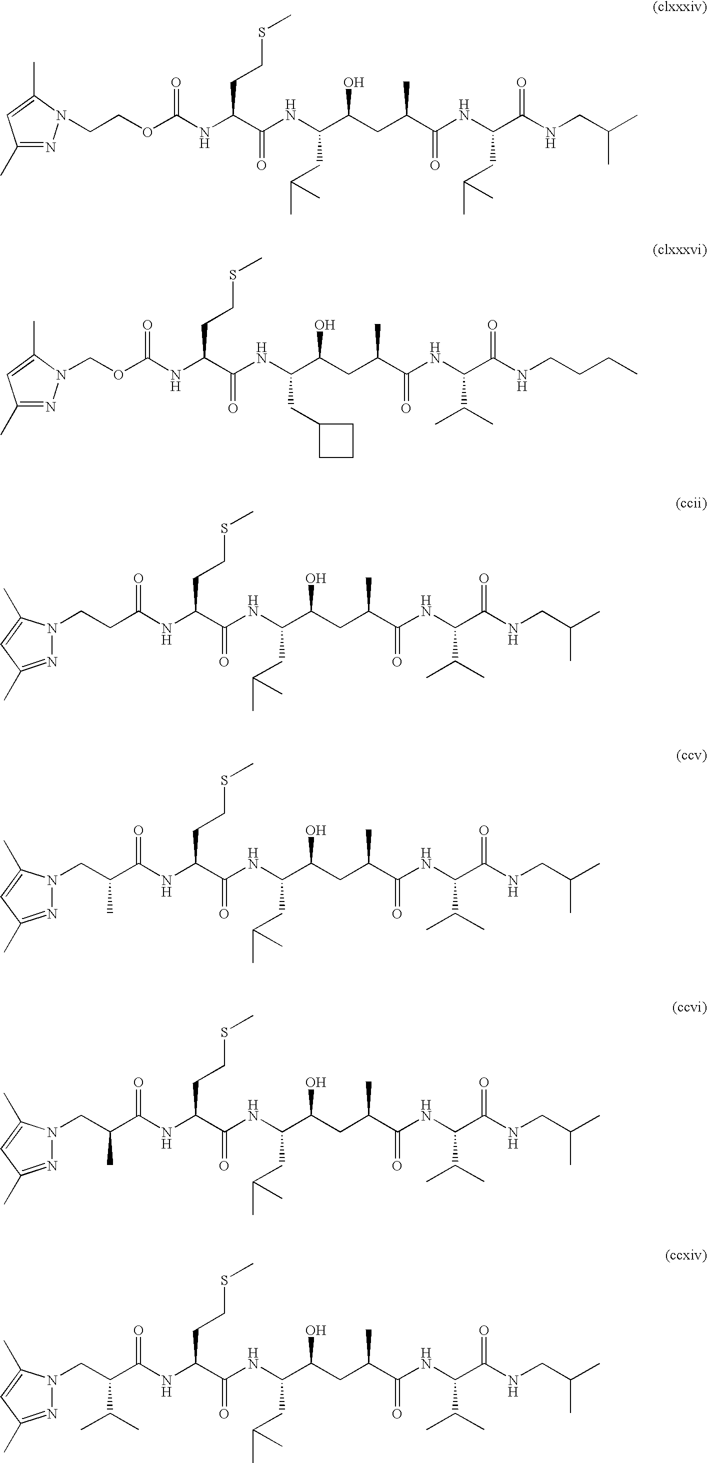 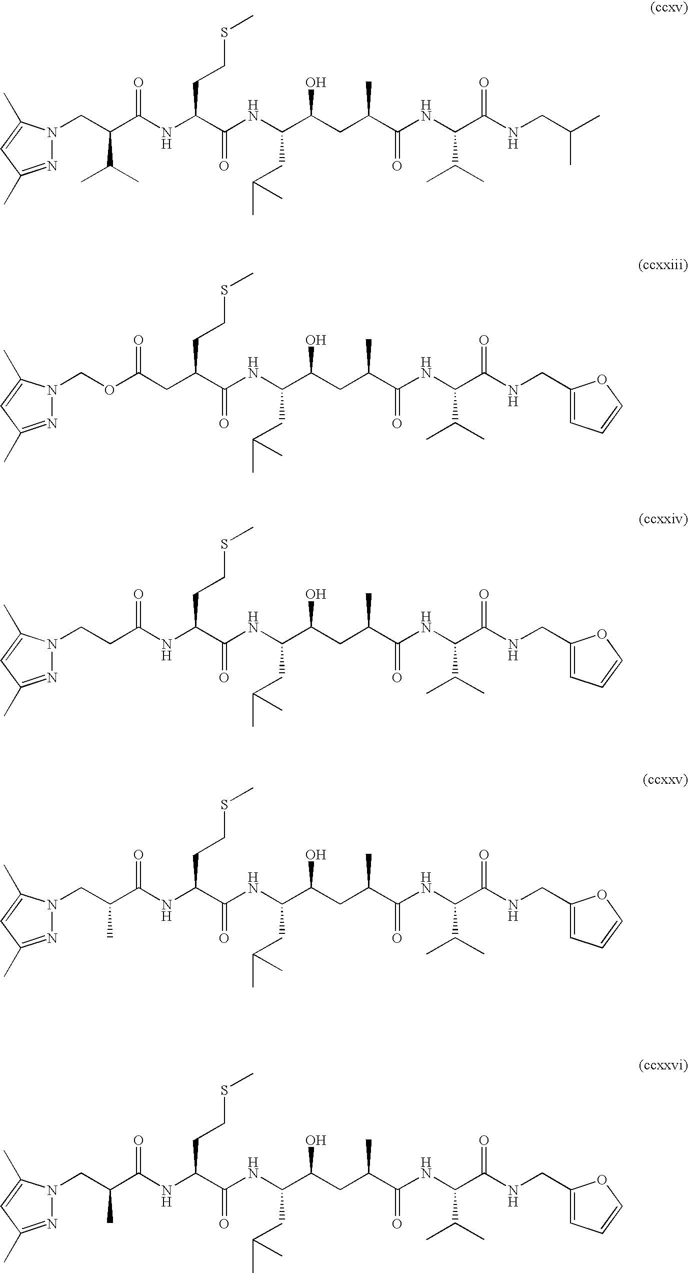
and pharmaceutically acceptable salts thereof.

86. A method of selectively inhibiting memapsin 2 β-secretase activity relative to memapsin 1 β-secretase activity in an in vitro sample, comprising the step of administering to the in vitro sample a compound of claim 1, 5, 18, 21, 23, 24, 35, 38, 40, 63, 65, 67, 69, 78, 81, 83, or 85.

87. A method of selectively inhibiting memapsin 2 β-secretase activity relative to memapsin 1β-secretase activity in a mammal, comprising the step of administering to the mammal a compound of claim 1, 5, 18, 21, 23, 24, 35, 38, 40, 63, 65, 67, 69, 78, 81, 83, or 85.

88. A method of treating Alzheimer's disease in a mammal, comprising the step of administering to the mammal a compound of claim 1, 5, 18, 21, 23, 24, 35, 38, 40, 63, 65, 67, 69, 78, 81, 83, or 85.

89. A method of inhibiting hydrolysis of a O-secretase site of a β-amyloid precursor protein in a mammal, comprising the step of administering to the mammal a compound of claim 1, 5, 18, 21, 23, 24, 35, 38, 40, 63, 65, 67, 69, 78, 81, 83, or 85.

90. The method of claim 89, wherein the β-secretase site includes an amino acid sequence selected from the group consisting of SEQ ID NO: 11 and SEQ ID NO: 12.

91. A method of inhibiting hydrolysis of a β-secretase site of a β-amyloid precursor protein in an in vitro sample, comprising the step of administering to the in vitro sample a compound of claim 1, 5, 18, 21, 23, 24, 35, 38, 40, 63, 65, 67, 69, 78, 81, 83, or 85.

92. The method of claim 91, wherein the β-secretase site includes an amino acid sequence selected from the group consisting of SEQ ID NO: 11 and SEQ ID NO: 12.

93. A method of decreasing β-amyloid protein in an in vitro sample, comprising the step of administering to the in vitro sample a compound of claim 1, 5, 18, 21, 23, 24, 35, 38, 40, 63, 65, 67, 69, 78, 81, 83, or 85.

94. A method of decreasing β-amyloid protein in a mammal, comprising the step of administering to the mammal a compound of claim 1, 5, 18, 21, 23, 24, 35, 38, 40, 63, 65, 67, 69, 78, 81, 83, or 85.

95. A pharmaceutical composition comprising a compound of claim 1, 5, 18, 21, 23, 24, 35, 38, 40, 63, 65, 67, 69, 78, 81, 83, or 85.

96. A crystallized protein comprising:
a) a protein that includes an amino acid sequence selected from the group consisting of amino acid residues 1-456 of SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and
b) a compound, wherein said compound is a compound of claim 1, 5, 18, 21, 23, 24, 35, 38, 40, 63, 65, 67, 69, 78, 81, 83, or 85, and wherein said crystallized protein has an x-ray diffraction resolution limit not greater than about 4.0 Å.

97. The crystallized protein of claim 96, wherein the x-ray diffraction resolution limit is not greater than about 2 Å.

98. A crystallized protein comprising:
a) a protein that includes an amino acid sequence of SEQ ID NO: 6; and
b) a compound, wherein said compound is a compound of claim 1, 5, 18, 21, 23, 24, 35, 38, 40, 63, 65, 67, 69, 78, 81, 83, or 85, and wherein said crystallized protein has an x-ray diffraction resolution limit not greater than about 4.0 Å.

99. The crystallized protein of claim 98, wherein the x-ray diffraction resolution limit is not greater than about 2 Å.

100. The crystallized protein of claim 98, wherein SEQ ID NO: 6 lacks a transmembrane domain.

101. A crystallized protein comprising:
a) a protein that includes an amino acid sequence encoded by SEQ ID NO: 5; and
b) a compound, wherein said compound is a compound of claim 1, 5, 18, 21, 23, 24, 35, 38, 40, 63, 65, 67, 69, 78, 81, 83, or 85, and wherein said crystallized protein has an x-ray diffraction resolution limit not greater than about 4.0 Å.

102. The crystallized protein of claim 101, wherein the x-ray diffraction resolution limit is not greater than about 2 Å.

103. The crystallized protein of claim 101, wherein the protein encoded by SEQ ID NO: 5 lacks a transmembrane domain.

104. A crystallized complex comprising:
a) a protein that includes an amino acid sequence selected from the group consisting of amino acid residues 1-456 SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and
b) a compound in association with said protein, wherein said compound is in association with said protein at an S3′ binding pocket, an S4′ binding pocket or an S4 binding pocket.

105. The crystallized complex of claim 104, wherein the compound is in association with said protein at at least two binding pockets selected from the group consisting of the S3′ binding pocket, the S4′ binding pocket and the S4 binding pocket.

106. The crystallized complex of claim 105, wherein the compound is in association with said protein at the S3′ binding pocket, the S4′ binding pocket and the S4 binding pocket.

107. The crystallized complex of claim 104, wherein said S4′ binding pocket comprises at least two amino acid residues selected from the group consisting of Glu¹⁸⁸, Ile¹⁸⁹, Trp²⁶⁰ and Tyr²⁶¹ of SEQ ID NO: 8.

108. The crystallized complex of claim 104, wherein said S3′ binding pocket comprises at least two amino acid residues selected from the group consisting of Pro¹³³, Tyr¹³⁴, Arg¹⁹¹ and Tyr²⁶¹ of SEQ ID NO: 8.

109. The crystallized complex of claim 104, wherein said S4 binding pocket comprises at least two amino acid residues selected from the group consisting of Gly⁷⁴, Gln¹³⁶, Thr²⁹⁵, Arg³⁷⁰ and Lys³⁸⁴ of SEQ ID NO: 8.

110. The crystallized complex of claim 104, wherein said compound is a compound of claim 1, 5, 18, 21, 23, 24, 35, 38, 40, 63, 65, 67, 69, 78, 81, 83, or 85.

111. A crystallized complex comprising:
a) a protein that includes an amino acid sequence selected from the group consisting of amino acid residues 1-456 SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and
b) a compound in association with said protein, wherein said compound is in association with said protein at an S3 binding pocket.

112. The crystallized complex of claim 111, wherein said S3 binding pocket comprises at least two amino acid residues selected from the group consisting of Gly⁷⁴, Gln⁷⁵, Gly⁷⁶, Leu⁹³, Ile¹⁷⁵, Trp¹⁷⁸, Gly²⁹³, Thr²⁹⁴ and Thr²⁹⁵ of SEQ ID NO: 8.

113. The crystallized complex of claim 112, wherein the compound is in association with said protein at S3′ binding pocket and an S4′ binding pocket.

114. The crystallized complex of claim 113, wherein said S4′ binding pocket comprises at least two amino acid residues selected from the group consisting of Glu¹⁸⁸, Ile¹⁸⁹, Trp²⁶⁰ and Tyr²⁶¹ of SEQ ID NO: 8.

115. The crystallized complex of claim 113, wherein said S3′ binding pocket comprises at least two amino acid residues selected from the group consisting of Pro¹³³, Tyr¹³⁴, Arg¹⁹¹ and Tyr²⁶¹ of SEQ ID NO: 8.

116. A crystallized complex comprising:
a) a protein that includes an amino acid sequence selected from the group consisting of amino acid residues 1-456 SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8; and
b) a compound of claim 40, 63, 65, 67, 69, 78, 81, or 85 in association with said protein, wherein said compound is in association with said protein at an S3 binding pocket.

117. The crystallized complex of claim 116, wherein said S3 binding pocket comprises at least two amino acid residues selected from the group consisting of Gly⁷⁴, Gln⁷⁵, Gly⁷⁶, Leu⁹³, Ile¹⁷⁵, Trp¹⁷⁸, Gly²⁹³, Thr²⁹⁴ and Thr²⁹⁵ of SEQ ID NO: 8.

118. A crystallized protein comprising:
a) a memapsin 2 protein; and
b) a compound, wherein said compound is a compound of claim 1, 5, 18, 21, 23, 24, 35, 38, 40, 63, 65, 67, 69, 78, 81, 83, or 85, and wherein said crystallized protein has an x-ray diffraction resolution limit not greater than about 4.0 Å.

119. The crystallized protein of claim 118, wherein the x-ray diffraction resolution limit is not greater than about 2 Å.

120. The crystallized protein of claim 118 or 119, wherein the memapsin 2 protein consists essentially of amino acid residues 16-456 of SEQ ID NO: 8.

121. The crystallized protein of claim 118 or 119, wherein the memapsin 2 protein consists essentially of amino acid residues selected from the group consisting of amino acid residues 1-456 of SEQ ID NO: 8, amino acid residues 16-456 of SEQ ID NO: 8, amino acid residues 27-456 of SEQ ID NO: 8, amino acid residues 43-456 of SEQ ID NO: 8 and amino acid residues 45-456 of SEQ ID NO: 8.


SEQUENCE LISTING (73 sequences)

SEQ ID NO: 1 - DNA, 1557 bp, Homo sapiens
  memapsin 1
atgggcgcactggcccgggcgctgctgctgcctctgctggcccagtggctcctgcgcgccgccccggagctggcccccgcgcccttcacgctgcccctccgggtggccgcggccacgaaccgcgtagttgcgcccaccccgggacccgggacccctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagacgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaatga

SEQ ID NO: 2 - protein, 518 aa, Homo sapiens
  memapsin 1
MGALARALLLPLLAQWLLRAAPELAPAPFTLPLRVAAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEPILWIVSYALMSVCGAILLVLIVLLLLPFRCQRRPRDPEVVNDESSLVRHRWK

SEQ ID NO: 3 - DNA, 1386 bp, Homo sapiens
  promemapsin 1-T1
atggctagcatgactggtggacagcaaatgggtcgcggatccgccggcccggagctggcccccgcgcccttcacgctgcccctccgggtgaccgcggccacgaaccgcgtagttgcgcccaccccgggacccgggacccctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagacgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagccctga

SEQ ID NO: 4 - protein, 461 aa, Homo sapiens
  promemapsin 1-T1
MASMTGGQQMGRGSAGPELAPAPFTLPLRVTAATNRVVAPTPGPGTPAERHADGLALALEPALASPAGAANFLAMVDNLQGDSGRGYYLEMLIGTPPQKLQILVDTGSSNFAVAGTPHSYIDTYFDTERSSTYRSKGFDVTVKYTQGSWTGFVGEDLVTIPKGFNTSFLVNIATIFESENFFLPGIKWNGILGLAYATLAKPSSSLETFFDSLVTQANIPNVFSMQMCGAGLPVAGSGTNGGSLVLGGIEPSLYKGDIWYTPIKEEWYYQIEILKLEIGGQSLNLDCREYNADKAIVDSGTTLLRLPQKVFDAVVEAVARASLIPEFSDGFWTGSQLACWTNSETPWSYFPKISIYLRDENSSRSFRITILPQLYIQPMMGAGLNYECYRFGISPSTNALVIGATVMEGFYVIFDRAQKRVGFAASPCAEIAGAAVSEISGPFSTEDVASNCVPAQSLSEP

SEQ ID NO: 5 - DNA, 1506 bp, Homo sapiens
  memapsin 2
atggcccaagccctgccctggctcctgctgtggatgggcgcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagtga

SEQ ID NO: 6 - protein, 501 aa, Homo sapiens
  memapsin 2
MAQALPWLLLWMGAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLMTIAYVMAAICALFMLPLCLMVCQWRCLRCLRQQHDDFADDISLLK

SEQ ID NO: 7 - DNA, 1371 bp, Homo sapiens
  promemapsin 2
atggctagcatgactggtggacagcaaatgggtcgcggatctatggcgggagtgctgcctgcccacggcacccagcacggcatccggctgcccctgcgcagcggcctggggggcgcccccctggggctgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtcatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaacctga

SEQ ID NO: 8 - protein, 456 aa, Homo sapiens
  promemapsin 2
MASMTGGQQMGRGSMAGVLPAHGTQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 9 - protein, 414 aa, Homo sapiens
  memapsin 2 in crystal structure determinations
LRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDEST

SEQ ID NO: 10 - protein, 770 aa, Homo sapiens
  beta-amyloid precursor protein (APP)
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 11 - protein, 10 aa, Artificial Sequence
  Description of Artificial Sequencebeta- secretase site of native beta-amyloid precursor protein (APP)
SEVNLDAEFR

SEQ ID NO: 12 - protein, 10 aa, Artificial Sequence
  Description of Artificial Sequencebeta- secretase site of Swedish mutation of beta-amyloid precursor protein (APPsw)
SEVKMDAEFR

SEQ ID NO: 13 - protein, 11 aa, Artificial Sequence
  Description of Artificial SequenceHIV tat protein (residues 47-57) carrier molecule peptide
YGRKKRRQRRR

SEQ ID NO: 14 - protein, 9 aa, Artificial Sequence
  Description of Artificial Sequencenine arginine residue carrier moleule peptide
RRRRRRRRR

SEQ ID NO: 15 - protein, 8 aa, Artificial Sequence
  Description of Artificial Sequencememapsin 2 substrate base template peptide
EVNLAAEF

SEQ ID NO: 16 - protein, 12 aa, Artificial Sequence
  Description of Artificial Sequencememapsin 2 substrate base template extended by 4 residues at C-terminus, downstream APP sequence extension
EVNLAAEFWHDR

SEQ ID NO: 17 - protein, 12 aa, Artificial Sequence
  Description of Artificial Sequencememapsin 2 substrate base template extended by 4 residues at N-terminus, upstream APP sequence extension
RWHHEVNLAAEF

SEQ ID NO: 18 - protein, 12 aa, Artificial Sequence
  Description of Artificial Sequencememapsin 2 substrate peptide mixture
EVNLXAEFWHDR

SEQ ID NO: 19 - protein, 8 aa, Artificial Sequence
  Description of Artificial Sequencememapsin 2 substrate peptide mixture hydrolytic product
XAEFWHDR

SEQ ID NO: 20 - protein, 8 aa, Artificial Sequence
  Description of Artificial Sequencetransition-state isosteric inhibitor compound OM99-2
EVNXXAEF

SEQ ID NO: 21 - protein, 12 aa, Artificial Sequence
  Description of Artificial Sequenceoverall combinatorial inhibitor library sequence
GXXXXXXFRMGX

SEQ ID NO: 22 - protein, 7 aa, Artificial Sequence
  Description of Artificial Sequenceshorter version of combinatorial inhibitor library peptide sequence
XXFRMGX

SEQ ID NO: 23 - protein, 8 aa, Artificial Sequence
  Description of Artificial Sequencetransition-state isosteric inhibitor compound OM00-3
ELDXXVEF

SEQ ID NO: 24 - protein, 26 aa, Artificial Sequence
  Description of Artificial Sequencememapsin 2 transmembrane domain
LMTIAYVMAAICALFMLPLCLMVCQW

SEQ ID NO: 25 - protein, 6 aa, Artificial Sequence
  Description of Artificial SequenceFormula XI peptide
XXWXXX

SEQ ID NO: 26 - protein, 6 aa, Artificial Sequence
  Description of Artificial SequenceFormula XI peptide
WWWWXX

SEQ ID NO: 27 - protein, 12 aa, Artificial Sequence
  Description of Artificial Sequencememapsin 1 combinatorial inhibitor library template
GXXXXVXFRMGX

SEQ ID NO: 28 - protein, 12 aa, Artificial Sequence
  Description of Artificial Sequencememapsin 2 template substrate sequence
WHDREVNLAAEF

SEQ ID NO: 29 - protein, 4 aa, Artificial Sequence
  Description of Artificial Sequencefour N-terminal residues added to memapsin 2 template substrate
WHDR

SEQ ID NO: 30 - protein, 12 aa, Artificial Sequence
  Description of Artificial Sequencememapsin 2 peptide substrate
EVNLAVEFWHDR

SEQ ID NO: 31 - protein, 12 aa, Artificial Sequence
  Description of Artificial Sequencememapsin 2 peptide substrate
EVNLAAEFWHDR

SEQ ID NO: 32 - protein, 4 aa, Artificial Sequence
  Description of Artificial Sequenceautomated Edman degradation sequence 2, from the short sequence
XXFR

SEQ ID NO: 33 - protein, 9 aa, Artificial Sequence
  Description of Artificial Sequenceoligo-D-arginine carrier molecule peptide, (D-Arg)-9 carrier molecule
XXXXXXXXX

SEQ ID NO: 34 - protein, 21 aa, Artificial Sequence
  Description of Artificial SequenceCPI-1 carrier peptide inhibitor conjugate
XEVNXXAEFGYGRKKRRQRRR

SEQ ID NO: 35 - protein, 20 aa, Artificial Sequence
  Description of Artificial SequenceCPI-2 carrier peptide inhibitor conjugate
XELDXXVEFGGXXXXXXXXX

SEQ ID NO: 36 - protein, 15 aa, Artificial Sequence
  Description of Artificial SequenceCP-1 fluorescein-labeled control carrier peptide excluding a conjugated inhibitor moiety
XGGGYGRKKRRQRRR

SEQ ID NO: 37 - protein, 19 aa, Artificial Sequence
  Description of Artificial SequenceCPI-3 carrier peptide inhibitor conjugate
ELDXXVEFGGXXXXXXXXX

SEQ ID NO: 38 - DNA, 17 bp, Artificial Sequence
  Description of Artificial Sequencepositive control PCR amplification primer beta actin XAHR17
cggaaccgctcattgcc

SEQ ID NO: 39 - DNA, 20 bp, Artificial Sequence
  Description of Artificial Sequencepositive control PCR amplification primer beta actin XAHR 20
acccacactgtgcccatcta

SEQ ID NO: 40 - DNA, 25 bp, Artificial Sequence
  Description of Artificial SequencePCR amplification primer 1503 APP
ctgaccactcgaccaggttctgggt

SEQ ID NO: 41 - DNA, 29 bp, Artificial Sequence
  Description of Artificial SequencePCR amplification primer 1502 APP
gtggataacccctcccccagcctagacca

SEQ ID NO: 42 - protein, 8 aa, Artificial Sequence
  Description of Artificial Sequencememapsin 1 APP substrate cleavage site
KLVFFAED

SEQ ID NO: 43 - protein, 4 aa, Artificial Sequence
  Description of Artificial SequenceN-terminal sequence addition to memapsin 2 substrate, upstream memapsin 2 substrate template sequence extension
RWHH

SEQ ID NO: 44 - protein, 12 aa, Artificial Sequence
  Description of Artificial Sequencememapsin 2 template substrate sequence
WHDREVNLAVEF

SEQ ID NO: 45 - protein, 8 aa, Artificial Sequence
  Description of Artificial Sequencememapsin 2 substrate
EVNLAVEF

SEQ ID NO: 46 - protein, 12 aa, Artificial Sequence
  Description of Artificial Sequencememapsin 2 substrate base template extended by 4 residues at N-terminus, native APP sequence extension
EEISEVNLAAEF

SEQ ID NO: 47 - protein, 14 aa, Artificial Sequence
  Description of Artificial SequenceP5 memapsin 2 substrate base template extended by 4 residues with Arg at N-terminus, native APP sequence extension
RTEEIXEVNLAAEF

SEQ ID NO: 48 - protein, 14 aa, Artificial Sequence
  Description of Artificial SequenceP6 memapsin 2 substrate base template extended by 4 residues with Arg at N-terminus, native APP sequence extension
RTEEXSEVNLAAEF

SEQ ID NO: 49 - protein, 14 aa, Artificial Sequence
  Description of Artificial SequenceP7 memapsin 2 substrate base template extended by 4 residues with Arg at N-terminus, native APP sequence extension
RTEXISEVNLAAEF

SEQ ID NO: 50 - protein, 14 aa, Artificial Sequence
  Description of Artificial SequenceP8 memapsin 2 substrate base template extended by 4 residues with Arg at N-terminus, native APP sequence extension
RTXEISEVNLAAEF

SEQ ID NO: 51 - protein, 4 aa, Artificial Sequence
  Description of Artificial SequenceN-terminal sequence addition to memapsin 2 substrate, upstream native human APP memapsin 2 substrate template sequence extension
EEIS

SEQ ID NO: 52 - protein, 10 aa, Artificial Sequence
  Description of Artificial Sequence1-10 lysine residue carrier molecule peptide
KKKKKKKKKK

SEQ ID NO: 53 - protein, 10 aa, Artificial Sequence
  Description of Artificial Sequence1-10 arginine residue carrier molecule peptide
RRRRRRRRRR

SEQ ID NO: 54 - protein, 10 aa, Artificial Sequence
  Description of Artificial Sequence1-10 histidine residue carrier molecule peptide
HHHHHHHHHH

SEQ ID NO: 55 - protein, 12 aa, Artificial Sequence
  Description of Artificial Sequencememapsin 2 substrate mixture APP-P-1 A
RWHHEVNXAAEF

SEQ ID NO: 56 - protein, 12 aa, Artificial Sequence
  Description of Artificial Sequencememapsin 2 substrate mixture APP-P-1 B
RWHHEVNXAAEF

SEQ ID NO: 57 - protein, 12 aa, Artificial Sequence
  Description of Artificial Sequencememapsin 2 substrate mixture APP-P-1 C
RWHHEVNXAAEF

SEQ ID NO: 58 - protein, 12 aa, Artificial Sequence
  Description of Artificial Sequencememapsin 2 substrate mixture APP-P-2 A
RWHHEVXLAAEF

SEQ ID NO: 59 - protein, 12 aa, Artificial Sequence
  Description of Artificial Sequencememapsin 2 substrate mixture APP-P-2 B
RWHHEVXLAAEF

SEQ ID NO: 60 - protein, 12 aa, Artificial Sequence
  Description of Artificial Sequencememapsin 2 substrate mixture APP-P-2 C
RWHHEVXLAAEF

SEQ ID NO: 61 - protein, 12 aa, Artificial Sequence
  Description of Artificial Sequencememapsin 2 substrate mixture APP-P-3 A
RWHHEXNLAAEF

SEQ ID NO: 62 - protein, 12 aa, Artificial Sequence
  Description of Artificial Sequencememapsin 2 substrate mixture APP-P-3 B
RWHHEXNLAAEF

SEQ ID NO: 63 - protein, 12 aa, Artificial Sequence
  Description of Artificial Sequencememapsin 2 substrate mixture APP-P-3 C
RWHHEXNLAAEF

SEQ ID NO: 64 - protein, 12 aa, Artificial Sequence
  Description of Artificial Sequencememapsin 2 substrate mixture APP-P-4 A
RWHHXVNLAAEF

SEQ ID NO: 65 - protein, 12 aa, Artificial Sequence
  Description of Artificial Sequencememapsin 2 substrate mixture APP-P-4 B
RWHHXVNLAAEF

SEQ ID NO: 66 - protein, 12 aa, Artificial Sequence
  Description of Artificial Sequencememapsin 2 substrate mixture APP-P-4 C
RWHHXVNLAAEF

SEQ ID NO: 67 - protein, 8 aa, Artificial Sequence
  Description of Artificial Sequencememapsin 1 substrate subsite best preferred residues, most preferred residues
ELNFMIFW

SEQ ID NO: 68 - protein, 8 aa, Artificial Sequence
  Description of Artificial Sequencememapsin 1 substrate subsite 2nd preferred residues
QIDLLVXF

SEQ ID NO: 69 - protein, 8 aa, Artificial Sequence
  Description of Artificial Sequencememapsin 1 substrate subsite 3rd and other preferred residues
XVXYXXXX

SEQ ID NO: 70 - protein, 8 aa, Artificial Sequence
  Description of Artificial Sequencememapsin 2 substrate subsite best preferred residues
EIDLMVXX

SEQ ID NO: 71 - protein, 8 aa, Artificial Sequence
  Description of Artificial Sequencememapsin 2 substrate subsite 2nd preferred residues
QVNFEIXW

SEQ ID NO: 72 - protein, 8 aa, Artificial Sequence
  Description of Artificial Sequencememapsin 2 substrate subsite 3rd and other preferred residues
XXXXXXXX

SEQ ID NO: 73 - protein, 8 aa, Artificial Sequence
  Description of Artificial Sequencememapsin 1 substrate subsite 2nd preferred residues
QIDLLVWF